(19) United States
(12) Patent Application Publication
(10) Pub. No.: US 2024/0336617 A1
(43) Pub. Date: Oct. 10, 2024

(54) Substituted Bimane Compounds and Methods of Use Thereof

(71) Applicant: THE TRUSTEES OF THE UNIVERSITY OF PENNSYLVANIA, Philadelphia, PA (US)

(72) Inventors: E. James Petersson, Wynnewood, PA (US); Venkatesh Yarra, Philadelphia, PA (US)

(21) Appl. No.: 18/611,505

(22) Filed: Mar. 20, 2024

Related U.S. Application Data
(60) Provisional application No. 63/491,184, filed on Mar. 20, 2023.

(51) Int. Cl. C07D 487/04; A61K 31/4162; A61K 31/5377; A61K 31/5415; G01N 21/64; G01N 33/58

(52) U.S. Cl. CPC C07D 487/04; A61K 31/4162; A61K 31/5377; A61K 31/5415; G01N 21/6428; G01N 33/582; G01N 2021/6439

(57) ABSTRACT

In one aspect, the present disclosure relates to compounds of Formula (I). In certain embodiments, the compounds of Formula (I) comprise fluorescent dyes. In another aspect, the present disclosure provides a method for diagnosing a protein aggregation disease in a subject. In certain embodiments, the protein aggregation disease is selected from the group consisting of Parkinson's disease (PD) and Alzheimer's disease (AD).

### CROSS-REFERENCE TO RELATED APPLICATIONS

The present application claims priority under 35 U.S.C. § 119(e) to U.S. Provisional Application No. 63/491,184, filed Mar. 20, 2023, all of which is incorporated herein by reference in its entirety.

### STATEMENT REGARDING FEDERALLY SPONSORED RESEARCH AND DEVELOPMENT

This invention was made with government support under NS110456, and NS103873 awarded by the National Institutes of Health. The government has certain rights in the invention.

### BACKGROUND

Fluorescence spectroscopy has become one of the most powerful and widely used methods in the life sciences, from fundamental research to clinical applications. Fluorescence-based technologies have been used for the characterization of protein/protein, protein/nucleic acid, protein/substrate, and membrane/biomolecule interactions, which play crucial roles in the regulation of cellular pathways. Small molecule fluorescent probes have significant advantages over protein-based probes in optical imaging and analytical sensing due to their small size (minimizing disruption to the protein target), high sensitivity, and fast response time.

Because they have ubiquitous applications as cellular stains, environmental sensors, or biomolecular labels, a plethora of fluorescent probes have been developed over the past decades. Although numerous fluorescent probes are known, most of these probes are developed through structural modification of a diminutive set of classical “core” dyes such as coumarin, fluorescein, boron dipyrromethene (BODIPY), and cyanine. This underscores the importance of intrinsic modularity of the core fluorophores in the elaboration and modification of dyes, but also reveals the limited number of available core scaffolds. Moreover, within the context of biological systems, “ideal” fluorescent probes with drug-like uptake and selectivity in the cellular environment are still in high demand.

Recently, small fluorogenic probes with high sensitivity and selectivity have emerged as powerful tools to study amyloid fibrils accumulating in cells or tissue samples from patients with Alzheimer's disease (AD), Parkinson's disease (PD), and many other neurodegenerative disorders. Thioflavin-T (ThT) has long been a widely used dye for staining amyloid fibrils, due to a significant increase in fluorescence intensity upon its binding to amyloid fibrils in comparison to that of free ThT in aqueous solution. Despite the wide usage of ThT for studying amyloid fibrils, there are limitations to its application.

In particular, the fact that ThT has a relatively small Stokes shift (˜50 nm), which can be problematic when used in conjunction with other spectrally similar molecules, low specificity among different types of amyloids and poor selectivity for fibrils over other types of aggregates, as well as poor cellular uptake. The limitations of ThT have prompted a search for fluorogenic amyloid dyes with improved photophysical characteristics: large Stokes shifts, higher selectivity, and low background fluorescence.

There is thus a need in the art for novel fluorogenic compounds, methods of preparation thereof, and methods of using the same. The present disclosure addresses this need.

### BRIEF DESCRIPTION OF THE FIGURES

The drawings illustrate generally, by way of example, but not by way of limitation, various embodiments of the present application.

FIGS. 1A-1B: Structure-photophysical property relationship of 7a, 7b, 7c, 7e, 7f, 7g, 7h, 7j, and 7l. FIG. 1A: correlation between absorption maximum (λ_{abs}) and Hammett constant (σₚ) of certain substituents in styrl bimanes. FIG. 1B: HOMO and LUMO of 7l and 7b, showing the movement in electron density upon excitation from S₀ to S₁.

FIG. 2A: absorption spectra of 6 and 7b, 7e, and 7k. FIG. 2B: emission spectra of 6 and 7b, 7e, and 7k. FIG. 2C: absorption spectra of 7i-7m. FIG. 2D: normalized and smoothed fluorescence spectra of 7i-7m. FIG. 2E: absorption of 7i and 7j. FIG. 2F: fluorescence spectra of 7i and 7j. FIG. 2G: solvatochromism of 7k, for polarity screening toluene, dichloromethane (DCM), dimethylformamide (DMF), DMSO, and ethanol (EtOH) were used as solvents. Final concentration of 6 and 7a-7m is 25 μM in ACN and PBS buffer (50:50 v/v). Emission spectra were measured with excitation at their optimal max absorption.

FIGS. 3A-3D: effect of Rotational Restriction on Styrl Bimane Fluorescence. FIG. 3A: schematic representation of fluorescence turn on mechanism. FIG. 3B: red shift of 7k absorption maximum with increasing glycerol % in glycerol/methanol mixtures. FIG. 3C: increase in fluorescence intensity of 7k at λₑₘ with increasing glycerol; inset: Tubes with varying glycerol % illuminated with a 365 nm handheld UV lamp). FIG. 3D: fluorescence intensity of probes 7j, 7k, 7l, and 7m (1 μM) in the presence of varying concentrations of BSA.

FIGS. 4A-4D: fluorescence spectral changes of probes in the presence and absence of αS PFFs 7j (FIG. 4A), 7k (FIG. 4B), 7l (FIG. 4C), and 7m (FIG. 4D). Concentrations of dyes and PFFs were 10 and 50 μM, respectively. FIG. 4E: schematic representation of probe binding to αS PFFs. FIG. 4F: K_{d} determination of probes 7k, 7l, and 7m (10 nM-10 μM) with 100 μM αS PFFs. FIG. 4G: fluorescence fold change of 7k, 7l, and 7m (1 μM) with αS PFFs in the presence of 10 mg/mL cytosolic HEK cell lysate. FIG. 4H: amyloid selectivity of 7k, 7l, and 7m (1 μM) with varying the concentration of αS, tau and amyloid-β (Aβ₁₋₄₂) fibrils. λ_{Jex}/λₑₘ=435/575 nm for 7j, λₑₓ/λₑₘ=424/570 nm for 7k, λₑₓ/λₑₘ=463/580 nm for 7l, and λₑₓ/λₑₘ=484/620 nm for 7m. Error bars represent SD of 3 measurements.

FIG. 5: fluorescence detection of αS fibrils amplified from AD and PDD lysates. Top: Workflow for detection of amplified fibrils (AFs) generated from seeds extracted from AD or PDD patient brain samples, as well as control samples containing lysate only (Lysate, shows fluorescence deriving from seeds or other proteins) or pre-formed fibrils (PFFs, same αS fibril concentration as AF, shows selectivity of binding to templated AF fibrils). Bottom: Fluorescence fold change relative to dye alone for 7k, 7l, 7m, or ThT incubated with Lysate, PFF, and AF samples. Final lysate and probe concentrations were 1 μM. Error bars represent SD of 3 measurements. λₑₓ/λₑₘ=424/570 nm for 7k, 463/580 nm for 7l, 484/620 nm for 7m, and 450/482 nm for ThT.

FIGS. 6A-6C: Liquid Chromatography (LC) chromatogram for the conversion of 7l under different optimized reaction conditions.

FIG. 7: ORTEP drawing of 7a with 50% thermal ellipsoids.

FIG. 8: ORTEP drawing of 7c with 50% thermal ellipsoids.

FIG. 9: ORTEP drawing of 7l with 50% thermal ellipsoids.

FIGS. 10A-10D: photophysical properties of 6 and 7a-7m in ACN solvent. FIG. 10A: absorption spectra of 6 and 7a-7h. FIG. 10B: absorption spectra of 7i-7m. FIG. 10C: fluorescence spectra of 6 and 7a-7h. FIG. 10D: fluorescence spectra of 7i-7m. Final probe concentration is 25 μM. Fluorescence spectra were measured at their maximum absorption wavelength.

FIGS. 11A-11C: solvatochromism of 6 (FIG. 11A), 7b (FIG. 11B), and 7e (FIG. 11C). Left: Normalized fluorescence spectra in different solvents. Right: Fluorescence image taken under handheld UV lamp at 365 nm. Probe concentration 25 mM and excitation wavelength is 384 nm for 6, 320 nm for 7b and 7e.

FIGS. 12A-12C: solvatochromism of 7j (FIG. 12A), 7l (FIG. 12B), and 7m (FIG. 12C). Left: Normalized fluorescence spectra in different solvents. Probe concentration 25 mM. Right: Fluorescence image taken under handheld UV lamp at 365 nm. Emission spectra were measured by the excitation wavelength 385 nm for 7j, 418 nm for 7l, and 436 nm for 7m.

FIGS. 13A-13C: representative QY acquisitions of 6 and 7a-7m in ACN/PBS buffer (50:50). All had consistent excitation (360 nm) and spectral collection (350-800 nm) parameters. Final concentration of the probe is 500 mM. Black line indicates incident light and blue line indicates sample spectrum.

FIGS. 14A-14C: Representative QY acquisitions of 6 and 7a-7m in ACN solvent. All had consistent excitation (360 nm, slit width 10 nm) and spectral collection (350-800 nm) parameters. Final concentration of the probe is 500 mM. Black line indicates incident light and blue line indicates sample spectrum.

FIGS. 15A-15C: effect of pH on absorption and emission of 7h (FIG. 15A), 7j (FIG. 15B), and 7k (FIG. 15C). Final concentration of the probe is 25 mM. For pH screening of 7h and 7j, PBS buffer solutions were prepared, then adjusted the pH 2, 4, and 7 by adding 6 M hydrochloric acid, pH 9 and 11 by adding 5 M sodium hydroxide. For pH screening of 7k, pH 4.4 and 7.4 were prepared by citric acid/phosphate buffer. Emission spectra were measured by the excitation wavelength 354 nm for 7h, 385 nm for 7j, and 386 nm for 7k.

FIGS. 16A-16E: fluorescence lifetime measurements for 6 (FIG. 16A), 7c (FIG. 16B), 7e (FIG. 16C), 7f (FIG. 16D), and 7g (FIG. 16E) in ACN/PBS buffer (50:50 v/v). Lifetime measurements with exponential fits shown at left and residuals after fitting shown at right. TCSPC data were collected at 540 nm emission for 7c, 7e, 7f, 7g and 464 nm emission for 6. Excitation wavelength 340 nm.

FIGS. 17A-17B: absorption (FIG. 17A) and emission (FIG. 17B) spectral change of 7k in glycerol/MeOH mixtures. Final probe concentration is 25 mM. Excitation wavelength: 386 nm.

FIG. 18: correlation between HOMO-LUMO energy gap (eV) compared to experimentally measured UV-Vis absorption maximum (nm). Maximum UV-Vis absorption wavelengths were obtained in ACN/PBS buffer (50:50 v/v).

FIG. 19: relative HOMO-LUMO energy gap (eV) of selected bimane derivatives.

FIGS. 20A-20B: HOMO (FIG. 20A) and LUMO (FIG. 20B) molecular orbitals (Mos) of 7a, 7b, 7c, 7e, 7f, 7g, 7h, 7j, 7l, and 7m.

FIGS. 21A-21B: protein purification and cleavage of 1N4R tau. FIG. 21A: nickel purification visualized by a Coomassie stained 10% SDS-Page gel. FIG. 21B: FPLC purification fractions by SDS-PAGE gel.

FIGS. 22A-22B: MALDI MS characterization of WT αS (FIG. 22A) and tau 1N4R (FIG. 22B). On each plot matrix adduct peaks are marked with *.

FIGS. 23A-23C: Gel-based quantification of protein in αS (FIG. 23A), 1N4R tau (FIG. 23B), and Aβ₁₋₄₂ (FIG. 23C) fibrils.

FIG. 24: fluorescence spectra of probes (7b-7h) with and without αS fibrils. Final concentrations of probes and αS fibrils were 10 μM and 50 μM, respectively. Emission spectra collected at each dye's maximum absorption wavelength. Ex/Em slit widths: 3 nm/3 nm.

FIG. 25: absorbance of probes (7j, 7k, 7l and 7m) with and without αS fibrils. Final probe and fibrils concentrations were 10 μM and 50 μM, respectively.

FIG. 26: excitation spectra of probes (7j, 7k, 7l and 7m) with and without αS fibrils. Final probe and fibril concentrations were 10 μM and 50 μM, respectively. Emission wavelengths were 575 nm for 7j, 570 nm for 7k, 580 nm for 7l, and 620 nm for 7m. Ex/Em slit widths: 3 nm/3 nm.

FIG. 27: representative QY acquisitions of 7j, 7k, 7l and 7m with αS fibrils. Black line indicates incident light (αS fibrils without probe) and blue line indicates sample (αS fibrils with probe). Excitation at λ_{exc}=435 nm for 7j, λ_{exc}=424 nm for 7k, λ_{exc}=463 nm for 7l, and λ_{exc}=484 nm for 7m and spectral collection (400-800 nm). Final concentrations of the probe and αS fibrils are the 100 μM. Ex/Em slit widths: 5 nm/5 nm.

FIGS. 28A-28D: fluorescence lifetime measurements in ACN/PBS buffer (50:50 v/v). Lifetime measurements with exponential fits shown at left and residuals after fitting shown at right. TCSPC data were collected at 540 nm emission for 7j (FIG. 28A), 7k (FIG. 28B), 7l (FIG. 28C), and 7m (FIG. 28D) and 464 nm emission for 6. Excitation wavelength 340 nm. Final concentrations of the probe and αS fibrils were 100 μM.

FIG. 29A: Turn on fluorescence of 7k with respect to different concentration of αS fibrils and dye, λ_{exc}=420 nm, λₑₘ=580 nm; fluorescence image taken under handheld UV lamp at 365 nm with αS fibrils as well as monomer. FIG. 29B: turn on fluorescence of 7k with respect to different concentration of αS monomer, λ_{exc}=420 nm, λₑₘ=580 nm; fluorescence image taken under handheld UV lamp at 365 nm with αS monomer.

FIG. 30A: turn on fluorescence of 7k in presence of αS fibrils with respect to different concentration of HEK cell lysate. Final concentration of 7k and αS fibrils were 1 μM and 25 μM, respectively. FIG. 30B: turn on fluorescence of 7k with respect to different concentration of αS fibrils at 10 mg/mL HEK cell lysate concentration. Final probe 7k concentration 1 μM. λ_{exc}=420 nm, λₑₘ=580 nm.

FIGS. 31A-31C: fibril-specific turn on of 7l fluorescence. FIG. 31A: turn on fluorescence of 7l with respect to different concentration of αS fibrils. FIG. 31B: fluorescence image taken under handheld UV lamp at 365 nm with αS fibrils as well as monomer. FIG. 31C: turn on fluorescence of 7l with respect to different concentrations of αS monomer. Final probe 7l concentration 10 μM for both fibrils and monomer. λ_{exc}=420 nm, λₑₘ=580 nm.

FIG. 32: fluorescence spectra of probes (7j, 7k, 7l, 7m and ThT) with and without tau fibrils. Final probe and tau fibril concentrations were 5 μM and 25 μM, respectively. Excitation at λ_{exc}=435 nm for 7j, λ_{exc}=424 nm for 7k, λ_{exc}=463 nm for 7l, λ_{exc}=484 nm for 7m, λ_{exc}=450 nm for ThT and spectral collection (400-800 nm). Ex/Em slit widths: 3 nm/3 nm.

FIG. 33: excitation spectra of probes (7j, 7k, 7l, 7m and ThT) with and without tau fibrils. Final probe and fibril concentrations were 5 μM and 25 μM, respectively. Emission wavelengths were 575 nm for 7j, 570 nm for 7k, 580 nm for 7l, and 620 nm for 7m. Ex/Em slit widths: 3 nm/3 nm.

FIG. 34: fluorescence spectra of probes (7j, 7k, 7l, 7m and ThT) with and without Aβ₁₋₄₂ fibrils. Final probe and fibrils concentrations were 10 μM and 50 μM. Excitation at λ_{exc}=435 nm for 7j, λ_{exc}=424 nm for 7k, λ_{exc}=463 nm for 7l, λ_{exc}=484 nm for 7m, λ_{exc}=450 nm fot ThT. Ex/Em slit widths: 3 nm/3 nm.

FIG. 35: excitation spectra of probes (7j, 7k, 7l, 7m and ThT) with and without Aβ₁₋₄₂ fibrils. Final probe and Aβ₁₋₄₂ fibrils concentrations were 10 μM and 50 μM, respectively. Emission wavelengths were 575 nm for 7j, 570 nm for 7k, 580 nm for 7l, and 620 nm for 7m. Ex/Em slit widths: 3 nm/3 nm.

FIGS. 36A-36C: competition binding of the probe 7k and ThT. FIG. 36A: schematic of ThT binding studies. FIG. 36B: fluorescence spectra of probe 7k in the presence of αS fibrils and consecutive addition of ThT. FIG. 36C: fluorescence spectra of probe ThT in the presence of αS fibrils and consecutive addition of probe 7k. Excitation wavelength: 424 nm for 7k and 450 nm for ThT.

FIG. 37: fluorescence fold change of ThT (1 μM) with different concentration of αS, tau, and Aβ₁₋₄₂ fibrils in PBS buffer. λ_{exc}=450 nm, λₑₘ=482 nm.

FIGS. 38A-38B: preparation of fibrils amplified from patient-derived α-synuclein aggregates. FIG. 38A: Western blots of the final brain lysate samples extracted from AD and PDD patient brains. Membranes were probed with anti-pSer-129 αS (81A) or total αS (HuA).

FIG. 38B: Western blot of sedimentation results for amplified fibrils (AFs) after seven days of incubation. Supernatant (S) and pellet (P) fractions were run for each reaction with monomer (M) and PFF (F) as loading controls. The membrane was probed with HuA.

FIGS. 39A-39B: representative images of pathology induced in primary mouse hippocampal neurons for seeds and AFs (Amp) for each case, or PFFs. Probed with 81A (green), anti-MAP2 (red) and DAPI (blue). Scale bar 50 μm.

FIG. 40: analysis of bimane literature relative to similar fluorophores such as BODIPY and Danysl fluorophores.

FIGS. 41A-41C: photophysical properties of 4a and 7a-7m. FIG. 41A: photograph under ambient light. FIG. 41B: solution state fluorescence of 25 μM probe in ACN/PBS buffer (50:50 v/v) under handheld UV lamp (365 nm). FIG. 41C: amorphous solid-state fluorescence under handheld UV lamp (365 nm).

FIG. 42: non-limiting amyloid-binding bimane-benzothiazole compounds of the present disclosure, methods of preparation thereof, and X-ray structure of compound 8f.

FIGS. 43A-43B: non-limiting amyloid binding data for bimane-benzothiazole compound 8f of the present disclosure, demonstrating selective fluorescence increases in the presence of Aβ₁₋₄₂ fibrils.

FIG. 44A: Probe structures of 1, 2a, and 2b by varying either the substitution or the linker length (n). Normalized absorption spectra.

FIG. 44B: Normalized absorbance spectra measured with excitation at their optimal max absorption.

FIG. 44C: Normalized and smoothed fluorescence spectra measured with excitation at optimal max absorption.

FIG. 44D: TD-DFT calculated fluorescence emission energy of the electron from the excited S₁ state (p* orbital) back to the ground S₀ state (p orbital) of 1, 2a, and 2b at excited S₁ state in water.

FIG. 44E: Electrostatic potential maps based on the electron density of 1, 2a, and 2b in water and their dipole moments at excited state. Final concentration of probes is 25 mM in ACN and PBS buffer (50:50 v/v).

FIGS. 45A-45F: Structural modification affects the polarity sensitivity. FIG. 45A: Fluorescence spectral changes of probes 2a and 2b in the presence of αS monomer and PFFs. Increasing the electron donation and extension of conjugation regulates fluorescence colors but retains the fluorogenicity upon protein aggregation. The concentrations of dyes and PFFs were 10 and 50 μM, respectively. FIG. 45B: Comparison of experimental Intensity (counts) versus fitting data for 2a and 2b. FIG. 45C: image of 2a and 2b fibrils before and after incubation with fibrils. FIG. 45D: Schematic representation of the “turn on” fluorescence of 2a and 2b probes upon binding to αS PFFs, which resulting from the restricted internal rotation. FIG. 45E: Determination of the dissociation constant of probes 2a and 2b (10 nM-10 mM) binding to 100 mM αS-PFFs. FIG. 45F: Displacement of the known amyloid-binding dye ThT by 2a. Excitation and emission wavelength for fluorescence measurements: Ex/Em: 463 nm/580 nm for 2a, 484 nm/620 nm for 2b, and 450 nm/482 nm for ThT.

FIGS. 46A-46C: Polarity within different aggregated proteins spans over a wide range. FIG. 46A: Fluorescence emission wavelength of 2a quantifies the dielectric constant inside different protein aggregates. 2a (10 μM) was mixed with 50 μM protein fibrils. FIG. 46B: On the top is a schematic representation of αS fibrils formation involves stepwise pathway. On the bottom is detection of αS aggregation by 2a, 2b and ThT shows RBFs with extended linkages enable the detection of misfolded protein oligomers in vitro. FIG. 46C: Relative intensity (a.u.) of 2a, 2b, and ThT over 160 hour time span, indicating monomer evolution towards mature fibrils.

FIG. 47A: On the left is a schematic representation of selective detection of αS fibrils in the presence of cellular proteins that are abundant in the cytosolic HEK cell lysate; On the right is fluorescence fold change of 2a and 2b (1 μM) with αS PFFs (0-50 μM) at 10 mg/mL BEK lysate concentration. FIG. 47B: Amyloid selectivity of the probes 2a and 2b (1 μM) compared to the standard amyloid binding dye ThT with different amyloid fibrils of αS, tau and Aβ1-42 that are present in patients with neurodegenerative diseases. FIG. 47C: Fractionation of αS-PFFs revealed that 2b retained its fluorescence in the pellet fraction more than 2a, T: total; S: supernatant; P: Pellet; top: Coomassie blue gel of T, S, P fractions and their photographs fractions under handheld UV light (365 nm). FIG. 47D: left: ssNMR of αS fibrils structure with mode of probes 2a and 2b binding at different regions such as disordered regions and β-sheets, right: Concentration-dependent shift in the emission maximum of 2b with αS fibrils indicates binding at multiple sites, whereas 2a, which shows a minimal shift, suggests binding at limited sites with high affinity. λex/λem=463/580 nm for 2a, and λex/λem=484/620 nm for 2b. λex/λem=450/482 nm for ThT Error bars represent SD of 3 measurements.

FIG. 48A: Workflow for patient sample preparation and diagnostic detection of amplified fibrils by a plate reader-based fluorescence assay. Three different AD and PDD patient samples were extracted, so-called AD or PDD lysates, used as controls, containing a large number of cellular proteins and a low concentration of amyloid fibrils. Subsequently, amplified fibrils (AFs) were prepared from AD and PDD lysates by adding 1 μM αS monomer. The final brain lysate samples extracted from the brains of AD and PDD patients were characterized by Western blots. FIG. 48B: Fluorescence fold change of the probes 2a with brain lysates and AFs derived from AD and PDD patients. The florescence bar graph represents the averaged data based on three different AD and PDD patients derived AFs and lysates. The final lysate, AFs and probe concentrations were 1 μM each. Fluorescence fold change was subjected to subtraction of free probe fluorescence in PBS buffer. Fluorescence measurements were performed on a Tecan plate reader with the following excitation and emission wavelengths: 463/580 nm for 2a, 484/620 nm for 2b and 450/482 nm for ThT. Error bars represent the SD of 3 measurements. Statistical analysis: *p<0.05, **p<0.01, and ***p<0.005. FIG. 48C: Fluorescence fold change of the probes 2b with brain lysates and AFs derived from AD and PDD patients. The florescence bar graph represents the averaged data based on three different AD and PDD patients derived AFs and lysates. The final lysate, AFs and probe concentrations were 1 μM each. Fluorescence fold change was subjected to subtraction of free probe fluorescence in PBS buffer. Fluorescence measurements were performed on a Tecan plate reader with the following excitation and emission wavelengths: 463/580 nm for 2a, 484/620 nm for 2b and 450/482 nm for ThT. Error bars represent the SD of 3 measurements. Statistical analysis: *p<0.05, **p<0.01, and ***p<0.005. FIG. 48D: Fluorescence fold change of the probes ThT with brain lysates and AFs derived from AD and PDD patients. The florescence bar graph represents the averaged data based on three different AD and PDD patients derived AFs and lysates. The final lysate, AFs and probe concentrations were 1 μM each. Fluorescence fold change was subjected to subtraction of free probe fluorescence in PBS buffer. Fluorescence measurements were performed on a Tecan plate reader with the following excitation and emission wavelengths: 463/580 nm for 2a, 484/620 nm for 2b and 450/482 nm for ThT. Error bars represent the SD of 3 measurements. Statistical analysis: *p<0.05, **p<0.01, and ***p<0.005.

FIG. 49: Molecular representation of compounds of the disclosure.

FIG. 50A: Absorption spectra of 3a-3f FIG. 50B: Emission spectra of 3a-e. FIG. 50C: Emission spectra of 3f. FIG. 50D: Solvatochromism of 3f in different polarity solvents. FIG. 50E: X-ray and optimized structure of 3f in non-polar and polar solvent. FIG. 50F: Solid state fluorescence of 3f.

FIG. 51: Fluorescence fold changes of probes (3b-f) in the presence of different amyloid fibrils.

FIG. 52: Kd determination of probes 3f (10 nM-5 mM) with 25 mM αS PFFs.

FIG. 53: Fluorescence fold change of 3f (1 μM) with Ab1-42 fibrils in the presence of 1 mg/mL cytosolic HEK cell lysate and E. coli lysate.

FIG. 54A: Highly tunable photophysical properties of bimane probes by varying substituent effects. (a) Absorption spectra of selected bimane probes 5b, 5d, 5f, 5j, 5k and 3. Structure-photophysical property relationship of 5a-f and 5j, 5k. FIG. 54B: Correlation between absorption maximum (λabs) and Hammett constant (σp) of R substituents in styryl bimanes. FIG. 54C: Normalized and smoothed fluorescence spectra of selected bimane probes 5b, 5d, 5f, 5j, 5k and 3, measured with excitation at their optimal max absorption. FIG. 54D Top: photographs of the tunable emission in ACN/PBS solution, Bottom: solid state fluorescence under handheld UV lamp at 365 nm. FIG. 54E: Top: Schematic representation of the bimane probes showing “push-pull” D-π-A or A-π-D type systems based on the aryl substituent R; D: donor, A: acceptor, Bottom: HOMO and LUMO of 5b (R=CN), 5d (R=H), and 5k (R=NMe₂), showing the movement in electron density upon excitation from S₀ to S₁. Final concentration of probes is 25 M in ACN and PBS buffer (50:50 v/v).

FIG. 55A: Dual sensitivity in polarity and viscosity of a solvatochromic fluorophore 5k. D-p-A system shows the phenomenon of ICT from the donor to the acceptor in the excited state and excited fluorophore could either return to the ground state through fluorescence or through internal rotation which leads to non-radiative TICT. FIG. 55B: Solvatochromism of 5k in organic solvents with different polarity. 5k was prepared at 25 μM in indicated solvents. FIG. 55C: Viscosity sensitivity of 5k with varying the glycerol concentration in ethylene glycol. Inset: Tubes with varying glycerol % illuminated with a 365 nm handheld UV lamp. FIG. 55D: Fluorescence intensity of probe 5k (1 μM) in the presence of varying concentrations of BSA protein. Inset: Schematic representation of the fluorescence “turn on” mechanism through rotational restriction upon binding to the hydrophobic pockets on the protein surface. λex/λem=463/580 nm for 5k. Error bars represent SD of 3 measurements.

FIG. 56A: Fluorogenic probes for the detection of αS fibrils. Fluorescence spectral changes of probes in the presence and absence of αS PFFs 5j. FIG. 56B: Fluorogenic probes for the detection of αS fibrils. Fluorescence spectral changes of probes in the presence and absence of αS PFFs 5k; bottom: Structure and photographs of the probes with and without αS PFFs under handheld UV light at 365 nm. Concentrations of dyes and PFFs were 10 and 50 μM, respectively. FIG. 56C: Fluorescence lifetime (t) measurements of 5j and 5k with 100 μM αS PFFs by time correlated single photon counting (TCSPC) technique and lifetime was not detected for probe alone. FIG. 56D: Fluorescence lifetime (t) measurements of 5j and 5k with 100 μM αS PFFs by time correlated single photon counting (TCSPC) technique and lifetime was not detected for probe alone. FIG. 56E: Schematic representation of the fluorescence “turn on” mechanism by rotational restriction upon binding to αS-PFFs. FIG. 56F: Dissociation constant (Kd) determination of 5k (10 nM-10 μM) with 100 μM αS PFFs (see the ESI for the Kd fitting parameters and constraints). λex/λem=435/575 nm for 5j and 463/580 nm for 5k. Error bars represent SD of 3 measurements.

FIG. 57A: Selectivity of 5k for αS fibrils. Schematic representation of selective detection of αS fibrils over monomer. FIG. 57B: Dose-dependent fluorescence “turn-on” with αS fibrils and monomer. FIG. 57C: Fractionation of 5k (1. Control with only fibrils 2. Fibrils spiked with monomer) confirms the selective binding to αS fibrils over αS monomer revealed by SDS-PAGE and fluorescence of T, S, P fractions under handheld UV light (365 nm); T: total; S: supernatant; P: Pellet. FIG. 57D: On the left is a schematic representation of selective detection of αS fibrils in the presence of cellular proteins that are abundant in the cytosolic HEK cell lysate; on the right, fluorescence fold change of 5k (1 μM) with αS fibrils (0-50 μM) at 10 mg/mL BEK lysate concentration. FIG. 57E: Amyloid selectivity of the probe 5k (1 μM) compared to the standard amyloid binding dye ThT with different amyloid fibrils of αS, tau and Aβ1-42 that are present in patients with neurodegenerative diseases. λex/λem=463/580 nm for 5k. Error bars represent SD of 3 measurements.

FIG. 58: Selective detection of αS fibrils amplified from PDD lysates over AD. Workflow for detecting amplified fibrils (AFs) generated from seeds extracted from brain samples of AD or PDD patients, as well as control samples containing only lysate.

FIG. 59: Fluorescence fold change relative to dye alone for 5k or ThT incubated with lysate or AF samples. The fluorescence bar graph represents the averaged data based on samples from three different AD or PDD patients. The final lysate or AFs and probe concentrations were 1 μM. The fold change in fluorescence fold was determined relative to the fluorescence of the free probe in PBS buffer. Fluorescence measurements were performed on a Tecan plate reader with the following excitation and emission wavelengths. λex/λem=463/580 nm for 5k and 450/482 nm for ThT. Error bars represent the SD of 3 measurements. Statistical analysis: * p<0.05 and ** p<0.005.

FIGS. 60A-60G: Liquid Chromatography (LC) chromatogram for the conversion of 5k under different optimized reaction conditions (Entry 1, FIG. 60A; Entry 3, FIG. 60B; Entry 4, FIG. 60C; Entry 5, FIG. 60D; Entry 6, FIG. 60E; Entry 7, FIG. 60F; Entry 8, FIG. 60G).

FIG. 61: X-ray diffraction data and ORTEP drawing of 5a with 50% thermal ellipsoids.

FIG. 62: X-ray diffraction data and ORTEP drawing of 5c with 50% thermal ellipsoids.

FIG. 63: X-ray diffraction data and ORTEP drawing of 5k with 50% thermal ellipsoids.

FIG. 64: Photophysical properties of 3 and 5a-5k in ACN solvent. (a) absorption spectra of 3 and 5a-5f and 5h. (b) shows absorption spectra of 5i-5k, (c) shows fluorescence spectra of 3 and 5a-5f and 5h. (d) Fluorescence spectra of 5i-5k. Final probe concentration is 25 μM. Fluorescence spectra were measured at their maximum absorption wavelength.

FIG. 65: Solvatochromism of 3, 5b, 5d and 5j. Left: Normalized fluorescence spectra in different solvents. Right: Fluorescence image taken under handheld UV lamp at 365 nm. Probe concentration 25 μM and excitation wavelength is 384 nm for 3, 320 nm for 5b and 5d, and 418 nm for 5j.

FIGS. 66A-66K: Representative QY acquisitions of 3 and 5a-f, 5h-k (3, FIG. 66A; 5a, FIG. 66B; 5b, FIG. 66C; 5c, FIG. 66D; 5d, FIG. 66E; 5e, FIG. 66F; 5f, FIG. 66G; 5h, FIG. 66H; 5i, FIG. 66I; 5j, FIG. 66J; 5k, FIG. 66K) in ACN/PBS buffer (50:50). All had consistent excitation (360 nm) and spectral collection (350-800 nm) parameters. Final concentration of the probe is 500 μM. Black line indicates incident light and blue line indicates sample spectrum.

FIGS. 67A-67K: Representative QY acquisitions of 3 and 5a-f, 5h-k (3, FIG. 67A; 5a, FIG. 67B; 5b, FIG. 67C; 5c, FIG. 67D; 5d, FIG. 67E; 5e, FIG. 67F; 5f, FIG. 67G; 5h, FIG. 67H; 5i, FIG. 67I; 5j, FIG. 67J; 5k, FIG. 67K) in ACN solvent. All had consistent excitation (360 nm, slit width 10 nm) and spectral collection (350-800 nm) parameters. Final concentration of the probe is 500 μM. Black line indicates incident light and blue line indicates sample spectrum.

FIG. 68: Effect of pH on absorption and emission of 5h and 5j. Final concentration of the probe is 25 μM. For pH screening of 5h and 5j, PBS buffer solutions were prepared, then adjusted the pH 2, 4, and 7 by adding 6 M hydrochloric acid, pH 9 and 11 by adding 5 M sodium hydroxide. Emission spectra were measured by the excitation wavelength 354 nm for 5h and 385 nm for 5j

FIG. 69: Fluorescence lifetime measurements in ACN/PBS buffer (50:50 v/v). Lifetime measurements with exponential fits shown at left and residuals after fitting shown at right. TCSPC data were collected at 540 nm emission for 5c-5f and 464 nm emission for 3. Excitation wavelength 340 nm.

FIG. 70: Correlation between HOMO-LUMO energy gap (eV) compared to experimentally measured UV-Vis absorption maximum (nm) of 5a-5f, 5h, 5j, and 5k. Maximum UV-Vis absorption wavelengths were obtained in ACN/PBS buffer (50:50 v/v).

FIG. 71: Relative HOMO-LUMO energy gap (eV) of selected bimane derivatives.

FIG. 72: HOMO and LUMO MOs of 5a-5e.

FIG. 73: HOMO and LUMO MOs of 5f, 5h, 5j, and 5k.

FIGS. 74A-74B: Protein purification and cleavage of 1N4R tau. FIG. 74A shows nickel purification visualized by a Coomassie stained 10% SDS-Page gel. FIG. 74B shows FPLC purification fractions by SDS-PAGE gel.

FIG. 75: MALDI MS characterization of WT αS (Top) and tau 1N4R (Bottom). On each plot matrix adduct peaks are marked with *.

FIGS. 76A-76C: Gel-based quantification of proteins. FIG. 76A is protein in αS, FIG. 76B shows 1N4R tau, and FIG. 76C shows Ab1-42 fibrils.

FIGS. 77A-77F: Fluorescence spectra of probes (5b, FIG. 77A; 5c, FIG. 77B; 5d, FIG. 77C; 5e, FIG. 77D; 5f, FIG. 77E; 5h, FIG. 77F) with and without αS fibrils. Final concentrations of probes and αS fibrils were 10 μM and 50 μM, respectively. Emission spectra collected at each dye's maximum absorption wavelength. Ex/Em slit widths: 3 nm/3 nm.

FIGS. 78A-78D: Absorbance is shown in FIG. 78A and FIG. 78B, whereas FIG. 78C and FIG. 78D show excitation spectra of probes 5j and 5k with and without αS fibrils. Final probe and fibrils concentrations were 10 μM and 50 μM, respectively. Final probe and fibril concentrations were 10 μM and 50 μM, respectively. Emission wavelengths were 575 nm for 5j, 580 nm for 5k. Ex/Em slit widths: 3 nm/3 nm.

FIG. 79: Representative QY acquisitions of 5j and 5k with αS fibrils. Black line indicates incident light (αS fibrils without probe) and blue line indicates sample (αS fibrils with probe). Excitation at λexc=435 nm for 5j and λexc=463 nm for 5k and spectral collection (400-800 nm). Final concentrations of the probe and αS fibrils are the 100 μM. Ex/Em slit widths: 5 nm/5 nm.

FIGS. 80A-80C: Fluorescence spectra (top row) and excitation spectra (bottom row) of 5j (FIG. 80A), 5k (FIG. 80B), and ThT (FIG. 80C) with and without tau fibrils. Final probe and tau fibril concentrations were 5 μM and 25 μM, respectively. Excitation at λexc=435 nm for 5j, λexc=463 nm for 5k, λexc=450 nm for ThT. Emission wavelengths were 575 nm for 5j, 580 nm for 5k, and 482 nm for ThT. Ex/Em slit widths: 3 nm/3 nm.

FIGS. 81A-81F: Fluorescence spectra (top row) and excitation spectra (bottom row) of 5j (FIG. 81A, FIG. 81D), 5k (FIG. 81B, FIG. 81E) and ThT (FIG. 81C, FIG. 81F) with and without Ab1-42 fibrils. Final probe and fibrils concentrations were 10 μM and 50 μM. Excitation at λexc=435 nm for 5j, λexc=463 nm for 5k, and λexc=450 nm fot ThT. Emission wavelengths were 575 nm for 5j, 580 nm for 5k, and 482 nm for ThT. Ex/Em slit widths: 3 nm/3 nm.

FIGS. 82A-82C: Fluorescence spectra of probe 5k (10 μM) with and without αS monomer (50 μM) in different TMAO concentrations. FIG. 82A shows 2 M TMAO, FIG. 82B shows 4 M TMAO, FIG. 82C shows 6 M TMAO, and tabulated λmax (λem) of 5k with increasing TMAO concentrations. Excitation wavelength for 5k is 418 nm. λmax (nm) of 5k was 567, 566, and 560 respectively. Ex/Em slit widths: 3 nm/3 nm.

FIG. 83: Fluorescence spectra of probe 5k (10 μM) with BSA (50 μM). λmax (λem) of 5k found was 555 nm. Excitation wavelength for 5k is 418 nm. Ex/Em slit widths: 3 nm/3 nm.

FIGS. 84A-84B. Preparation of fibrils amplified from patient-derived α-synuclein aggregates. FIG. 84A shows Western blots of the final brain lysate samples extracted from AD and PDD patient brains. Membranes were probed with anti-pSer-129 αS (81A) or total αS (HuA). FIG. 84B shows Western blot of sedimentation results for amplified fibrils (AFs) after seven days of incubation. Supernatant (S) and pellet (P) fractions were run for each reaction with monomer (M) and PFF (F) as loading controls. The membrane was probed with HuA.

FIG. 85: Representative images of pathology induced in primary mouse hippocampal neurons for seeds and AFs (Amp) for each case, or PFFs. Probed with 81A (green), anti-MAP2 (red) and DAPI (blue). Scale bar 50 m.

FIGS. 86A-86C: Photophysical properties of 1 and 5a-f, 5h-k. FIG. 86A shows a photograph under ambient light. FIG. 86B Solution state fluorescence of 25 μM probe in ACN/PBS buffer (50:50 v/v) under handheld UV lamp (365 nm). FIG. 86C shows amorphous solid-state fluorescence under handheld UV lamp (365 nm).

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

As used herein, the term “alkenylenyl” or “alkenylene” refers to a divalent unsaturated contiguously conjugated hydrocarbon group which may be linear or branched and which comprises at least one carbon-carbon double bond. Representative examples of alkynylenyl groups include, but are not limited to, —CH═CH—, —CH═CH—CH═CH—, —CH═CH—C≡C—, and the like. An alkenylenyl group may be unsubstituted or substituted by one or more suitable substituents as defined elsewhere herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like.

Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

As used herein, the term “alkynylenyl” or “alkynylene” refers to a divalent unsaturated contiguously conjugated hydrocarbon group which may be linear or branched and which comprises at least one carbon-carbon triple bond. Representative examples of alkynylenyl groups include, but are not limited to, —C≡C—, —C≡C—C≡C—, —C≡C—CH═CH—, and the like. An alkynylenyl group may be unsubstituted or substituted by one or more suitable substituents as defined elsewhere herein.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-, 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical

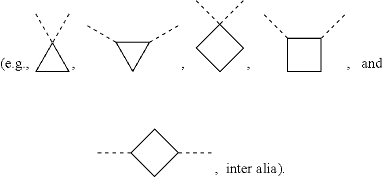

In certain embodiments, the term may be regarded as a product of removal of two hydrogen atoms from the corresponding cycloalkane (e.g., cyclobutyl) by removal of two hydrogen atoms from the same

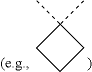

different

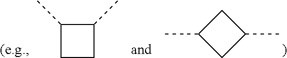

carbon atoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “fibrillar protein” as used herein refers to proteins comprising polypeptide chains which are arranged to form long fibers and/or sheets in a matrix. In certain embodiments, amyloid beta (Aβ) protein may misfold and aggregate to form fibrils that contribute to the formation of amyloid plaques, which are one of the pathological hallmarks of Alzheimer's disease. In other embodiments, alpha-synuclein (αS) may misfold and aggregate to form fibrils (e.g., Lewy bodies), which are abnormal protein aggregates that are associated with Parkinson's disease.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroalkyl” as used herein refers to a non-cyclic stable straight or branched chain, or combinations thereof, including at least one carbon atom and at least one heteroatom selected from the group consisting of O, S, N, P, and Si, and wherein the nitrogen, sulfur, and silicon atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Thus, in one aspect, the term “heteroalkyl” includes alkoxy substituents, as defined elsewhere herein. Non-limiting examples include —CH₂—CH₂—O—CH₃, —CH₂—CH₂—NH—CH₃, —CH₂—CH₂—N(CH₃)—CH₃, —CH₂—S—CH₂—CH₃, —CH₂—S(═O)—CH₃, and —Si(CH₃)₃.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

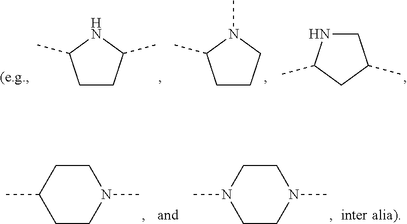

In certain embodiments, the term may be regarded as a product of removal of two hydrogen atoms from the corresponding heterocycloalkane (e.g., piperidine) by removal of two hydrogen atoms from the same

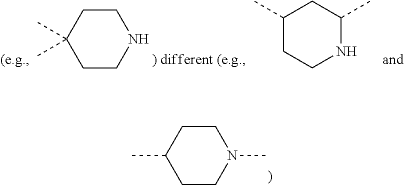

carbon atom(s) and/or heteroatom(s).

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “monovalent” as used herein refers to a substituent connecting via a single bond to a substituted molecule. When a substituent is monovalent, such as, for example, F or Cl, it is bonded to the atom it is substituting by a single bond.

The term “phenylene” or “phenylenyl” as used herein refers to a bivalent phenyl radical (e.g., 1,4-phenylene). In certain embodiments, the term may be regarded as a divalent radical formed by the removal of two hydrogen atoms from a benzene moiety.

The term “protein aggregate” as used herein refers to two or more proteins (e.g., two or more identical proteins, two or more different proteins, etc.) that aggregate together. In certain embodiments, the aggregation may occur in a tissue of a subject. In certain embodiments, this aggregation may produce or place the subject at risk for a pathological condition. In some embodiments, the protein aggregate may be or include one or more of: misfolded protein(s), otherwise improperly formed/misshapen protein(s) (e.g., due to mutations that may not affect folding but do affect function), and/or aggregation of protein and non-protein components (e.g., nucleic acids, small molecules, etc.). Non-limiting examples of such protein aggregates include aggregates of amyloid protein, aggregates of alpha-synuclein, aggregates of tau protein, aggregates of TDP-43 protein, aggregates of immunoglobulin light chain or thyroxine transporter, aggregates of prion protein, and the like.

The term “room temperature” as used herein refers to a temperature of about 15° C. to 28° C.

The term “specifically binds”, or “specifically binds”, or the like, means that a small molecule, an antibody, and/or antigen-binding fragment forms a complex with a target and/or an antigen that is relatively stable under physiological conditions. The specific bond can be characterized by an equilibrium dissociation constant (for example, a smaller K_{D} denotes a firmer bond). Methods for determining whether two molecules specifically bind to each other are well known in the art and include, for example, equilibrium dialysis, surface plasmon resonance, and the like.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

### Description

Among many fluorescent scaffolds, bimane is a highly attractive structure for its small size, high quantum yield, and relatively low toxicity. Bimane is widely used in protein labeling as cysteine-reactive monobromobimane. Recently, it has been shown that a syn-bimane adduct of α-aminobutyric acid can cross the blood-brain barrier in rodents. Unfortunately, the potential for practical applications of bimane derivatives has thus far been limited due to synthetic routes requiring hazardous reagents that can be difficult to handle.

The present disclosure relates, in one aspect, to tunable compounds comprising a bimane scaffold which is compatible with the generation of libraries of fluorescent molecules via a modular three step synthesis. In certain embodiments, the derivatives permit excitation with visible light. In certain embodiments, the derivatives permit tuning of photophysical properties. In certain embodiments, the derivatives permit large (˜200 nm) Stokes shifts. In certain embodiments, the derivatives permit solvatochromism.

The present disclosure further describes the utility of certain exemplary bimane compounds as fluorogenic “turn on” probes for the selective binding of the α-synuclein (αS) protein that aggregates to form amyloid fibrils which play an important role in PD and related neurodegenerative disorders. The designed probes can bind αS fibrils in cell lysates with selectivity over amyloid fibrils of other proteins, such as tau and amyloid-β.

The present disclosure further demonstrates the potential and/or utility of certain exemplary bimane compounds for use in a diagnostic setting, including but not limited to use in distinguishing among amplified fibrils in clinical samples from patients with various neurodegenerative diseases.

### Compounds

In one aspect, the present disclosure provides a compound of Formula (I), or a salt, solvate, stereoisomer, or isotopologue thereof:

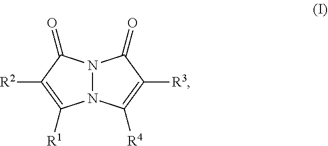

wherein:
R¹, R², and R³ are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{A}, N(R^{A})(R^{B}), SR^{A}, C(═O)R^{A}, C(═O)OR^{A}, C(═O)N(R^{A})(R^{B}), OC(═O)R^{A}, OC(═O)OR^{A}, OC(═O)N(R^{A})(R^{B}), NC(═O)R^{A}, NC(═O)OR^{A}, NC(═O)N(R^{A})(R^{B}), S(═O)R^{A}, S(═O)OR^{A}, S(═O)N(R^{A})(R^{B}) S(═O)₂R^{A}, S(═O)₂OR^{A}, and S(═O)₂N(R^{A})(R^{B});
R⁴ is

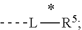

L is selected from the group consisting of optionally substituted C₂-C₆ alkenylenyl and optionally substituted C₂-C₆ alkynylenyl;
R⁵ is selected from the group consisting of optionally substituted C₆-C₁₀ aryl and optionally substituted C₂-C₁₀ heteroaryl;
* indicates a bond between L and R⁵; and
R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

In certain embodiments, at least one of R¹, R², and R³ is C₁-C₆ alkyl. In certain embodiments, at least two of R¹, R², and R³ are independently C₁-C₆ alkyl. In certain embodiments, each of R¹, R², and R³ are independently C₁-C₆ alkyl.

In certain embodiments, at least one of R¹, R², and R³ is CH₃. In certain embodiments, at least two of R¹, R², and R³ are CH₃. In certain embodiments, each of R¹, R², and R³ are CH₃.

In certain embodiments, L is optionally substituted C₂-C₆ alkenylenyl.

In certain embodiments, L is

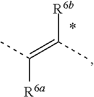

wherein each occurrence of R^{6a} and R^{6b} is independently selected from the group consisting of H and optionally substituted C₁-C₆ alkyl.

In certain embodiments, L is

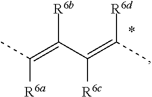

wherein each occurrence of R^{6a}, R^{6b}, R^{6c}, and R^{6d} is independently selected from the group consisting of H and optionally substituted C₁-C₆ alkyl.

In certain embodiments, L is

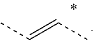

In certain embodiments, L is

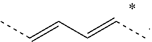

In certain embodiments, R⁵ is:

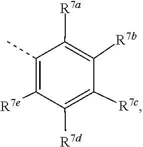

wherein:
R^{7a}, R^{7b}, R^{7c}, R^{7d}, and R^{7e} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{C}, N(R^{C})(R^{D}), SR^{C}, C(═O)R^{C}, C(═O)OR^{C}, C(═O)N(R^{C})(R^{D}), OC(═O)R^{C}, OC(═O)OR^{C}, OC(═O)N(R^{C})(R^{D}), NC(═O)R^{C}, NC(═O)OR^{C}, NC(═O)N(R^{C})(R^{D}), S(═O)R^{C}, S(═O)OR^{C}, S(═O)N(R^{C})(R^{D}), S(═O)₂R^{C}, S(═O)₂OR^{C}, and S(═O)₂N(R^{C})(R^{D}); and
R^{C} and R^{D} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

In certain embodiments, R⁵ is:

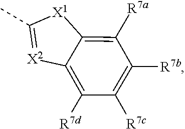

wherein:
X¹ is selected from the group consisting of S, O, and NR^{E};
X² is selected from the group consisting of N and CR^{7e};
R^{7a}, R^{7b}, R^{7c}, R^{7d}, and R^{7e}, if present, are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{C}, N(R^{C})(R^{D}), SR^{C}, C(═O)R^{C}, C(═O)OR^{C}, C(═O)N(R^{C})(R^{D}), OC(═O)R^{C}, OC(═O)OR^{C}, OC(═O)N(R^{C})(R^{D}), NC(═O)R^{C}, NC(═O)OR^{C}, NC(═O)N(R^{C})(R^{D}), S(═O)R^{C}, S(═O)OR^{C}, S(═O)N(R^{C})(R^{D}), S(═O)₂R^{C}, S(═O)₂OR^{C}, and S(═O)₂N(R^{C})(R^{D}); and
R^{C}, R^{D}, and R^{E} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

In certain embodiments, R^{7a} is H. In certain embodiments, R^{7a} is F. In certain embodiments, R^{7a} is NO₂. In certain embodiments, R^{7a} is CN. In certain embodiments, R^{7a} is N₃. In certain embodiments, R^{7a} is CH₃. In certain embodiments, R^{7a} is OH. In certain embodiments, R^{7a} is OCH₃. In certain embodiments, R^{7a} is NH₂. In certain embodiments, R^{7a} is N(CH₃)₂. In certain embodiments, R^{7a} is morpholinyl. In certain embodiments, R^{7a} is NHC(═O)OC(CH₃)₃.

In certain embodiments, R^{7b} is H. In certain embodiments, R^{7b} is F. In certain embodiments, R^{7b} is NO₂. In certain embodiments, R^{7b} is CN. In certain embodiments, R^{7b} is N₃. In certain embodiments, R^{7b} is CH₃. In certain embodiments, R^{7b} is OH. In certain embodiments, R^{7b} is OCH₃. In certain embodiments, R^{7b} is NH₂. In certain embodiments, R^{7b} is N(CH₃)₂. In certain embodiments, R^{7b} is morpholinyl. In certain embodiments, R^{7b} is NHC(═O)OC(CH₃)₃.

In certain embodiments, R^{7c} is H. In certain embodiments, R^{7c} is F. In certain embodiments, R^{7c} is NO₂. In certain embodiments, R^{7c} is CN. In certain embodiments, R^{7c} is N₃. In certain embodiments, R^{7c} is CH₃. In certain embodiments, R^{7c} is OH. In certain embodiments, R^{7c} is OCH₃. In certain embodiments, R^{7c} is NH₂. In certain embodiments, R^{7c} is N(CH₃)₂. In certain embodiments, R^{7c} is morpholinyl. In certain embodiments, R^{7c} is NHC(═O)OC(CH₃)₃.

In certain embodiments, R^{7d} is H. In certain embodiments, R^{7d} is F. In certain embodiments, R^{7d} is NO₂. In certain embodiments, R^{7d} is CN. In certain embodiments, R^{7d} is N₃. In certain embodiments, R^{7d} is CH₃. In certain embodiments, R^{7d} is OH. In certain embodiments, R^{7d} is OCH₃. In certain embodiments, R^{7d} is NH₂. In certain embodiments, R^{7d} is N(CH₃)₂. In certain embodiments, R^{7d} is morpholinyl. In certain embodiments, R^{7d} is NHC(═O)OC(CH₃)₃.

In certain embodiments, R^{7e} is H. In certain embodiments, R^{7e} is F. In certain embodiments, R^{7e} is NO₂. In certain embodiments, R^{7e} is CN. In certain embodiments, R^{7e} is N₃. In certain embodiments, R^{7e} is CH₃. In certain embodiments, R^{7e} is OH. In certain embodiments, R^{7e} is OCH₃. In certain embodiments, R^{7e} is NH₂. In certain embodiments, R^{7e} is N(CH₃)₂. In certain embodiments, R^{7e} is morpholinyl. In certain embodiments, R^{7e} is NHC(═O)OC(CH₃)₃.

In certain embodiments, X¹ is S.

In certain embodiments, X² is N.

In certain embodiments, R⁵ is

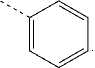

In certain embodiments, R⁵ is

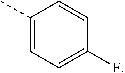

In certain embodiments, R⁵ is

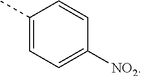

In certain embodiments, R⁵ is

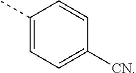

In certain embodiments, R⁵ is

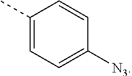

In certain embodiments, R⁵ is

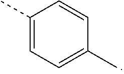

In certain embodiments, R⁵ is

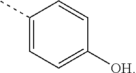

In certain embodiments, R⁵ is

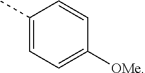

In certain embodiments, R⁵ is

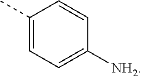

In certain embodiments, R⁵ is

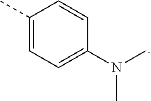

In certain embodiments, R⁵ is

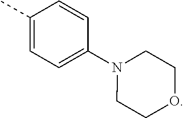

In certain embodiments, R⁵ is

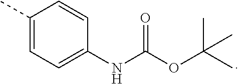

In certain embodiments, R⁵ is

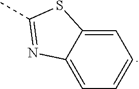

In certain embodiments, R⁵ is

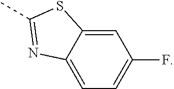

In certain embodiments, R⁵ is

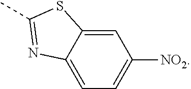

In certain embodiments, R⁵ is

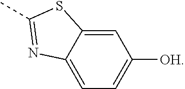

certain embodiments, R⁵ is

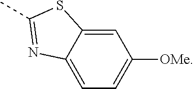

In certain embodiments, R⁵ is

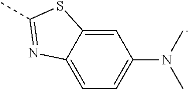

In certain embodiments, R⁴ is

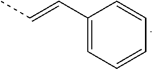

In certain embodiments, R⁴ is

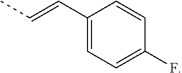

In certain embodiments, R⁴ is

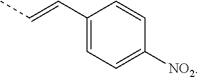

In certain embodiments, R⁴ is

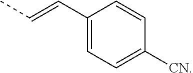

In certain embodiments, R⁴ is

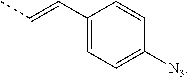

In certain embodiments, R⁴ is

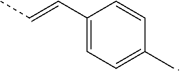

In certain embodiments, R⁴ is

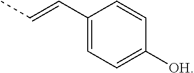

In certain embodiments, R⁴ is

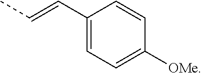

In certain embodiments, R⁴ is

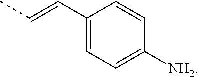

In certain embodiments, R⁴ is

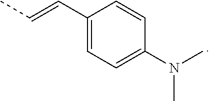

In certain embodiments, R⁴ is

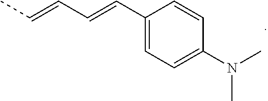

In certain embodiments, R⁴ is

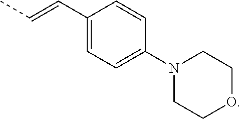

In certain embodiments, R⁴ is

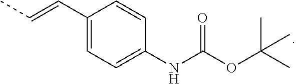

In certain embodiments, R⁴ is

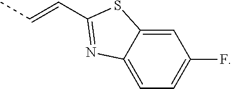

In certain embodiments, R⁴ is

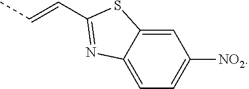

In certain embodiments R⁴ is

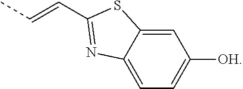

In certain embodiments, R⁴ is

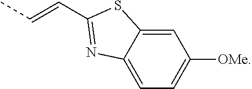

In certain embodiments, R⁴ is

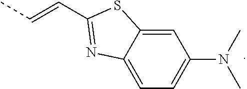

In certain embodiments, each occurrence of optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₂-C₆ alkenylenyl, optionally substituted C₂-C₆ alkynylenyl, optionally substituted aryl, and optionally substituted heteroaryl is independently optionally substituted with at least one selected from the group consisting of C₁-C₆ alkyl, C₃-C₈ cycloalkyl, C₂-C₁₂ heterocycloalkyl, C₁-C₆ hydroxyalkyl, halogen, CN, N₃, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

In certain embodiments, the compound is selected from the group consisting of:
(E)-2,3,6-trimethyl-5-(4-nitrostyryl)-1H,7H-pyrazolo[1,2-a]pyrazole-1,7-dione;
(E)-4-(2-(2,5,6-trimethyl-1,7-dioxo-1H,7H-pyrazolo[1,2-a]pyrazol-3-yl)vinyl)benzonitrile;
(E)-3-(4-fluorostyryl)-2,5,6-trimethyl-1H,7H-pyrazolo[1,2-a]pyrazole-1,7-dione;
(E)-3-(4-azidostyryl)-2,5,6-trimethyl-1H,7H-pyrazolo[1,2-a]pyrazole-1,7-dione;
(E)-2,3,6-trimethyl-5-styryl-1H,7H-pyrazolo[1,2-a]pyrazole-1,7-dione;
(E)-2,3,6-trimethyl-5-(4-methylstyryl)-1H,7H-pyrazolo[1,2-a]pyrazole-1,7-dione;
(E)-3-(4-methoxystyryl)-2,5,6-trimethyl-1H,7H-pyrazolo[1,2-a]pyrazole-1,7-dione;
(E)-3-(4-hydroxystyryl)-2,5,6-trimethyl-1H,7H-pyrazolo[1,2-a]pyrazole-1,7-dione;
tert-butyl (E)-(4-(2-(2,5,6-trimethyl-1,7-dioxo-1H,7H-pyrazolo[1,2-a]pyrazol-3-yl)vinyl)phenyl)carbamate;
(E)-3-(4-aminostyryl)-2,5,6-trimethyl-1H,7H-pyrazolo[1,2-a]pyrazole-1,7-dione;
(E)-2,3,6-trimethyl-5-(4-morpholinostyryl)-1H,7H-pyrazolo[1,2-a]pyrazole-1,7-dione;
(E)-3-(4-(dimethylamino)styryl)-2,5,6-trimethyl-1H,7H-pyrazolo[1,2-a]pyrazole-1,7-dione;
3-((1E,3E)-4-(4-(dimethylamino)phenyl)buta-1,3-dien-1-yl)-2,5,6-trimethyl-1H,7H-pyrazolo[1,2-a]pyrazole-1,7-dione;
(E)-2,3,6-trimethyl-5-(2-(6-nitrobenzo[d]thiazol-2-yl)vinyl)-1H,7H-pyrazolo[1,2-a]pyrazole-1,7-dione;
(E)-3-(2-(6-fluorobenzo[d]thiazol-2-yl)vinyl)-2,5,6-trimethyl-1H,7H-pyrazolo[1,2-a]pyrazole-1,7-dione;
(E)-3-(2-(benzo[d]thiazol-2-yl)vinyl)-2,5,6-trimethyl-1H,7H-pyrazolo[1,2-a]pyrazole-1,7-dione;
(E)-3-(2-(6-methoxybenzo[d]thiazol-2-yl)vinyl)-2,5,6-trimethyl-1H,7H-pyrazolo[1,2-a]pyrazole-1,7-dione;
(E)-3-(2-(6-hydroxybenzo[d]thiazol-2-yl)vinyl)-2,5,6-trimethyl-1H,7H-pyrazolo[1,2-a]pyrazole-1,7-dione; and
(E)-3-(2-(6-(dimethylamino)benzo[d]thiazol-2-yl)vinyl)-2,5,6-trimethyl-1H,7H-pyrazolo[1,2-a]pyrazole-1,7-dione.

TABLE 1
Exemplary compounds

| Cmpd | Structure | Nomenclature |
|---|---|---|
| 7a | 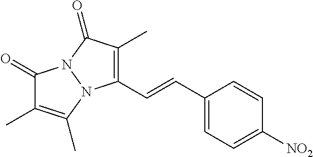 [SMILES: Cc1c(C)n2c(/C=C/c3ccc([N+](=O)[O-])cc3)c(C)c(=O)n2c1=O] | (E)-2,3,6-trimethyl-5-(4-nitrostyryl)- 1H, 7H-pyrazolo[1,2-a]pyrazole-1,7- dione |
| 7b | 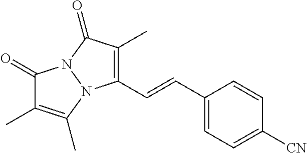 [SMILES: Cc1c(C)n2c(/C=C/c3ccc(C#N)cc3)c(C)c(=O)n2c1=O] | (E)-4-(2-(2,5,6-trimethyl-1,7-dioxo- 1H, 7H-pyrazolo[1,2-a]pyrazol-3- yl)vinyl)benzonitrile |
| 7c | 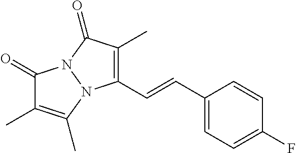 [SMILES: Cc1c(C)n2c(/C=C/c3ccc(F)cc3)c(C)c(=O)n2c1=O] | (E)-3-(4-fluorostyryl)-2,5,6- trimethyl-1H, 7H-pyrazolo[1,2- a]pyrazole-1,7-dione |
| 7d | 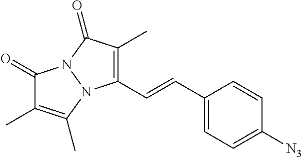 [SMILES: Cc1c(C)n2c(/C=C/c3ccc(N=[N+]=[N-])cc3)c(C)c(=O)n2c1=O] | (E)-3-(4-azidostyryl)-2,5,6-trimethyl- 1H, 7H-pyrazolo[1,2-a]pyrazole-1,7- dione |
| 7e | 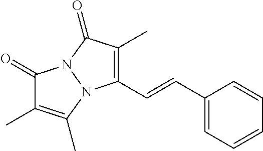 [SMILES: Cc1c(C)n2c(/C=C/c3ccccc3)c(C)c(=O)n2c1=O] | (E)-2,3,6-trimethyl-5-styry1-1H,7H- pyrazolo[1,2-a]pyrazole-1,7-dione |
| 7f | 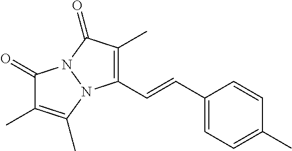 [SMILES: Cc1ccc(/C=C/c2c(C)c(=O)n3c(=O)c(C)c(C)n23)cc1] | (E)-2,3,6-trimethyl-5-(4- methylstyry1)-1H, 7H-pyrazolo[1,2- a]pyrazole-1,7-dione |
| 7g | 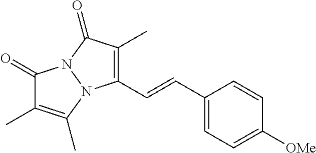 [SMILES: COc1ccc(/C=C/c2c(C)c(=O)n3c(=O)c(C)c(C)n23)cc1] | (E)-3-(4-methoxystyryl)-2,5,6- trimethyl-1H, 7H-pyrazolo[1,2- a]pyrazole-1,7-dione |
| 7h | 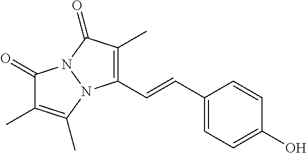 [SMILES: Cc1c(C)n2c(/C=C/c3ccc(O)cc3)c(C)c(=O)n2c1=O] | (E)-3-(4-hydroxystyryl)-2,5,6- trimethyl-1H,7H-pyrazolo[1,2- a]pyrazole-1,7-dione |
| 7i | 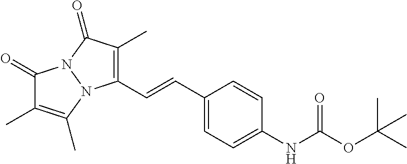 [SMILES: Cc1c(C)n2c(/C=C/c3ccc(NC(=O)OC(C)(C)C)cc3)c(C)c(=O)n2c1=O] | tert-butyl (E)-(4-(2-(2,5,6-trimethyl- 1,7-dioxo-1H,7H-pyrazolo[1,2- alpyrazol-3- yl)vinyl)phenyl)carbamate |
| 7j | 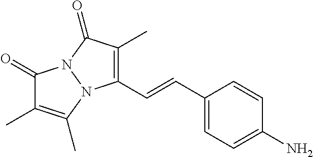 [SMILES: Cc1c(C)n2c(/C=C/c3ccc(N)cc3)c(C)c(=O)n2c1=O] | (E)-3-(4-aminostyryl)-2,5,6- trimethyl-1H, 7H-pyrazolo[1,2- a]pyrazole-1,7-dione |
| 7k | 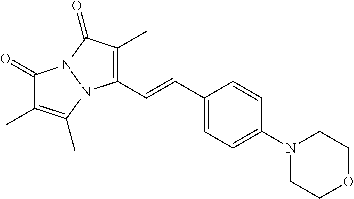 [SMILES: Cc1c(C)n2c(/C=C/c3ccc(N4CCOCC4)cc3)c(C)c(=O)n2c1=O] | (E)-2,3,6-trimethyl-5-(4- morpholinostyry1)-1H,7H- pyrazolo[1,2-a]pyrazole-1,7-dione |
| 7l | 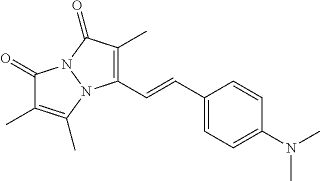 [SMILES: Cc1c(C)n2c(/C=C/c3ccc(N(C)C)cc3)c(C)c(=O)n2c1=O] | (E)-3-(4-(dimethylamino)styryl)- 2,5,6-trimethyl-1H,7H-pyrazolo[1,2- a]pyrazole-1,7-dione |
| 7m | 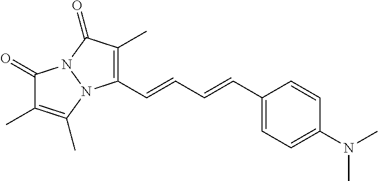 [SMILES: Cc1c(C)n2c(/C=C/C=C/c3ccc(N(C)C)cc3)c(C)c(=O)n2c1=O] | 3-((1E,3E)-4-(4- (dimethylamino)phenyl)buta-1,3- dien-1-yl)-2,5,6-trimethyl-1H,7H- pyrazolo[1,2-a]pyrazole-1,7-dione |
| 8a | 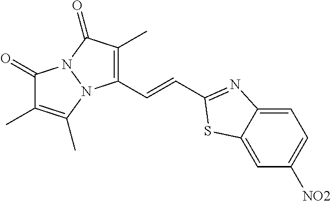 [SMILES: Cc1c(C)n2c(/C=C/c3nc4ccc([N+](=O)[O-])cc4s3)c(C)c(=O)n2c1=O] | (E)-2,3,6-trimethyl-5-(2-(6- nitrobenzo[d]thiazol-2-yl)vinyl)- 1H, 7H-pyrazolo[1,2-a]pyrazole-1,7- dione |
| 8b | 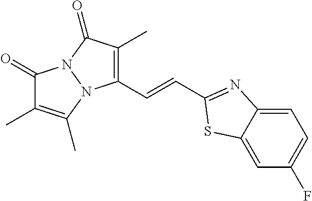 [SMILES: Cc1c(C)n2c(/C=C/c3nc4ccc(F)cc4s3)c(C)c(=O)n2c1=O] | (E)-3-(2-(6-fluorobenzo[d]thiazol-2- yl)vinyl)-2,5,6-trimethyl-1H,7H- pyrazolo[1,2-a]pyrazole-1,7-dione |
| 8c | 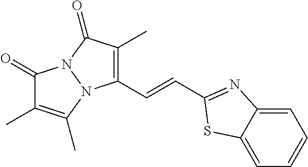 [SMILES: Cc1c(C)n2c(/C=C/c3nc4ccccc4s3)c(C)c(=O)n2c1=O] | (E)-3-(2-(benzo[d]thiazol-2- yl)vinyl)-2,5,6-trimethyl-1H,7H- pyrazolo[1,2-a]pyrazole-1,7-dione |
| 8d | 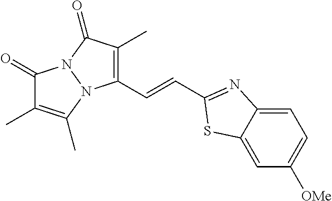 [SMILES: COc1ccc2nc(/C=C/c3c(C)c(=O)n4c(=O)c(C)c(C)n34)sc2c1] | (E)-3-(2-(6-methoxybenzo[d]thiazol- 2-y1)vinyl)-2,5,6-trimethyl-1H,7H- pyrazolo[1,2-a]pyrazole-1,7-dione |
| 8e | 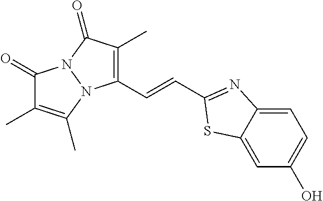 [SMILES: Cc1c(C)n2c(/C=C/c3nc4ccc(O)cc4s3)c(C)c(=O)n2c1=O] | (E)-3-(2-(6-hydroxybenzo[d]thiazol- 2-yl)vinyl)-2,5,6-trimethyl-1H, 7H- pyrazolo[1,2-a]pyrazole-1,7-dione |
| 8f | 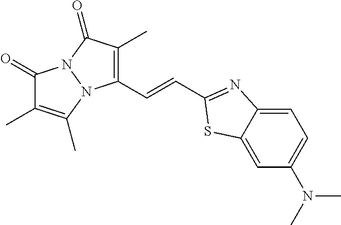 [SMILES: Cc1c(C)n2c(/C=C/c3nc4ccc(N(C)C)cc4s3)c(C)c(=O)n2c1=O] | (E)-3-(2-(6- yl)vinyl)-2,5,6-trimethyl-1H,7H- (dimethylamino)benzo[d]thiazol-2- pyrazolo[1,2-a]pyrazole-1,7-dione |

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C ³⁶Cl, ¹⁸F, ¹²³I, ¹²⁵I, ¹³N, ¹⁵N, ¹⁵O, ¹⁷O ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl)ethoxycarbonyl (Teoc), t-butyloxycarbonyl (Boc), para-methoxybenzyl (PMB), triphenylmethyl (trityl), acetyl, and fluorenylmethoxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods

In one aspect, the present disclosure provides a method for identifying a protein aggregation disease in a subject, the method comprising:
(a) contacting a sample obtained from a subject with at least one compound of Formula (I) to prepare a mixture; and
(b) detecting whether a fluorescence signal is obtained in the mixture, wherein detecting a fluorescence signal identified the subject as having a protein aggregation disease.

In certain embodiments, the protein aggregation disease is a neurodegenerative disease. In certain embodiments, the neurodegenerative disease is Parkinson's disease (PD). In certain embodiments, the neurodegenerative disease is Alzheimer's disease (AD).

In certain embodiments, the detecting comprises:
(a) measuring fluorescence of the mixture to obtain a sample fluorescence value;
(b) measuring fluorescence of a first control sample, which lacks a biomarker of interest, to obtain a first control fluorescence value, optionally wherein the first control sample comprises one or more protein aggregates which are distinct from the biomarker of interest; and
(c) comparing the sample fluorescence value and the first control fluorescence value, wherein a greater sample fluorescence value is indicative of the protein aggregate disease in the subject.

In certain embodiments, the detecting comprises:
(a) measuring fluorescence of the mixture to obtain a sample fluorescence value;
(b) measuring fluorescence of a first control sample, which lacks a biomarker of interest, to obtain a first control fluorescence value, optionally wherein the first control sample comprises one or more protein aggregates which are distinct from the biomarker of interest;
(c) measuring fluorescence of a second control sample which comprises the biomarker of interest to obtain a second control fluorescence value; and
(d) comparing the sample fluorescence value and the first control fluorescence value, wherein a greater sample fluorescence value is indicative of the protein aggregate disease in the subject if the second control fluorescence value is positive.

In certain embodiments, the protein aggregation disease is Parkinson's disease (PD). In certain embodiments, the biomarker of interest is α-synuclein (αS). In certain embodiments, the α-synuclein (αS) is fibrillar. In certain embodiments, the one or more protein aggregates which are distinct from the biomarker of interest comprise amyloid-beta (Aβ, also known as beta amyloid) and/or tau protein, optionally wherein the Aβ is Aβ₁₋₄₂, and optionally wherein the amyloid-beta (Aβ) and/or tau protein are fibrillar aggregates. In certain embodiments, the first control sample further comprises non-aggregated and/or non-fibrillar α-synuclein (αS) (e.g., monomeric αS).

In certain embodiments, the protein aggregation disease is Alzheimer's disease (AD). In certain embodiments, the biomarker of interest is amyloid-beta (Aβ), optionally wherein the Aβ is Aβ₁₋₄₂. In certain embodiments, the amyloid-beta (Aβ) is fibrillar. In certain embodiments, the one or more protein aggregates which are distinct from the biomarker of interest comprise α-synuclein (αS) and/or tau protein, optionally wherein the αS and/or tau protein are fibrillar aggregates. In certain embodiments, the first control sample further comprises non-aggregated and/or non-fibrillar amyloid-beta (Aβ) (e.g., monomeric Aβ).

In another aspect, the present disclosure provides a method of measuring a concentration of a fibrillar protein aggregate in a sample, the method comprising:
(a) contacting a sample comprising the fibrillar protein aggregate with at least one compound of Formula (I) to prepare a mixture; and
(b) measuring fluorescence in the mixture to obtain a mixture fluorescence value;
(c) contacting each of two or more control samples with at least one compound of Formula (I) to prepare two or more control sample mixtures, wherein each control sample comprises a known concentration of the fibrillar protein aggregate of interest;
(d) measuring fluorescence of each of the two or more control samples to obtain two or more control sample fluorescence values;
(e) calculating the concentration of fibrillar protein aggregate in the sample by comparing the mixture fluorescence value to the two or more control sample fluorescence values.

In certain embodiments, the fibrillar protein aggregate is α-synuclein (αS).

In certain embodiments, the fibrillar protein aggregate is amyloid-beta (Aβ).

### EXAMPLES

Various embodiments of the present application can be better understood by reference to the following Examples which are offered by way of illustration. The scope of the present application is not limited to the Examples given herein.

### Materials and Methods

### Materials

Trichloroisocyanuric acid (TCCA), trimethyl phosphite, 4-(dimethylamino)cinnamaldehyde and all other derivatives of aryl aldehydes were purchased from Millipore Sigma (St. Louis, MO, USA). Flash column chromatography was performed using Silicycle silica gel (40-63 m (230-400 mesh), 60 Å irregular pore diameter). Thin-layer chromatography (TLC) was performed on TLC Silica gel 60G F254 plates from Millipore Sigma. Reagents were purchased at the highest commercial quality and used without further purification, unless otherwise stated. The β-amyloid (1-42) peptide, human (Cat. No. RP10017) was purchased from Genscript (Piscataway, NJ).

### Instruments

Nuclear magnetic resonance (NMR) spectra were obtained on a Bruker UNI-400 MHz or UNI-600 MHz instrument (Billerica, MA, USA) and are calibrated using peaks from residual protic solvent in deuterated solvent. The following abbreviations were used to denote multiplicities: s=singlet, d=doublet, t=triplet, q=quartet, m=multiplet, br=broad). Low-resolution mass spectra (LRMS) were obtained on a Waters Acquity Ultra Performance LC connected to a single quadrupole detector mass spectrometer (Waters Corp.; Milford, MA, USA). High-resolution mass spectra (HRMS) were obtained on a High-resolution electrospray ionization mass spectra (ESI-HRMS) using Waters LCT Premier XE liquid chromatograph/mass spectrometer. X-ray diffraction data obtained on a Rigaku XtaLAB Synergy-S diffractometer equipped with an HPC area detector (HyPix-6000HE) and employing confocal multilayer optic-monochromated Mo-Kα radiation (λ=0.71073 Å) or Cu=Kα radiation (λ=1.54184 Å) at a temperature of 100K. Absorbance readings for the DC assay were made on a Tecan M1000 plate reader (Mannedorf, Switzerland). UV-Vis absorption spectra were acquired on a Thermo Scientific Genesys 150 UV-Vis spectrometer (Waltham, MA, USA) using quartz cells with a 1 cm cell path length (Starna Cells, Inc 120 ul UV cells). Fluorescence spectra were acquired on a Tecan M1000 plate reader. Quantum yield (QY) measurements were performed using a Jasco FP-8300 Fluorimeter with ILF-835 integrating sphere attachment (Easton, MD, USA). Fluorescence lifetime measurements were made using a Photon Technology International (PTI) QuantaMaster™ 40 fluorescence spectrometer (Birmingham, NJ, USA).

### Absorbance Measurements

Bimane derivatives 6 and 7a-7m were dissolved in DMSO to make 10 mM starting stock solutions. Samples were individually diluted to the final concentration of 25 μM (500 μL) using 50:50 acetonitrile in phosphate buffered saline (ACN/PBS). The absorbance spectrum of each derivative (150 μL) was measured on a Thermo Scientific Genesys 150 UV-Vis spectrometer, using 50:50 ACN/PBS as a blank.

### Fluorescence Measurements

10 mM stock solutions of 6 and 7a-7m in DMSO were individually diluted to the final concentration of 25 μM (500 μL) using 50:50 ACN/PBS. The fluorescence spectrum of each derivative (150 μL) was measured on a Photon Technology International (PTI) QuantaMaster™ 40 fluorescence spectrometer by excitation at the maximum absorption wavelength for each compound.

### Molar Absorptivity Determination

10 mM stock solutions of 6 and 7a-7m in DMSO were individually diluted to the final concentration of 25 μM (500 μL) using 50:50 ACN/PBS. The absorbance spectrum of each derivative (150 μL) was measured on a Thermo Scientific Genesys 150 UV-Vis spectrometer, using 50:50 ACN/PBS as a blank. Then, the molar absorptivity of each derivative was calculated by using the Beer-Lambert law for solutions, A=εlc, where A=absorbance at maximum wavelength, 1=optical path length in cm, c=concentration of the solution (25 μM), 8=molar absorptivity.

### Solvatochromic Measurements

10 mM stock solution of 6, 7b, 7e, 7j, 7l, and 7m in DMSO were individually diluted into six different organic solvents (toluene, dichloromethane, ACN, dimethyl formamide, dimethyl sulfoxide, and ethanol) to the final concentration of 25 μM (1000 μL). The fluorescence spectrum of each derivative (150 μL) was measured on a PTI QuantaMaster™ 40 fluorescence spectrometer by excitation at the maximum absorption wavelength for each compound. The remaining 850 μL solution was used to obtain fluorescence images under a handheld 365 nm UV lamp.

### pH Measurements

10 mM stock solutions of 7h, 7j, and 7k in DMSO were individually diluted into ACN/PBS buffer (50:50 v/v) having different pH ranging from pH 2-11 with 25 μM (500 μL) of final concentration. For pH screening of 7h and 7j, PBS buffer solutions were prepared, then adjusted the pH 2, 4, and 7 by adding 6 M hydrochloric acid, pH 9 and 11 by adding 5 M sodium hydroxide. For pH screening of 7k, pH 4.4 and 7.4 were prepared by citric acid/phosphate buffer. Then, the absorption and fluorescence spectra were recorded by using Thermo Scientific Genesys 150 UV-Vis spectrometer, followed by PTI QuantaMaster™ 40 fluorescence spectrometer by excitation with maximum absorption wavelength.

### Viscosity Measurements

As a representative example, the viscosity of 7k (25 μM, 10 mM stock in DMSO) in glycerol/methanol mixtures were measured, varying the glycerol percentage 0-99%. Then, the absorption spectra of 7k in these mixtures were recorded using a Thermo Scientific Genesys 150 UV-Vis spectrometer, and the emission spectra were recorded using a PTI QuantaMaster™ 40 fluorescence spectrometer by excitation with 386 nm wavelength.

### Fluorescent Quantum Yield (QY) Measurements

For each QY measurement, the incident excitation light spectrum was collected with 1 mL of solvent. After measuring the incident light intensity, dye (7a-7m) was added to the solvent from a concentrated stock solution to a final concentration of 500 μM and the new spectrum (fluorescence intensity and new incident light intensity) was collected. Using the JASCO Quantum Yield Software, the dye QY was calculated by dividing the dye fluorescence intensity by the difference in incident light intensity in the presence and absence of dye. The minimum excitation wavelength for a full excitation incident spectrum using this setup is 360 nm. QY values are reported in Tables 2-3.

TABLE 2
Photophysical properties of 6 and 7a-7m in ACN.

| | | | | | Fluores- |
|---|---|---|---|---|---|
| | Absor- | | Molar | Stokes | cence |
| | bance | Emission | Absorptivity | shift | quantum |
| Cmpd | λₘₐₓ(nm)^{a} | λₘₐₓ(nm)^{b} | (ε)(M⁻¹cm⁻¹)^{c} | (nm) | yield (%) |
| 6 | 375 | 432 | 6121 | 57 | 77.7 |
| 7a | 332 | 432 | 37871 | 100 | 0.2 |
| 7b | 316 | 522 | 43238 | 206 | 1.4 |
| 7c | 315 | 492, 520 | 28940 | 177 | 2.8 |
| 7d | 339 | 440, 490 | 36055 | 101 | 1.5 |
| 7e | 316 | 492, 522 | 32724 | 176 | 3.1 |
| 7f | 325 | 490, 520 | 24426 | 165 | 3.4 |
| 7g | 342 | 488, 518 | 34554 | 146 | 3.5 |
| 7h | 342 | 486, 518 | 29910 | 144 | 3.1 |
| 7i | 348 | 490, 520 | 37555 | 142 | 3.4 |
| 7j | 378 | 540 | 32091 | 162 | 1.5 |
| 7k | 378 | 565 | 28890 | 187 | 6.9 |
| 7l | 402 | 568 | 24742 | 166 | 7.5 |
| 7m | 410 | 620 | 28639 | 210 | 13.5 |

^{a}Maximum absorption wavelength,
^{b}Maximum emission wavelength,
^{c}Molar absorption coefficients at maximum absorption wavelength,
^{d}Difference between maximum absorption wavelength and maximum emission wavelength,
^{e}Fluorescence quantum yield (error limit within ±5).
^{f}Probe with diene linker, Final probe concentration is 25 μM in ACN.

TABLE 3
Photophysical properties of 6 and 7a-7m in ACN/PBS Buffer (1:1 v/v)

| | | | Molar | Stokes | | Fluorescence |
|---|---|---|---|---|---|---|
| | Absorbance | Emission | Absorptivity | shift | Fluorescence | Lifetime |
| Cmpd | λₘₐₓ(nm)^{a} | λₘₐₓ(nm)^{b} | (ε)(M⁻¹cm⁻¹)^{c} | (nm) | QY (%) | (ns) |
| 6 | 384 | 464 | 7902 | 80 | 64.6 | 2.32 |
| 7a | 335 | 468 | 36955 | 133 | 0.1 | — |
| 7b | 320 | 520 | 38995 | 200 | 0.8 | — |
| 7c | 321 | 502 | 23796 | 181 | 2.5 | 1.07 |
| 7d | 350 | 502 | 27699 | 152 | 1.3 | — |
| 7e | 321 | 503 | 23959 | 182 | 2.5 | 1.02 |
| 7f | 332 | 503 | 21164 | 171 | 3.0 | 1.28 |
| 7g | 348 | 504 | 25659 | 156 | 3.3 | 1.31 |
| 7h | 354 | 506 | 31514 | 152 | 2.3 | — |
| 7i | 356 | 505 | 34826 | 149 | 3.4 | — |
| 7j | 385 | 583 | 30834 | 198 | 0.7 | — |
| 7k | 386 | 603 | 29562 | 217 | 1.0 | — |
| 7l | 418 | 604 | 30420 | 186 | 0.5 | — |
| 7m | 436 | 640 | 36689 | 204 | 0.6 | — |

^{a}For all probes, final concentration is 25 μM in ACN/PBS buffer at pH 7.4.
^{b}Maximum absorption wavelength.
^{c}Maximum emission wavelength.
^{d}Molar absorption coefficients at maximum absorption wavelength.
^{e}Difference between maximum absorption wavelength and maximum emission wavelength.
^{f}Fluorescence quantum yield (error limit within ±5).
^{g}Probe with diene linker.

### Fluorescence Lifetime Measurements

Time correlated single photon counting (TCSPC) measurements of fluorescence lifetime decays for 100 μM solutions of dyes (6, 7c, 7e, 7f, and 7g) in ACN/PBS buffer (50:50 v/v) were collected with the PTI Quantamaster™ 40 using a pulsed LED with a maximum emission at 340 nm. Fluorescence emission was collected at the indicated wavelength for each dye with 20 nm slit widths. The instrument response function (IRF) was collected under identical conditions. Data analysis was performed with FluoFit software (PicoQuant GmbH; Berlin, Germany) using an exponential decay model. Lifetime values are reported in Tables 3-4.

TABLE 4
Fluorescence lifetime measurements of 6 and selected probes 7c,
7e, 7f, and 7g in ACN/PBS buffer (50:50 v/v) and fit χ² values^{a}

| Compound | Lifetime (ns) | χ² |
|---|---|---|
| 6 | 2.32 ± 0.01 | 1.099 |
| 7c | 1.07 ± 0.003 | 1.017 |
| 7e | 1.02 ± 0.004 | 0.942 |
| 7f | 1.28 ± 0.004 | 1.089 |
| 7g | 1.31 ± 0.004 | 0.932 |

^{a}TCSPC data were collected at 540 nm emission for 7c, 7e, 7f, 7g and 464 nm emission for 6.
Excitation wavelength 340 nm.

### α-Synuclein Production

α-Synuclein (αS) was expressed and purified. Human αS with a C-terminal intein-His₆ fusion was transformed into Escherichia coli (E. coli) BL21 cells and plated on ampicillin plates (100 μg/mL). A single colony was then inoculated into a 5 ml primary culture containing ampicillin (100 μg/mL) in Luria-Bertain (LB) media and grown for 5-6 h with shaking (250 rpm) at 37° C. The primary culture was then transferred to 1 L LB containing ampicillin (100 μg/mL) and grown until reaching an optical density (OD₆₀₀) of 0.8-1.0. At this stage, protein production was induced by the addition of isopropyl β-D-1-thiogalactopyranoside (IPTG) to 1 mM final concentration, and the culture was grown with shaking (250 rpm) overnight at 18° C. The following day, cells were harvested by centrifugation at 4° C. for 20 min at 4000 rpm (Sorvall GS3 rotor). Pellets were then re-suspended in 20 mL/L culture of 40 mM Tris, pH 8.3 supplemented with EDTA-free protease inhibitor tablets (Pierce Biotechnology; Waltham, MA, USA) and transferred to a metal cup for sonication. Cells were lysed by sonication on ice with a Q700 probe sonicator (QSonica LLC; Newtown, CT, USA) with the following settings: Amplitude 50, Process Time 2-3 min, Pulse-ON Time 1 s, Pulse-OFF Time 1 s. Crude lysate was then transferred to 50 mL centrifugation tubes and clarified via centrifugation at 14,000 rpm for 45 min (Sorvall SS34 rotor). Following centrifugation, supernatant was removed and transferred to a 50 mL Falcon tube. Then, 5 ml of nickel agarose resin (GoldBio; St. Louis, MO, USA) was added, and the lysate-nickel mixture was incubated with nutation at 4° C. for 1 h. The lysate-nickel mixture was then poured into a 20 mL fritted column, and the flowthrough was saved. The remaining resin was then washed with ˜20 mL Wash Buffer 1 (50 mM HEPES buffer, pH 7.5), ˜20 mL Wash Buffer 2 (50 mM HEPES, 5 mM imidazole, pH 7.5), and eluted with 12 mL Elution Buffer (50 mM HEPES, 300 mM imidazole, pH 7.5). Then 2-mercaptoethanol (Bio-Rad Laboratories; Hercules, CA, USA) was added to crude lysate (200 mM final concentration), and the mixture was allowed to incubate with nutation at room temperature overnight. The resulting cleaved protein was dialyzed against 20 mM Tris pH 8.0 for 8-10 h. The resulting dialysate was then treated with 5 mL nickel agarose resin and incubated with nutation at 4° C. for 1-2 h. The mixture was then applied to a 20 mL fritted column and flowthrough containing αS was collected in a 15 mL Falcon tube. The resulting enriched protein mixture was then dialyzed against 20 mM Tris, pH 8.0 overnight and purified via FPLC using a 5 mL HiTrap Q-HP column (Cytiva; Marlborough, MA, USA) using the following method: Buffer A: 20 mM Tris, pH 8.0; Buffer B: 20 mM Tris, 1 M NaCl, pH 8.0; Gradient: 0% Buffer B—5 column volumes, 0-10% Buffer B—5 column volumes, 20-30% Buffer B—20 column volumes, 30-100% Buffer B—10 column volumes; flow rate 3 mL/min. The resulting fractions were then assessed for purity via MALDI MS, and pure fractions were combined. Protein was then concentrated, and buffer exchanged into PBS (NaCl 137 mM, KCl 2.7 mM, Na₂HPO₄ 10 mM, KH₂PO₄ 1.8 mM) to a final concentration of 100-200 μM via Amicon 3 kDa MWCO filters (Millipore Sigma; St. Louis, MO, USA). Purified protein was aliquoted into 1.5 mL tubes and stored at −80° C. until further use. MALDI MS [M+H]⁺ calcd: 14460, found: 14457.

### Tau 1N4R Protein Production

Tau (1N4R) (Tau) was expressed and purified. Using a fresh transformation of Tau 1N4R plasmid transformed into E. coli BL21 cells and plated on ampicillin plates (100 μg/mL), single colony was inoculated into a 5 mL primary culture containing ampicillin (100 μg/ml) in LB media and grown for 5-6 h with shaking (250 rpm) at 37° C. The primary culture was then transferred to 1 L LB containing ampicillin (100 μg/ml) and grown until reaching an OD₆₀₀ of 0.4-0.6. At this stage, protein production was induced by the addition of IPTG to 1 mM final concentration, and the culture was grown with shaking (250 rpm) overnight at 16° C. The following day, cells were harvested by centrifugation at 4° C. for 20 min at 4000 rpm (Sorvall GS3 rotor). Pellets were then re-suspended in 15 mL Ni-NTA Buffer A (50 mM Tris pH 8, 500 mM NaCl, and 10 mM Imidazole) with 1 mg/mL (chicken egg white) lysozyme, 1 tablet of EDTA-free protease inhibitor and 1 mM PMSF. The suspension was sonicated on ice for 1 minute 40 seconds, 1 second on/2 seconds off, power set to 50 W. Cellular debris was then transferred to 50 mL centrifugation tubes and clarified via centrifugation at 20,000×g for 30 min (Sorvall SS34 rotor). Following centrifugation, supernatant was removed and filtered with a 0.22 μm syringe filter into a 50 mL Falcon tube. Then, 5-7 mL of nickel agarose resin was prepared and equilibrated with ˜30 mL of Ni Buffer A. Then supernatant-nickel mixture was incubated with nutation at 4° C. for 1 h. Supernatant-nickel mixture was then poured into a 20 mL fritted column, and the flowthrough was saved. The remaining resin was then washed with ˜30 mL Ni-NTA Buffer A and eluted with ˜15 mL Ni-NTA Buffer B (50 mM Tris pH 8, 500 mM NaCl, and 400 mM imidazole). The nickel column was cleaned by running more Ni-NTA Buffer B through the column and then re-equilibrated with Ni-NTA Buffer A. The elution was then concentrated to ˜1 mL in an Amicon concentrator (10 kDa MWCO) and the following were added 25 μM TEV, and 1 mM DTT from fresh 1 M DTT stock. The mixture was allowed to incubate with nutation at 4° C. overnight and then the buffer was exchanged back to Ni-NTA Buffer A (2 cycles of 15 mL) using an Amicon concentrator. The resulting dialysate was then treated with 5-7 mL nickel agarose resin and incubated with nutation at 4° C. for 1 h. The mixture was then applied to a 20 mL fritted column and flowthrough containing 1N4R tau was collected in a 15 mL Falcon tube. The resulting enriched protein mixture was then concentrated, and buffer exchanged two times into Ni-NTA Buffer C (25 mM Tris pH 8, 100 mM NaCl, 1 mM EDTA, and 1 mM TCEP) using an Amicon concentrator and concentrated down to 1 mL before filtering the solution using a 0.22 μm filter. Then, the protein was purified via FPLC using a size exclusion column (S200, HiLoadc16/60cSuperdex 200 μg) using the following method: isocratic elution: 100% Ni-NTA Buffer C,—1.2 column volumes with flowrate 0.5 mL/min. The purity of the fractions was checked by SDS-PAGE gel and the clean fractions were combined, concentrated, and the buffer exchanged into PBS to a final concentration of 100-200 μM via Amicon 10 kDa MWCO filters and stored at −80° C. until further use. MALDI MS [M+H]⁺ calcd: 43111, found: 43109.

### αS Fibril Preparation

100 μM final concentration of αS monomer in PBS buffer (500 μL, 1.5 mL tube) at pH 7 was sealed with Teflon tape followed by parafilm and then incubated at 37° C. with shaking at 1300 rpm for 7 days in an IKA MS3 control orbital shaker (Wilmington, NC, USA) to get αS fibrils.

### 1N4R Tau Fibril Preparation

50 μM final concentration of 1N4R tau monomer in PBS buffer (200 μL) at pH 7.4 along with 100 μM of DTT and 12.5 μM of heparin was sealed with Teflon tape followed by parafilm incubated at 37° C. with shaking at 1300 rpm for 72 h in an IKA MS3 control orbital shaker (Wilmington, NC, USA) to get tau fibrils.

### Aβ₁₋₄₂ Fibril Preparation

Using 1 mg of the commercially available Aβ₁₋₄₂ ($-Amyloid (1-42), human, Genscript Cat. No. RP10017) 1 mg, a 1 mM stock solution was made by adding 222 μL hexafluoroisopropanol (HFIP) directly to the vial containing lyophilized powder through the rubber septum. After the peptide completely dissolved, the septum was pierced with a syringe needle to release the vacuum. The Aβ₁₋₄₂-HFIP solution was incubated for 30 min at room temperature. Using a positive displacement pipette, 100 μL aliquots of the solution (0.45 mg) were transferred into Eppendorf tubes and the HFIP was allowed to evaporate in the open tubes in a fume hood. The tubes were then dried on a vacuum centrifuge for 1 h without heating to remove any remaining trace amount of HFIP. To make a 1 mM stock solution of Aβ₁₋₄₂ fibrils in 10 mM Phosphate buffer at pH 7.4, 0.45 mg of Aβ₁₋₄₂ was dissolved in 10 μL DMSO with addition of 10 μL of 10 mM of NaOH and sealed in an Eppendorf tube with Teflon tape followed by parafilm, then incubated at 37° C. with shaking at 500 rpm for 5 days in an IKA MS3 control orbital shaker (Wilmington, NC, USA).

### Gel-Based Quantification of Protein in Fibrils

Following completion of aggregation, fibrils were pelleted by centrifugation in a bench top centrifuge at 13,200 rpm for 90 min at 4° C. The supernatant was then carefully removed, and the fibril pellet resuspended in aggregation buffer with vortexing. Then 10 μL from each sample was transferred into an individual 0.6 mL Eppendorf tube and 2 μL 150 mM SDS in water was added (final concentration: 25 mM SDS). The tubes were then capped and heated to −100° C. for 15-20 min. Then the tubes were placed on ice to cool for 5-10 min, and 3 μL 4× gel loading was added and the supernatant and pellet were run on an SDS-PAGE along with a monomer standard at a known concentration. After running the gel, the bands were quantified by ImageJ software to estimate the percentage in fibrils (pellet). This was used with the initial monomer concentration in the aggregation reactions to determine the concentration of fibrils for dye binding experiments.

Probe Binding (K_{d}) Measurements with αS Fibrils

αS fibrils (100 μM) in PBS buffer were incubated with varying 7k, 7l, and 7m concentrations (0, 0.01, 0.1, 0.5, 1, 3, 5, 7, and 10 μM) from concentrated 10 mM DMSO stock solutions in Greiner 96 well flat black 12 area plates at 37° C. with shaking at 500 rpm for 15 min in an IKA MS3 control orbital shaker (Wilmington, NC, USA). After 15 min incubation, fluorescence intensity measurements were obtained with a Tecan Spark plate reader (Mannedorf, Switzerland) by excitation with λₑₓ/λₑₘ=424/570 nm for 7k, λₑₓ/λₑₘ=463/580 nm for 7l, and λₑₓ/λₑₘ=484/620 nm for 7m, using the following parameters: excitation and emission bandwidth 5 nm, delay time 0 μs, integration time 40 μs. Averages and standard deviations were calculated from at least 3 independent measurements at each probe concentration. The resulting binding curve was fit to the following equation (modified from the One site—Total, accounting for ligand depletion model) in Graphpad Prism 9 (San Diego, CA, USA), from which the K_{d} was determined.

$$Y = \frac{a}{2} ( b - {( {b}^{2} - 4 c )}^{\frac{1}{2}} ) \qquad ( Eq . 1 )$$

$$b = {R}_{t} + X + {K}_{d} \qquad ( Eq . 2 )$$

$$C = {R}_{t} X \qquad ( Eq . 3 )$$

X=total probe added in μM, Y: measured fluorescence emission, K_{d}: Dissociation constant in μM, a: fluorescence counts/μM probe bound, and Rₜ=maximum probe occupancy (set to 3.3 μM based on Bₘₐₓ observed in prior radioligand binding studies). It is noted that variation of Rₜ from 1 to 10 does not significantly change the fitted K_{d} value (less than 2-fold change).
Fluorescence Spectra with αS Fibrils

Fluorescence measurements were performed using a PTI QuantaMaster™ 40 fluorescence spectrometer. Fluorescence spectra of 10 μM probes (concentrated stock solution prepared as 10 mM in DMSO) (7b, 7c, 7d, 7e, 7f, 7g and 7h) in PBS buffer were measured by excitation at their absorbance maximum wavelength in the absence and presence of 50 μM αS fibrils (stock solution 100 μM αS fibrils in PBS buffer).

Absorbance Spectra with αS Fibrils

Absorption measurements were performed using Thermo Scientific Genesys 150 UV Vis spectrometer. Absorption spectra of 10 μM probes (concentrated stock solution prepared as 10 mM in DMSO) (7j, 7k, 7l, and 7m) in PBS buffer were measured in the absence and presence of 50 μM αS fibrils (stock solution 100 μM αS fibrils in PBS buffer).

Excitation Spectra with αS Fibrils

Excitation spectral measurements were performed using a PTI QuantaMaster™ 40 fluorescence spectrometer. Excitation spectra of 10 μM probes (concentrated stock solution prepared as 10 mM in DMSO) (7j, 7k, 7l, and 7m) in PBS buffer were measured in the absence and presence of 50 μM αS fibrils (stock solution 100 μM αS fibrils in PBS buffer).

Fluorescent QY Measurements of 7j, 7k, 7l, and 7m with αS Fibrils

For each QY measurement, the incident excitation light spectrum was collected with 100 μL of probes (100 μM) in PBS buffer. After measuring the incident light intensity, probes (7j-7m) were added to the αS fibrils (100 μM) from a concentrated stock solution (10 mM in DMSO) to a final concentration of 100 μM and the new spectrum (fluorescence intensity and new incident light intensity) was collected. Using the JASCO Quantum Yield Software, the dye QY was calculated by dividing the dye fluorescence intensity by the difference in incident light intensity in the presence and absence of fibrils.

Fluorescence Lifetime Measurements of Probes with αS fibrils

TCSPC measurements of fluorescence lifetime decays for 100 μM dyes in the presence of 100 μM αS fibrils were collected with the PTI Quantamaster™ 40 using a pulsed LED with a maximum emission at 486 nm. Fluorescence emission was collected at the indicated wavelength for each dye with 20 nm slit widths. The IRF was collected under identical conditions. Data analysis was performed with FluoFit software using an exponential decay model.

TABLE 5
Fluorescence lifetime measurements of αS fibrils with probes
7j, 7k, 7l, and 7m in PBS buffer and fit χ² values.^{a}

| Compound | Lifetime (ns) | χ² |
|---|---|---|
| 7j | 1.90 ± 0.01 | 1.221 |
| 7k | 2.09 ± 0.01 | 1.117 |
| 7l | 2.27 ± 0.01 | 1.079 |
| 7m | 1.27 ± 0.01 | 1.161 |

^{a}Emission wavelength collected at 575 nm for 7j, 570 nm for 7k, 580 nm for 7l, and 620 nm for 7m.
Final concentrations of the probe and αS fibrils were 100 μM.

Fluorescence Analysis of 7k-7m Via Plate Reader

Experiments were performed on the Tecan Spark in Greiner 96 well flat black ½ area plates using the following parameters: excitation and emission band widths 5 nm, lag time 0 μs, integration time 40 μs. A 10 mM DMSO stock solution of probes (7j-7m) and 100 μM stock of αS fibrils were used in the study described herein.

### HEK Cell Lysate Preparation

HEK 293T cells were grown on 150 mm dishes in sterile-filtered Dulbecco's modified Eagle's medium (Gibco, Thermo Fisher) supplemented with 10% fetal bovine serum (FBS), 1% penicillin/streptomycin (Mediatech, 10 mg/ml each). Cells were then washed with PBS and harvested in 5 mL PBS by scraping. Cells were pelleted by centrifugation at 1000 rpm for 5 min, and the supernatant was removed. The resulting cell pellet was re-suspended in 300-500 μL PBS buffer with 0.1% Triton X-100 (Bio-Rad) and 1×HALT protease inhibitor cocktail without EDTA (Thermo Fisher), then lysed by sonication for two 30 s cycles (cycle 1: 2 s on 2 s off, amp 50; cycle 2: 2 s on 2 s off, amp 55) using a QSonica Q700 sonicator fitted with a microtip. Following sonication, lysate was centrifuged at 13,200 rpm for 60 min to separate membrane and cytosolic protein fractions. The resulting lysate cytosolic or membrane fraction was then used immediately after concentration determination by DC protein assay (Bio-Rad).

Selectivity of Probes (7k, 7l, and 7m) Binding with αS Fibrils in HEK Cell Lysate

Probes in PBS buffer (1 μM, the stock solution prepared as 10 mM in DMSO) were incubated with different αS fibril concentrations (0, 5, 10, 25 and 50 μM) in the presence of 10 mg/mL HEK cell lysate in Greiner 96 well flat black ½ area plates at 37° C. with shaking at 500 rpm for 15 min in an IKA MS3 control orbital shaker. After 15 min incubation, fluorescence intensity measurements were obtained with a Tecan Spark plate reader (Mannedorf, Switzerland) by excitation with λₑₓ/λₑₘ=424/570 nm for 7k, λₑₓ/λₑₘ=463/580 nm for 7l, λₑₓ/λₑₘ=484/620 nm for 7m, using the following parameters: excitation and emission bandwidth 5 nm, delay time 0 μs, integration time 40 μs.

Fluorescence Spectra with Tau Fibrils

Fluorescence measurements were performed using a PTI QuantaMaster™ 40 fluorescence spectrometer. Fluorescence spectra of 5 μM probes (concentrated stock solution prepared as 10 mM in DMSO) (7j, 7k, 7l, 7m, and ThT) in PBS buffer were measured in the absence and presence of 25 μM tau fibrils (stock solution 32 μM tau fibrils in PBS buffer).

Excitation Spectra with Tau Fibrils

Excitation spectral measurements were performed using a PTI QuantaMaster™ 40 fluorescence spectrometer. Excitation spectra of 5 μM probes (concentrated stock solution prepared as 10 mM in DMSO) (7j, 7k, 7l, 7m, and ThT) in PBS buffer were measured in the absence and presence of 25 μM tau fibrils (stock solution 32 μM tau fibrils in PBS buffer).

Fluorescence Spectra with Aβ₁₋₄₂ Fibrils

Fluorescence measurements were performed using a PTI QuantaMaster™ 40 fluorescence spectrometer. Fluorescence spectra of 10 μM probes (concentrated stock solution prepared as 10 mM in DMSO) (7j, 7k, 7l, 7m, and ThT) in PBS buffer were measured in the absence and presence of 50 μM Aβ₁₋₄₂ fibrils (stock solution 1 mM Aβ₁₋₄₂ in 10 mM phosphate buffer, pH 7.4).

Excitation Spectra with Aβ₁₋₄₂ Fibrils

Excitation spectral measurements were performed using a PTI QuantaMaster™ 40 fluorescence spectrometer. Excitation spectra of 10 μM probes (concentrated stock solution prepared as 10 mM in DMSO) (7j, 7k, 7l, 7m, and ThT) in PBS buffer were measured in the absence and presence of 50 μM Aβ₁₋₄₂ fibrils (stock solution 1 mM Aβ₁₋₄₂ in phosphate buffer).

Preparation of Sarkosyl-Insoluble αS from Disease and Control Brains

All procedures were performed in accordance with local institutional review board guidelines. Written informed consent for autopsy and analysis of tissue sample data was obtained either from patients themselves or their next of kin. Frozen postmortem human frontal cortex brain tissues from three patients diagnosed with Alzheimer's disease (AD) and three patients diagnosed with Parkinson's disease with dementia (PDD) were selected for sequential extraction of αS aggregates based on a high burden of αS pathology determined by immunohistochemical staining. In brief, 5-10 g of frontal cortical gray matter were homogenized in five volumes (W/V) of high-salt (HS) buffer (50 mM Tris-HCl pH 7.4, 750 mM NaCl, 10 mM NaF, 5 mM EDTA) with protease and protein phosphatase inhibitors, incubated on ice for 20 min and centrifuged at 100,000×g for 30 min. The pellets were then re-extracted with HS buffer, followed by sequential extractions with five volumes of 1% Triton X-100-containing HS buffer and 1% Triton X-100-containing HS buffer with 30% sucrose. The pellets were then re-suspended and homogenized in 1% sarkosyl-containing HS buffer, rotated at room temperature for 2 h or at 4° C. overnight and centrifuged at 100,000×g for 30 min. The resulting sarkosyl-insoluble pellets were washed once with Dulbecco's PBS (DPBS) and re-suspended in DPBS by sonication (QSonica Microson XL-2000; 50 pulses; setting 2; 0.5 s per pulse). These final sarkosyl-insoluble fractions are referred to as “brain extracts.: The amount of αS in the brain extracts was determined by sandwich ELISA using Syn9027, a mono-clonal antibody (Mab) to αS, and the protein concentrations were examined by bicinchoninic acid (BCA) assay. Total protein in AD cases ranged from 5 to 13 mg/mL and total protein in PDD cases ranged from 9 to 12 mg/mL. αS concentration in AD cases ranged from 36 to 43 μg/mL and total protein in PDD cases ranged from 24 to 33 μg/mL.

In Vitro Amplification of αS Fibrils from Brain Extracts

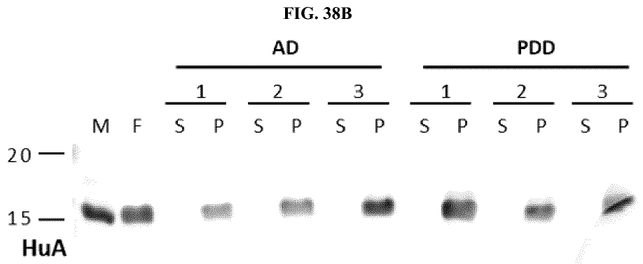

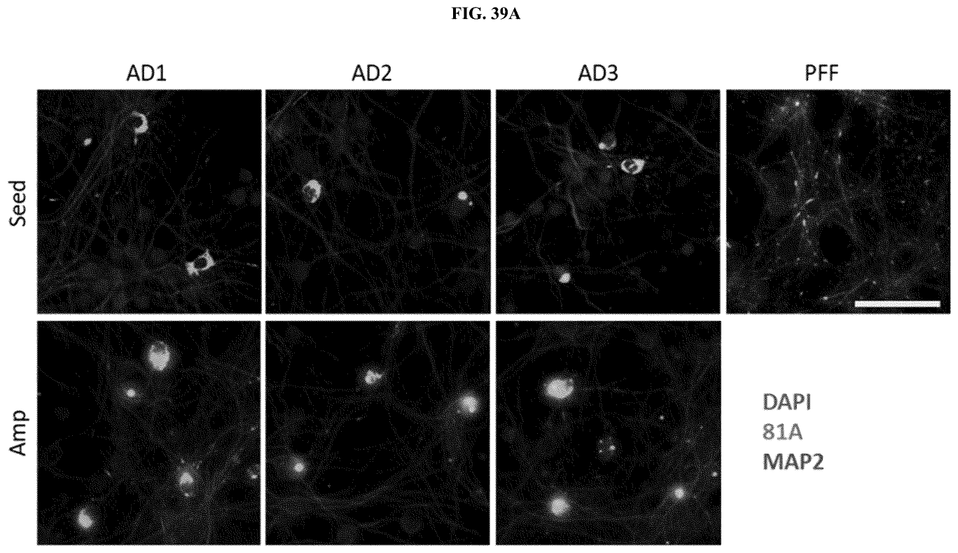

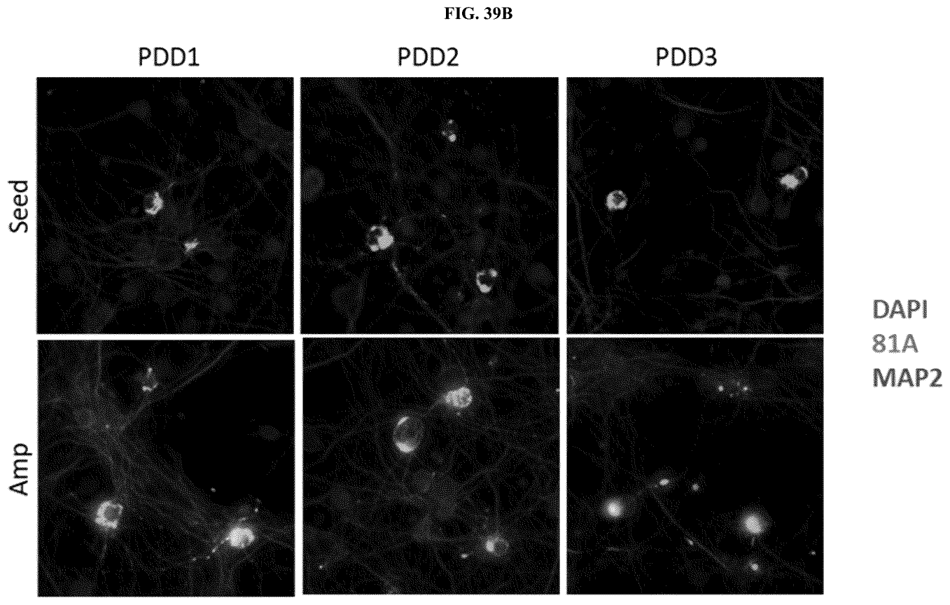

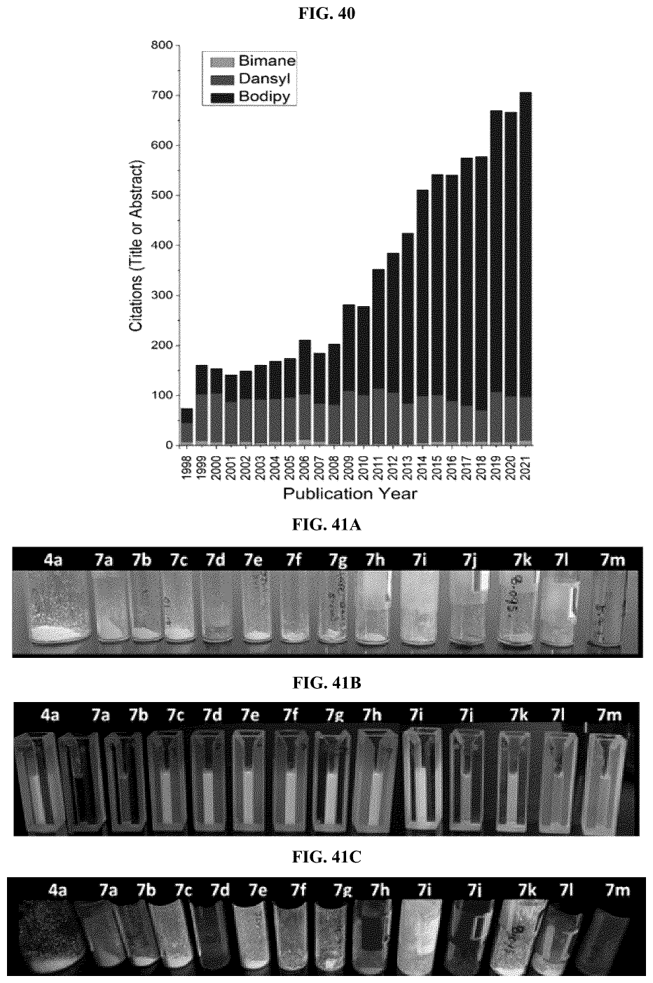

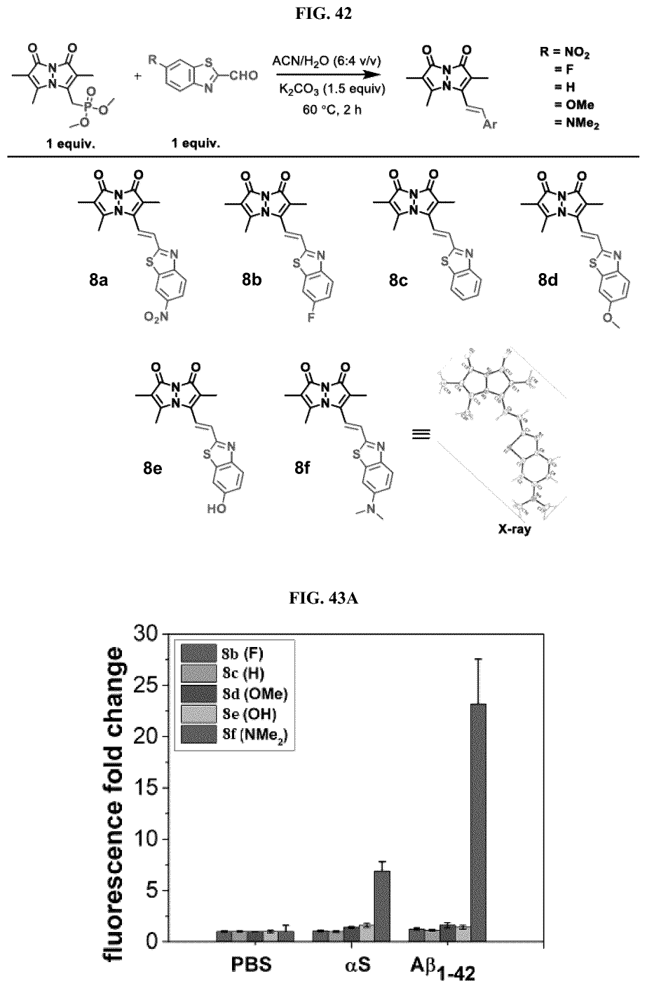

Brain-derived αS aggregates in extract samples from three AD and three PDD cases (FIG. 38A) were used as seeds for templated aggregation of recombinant αS monomer. Brain lysates were sonicated on high for 20 cycles of 30 s on, followed by 30 s off (Diagenode Biorupter UCD-300 bath sonicator) and diluted to 350 nM (based on sandwich ELISA) in DPBS (pH 7.4, without Mg²⁺ and Ca²⁺) containing sufficient monomer for a final αS concentration of 35 μM (1% patient-derived seed, 99% monomer) and incubated at 37° C. with constant shaking at 1000 rpm for 7 days. The resulting aggregates were termed amplified fibrils (AFs). After seven days of incubation, amplification progress was determined by removing an aliquot and diluting tenfold in DPBS to a final αS concentration of 3.5 μM. Samples were centrifuged at 45k rpm for 30 min at 10° C. in a TLA-55 rotor (Beckman Coulter). The supernatants were removed, and the pellets were resuspended in DPBS. Equal volumes of supernatant and pellet fractions were analyzed on SDS-PAGE gels and Western blot, with amplified aggregates migrating to the pellet fraction (FIG. 38B). Primary mouse hippocampal neurons were then treated with the AFs to confirm they retained the characteristic pathological phenotype of the original brain lysate and were distinct from recombinant PFFs. Primary mouse neurons were prepared from the hippocampus of embryonic day E16-E18 CD1 mouse embryos. Primary mouse hippocampal neurons were plated at 17.5k cells per well in 96-well plates and were treated with 50 ng per well of brain-derived, AFs, or recombinant PFFs at seven days in vitro and maintained for two weeks post-treatment. Cells were grown in Neurobasal medium supplemented with B27, 2 mM GlutaMax, and 100 U/ml penicillin/streptomycin (ThermoFisher). Following fixation with 4% paraformaldehyde, 4% sucrose, and 1% Triton-X100, cultures were probed with antibodies for MAP2 (17028) and pSer-129 αS (81A) Patient-derived fibrils and AFs induce discrete, dense inclusions in cell bodies with little neuritic pathology while PFF induce predominantly small neuritic puncta and threads (FIGS. 39A-38B).

### Control Sample Preparation

Control samples for comparison to AFs in fluorescence binding experiments below were prepared as follows. “Lysate” controls used a portion of the same brain extracts from AD cases 1-3 and PDD cases 1-3, but were not subjected to amplification. These should contain the same concentration of patient-derived αS and other insoluble proteins as the AF samples, but no seeded fibrils. Pre-formed fibril (PFF) controls took these lysates and added αS fibrils, generated as described above for binding studies of the pure fibrils, to match the αS monomer concentration used in generating AFs (35 μM). Thus, they would have the same fibril content as an amplification reaction which went to completion, but with a fibril morphology that is not templated by the patient-derived αS aggregates.

Fluorescence Measurements of 7k, 7l, 7m and ThT with AD and PDD Patient Cases

Probe binding was tested with 7 μM stock concentrations of patient tissue samples from 3 different AD patient cases (AD1, AD2, and AD3) with 3 different conditions: lysate only, amplified αS fibrils (AFs), preformed αS fibrils (PFFs), all prepared as described above. Final samples were prepared in a Greiner 384-well small volume microplate by diluting to 1 μM final concentration with PBS buffer from each condition's stock in a 5 μL final volume. To this was added bimane probes 7k, 7l, 7m and ThT at 1 μM probe concentration (10 mM stock of 7k-7m in DMSO and 5 mM stock of ThT in PBS buffer), then incubated at 37° C. with shaking at 500 rpm for 15 min in an IKA MS3 control orbital shaker. After 15 min of incubation, fluorescence intensity measurements were obtained with a Tecan Spark plate reader by excitation with λₑₓ/λₑₘ=424/570 nm for 7k, λₑₓ/λₑₘ=463/580 nm for 7l, λₑₓ/λₑₘ=484/620 nm for 7m, and λₑₓ/λₑₘ=450/482 nm for ThT, using the following parameters: excitation and emission bandwidth 5 nm, delay time 0 μs, integration time 40 μs. Identical experiments were performed for 7 μM stock concentrations of patient tissue samples from 3 different PDD patient cases (PDD1, PDD2, and PDD3) with 3 different conditions: lysate only, amplified αS fibrils (AFs), preformed αS fibrils (PFFs).

### Example 1: Compound Synthesis and Characterization

### Multi-Gram Scale Synthesis of Bimane Core

### Synthesis of 3,4-dimethyl-2-pyrazolin-5-one (2)

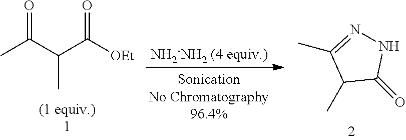

Ethyl 2-methylacetoacetate (30 g, 208.3 mmol, 1 equiv) was condensed with 4 equiv. of hydrazine (26.7 g, 833.2 mmol, 35 wt. % in H₂O) under sonication for 30 min to give a white precipitate. After filtration of the white precipitate and washing several times with ethyl acetate followed by dichloromethane to get rid of the unreacted starting materials, the precipitate was dried overnight to afford 22.485 g of compound 2 in 96.4% isolated yield. ¹H NMR and LRMS characterization data matched previous reports.

### Synthesis of 3,4-dimethyl-4-chloro-2-pyrazolin-5-one (3)

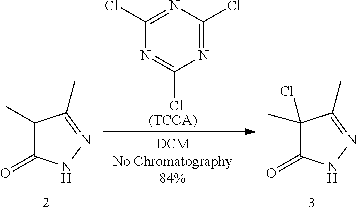

3,4-dimethyl-2-pyrazolin-5-one (2, 20 g, 178.4 mmol) was dissolved in 200 mL of dichloromethane (DCM). The solution was cooled to 0° C. Trichloroisocyanuric acid (TCCA, 13.7 g, 59.5 mmol) was slowly added to the reaction mixture over a period of 30 min. After completion of the addition, the reaction mixture was stirred overnight at room temperature and the cyanuric acid byproduct was filtered off. The filtrate was concentrated under reduced pressure at 40° C. to afford 22.05 g of 3,4-dimethyl-4-chloro-2-pyrazolin-5-one (3) in 84% isolated yield. ¹H NMR and LRMS characterization data matched previous reports.

### Synthesis of 2,3,5,6-tetramethyl-1H, 7H-pyrazolo[1,2-a]pyrazole-1,7-dione (4a)

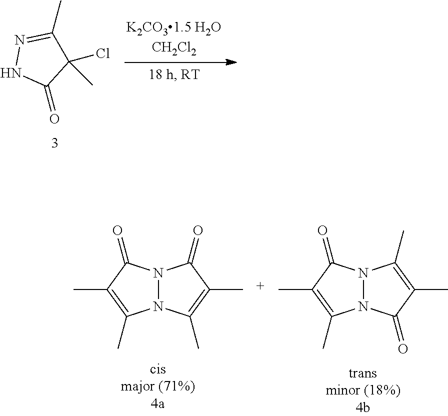

3,4-Dimethyl-4-chloro-2-pyrazolin-5-one (3, 15 g, 102.34 mmol, 1 equiv) was dissolved in 150 mL DCM and cooled to 0° C. Then add potassium carbonate hydrate (K₂CO₃·1.5H₂O) (59.32 g, 429.83 mmol, 4.2 equiv) and stirred for 18 hours at room temperature. Then the K₂CO₃ was filtered, and the filtrate concentrated to afford a mixture of cis (4a) and trans (4b) isomers of bimane. 2,3,5,6-tetramethyl-1H,7H-pyrazolo[1,2-a]pyrazole-1,7-dione, with 4a as the major product. The reaction mixture was purified by column chromatography eluting with 40-70% ethyl acetate in hexane to afford the 71% of 4a (R_{f}=0.1, 70% EtOAc in hexane) and 4b (R_{f}=0.64, 70% EtOAc in hexane) in 18% yield. ¹H NMR and LRMS characterization data matched previous reports.

Synthesis and Characterization of 6 and 7a-7m

### Synthesis of 3-(bromomethyl)-2,5,6-trimethyl-1H,7H-pyrazolo[1,2-a]pyrazole-1,7-dione (5)

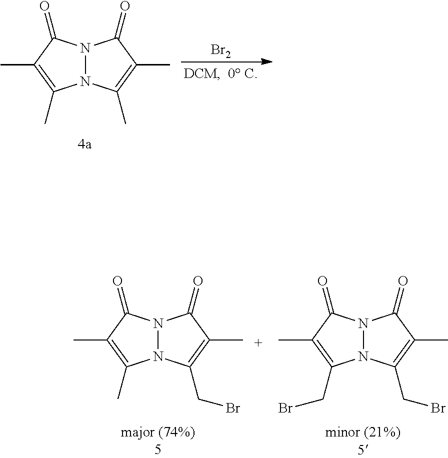

Compound 4a (3 g, 15.61 mmol) was dissolved in dry 120 mL of DCM. The solution was cooled to 0° C. and a bromine solution (0.8 mL, 15.61 mmol) in 30 mL of DCM solvent was added dropwise over a period of 30 min at 0° C. Upon completion of the addition, the reaction mixture was stirred at 0° C. for another 30 min. The progress of the reaction was monitored by TLC. Upon completion of the reaction, the resulting mixture was neutralized with cold water and then extracted with DCM. The organic layer was collected and concentrated by rotary evaporation. The crude product was purified by flash column chromatography eluting with 30-60% ethyl acetate in hexane to afford the desired monobrominated product 5 (R_{f}=0.17, 70% EtOAc in hexane) in 74% yield and the dibrominated product 5′ (R_{f}=0.45, 70% EtOAc in hexane) was also isolated in 21% yield. ¹H and ¹³C NMR and LRMS characterization data matched previous reports.

### Synthesis of dimethyl ((2,5,6-trimethyl-1,7-dioxo-1H,7H-pyrazolo[1,2-a]pyrazol-3-yl)methyl)phosphonate (6)

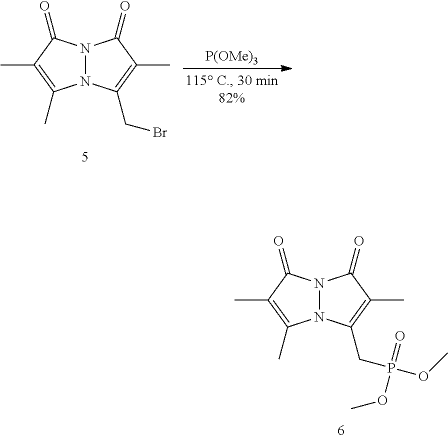

A mixture of monobromobimane (5, 2.0 g, 7.38 mmol) and trimethyl phosphite (2.85 mL) was heated at 115° C. for 30 min until it became homogeneous and then solidified. After trituration with hexane (50 mL), the mixture was slurried in ethyl acetate and filtered to remove the remaining trimethyl phosphite. Then, the solid product was redissolved in DCM and purified by flash column chromatography using 3% methanol in DCM as an eluent to afford 1.82 g of the phosphonate 3 (R_{f}=0.37, 3% methanol in DCM) as a light-yellow solid in 82% isolated yield. ¹H NMR (400 MHz, CDCl₃) δ 3.78 (d, J=11.1 Hz, 6H), 3.20 (d, J=22.0 Hz, 2H), 2.42 (s, 3H), 1.84 (d, J=4.1 Hz, 3H), 1.80 (s, 3H). ¹³C NMR (101 MHz, CDCl₃) δ 160.7, 160.2, 146.5, 140.0, 114.7, 113.2, 53.7, 25.2, 23.8, 11.9, 7.0. HRMS (ESI+) calcd for C₁₂H₁₇N₂O₅P [M+H]⁺, 301.0953; found: 301.0965.

### Horner-Wadsworth-Emmons (HWE) (General Procedure A)

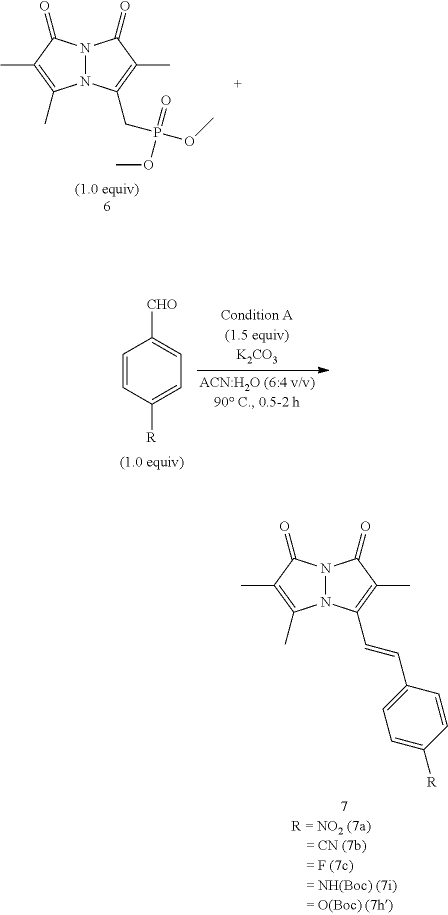

To a solution of dimethyl bimanephosphonate 6 (1 mmol) in acetonitrile (ACN)/H₂O (2 mL, 6:4 v/v), add the aryl aldehyde (1 mmol) and K₂CO₃ (2 mmol) and heat at 90° C. for 30 min-2 h. The reaction progress was checked by TLC, upon completion of the reaction, the solution was cooled to room temperature and the organic layer was extracted with DCM, washed with water (2×50 mL), dried using Na₂SO₄, and concentrated under vacuum to yield the crude mixture. Then, the product was purified by recrystallization from MeOH.

### Horner-Wadsworth-Emmons (HWE) (General Procedure B)

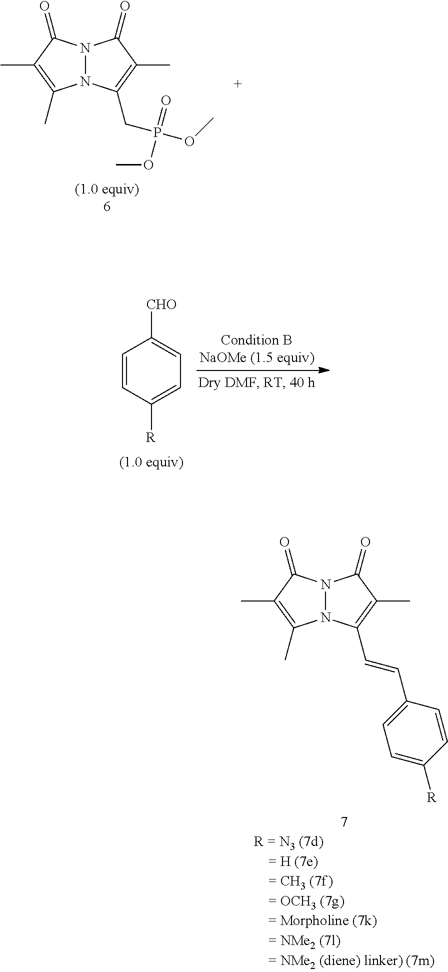

To a solution of phosphonate 6 (1 mmol) in dry DMF, the aryl aldehyde (1 mmol) and NaOMe (1.5 mmol) were added, and the resulting solution was stirred at RT for 40 h under argon atmosphere. The reaction progress was monitored by TLC, and upon completion, the organic layer was extracted with DCM, washed with water (2×50 mL), dried using Na₂SO₄, and concentrated under vacuum to yield the crude product. Then, the product was purified by flash column chromatography using 3% methanol in DCM as an eluent.

TABLE 6
Optimization of HWE reaction of 6 and 4-(dimethylamino)benzaldehydeª

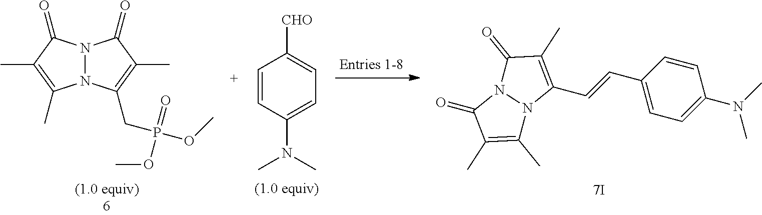

| Entry | Solvent | Base | Temp (° C.) | Time ( h) | Yield (%)^{b} |
|---|---|---|---|---|---|
| 1 | ACN/H₂O | K₂CO₃ | ~23 (RT) | 40 | 2 |
| 2 | ACN/H₂O | K₂CO₃ | 90 | 2 | 9 |
| 3 | DMF | KOtBu | ~23 (RT) | 40 | 27 |
| 4 | DMF | NaOMe | ~23 (RT) | 40 | 46 |
| 5 | DMF | NaOMe | 60 | 40 | 38 |
| 6 | DMSO | KOtBu | ~23 (RT) | 40 | 4 |
| 7 | DMSO | NaOMe | ~23 (RT) | 40 | 31 |
| 8 | DMSO | NaOMe | 60 | 40 | 12 |

^{a}Reaction conditions: 6 (1.0 equiv); aryl aldehyde 71 (1.0 equiv); KO/Bu, NaoMe, or K₂CO₃ (1.5 equiv).
^{b}Yields based on chromatogram peak areas.

### (E)-2,3,6-trimethyl-5-(4-nitrostyryl)-1H,7H-pyrazolo[1,2-a]pyrazole-1,7-dione (7a)

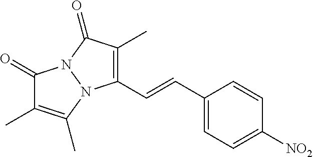 [SMILES: Cc1c(C)n2c(/C=C/c3ccc([N+](=O)[O-])cc3)c(C)c(=O)n2c1=O]

Following General Procedure A with 4-nitrobenzaldehyde (50.3 mg, 0.33 mmol, 1 equiv, purchased from Aldrich) at 90° C. for 30 min afforded 106 mg (98% isolated yield, yellow solid) of 7a. ¹H NMR (400 MHz, CDCl₃) δ 8.29 (d, J=8.9 Hz, 2H), 7.70 (d, J=8.8 Hz, 2H), 7.17 (d, J=16.5 Hz, 1H), 6.96 (d, J=17.3 Hz, 1H), 2.30 (s, 3H), 2.03 (s, 3H), 1.84 (s, 3H). ¹³C NMR (101 MHz, CDCl₃) δ 161.7, 161.3, 148.5, 146.4, 141.0, 138.5, 128.1, 124.7, 118.3, 115.2, 114.3, 12.7, 8.6, 7.2. HRMS (ESI⁺) calcd for C₁₇H₁₅N₃O₄ [M+H]⁺, 326.1141; found: 326.1156.

### (E)-4-(2-(2,5,6-trimethyl-1,7-dioxo-1H,7H-pyrazolo[1,2-a]pyrazol-3-yl)vinyl)benzonitrile (7b)

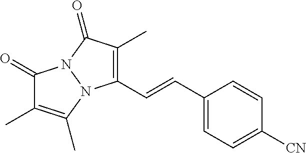 [SMILES: Cc1c(C)n2c(/C=C/c3ccc(C#N)cc3)c(C)c(=O)n2c1=O]

Following General Procedure A with 4-cyanobenzaldehyde (43.6 mg, 0.33 mmol, 1 equiv, purchased from Aldrich) at 90° C. for 1 h afforded 85.3 mg (83.6% isolated yield, yellow solid) of 7b. ¹H NMR (400 MHz, CDCl₃) δ 7.73 (d, J=8.4 Hz, 2H), 7.63 (d, J=8.5 Hz, 2H), 7.12 (d, J=16.5 Hz, 1H), 6.91 (d, J=16.5 Hz, 1H), 2.28 (s, 3H), 2.02 (s, 3H), 1.84 (s, 3H). ¹³C NMR (101 MHz, CDCl₃) δ 161.7, 161.3, 148.6, 146.6, 139.1, 139.0, 133.1, 127.9, 118.4, 117.6, 115.0, 114.2, 113.5, 12.7, 8.6, 7.2. HRMS (ESI⁺) calcd for C₁₈H₁₅N₃O₂ [M+H]⁺, 306.1243; found: 306.1235.

### (E)-3-(4-fluorostyryl)-2,5,6-trimethyl-1H,7H-pyrazolo[1,2-a]pyrazole-1,7-dione (7c)

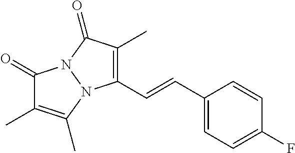 [SMILES: Cc1c(C)n2c(/C=C/c3ccc(F)cc3)c(C)c(=O)n2c1=O]

Following General Procedure A with 4-fluorobenzaldehyde (41.3 mg, 0.33 mmol, 1 equiv, purchased from Aldrich) at 90° C. for 2 h afforded 77.2 mg (78% isolated yield, yellow solid) of 7c. ¹H NMR (400 MHz, CDCl₃) δ 7.52 (dd, J=8.9, 5.3 Hz, 2H), 7.13 (t, J=8.6 Hz, 2H), 7.07 (d, J=16.4 Hz, 1H), 6.71 (d, J=16.5 Hz, 1H), 2.28 (s, 3H), 2.01 (s, 3H), 1.83 (s, 3H). ¹³C NMR (101 MHz, CDCl₃) δ 164.0 (d, ¹J_{C-F}=252.7 Hz), 161.8, 161.6, 148.6, 147.5, 140.1, 131.2, 129.4, 129.3, 116.6 (d, ²J_{C-F}=22.2 Hz), 113.9, 113.7, 12.7, 8.6, 7.2. HRMS (ESI⁺) calcd for C₁₇H₁₅FN₂O₂ [M+H]⁺, 299.1196; found: 299.1193.

### (E)-3-(4-azidostyryl)-2,5,6-trimethyl-1H,7H-pyrazolo[1,2-a]pyrazole-1,7-dione (7d)

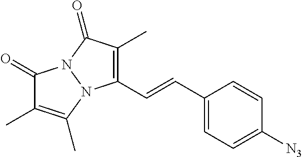 [SMILES: Cc1c(C)n2c(/C=C/c3ccc(N=[N+]=[N-])cc3)c(C)c(=O)n2c1=O]

Following General Procedure B with 4-azidobenzaldehyde (49 mg, 0.33 mmol, 1 equiv, purchased from Aldrich) at room temperature for 40 h afforded 43 mg (40% isolated yield, dark yellow solid) of product 7d after purification by column chromatography (3% methanol in dichloromethane, R_{f}=0.49). ¹H NMR (400 MHz, CDCl₃) δ 7.52 (d, J=8.6 Hz, 2H), 7.16-6.98 (m, 3H), 6.73 (d, J=16.5 Hz, 1H), 2.28 (s, 3H), 2.00 (s, 3H), 1.82 (s, 3H). ¹³C NMR (101 MHz, CDCl₃) δ 161.8, 161.6, 148.7, 147.6, 142.0, 140.2, 131.7, 129.0, 119.9, 113.8, 113.5, 113.5, 12.7, 8.6, 7.1. HRMS (ESI+) calcd for C₁₇H₁₅N₅O₂ [M+H]+, 322.1304; found: 322.1323.

### (E)-2,3,6-trimethyl-5-styryl-1H,7H-pyrazolo[1,2-a]pyrazole-1,7-dione (7e)

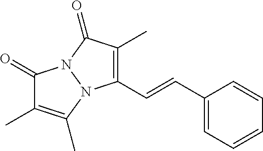 [SMILES: Cc1c(C)n2c(/C=C/c3ccccc3)c(C)c(=O)n2c1=O]

Following General Procedure A with benzaldehyde (35.3 mg, 0.33 mmol, 1 equiv, purchased from Aldrich) at room temperature for 40 h afforded 67 mg (72.4% isolated yield, yellow solid) of 7e after purification by column chromatography (3% methanol in dichloromethane, R_{f}=0.46). ¹H NMR (400 MHz, CDCl₃) δ 7.53 (dd, J=7.9, 1.9 Hz, 2H), 7.48-7.39 (m, 3H), 7.11 (d, J=16.5 Hz, 1H), 6.79 (d, J=16.4 Hz, 1H), 2.29 (s, 3H), 2.02 (s, 3H), 1.84 (s, 3H). ¹³C NMR (101 MHz, CDCl₃) δ 161.9, 161.7, 148.6, 147.7, 141.4, 135.0, 130.4, 129.4, 127.5, 114.0, 113.9, 113.8, 12.7, 8.6, 7.2. HRMS (ESI+) calcd for C₁₇H₁₆N₂O₂ [M+H]+, 281.1290; found: 281.1283.

### (E)-2,3,6-trimethyl-5-(4-methylstyryl)-1H,7H-pyrazolo[1,2-a]pyrazole-1,7-dione (7f)

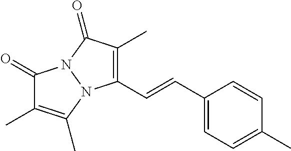 [SMILES: Cc1ccc(/C=C/c2c(C)c(=O)n3c(=O)c(C)c(C)n23)cc1]

Following General Procedure B with p-tolualdehyde (40 mg, 0.33 mmol, 1 equiv, purchased from Aldrich) at room temperature for 40 h afforded 67 mg (68% isolated yield, yellow solid) of 7f after purification by column chromatography (3% methanol in dichloromethane, R_{f}=0.47). ¹H NMR (400 MHz, CDCl₃) δ 7.45 (d, J=8.1 Hz, 2H), 7.28 (d, J=2.8 Hz, 2H), 7.10 (d, J=16.5 Hz, 1H), 6.76 (d, J=16.4 Hz, 1H), 2.43 (s, 3H), 2.31 (s, 3H), 2.04 (s, 3H), 1.86 (s, 3H). ¹³C NMR (101 MHz, CDCl₃) δ 161.9, 161.7, 148.7, 148.0, 141.4, 140.9, 132.2, 130.1, 127.4, 113.9, 113.4, 112.9, 77.5, 77.2, 76.9, 21.7, 12.7, 8.6, 7.2. HRMS (ESI⁺) calcd for C₁₈H₁₈N₂O₂ [M+H]⁺, 295.1447; found: 295.1447.

### (E)-3-(4-methoxystyryl)-2,5,6-trimethyl-1H,7H-pyrazolo[1,2-a]pyrazole-1,7-dione (7g)

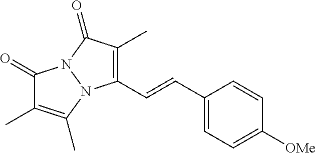 [SMILES: COc1ccc(/C=C/c2c(C)c(=O)n3c(=O)c(C)c(C)n23)cc1]

Following General Procedure B with p-anisaldehyde (45.3 mg, 0.33 mmol, 1 equiv, purchased from Aldrich) at room temperature for 40 h afforded 61 mg (59% isolated yield, yellow solid) of 7g after purification by column chromatography (3% methanol in dichloromethane, R_{f}=0.45). ¹H NMR (400 MHz, CDCl₃) δ 7.47 (d, J=8.8 Hz, 2H), 7.05 (d, J=16.5 Hz, 1H), 6.95 (d, J=8.8 Hz, 2H), 6.63 (d, J=17.1 Hz, 1H), 3.86 (s, 3H), 2.28 (s, 3H), 2.00 (s, 3H), 1.83 (s, 3H). ¹³C NMR (101 MHz, CDCl₃) δ 161.7, 161.6, 161.3, 148.5, 148.0, 140.9, 128.9, 127.5, 114.6, 113.6, 112.7, 111.3, 55.5, 12.5, 8.4, 7.0. HRMS (ESI⁺) calcd for C₁₈H₁₈N₂O₃ [M+H]+⁺, 311.1396; found: 311.1408.

### (E)-3-(4-hydroxystyryl)-2,5,6-trimethyl-1H,7H-pyrazolo[1,2-a]pyrazole-1,7-dione (7h)

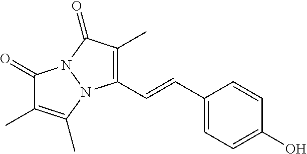 [SMILES: Cc1c(C)n2c(/C=C/c3ccc(O)cc3)c(C)c(=O)n2c1=O]

Following General Procedure A with tert-butyl 4-formylphenyl carbonate (74 mg, 0.33 mmol, 1 equiv, purchased from Aldrich) at 90° C. for 2 h afforded 100.3 mg (76% isolated yield) of tert-butyloxycarbonyl (Boc) protected 7h′ after purification by column chromatography (3% methanol in dichloromethane, R_{f}=0.41).

Next, the Boc deprotection of 7h′ (100 mg, 0.252 mmol, 1 equiv) was carried out under acidic conditions using trifluoroacetic acid (431.4 mg, 3.78 mmol, 15 equiv) in 2 mL DCM solvent for 6 h. The reaction progress was monitored by the TLC and upon completion, it was dried under vacuum to afford 55.3 mg of the 7h (74% isolated yield, yellow solid, R_{f}=0.34 in 3% methanol in dichloromethane). ¹H NMR (600 MHz, DMSO) δ 9.97 (s, 1H), 7.59 (d, J=8.7 Hz, 2H), 7.20 (d, J=16.5 Hz, 1H), 6.97 (d, J=16.5 Hz, 1H), 6.83 (d, J=8.7 Hz, 2H), 2.34 (s, 3H), 1.91 (s, 3H), 1.74 (s, 3H). ¹³C NMR (151 MHz, DMSO) δ 161.1, 160.9, 159.4, 150.1, 148.8, 141.2, 129.5, 126.3, 115.8, 111.8, 110.6, 110.5, 12.1, 8.0, 6.6. HRMS (ESI⁺) calcd for C₁₇H₁₆N₂O₃ [M+H]⁺, 297.1239; found: 297.1233.

### tert-butyl (E)-(4-(2-(2,5,6-trimethyl-1,7-dioxo-1H,7H-pyrazolo[1,2-a]pyrazol-3-yl)vinyl)phenyl)carbamate (7i)

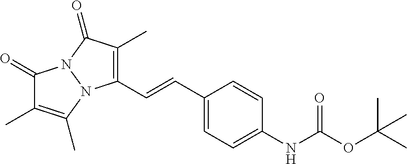 [SMILES: Cc1c(C)n2c(/C=C/c3ccc(NC(=O)OC(C)(C)C)cc3)c(C)c(=O)n2c1=O]

Following General Procedure A with 4-(Boc-amino)benzaldehyde (73.6 mg, 0.33 mmol, 1 equiv) at 90° C. for 2 h afforded 113.5 mg (86% isolated yield, yellow solid) of 7i after purification by column chromatography (3% methanol in dichloromethane, R_{f}=0.42). ¹H NMR (600 MHz, CDCl₃) δ 7.45 (s, 4H), 7.04 (d, J=16.4 Hz, 1H), 6.73-6.60 (m, 2H), 2.28 (s, 3H), 2.02 (s, 3H), 1.84 (s, 3H), 1.53 (s, 9H). ¹³C NMR (151 MHz, CDCl₃) δ 161.9, 161.8, 152.5, 148.7, 148.0, 129.6, 128.4, 118.8, 113.9, 113.2, 112.2, 81.4, 28.5, 12.7, 8.6, 7.2. HRMS (ESI+) calcd for C₂₂H₂₅N₃O₄ [M+H]⁺, 396.1923; found: 396.1930.

### (E)-3-(4-aminostyryl)-2,5,6-trimethyl-1H,7H-pyrazolo[1,2-a]pyrazole-1,7-dione (7j)

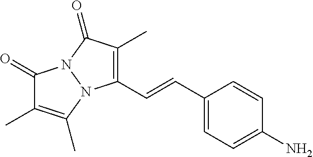 [SMILES: Cc1c(C)n2c(/C=C/c3ccc(N)cc3)c(C)c(=O)n2c1=O]

The deprotection of a Boc-protected 7i (100 mg, 0.253 mmol, 1 equiv) was carried out under acidic conditions using trifluoroacetic acid (432.5 mg, 3.79 mmol, 15 equiv) in 2 mL DCM solvent for 6 h. After completion of the reaction, the reaction progress was monitored by the TLC and concentrated the solvent and dried under vacuum to afford 62.8 mg of 7j (84% isolated yield, red solid, R_{f}=0.39). ¹H NMR (600 MHz, DMSO) δ 7.43 (d, J=8.6 Hz, 2H), 7.13 (d, J=16.3 Hz, 1H), 6.81 (d, J=16.3 Hz, 1H), 6.60 (d, J=8.5 Hz, 2H), 5.76 (s, 2H), 2.35 (s, 3H), 1.91 (s, 3H), 1.74 (s, 3H). ¹³C NMR (151 MHz, DMSO) δ 161.6, 161.5, 151.7, 150.5, 149.9, 142.6, 130.0, 122.9, 114.1, 112.2, 109.8, 108.0, 12.6, 8.6, 7.0. HRMS (ESI⁺) calcd for C₁₇H₁₇N₃O₂, [M+H]⁺, 296.1399; found: 296.1405.

### (E)-2,3,6-trimethyl-5-(4-morpholinostyryl)-1H,7H-pyrazolo[1,2-a]pyrazole-1,7-dione (7k)

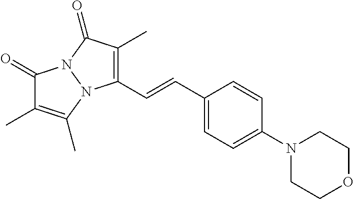 [SMILES: Cc1c(C)n2c(/C=C/c3ccc(N4CCOCC4)cc3)c(C)c(=O)n2c1=O]

Following General Procedure B with 4-(4-morpholinyl)benzaldehyde (63.4 mg, 0.33 mmol, 1 equiv, purchased from Aldrich) at room temperature for 40 h afforded 72.4 mg (59.5% isolated yield, orange solid) of the title compound after purification by column chromatography (3% methanol in dichloromethane, R_{f}=0.44). ¹H NMR (400 MHz, CDCl₃) δ 7.44 (d, J=8.9 Hz, 2H), 7.03 (d, J=16.3 Hz, 1H), 6.91 (d, J=8.9 Hz, 2H), 6.60 (d, J=16.3 Hz, 1H), 3.87 (t, J=4.9 Hz, 4H), 3.26 (t, J=4.9 Hz, 4H), 2.28 (s, 3H), 2.01 (s, 3H), 1.84 (s, 3H). ¹³C NMR (101 MHz, CDCl₃) δ 162.0, 152.6, 148.7, 148.5, 141.2, 128.9, 126.0, 115.1, 113.7, 112.6, 110.4, 66.8, 48.3, 12.7, 8.6, 7.2. HRMS (ESI⁺) calcd for C₂₁H₂₃N₃O₃ [M+H]⁺, 366.1818; found: 366.1811.

### (E)-3-(4-(dimethylamino)styryl)-2,5,6-trimethyl-1H,7H-pyrazolo[1,2-a]pyrazole-1,7-dione (7l)

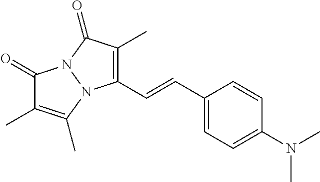 [SMILES: Cc1c(C)n2c(/C=C/c3ccc(N(C)C)cc3)c(C)c(=O)n2c1=O]

Following General Procedure B with 4-(dimethylamino)benzaldehyde (49.6 mg, 0.33 mmol, 1 equiv, purchased from Aldrich) at room temperature for 40 h afforded 46.3 mg (43% isolated yield, orange solid) of 7l after purification by column chromatography (3% methanol in dichloromethane, R_{f}=0.45). ¹H NMR (600 MHz, CDCl₃) δ 7.40 (d, J=8.9 Hz, 2H), 7.03 (d, J=16.3 Hz, 1H), 6.71 (d, J=8.9 Hz, 2H), 6.53 (d, J=16.3 Hz, 1H), 3.04 (s, 6H), 2.29 (s, 3H), 2.01 (s, 3H), 1.84 (s, 3H). ¹³C NMR (151 MHz, CDCl₃) δ 162.1, 162.0, 151.9, 149.0, 148.7, 141.8, 129.0, 122.8, 113.6, 112.2, 111.8, 108.4, 40.4, 12.8, 8.7, 7.2. HRMS (ESI⁺) calcd for C₁₉H₂₁N₃O₂, [M+H]⁺, 324.1712; found: 324.1712.

### 3-((1E,3E)-4-(4-(dimethylamino)phenyl)buta-1,3-dien-1-yl)-2,5,6-trimethyl-1H,7H-pyrazolo[1,2-a]pyrazole-1,7-dione (7m)

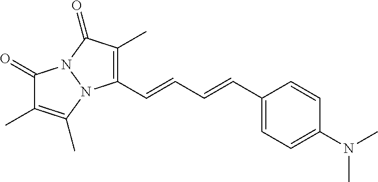 [SMILES: Cc1c(C)n2c(/C=C/C=C/c3ccc(N(C)C)cc3)c(C)c(=O)n2c1=O]

Following General Procedure B with 4-(dimethylamino)cinnamaldehyde (58.3 mg, 0.33 mmol, 1 equiv) at room temperature for 40 h afforded 51.6 mg (44.5% isolated yield, red solid) of 7m after purification by column chromatography (3% methanol in dichloromethane, R_{f}=0.48). ¹H NMR (400 MHz, CDCl₃) δ 7.37 (d, J=9.0 Hz, 2H), 6.94 (dd, J=15.1, 9.4 Hz, 1H), 6.83-6.74 (m, 2H), 6.68 (d, J=9.0 Hz, 2H), 6.23 (d, J=15.8 Hz, 1H), 3.02 (s, 6H), 2.30 (s, 3H), 2.01 (s, 3H), 1.84 (s, 3H). ¹³C NMR (151 MHz, DMSO) δ 161.0, 160.8, 150.7, 149.7, 148.3, 143.1, 139.5, 128.5, 123.7, 123.3, 114.1, 112.0, 111.6, 109.9, 39.9, 39.8, 39.6, 39.5, 39.4, 39.2, 39.1, 12.2, 8.2, 6.6. HRMS (ESI⁺) calcd for C₂₁H₂₃N₃O₂ [M+H]+, 350.1869; found: 350.1870.

TABLE 7
X-ray diffraction data for 7a

| Empirical formula | C₁₇H₁₅N₃O₄ |
|---|---|
| Formula weight | 325.32 |
| Diffractometer | Rigaku XtaLAB Synergy-S |
| Temperature/K | 100 |
| Crystal system | monoclinic |
| Space group | P2₁ |
| a | 3.85501(8)Å |
| b | 13.3190(3)Å |
| c | 14.7962(3)Å |
| β | 94.880(2)° |
| Volume | 756.96(3)Å³ |
| Z | 2 |
| d_{calc} | 1.427 g/cm³ |
| μ | 0.104 mm⁻¹ |
| F(000) | 340.0 |
| Crystal size, mm | 0.2 × 0.07 × 0.07 |
| 2θ range for data collection | 4.122-50.682° |
| Index ranges | −4 ≤ h ≤ 4, −16 ≤ k ≤ 16, −17 ≤ 1 ≤ 17 |
| Reflections collected | 19219 |
| Independent reflections | 2782[R(int) = 0.0272] |
| Data/restraints/parameters | 2782/1/220 |
| Goodness-of-fit on F² | 1.029 |
| Final R indexes | R₁ = 0.0237, wR₂ = 0.0626 |
| [I >= 2σ (I)] | |
| Final R indexes [all data] | R₁ = 0.0241, wR₂ = 0.0629 |
| Largest diff. peak/hole | 0.12/−0.15 eÅ⁻³ |
| Flack parameter | −0.3(3) |

TABLE 8
X-ray diffraction data for 7c

| Empirical formula | C₁₇H₁₅FN₂O₂ |
|---|---|
| Formula weight | 298.31 |
| Diffractometer | Rigaku XtaLAB Synergy-S |
| Temperature/K | 100 |
| Crystal system | orthorhombic |
| Space group | Fdd2 |
| a | 29.9395(3)Å |
| b | 27.5788(3)Å |
| c | 7.07968(9)Å |
| Volume | 5845.66(12)Å³ |
| Z | 16 |
| d_{calc} | 1.356 g/cm³ |
| μ | 0.818 mm⁻¹ |
| F(000) | 2496.0 |
| Crystal size, mm | 0.25 × 0.12 × 0.08 |
| 2θ range for data collection | 8.718-149.006° |
| Index ranges | −37 ≤ h ≤ 35, −33 ≤ k ≤ 34, −8 ≤ 1 ≤ 8 |
| Reflections collected | 35147 |
| Independent reflections | 2976[R(int) = 0.0563] |
| Data/restraints/parameters | 2976/1/202 |
| Goodness-of-fit on F² | 1.042 |
| Final R indexes | R1 = 0.0501, wR₂ = 0.1389 |
| [I >= 2σ (I)] | |
| Final R indexes [all data] | R1 = 0.0505, wR₂ = 0.1394 |
| Largest diff. peak/hole | 1.52/−0.24 eÅ⁻³ |
| Flack parameter | 0.09(6) |

TABLE 9
X-ray diffraction data for 7l

| Empirical formula | C₂₀H₂₅N₃O₃ | |
|---|---|---|
| Formula weight | 355.43 | |
| Diffractometer | Rigaku XtaLAB Synergy-S | |
| | (Dectris Pilatus3 R 200K) | |
| Temperature/K | 100 | |
| Crystal system | monoclinic | |
| Space group | P2₁/c | |
| a | 7.0514(6) | Å |
| b | 17.1123(10) | Å |
| c | 30.720(2) | Å |
| β | 91.352(8)° | |
| Volume | 3705.8(5) | Å³ |
| Z | 8 | |
| d_{calc} | 1.274 | g/cm³ |
| μ | 0.087 | mm⁻¹ |

| F(000) | 1520.0 |
|---|---|
| Crystal size, mm | 0.26 × 0.19 × 0.04 |
| 2θ range for data collection | 4.636-52.95° |
| Index ranges | −8 ≤ h ≤ 8, −21 ≤ k ≤ 21, −37 ≤ 1 ≤ 38 |
| Reflections collected | 49137 |
| Independent reflections | 13556[R(int) = 0.081] |
| Data/restraints/parameters | 13556/0/484 |
| Goodness-of-fit on F² | 1.047 |
| Final R indexes [I >= 2σ | R₁ = 0.0719, wR₂ = 0.1949 |
| (I)] | |
| Final R indexes [all data] | R₁ = 0.1047, wR₂ = 0.2144 |
| Largest diff. peak/hole | 0.40/−0.33 eÅ⁻³ |

### Example 2: Rational Design of Bimane Scaffold

To modulate the fluorescence of the bimane core, derivatization at the 3-position was selected, since studies with the commonly used 3-bromomethyl bimane (5) have shown that conversion to a thioether by reaction with thiols results in a turn-on of fluorescence. Thus, there was precedent for electronic communication with the bimane core by substituents at the 3-position. To access bimane derivatives, bromide 5 was synthesized as described elsewhere herein, and converted it to phosphonate ester 6 for use in Horner-Wadsworth-Emmons (HWE) reactions with diverse aldehydes (Scheme 1).

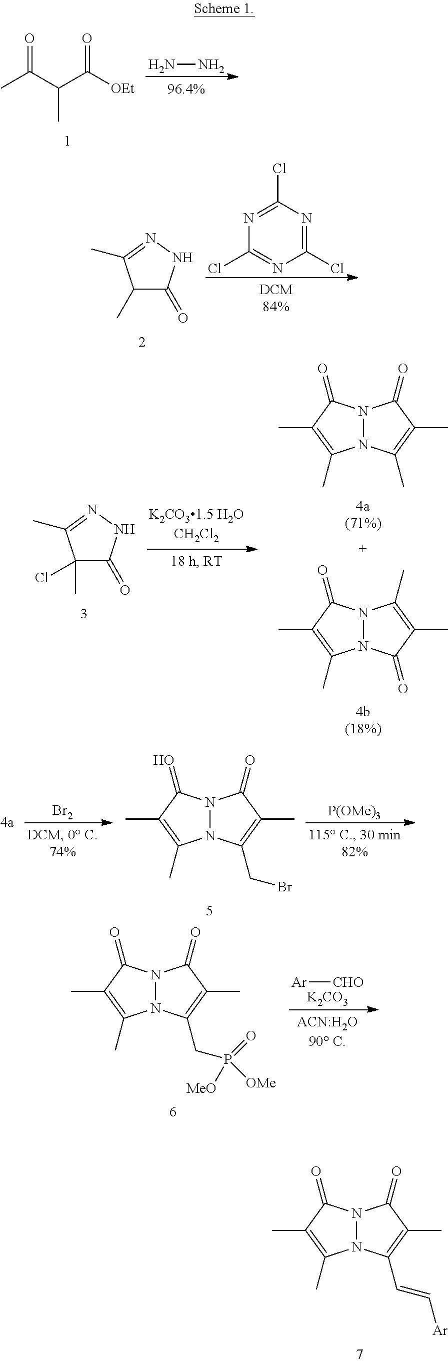

The bimane core was synthesized in three steps. Ethyl 2-methylacetoacetate (1) was condensed with 4 equiv. hydrazine under sonication to give 3,4-dimethyl-2-pyrazolin-5-one (2). After filtration to remove solids, 2 was chlorinated using TCCA to yield 3,4-dimethyl-4-chloro-2-pyrazolin-5-one (3), followed by basic treatment with aqueous K₂CO₃ in dichloromethane (DCM) under heterogeneous conditions to produce the required syn-bimane (4a), 2,3,5,6-tetramethyl-1H,7H-pyrazolo[1,2-a]pyrazole-1,7-dione, as the major product.

The synthesis of the key precursor, methyl bimane phosphonate 6, was performed in two steps, bromination followed by an Arbuzov reaction. Bimane 4a was treated with a bromine solution to afford bromobimane 5 in 74% yield and then heated with neat trimethyl phosphite to yield 6 in 82% yield.

The Horner-Wadsworth-Emmons (HWE) olefination reaction is one of the most useful methods for C═C bond formation with predominantly (E)-configuration. A range of commercially available simple aryl aldehydes, spanning electron-donating to electron-withdrawing para-substituted aryl aldehydes (Ar—CHO), were subjected to the HWE reaction with 6 in the presence of K₂CO₃ in an acetonitrile (ACN)/H₂O mixture (6:4 v/v) at reflux. All the electron-withdrawing aryl aldehydes afforded good to excellent isolated yields (>78%), but electron-rich aryl aldehydes afforded low isolated yields (<10%). The reaction completion time depended on the substitution of aryl aldehydes. Electron-poor substituents required a reaction time of only minutes compared to hours for the electron-rich substituents.

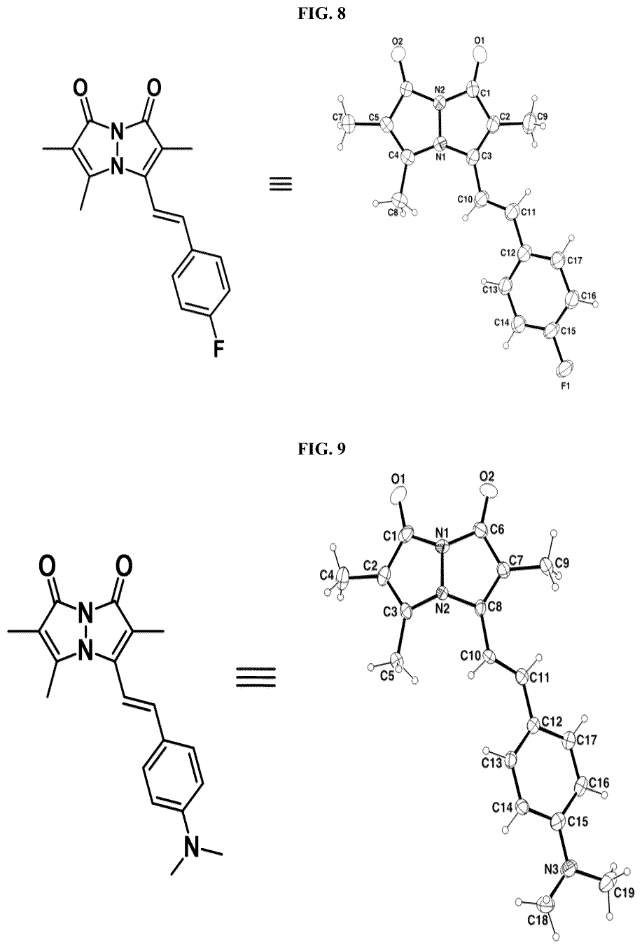

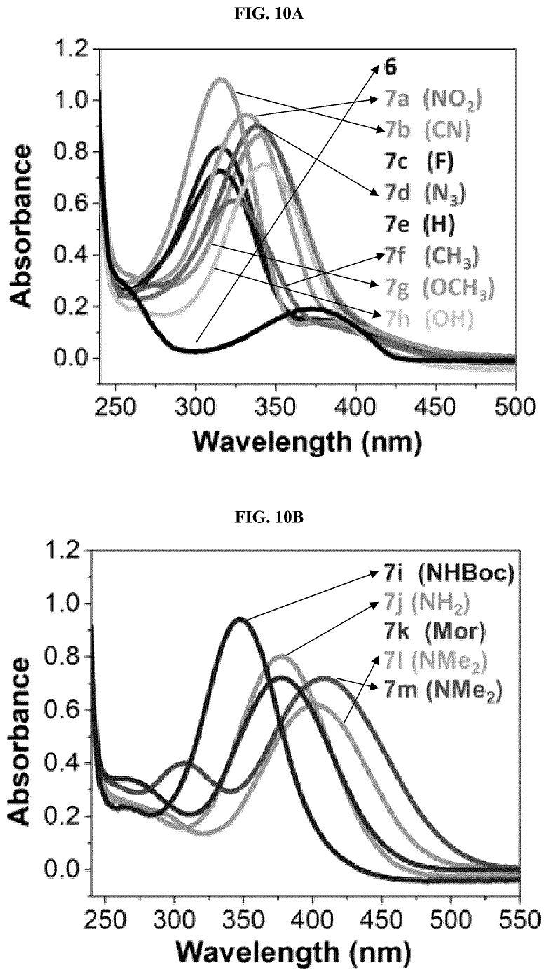

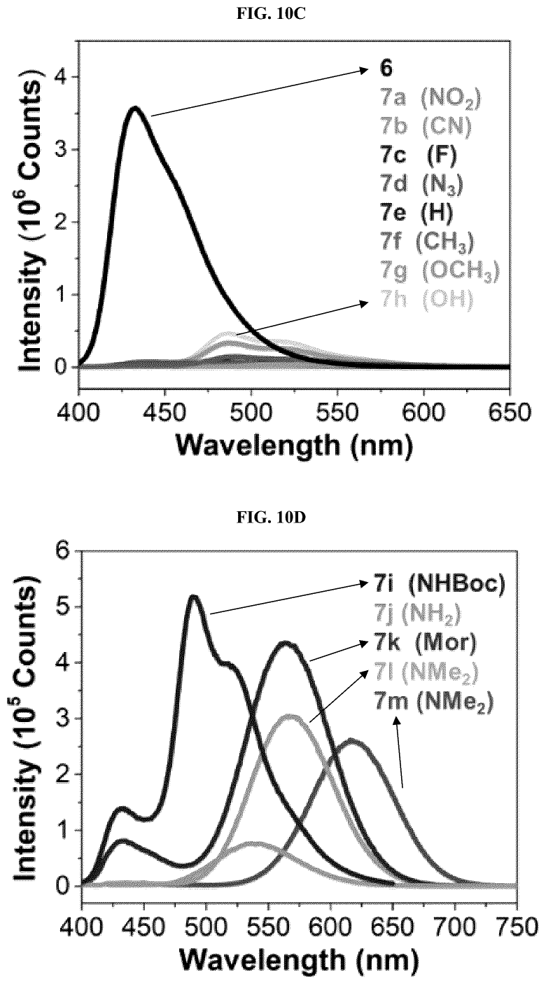

To improve the conversions for electron-rich aldehydes, the production of dimethylamino analog 7l was used as a model reaction (Table 6). The K₂CO₃ in ACN/H₂O conditions provided only a 9% yield. Using a stronger base, KOtBu, in DMF improved the yield to 27%, even at room temperature. Changing the base to NaOMe further improved the yield to 46%, albeit with a still long reaction time. Increasing the temperature to 60° C. did not improve the yield. These optimized conditions were then applied to other electron-rich aryl aldehydes, improving yields to 40-60%. All probes were characterized by ¹H-NMR, ¹³C-NMR, and high-resolution mass spectrometry (HRMS); 7a, 7c, and 7l were further characterized by X-ray analysis (Tables 7-9 and FIGS. 7-9).

### Example 3: Substituent Effects on Photophysical Properties

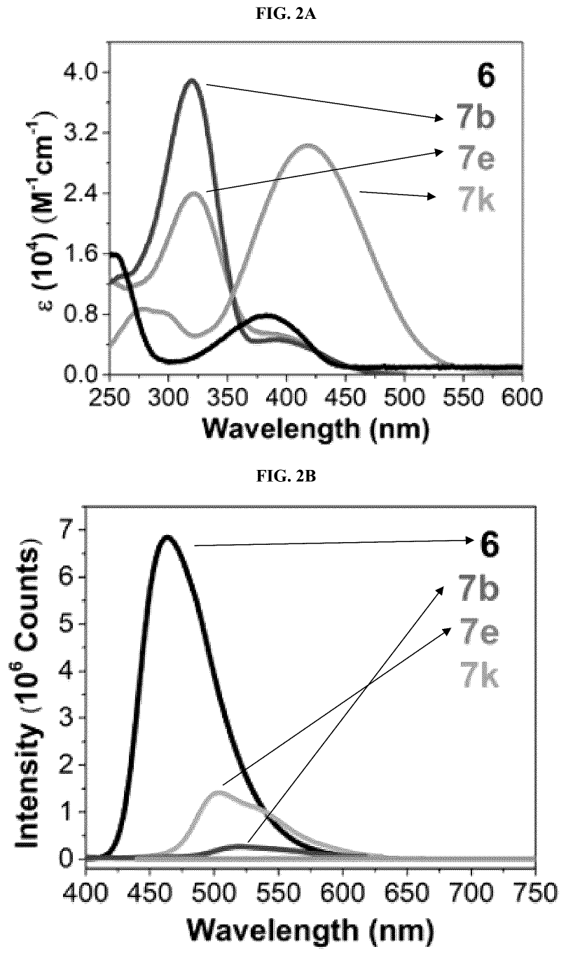

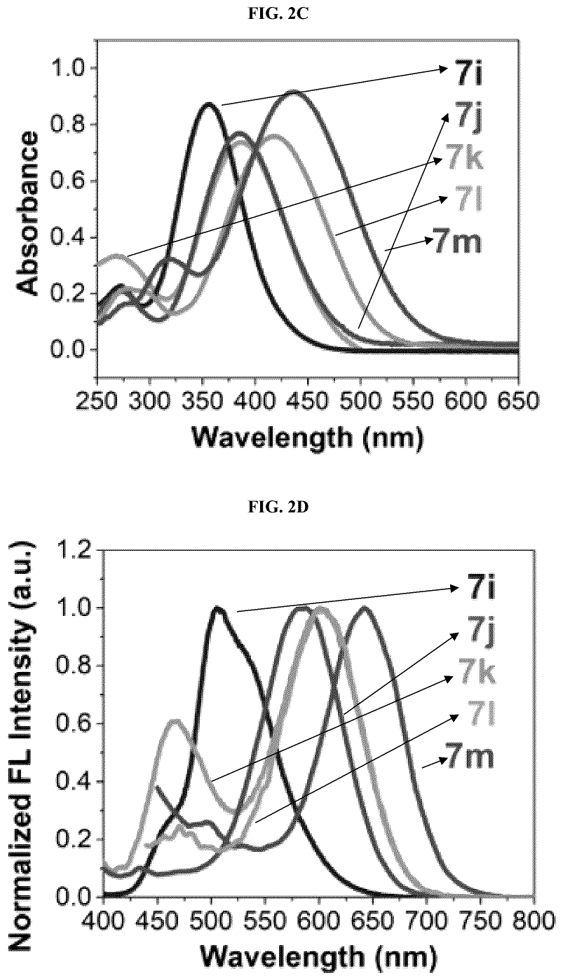

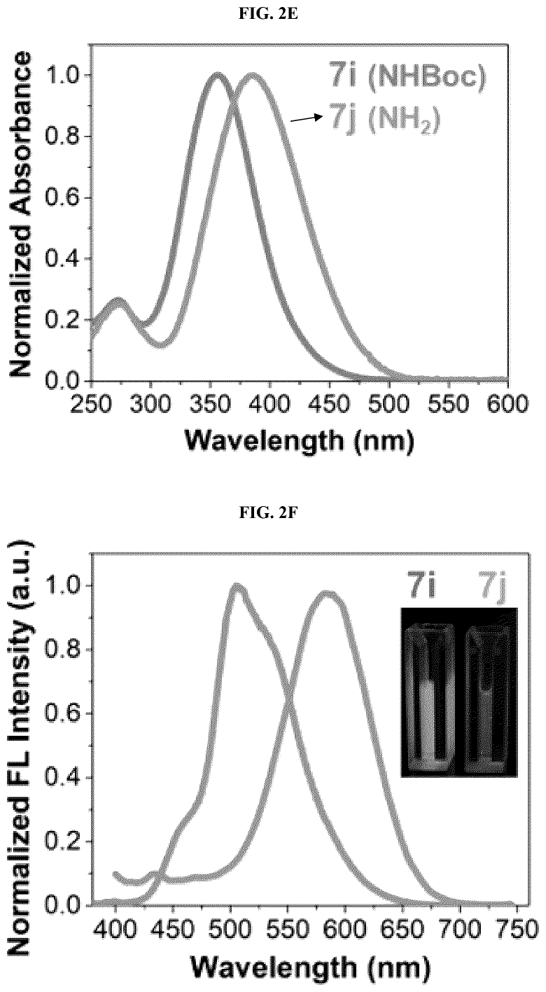

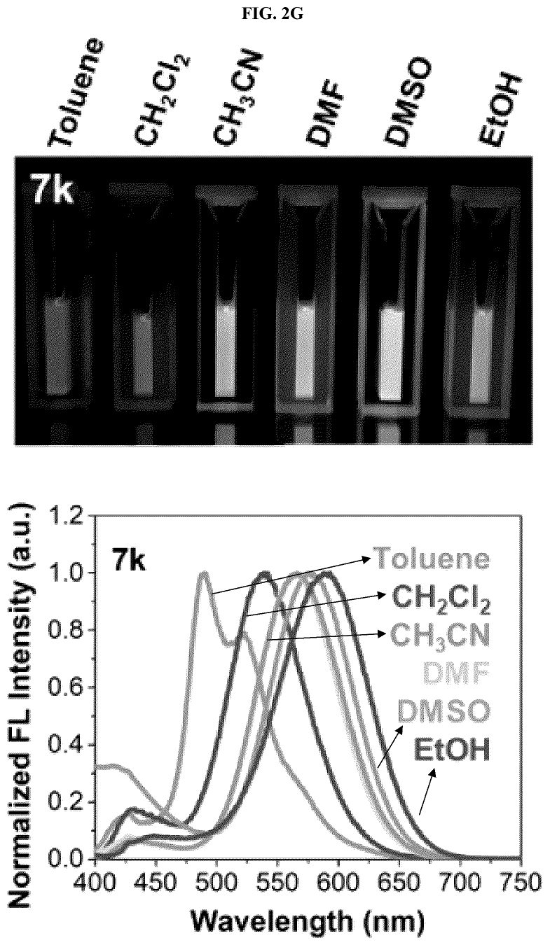

After successful synthesis of a styryl bimane probe library, the photophysical properties of each derivative were studied. In the absorption spectra, a more electron-donating aryl substitution on bimane revealed a more red-shifted absorption in 50:50 ACN/phosphate buffered saline (PBS), suggesting the formation of a donor-π-acceptor (D-π-A) system (Table 3 and FIGS. 2A-2D).

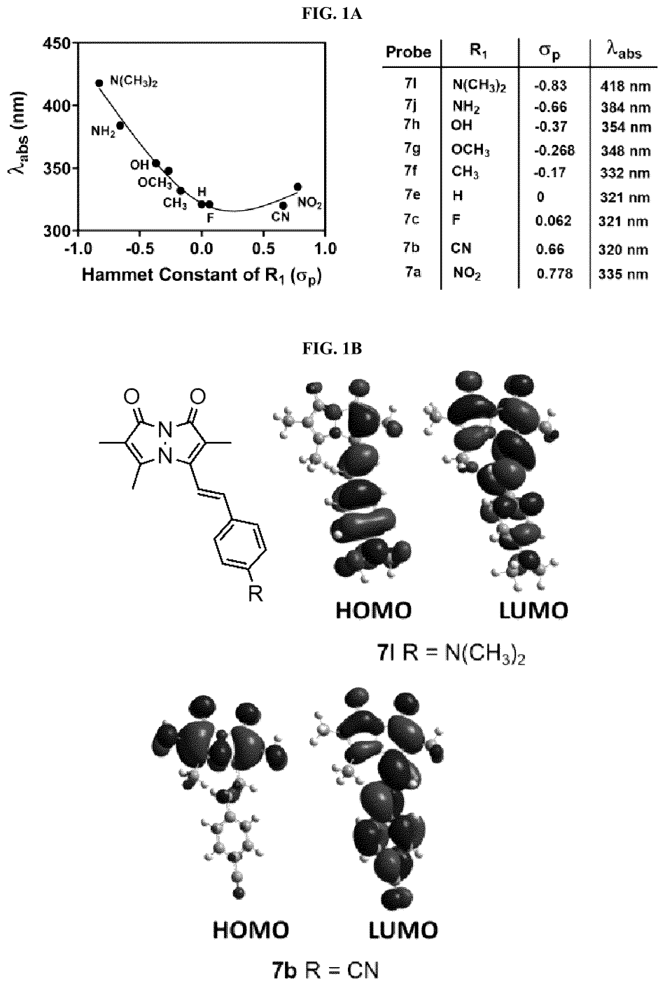

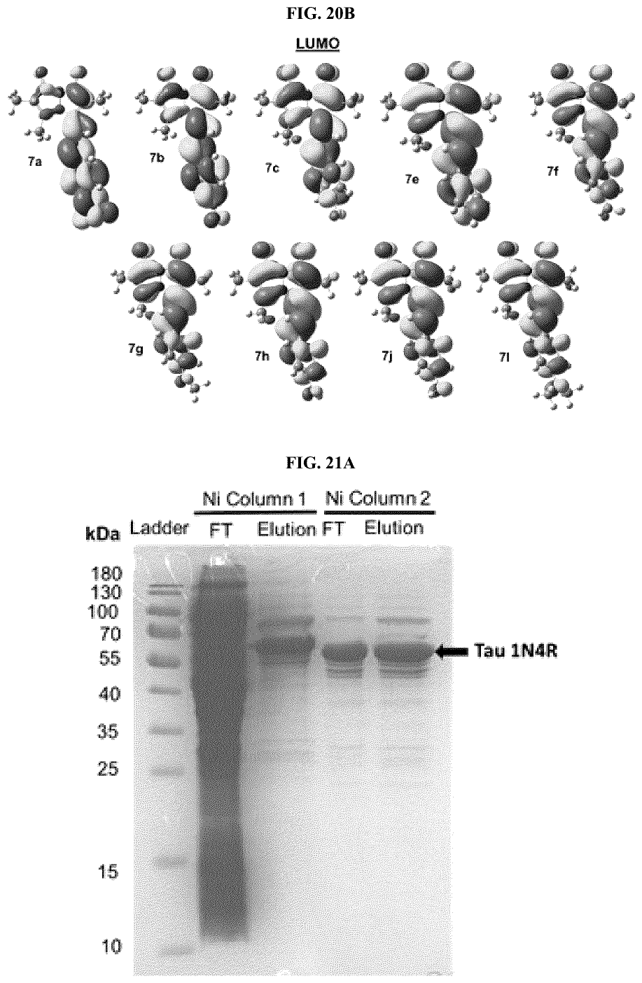

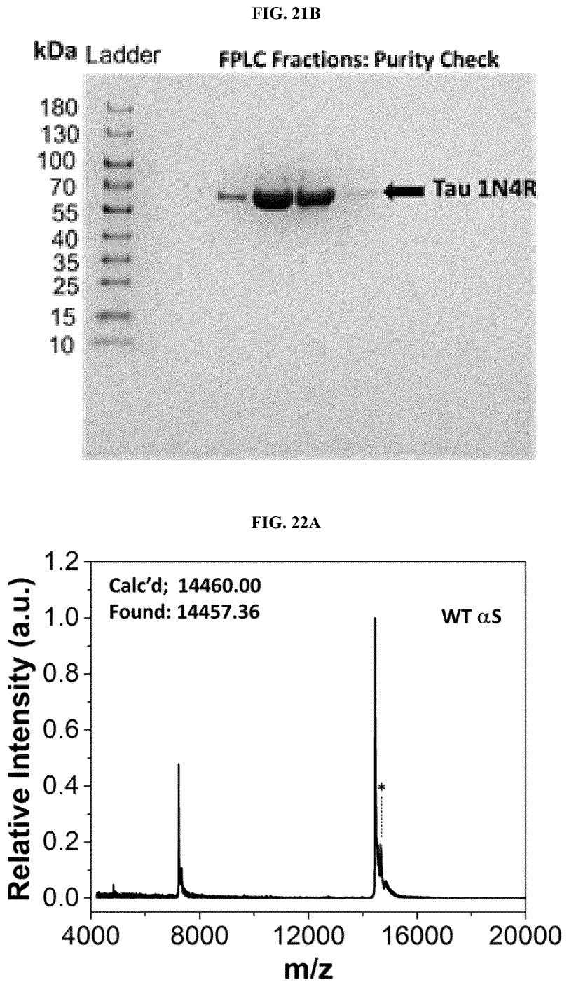

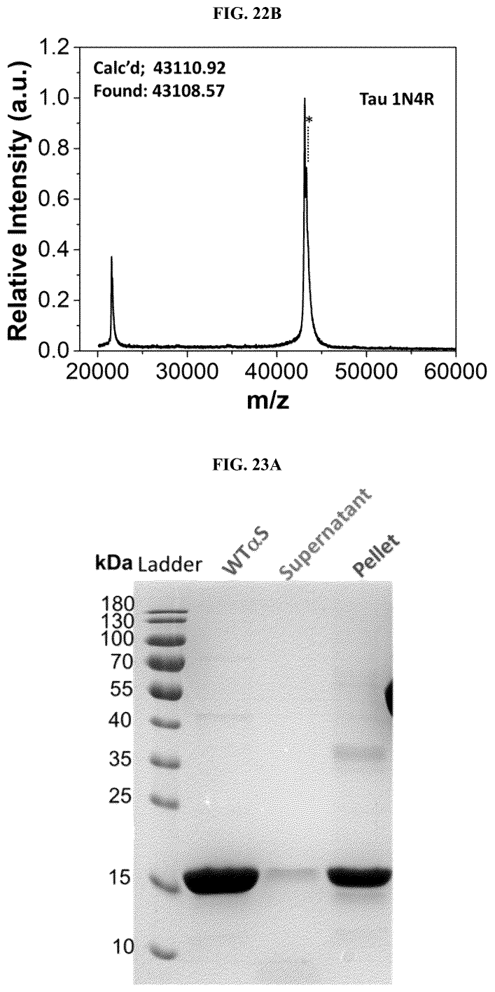

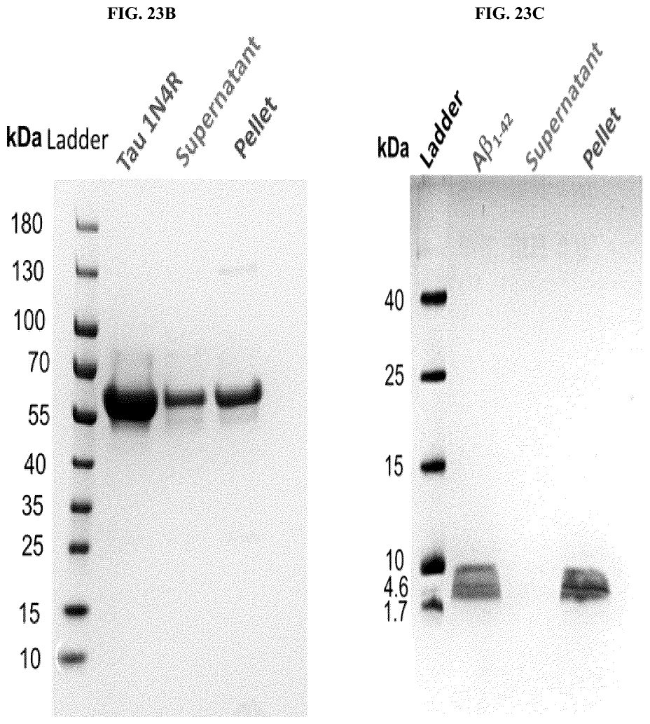

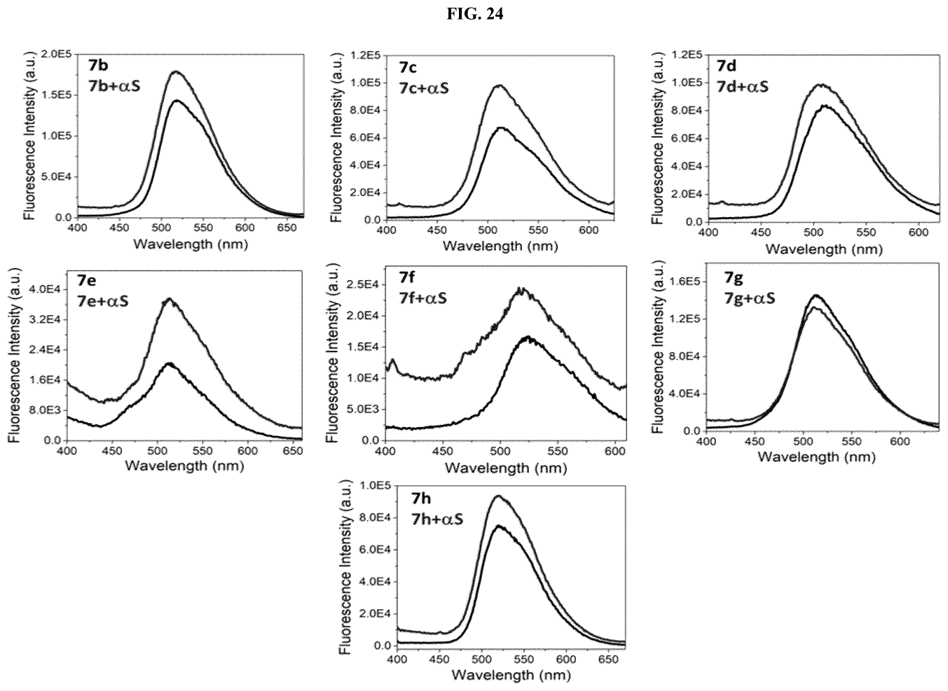

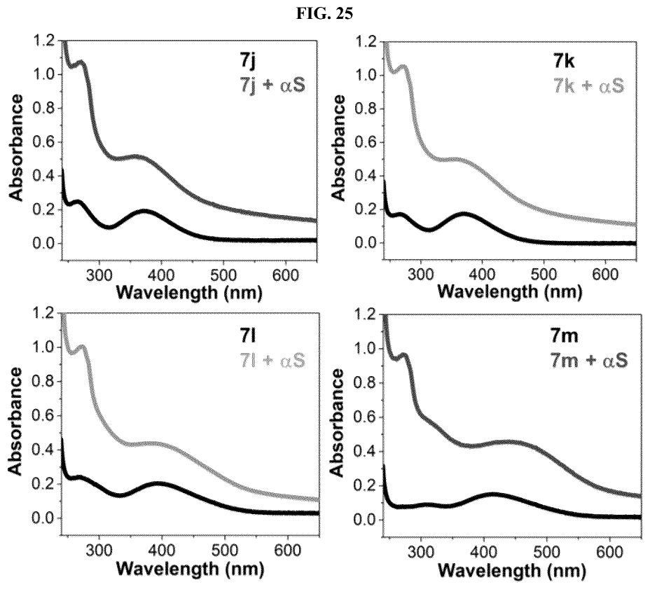

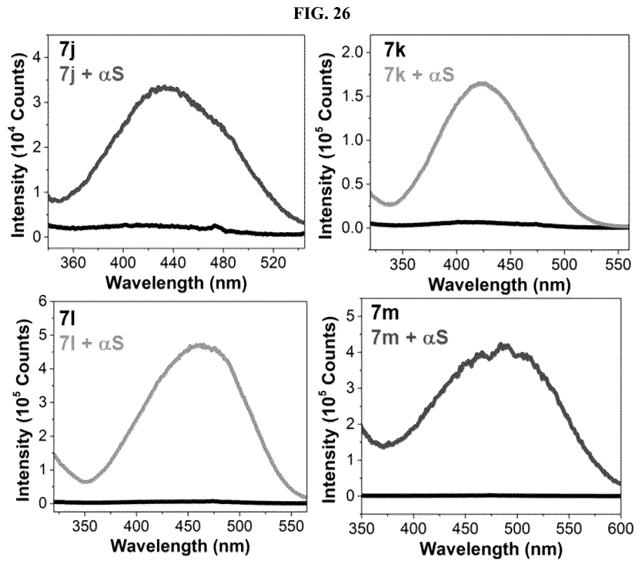

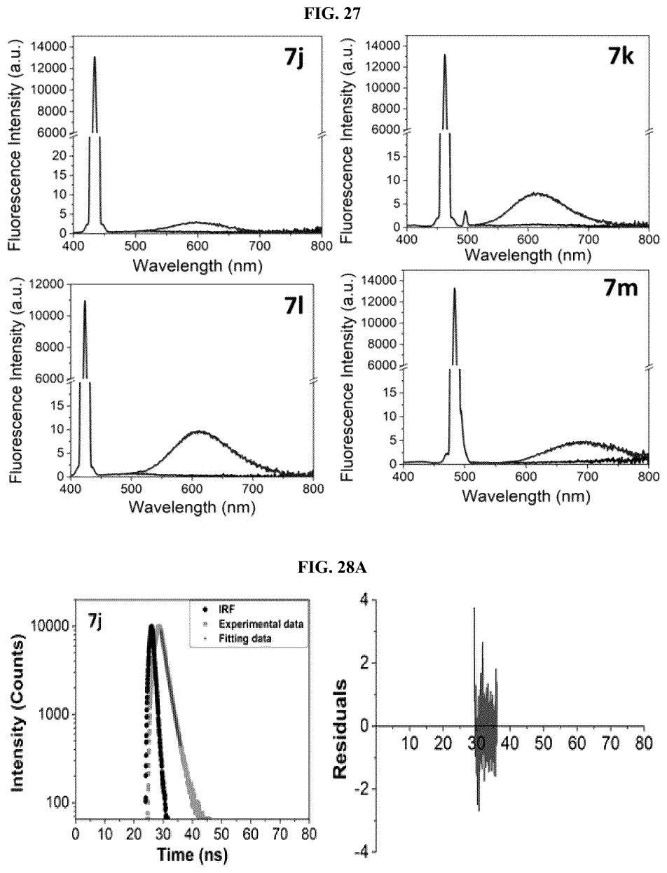

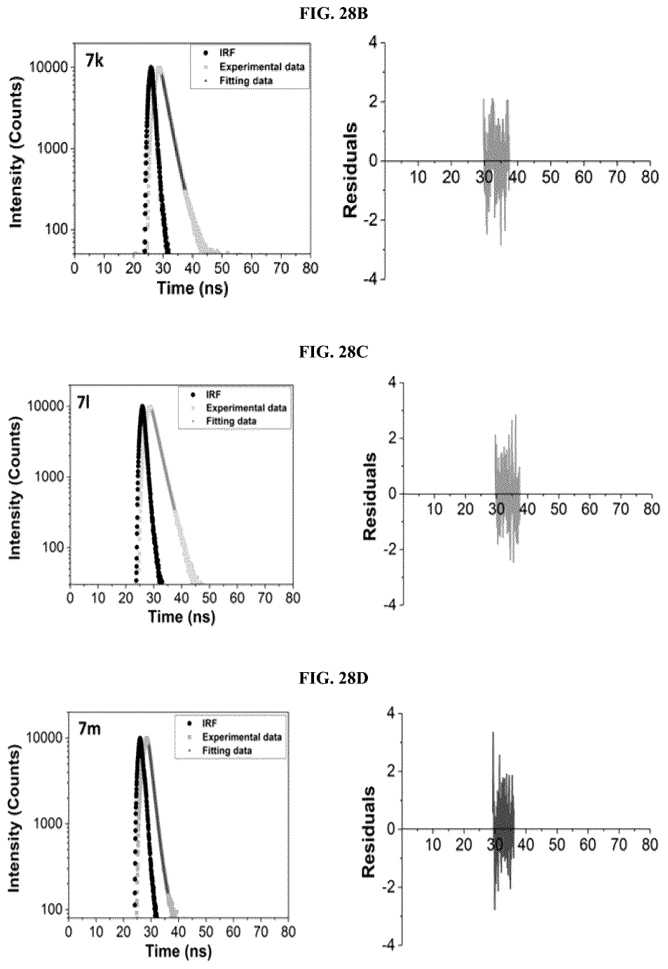

To provide guidelines for the rational choice of substituents for future bimane derivatives, compounds 7a-c, 7e-h, 7j, and 7l were analyzed in terms of the Hammett substituent constant for the para-functional group on the phenyl ring (FIGS. 1A-1B). Electron-donation showed a positive correlation with maximum absorption wavelength (λ_{abs}). This analysis was further supported by HOMO-LUMO calculations, which show a movement in electron density from the phenyl group of 7l to the bimane core upon excitation from S₀ to S₁. (Example 3, FIGS. 18-19, and FIGS. 20A-20B).

The importance of the amine lone pair in the D-π-A system is highlighted by comparing the spectra of 7i and 7j, which differ only by removal of the Boc group (FIGS. 2E-2F). In contrast, including bimane derivatives with electron withdrawing groups on the phenyl ring changed the slope of the Hammett plot, indicating a change in mechanism. This could also be explained through HOMO-LUMO calculations. For 7a-7c, movement of electron density from the bimane group to the electron withdrawing group on the phenyl ring is seen upon excitation from S₀ to S₁. Thus, the nature of the aryl substituent changes the direction of the bimane D-π-A system.

The emission spectrum of each compound was measured in 50:50 ACN/PBS under excitation at λ_{abs}. Parent phosphonate 6 showed blue emission with a maximum emission wavelength (λₑₘ) at 464 nm (FIG. 2B). The styryl bimane derivatives 7a-7l all showed a significant bathochromic shift in emission maximum (FIG. 2B and FIG. 2D). Derivatives 7b-7i had green emission spanning from 502-520 nm, while 7j and 7l had larger bathochromic shifts with λₑₘ of 583 and 604 nm, respectively. Moreover, the Stokes shifts for 7a-7l were calculated and values ranging from 133 to 217 nm were observed. These interesting fluorescent properties of styryl bimanes, such as high tunability and large Stokes shifts, make them valuable probes for a broad spectrum of biological applications.

### Example 4: Environmental Sensitivity of Probes

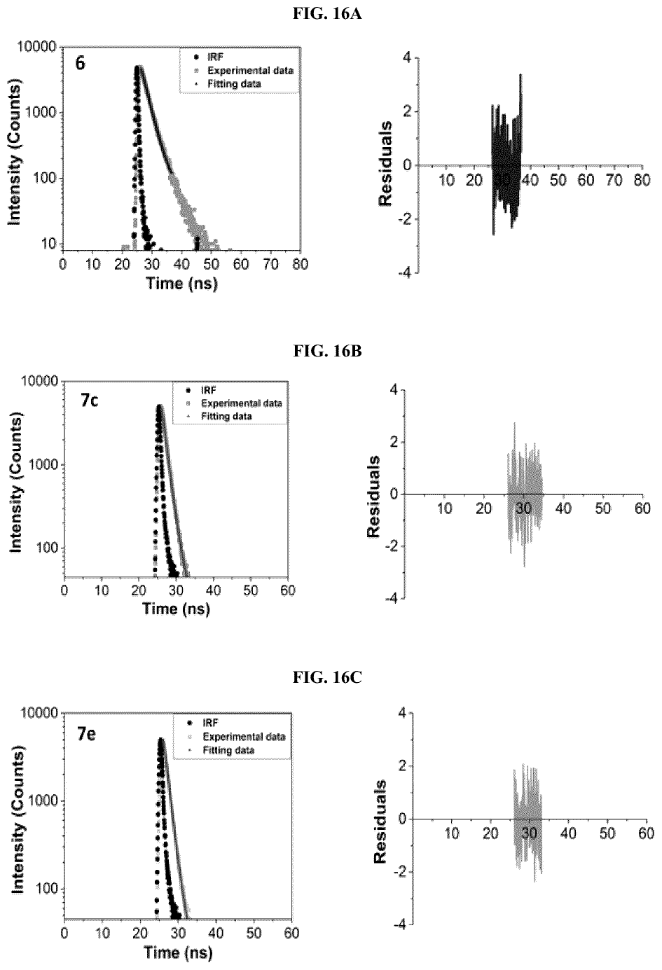

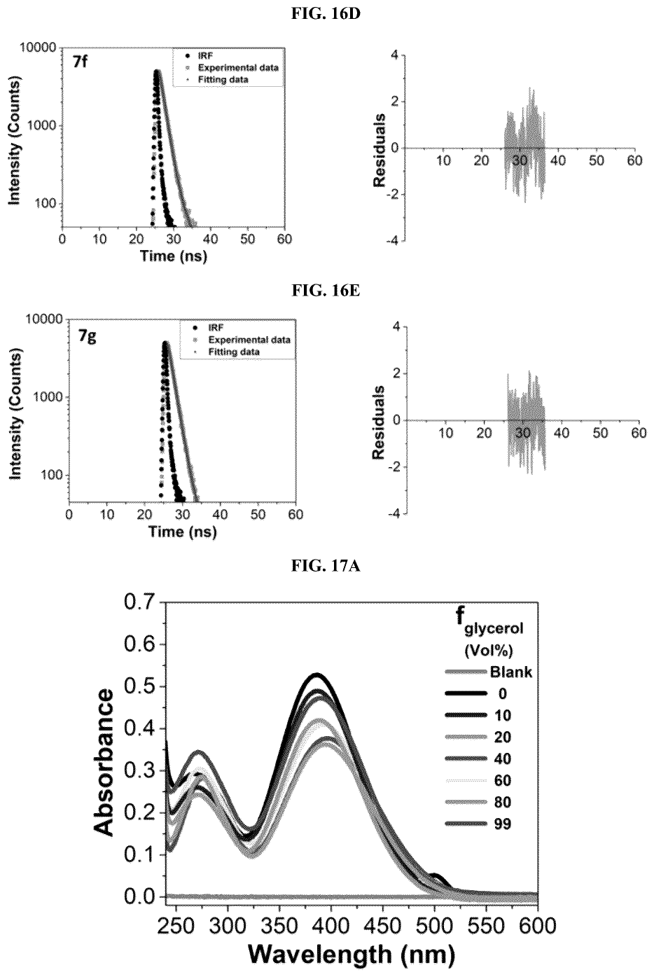

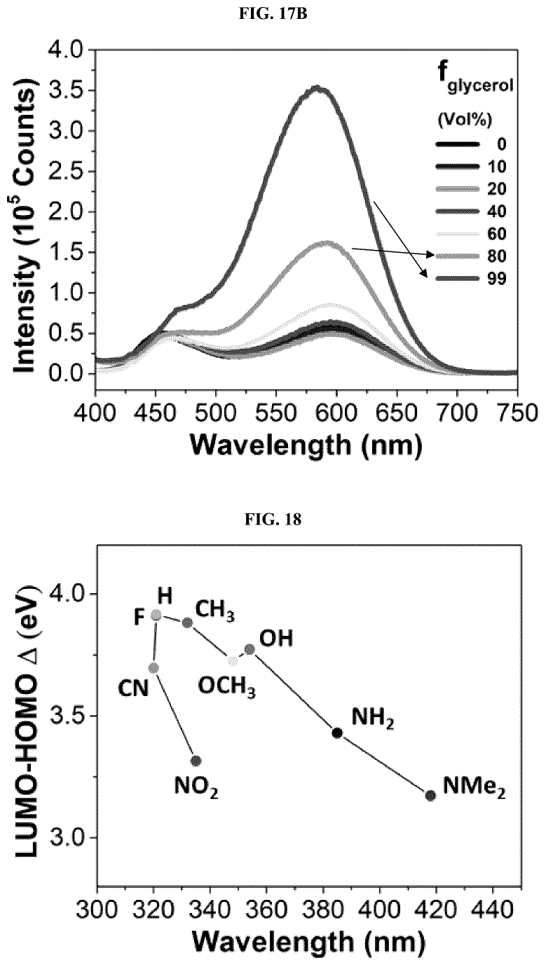

Measurement of the molar absorptivity (F) and fluorescence quantum yield (QY) of each compound revealed dramatic changes for the styryl bimanes compared to parent phosphonate 6. In ACN/PBS, an increase of 3-5 fold in F was accompanied by a 20-100 fold decrease in QY, with nitro derivative 7a essentially non-fluorescent, for a dramatic overall loss of brightness (ε·QY) compared to 6. The changes in QY are also reflected in shortened lifetimes for the derivatives compared to 6 (Table 3 and FIGS. 16A-16E). In pure ACN, F values were not dramatically affected, but the QY of some compounds increased significantly, particularly tertiary amine probes 7k and 7l, with QYs of about 7% in ACN. Notably, primary amine 7j did not experience as large an increase in QY (2%).

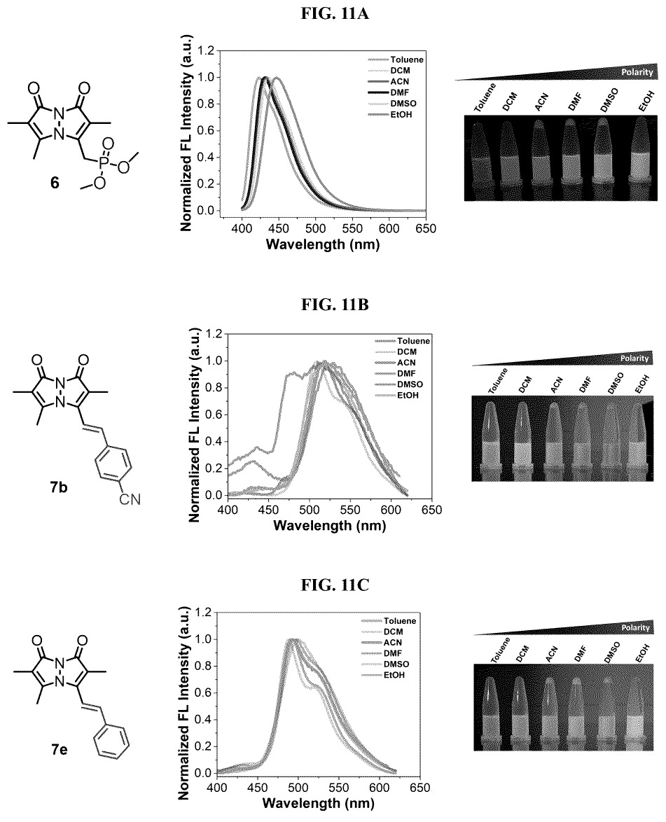

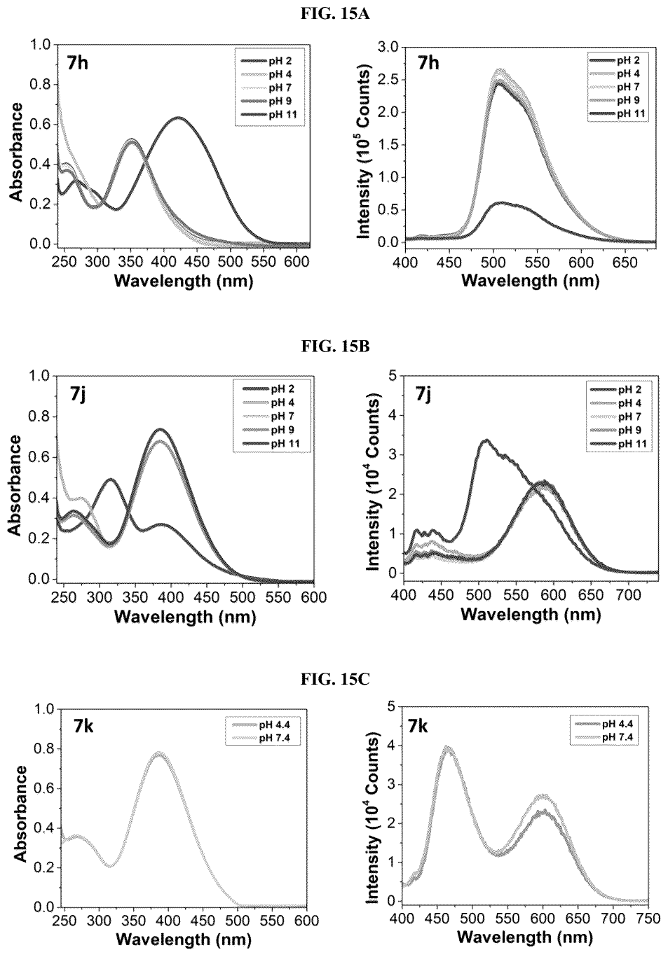

This dramatic effect on QY led us to investigate solvatochromism more generally, obtaining emission spectra in six organic solvents with varying properties: nonpolar aprotic (toluene, DCM), polar aprotic (ACN, DMF, DMSO), and polar protic (EtOH). Again, derivatives 7j-7l having electron donating groups showed the greatest sensitivity to solvent polarity, while parent compound 6 showed no significant environmental sensitivity (FIGS. 11A-11C). As illustrated for 7k in FIG. 2G, these probes have high solvatochromism, with emission spanning 490-590 nm and a red-shift in more polar solvents. pH effects were also studied (FIGS. 15A-15C) and it was found that although some compounds exhibited pH effects, these were not significant in physiological pH ranges. For example, phenolic compound 7h has red-shifted absorption at pH 11 with a reduced QY, but no shift in emission. Amine 7j has blue-shifted absorption and emission at pH 2. Taken together, these data show that the solvent effects are tunable based on the styryl substituent and motivate the use of amine-containing derivatives as probes of the local environment on a protein surface or in a cellular compartment. The lack of pH sensitivity in the physiological range or changes in protic solvents implies that these effects are modulated more by the general polarity of the environment than by specific hydrogen bonding or protonation.

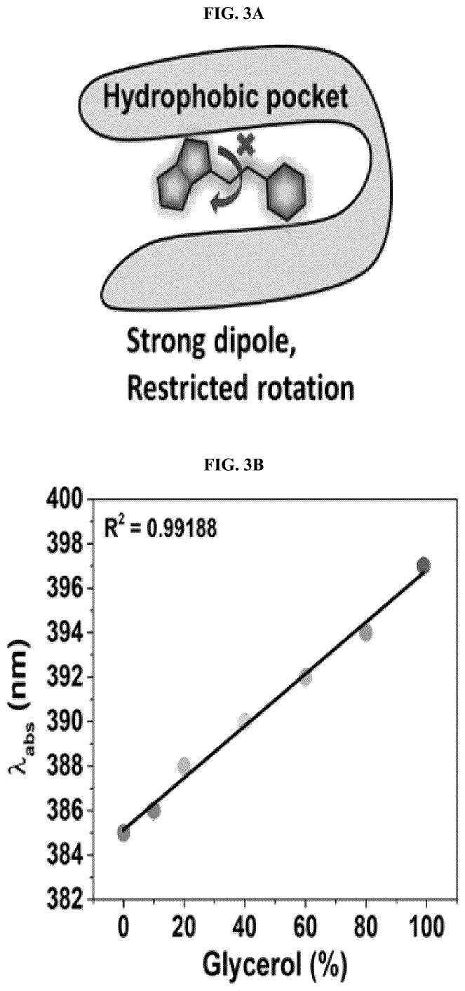

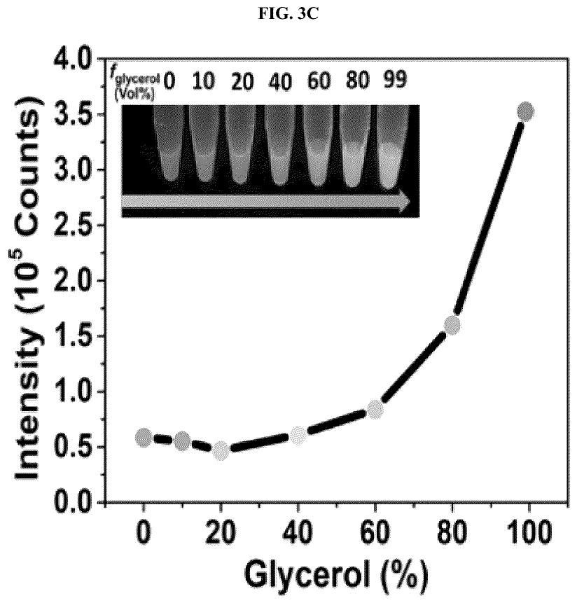

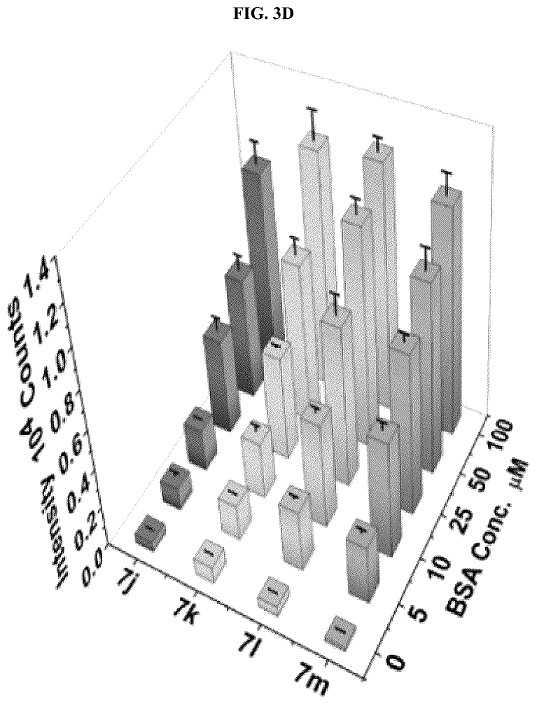

It was suspected that the low QY of the styryl bimane probes was due to energy dissipation through non-radiative pathways resulting from free intramolecular rotation around the vinyl bond. To investigate this, the emission spectra of 7k in glycerol/MeOH mixtures was recorded, where the increased viscosity of glycerol serves to restrict rotation without a significant change in polarity (FIGS. 3A-3C). A 12 nm red-shift and >6-fold increase in its fluorescence intensity with % glycerol indicates that indeed rotational restriction planarizes the π system. It was hypothesized that the combination of solvatochromic and rotor effects should lead to a dramatic fluorescence turn-on upon binding in a hydrophobic cavity on a protein. Indeed, testing certain exemplary compounds in the presence of bovine serum albumin (BSA) showed that “turn on” was possible (FIG. 3D), prompting us to test binding to targets of more physiological significance.

### Example 5: Rational Design of a Red-Shifted Probe

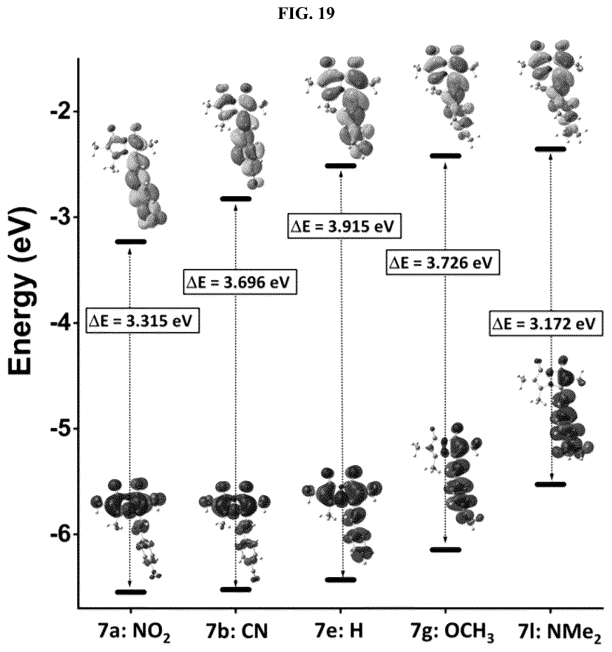

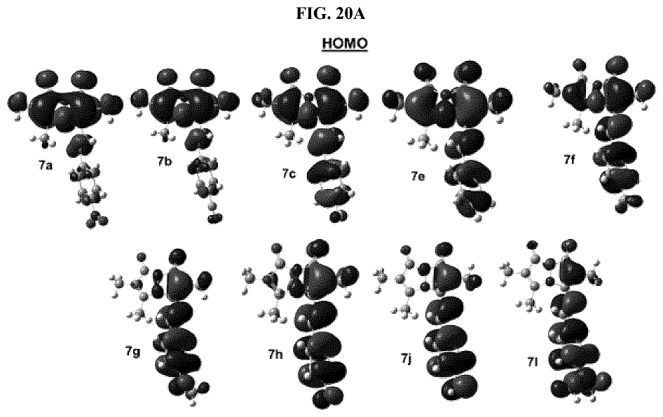

A second generation, red-shifted probe was designed by taking into account the following data from photophysical characterization of the styryl bimane probes. Although both electron-donating and electron-withdrawing groups led to large Stokes shifts, electron donating groups provided more-red-shifted dyes, with higher QYs and greater environmental sensitivity. Among these dyes, tertiary amines 7k and 7l had notably higher quantum yields in ACN than 7j, and greater red-shifting. Therefore, diene 7m was designed with the expectation, on the basis of the earlier results described herein, that the longer π system would afford red-shifted absorbance and emission within the same D-π-A framework as 7l, where the amine-centered HOMO, and the tertiary amine in particular, would provide a high QY and strong environmental sensitivity. Indeed, electronic structure calculations (FIG. 19) provided support for this hypothesis.

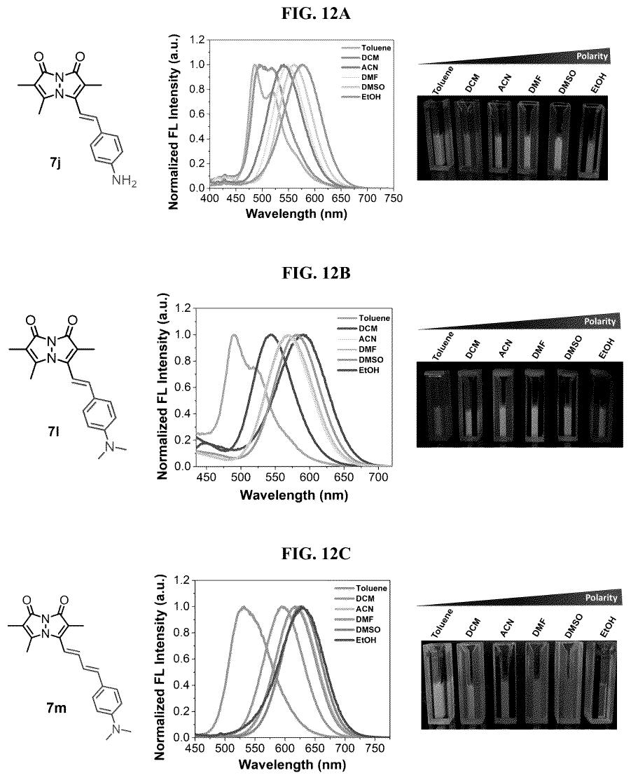

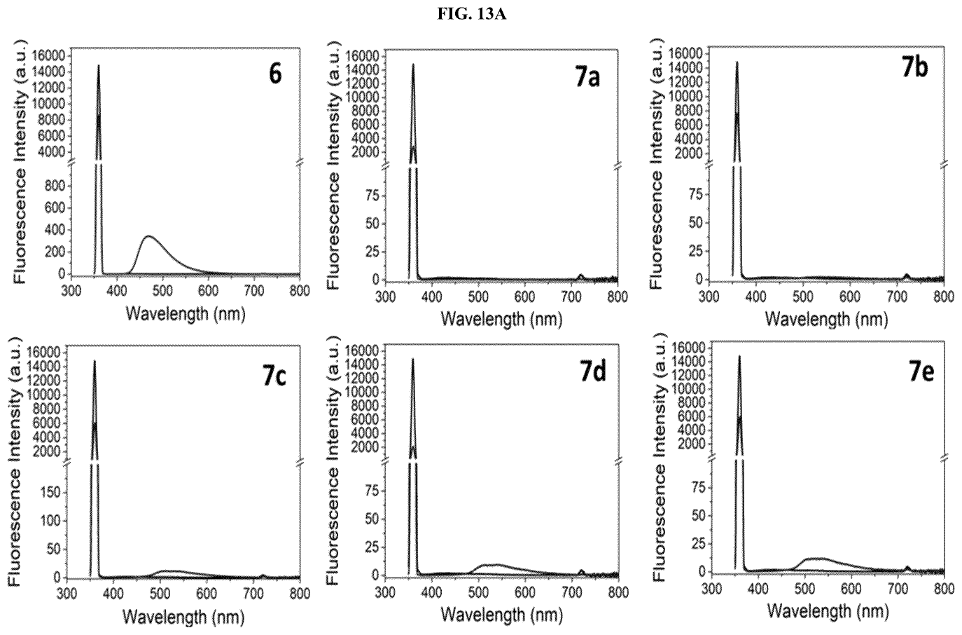

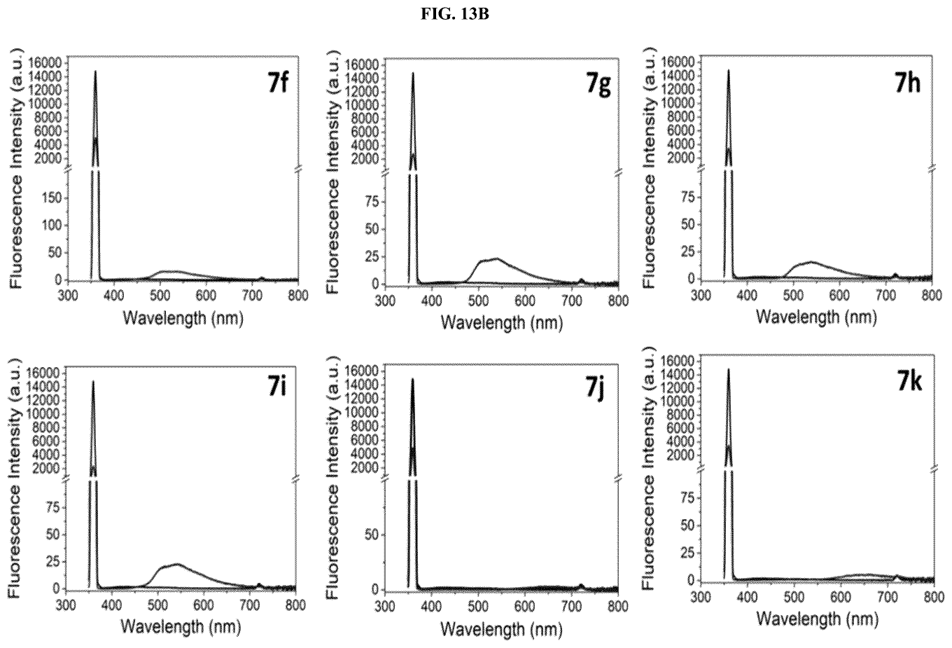

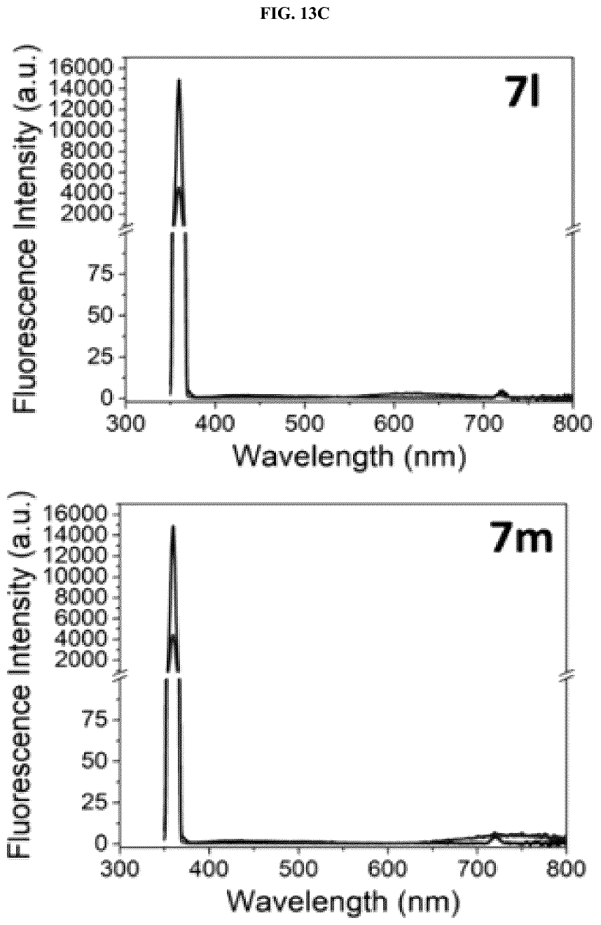

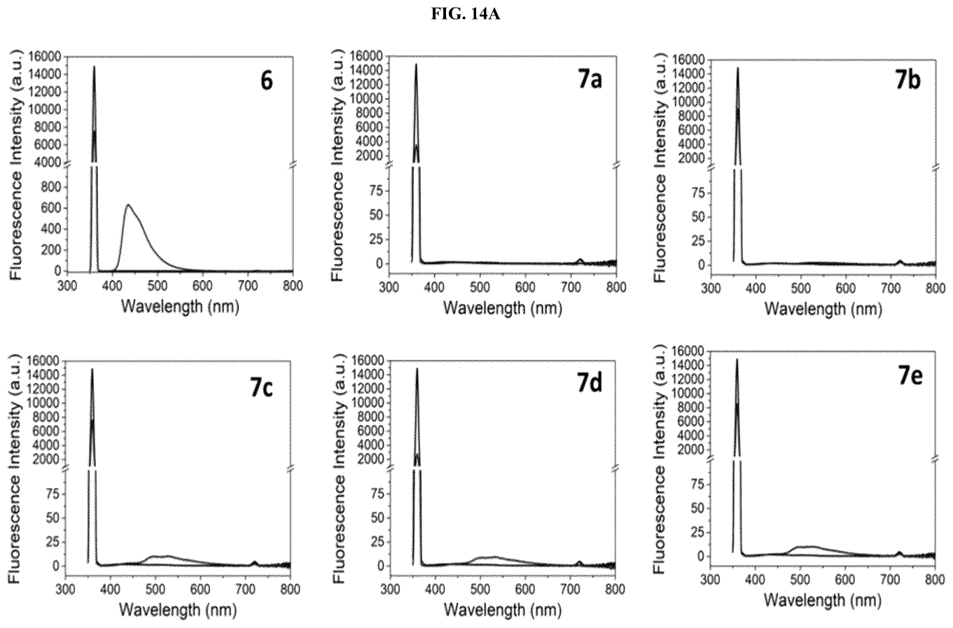

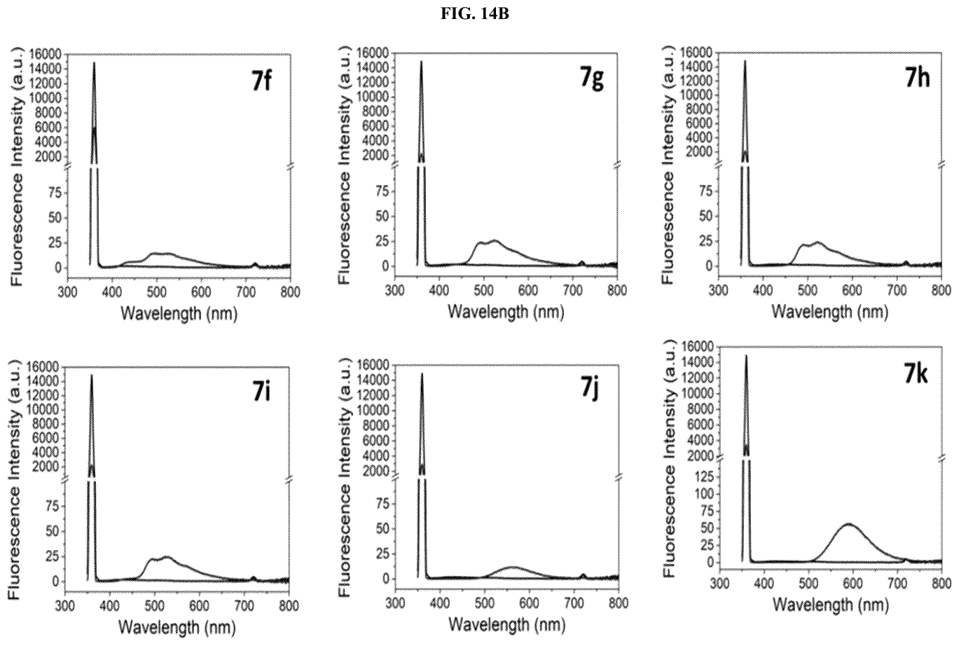

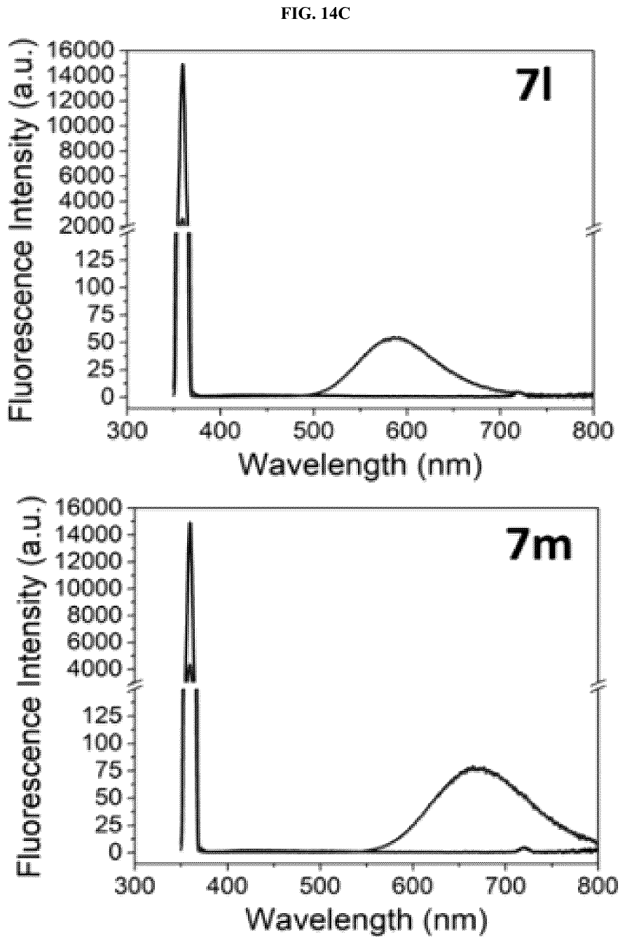

Using the optimize HWE conditions optimized for electron-donating aryl aldehydes like 7l, 7m was prepared in 45% yield from bimane phosphonate 6. The full set of photophysical assays were then performed on 7m and it was found that indeed it was highly red-shifted with λ_{abs}=436 nm and λₑₘ=640 nm in ACN/PBS for a Stokes shift of 204 nm (Table 3 and FIGS. 2F-2G). While it had a low QY in ACN/PBS like the other amine dyes, its QY in ACN was 14%, indicating an even stronger “turn on” than 7l (Table 2). Diene 7m also showed high levels of solvatochromism, like the other amine dyes (FIGS. 12A-12C).

### Example 6: Bimane Derivatives as “Turn On” Probes for αS Fibrils

The structural similarities of the styryl bimane compounds to amyloid binding dyes led to the exploration of the potential for using them in amyloid sensing. Initial efforts in this regard were focused on alpha synuclein (αS) due to its role in Parkinson's disease (PD).

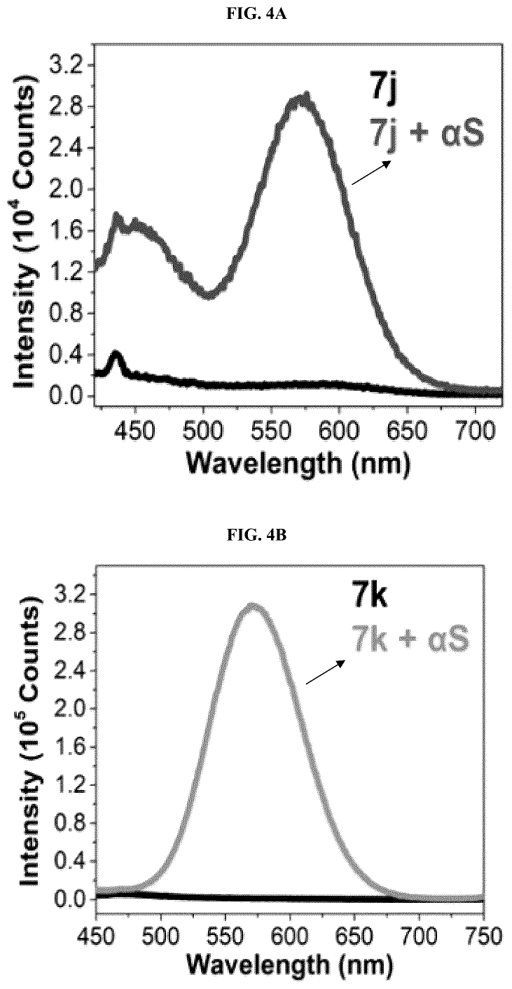

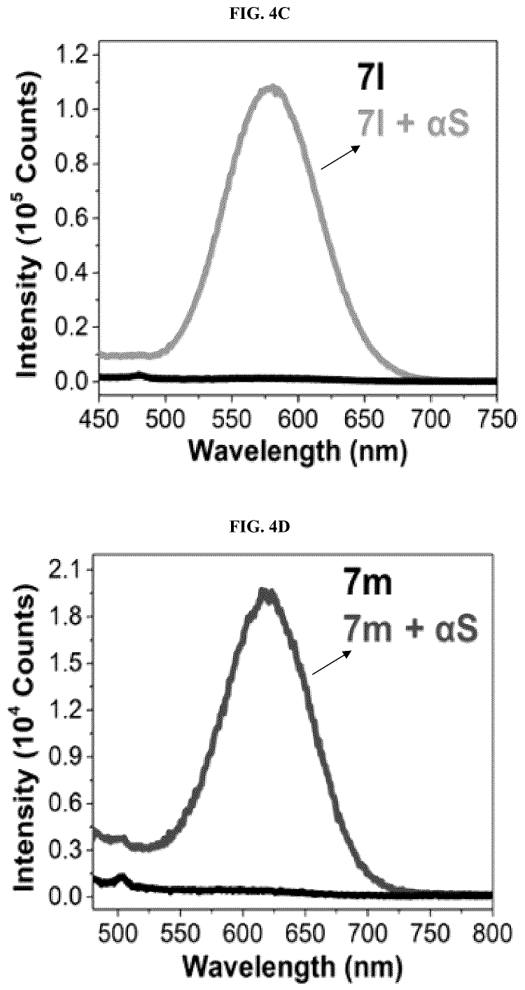

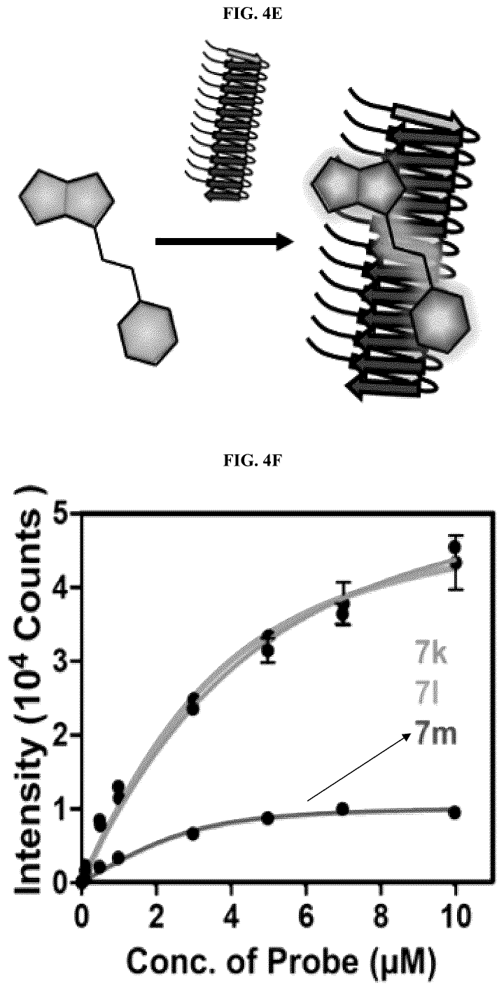

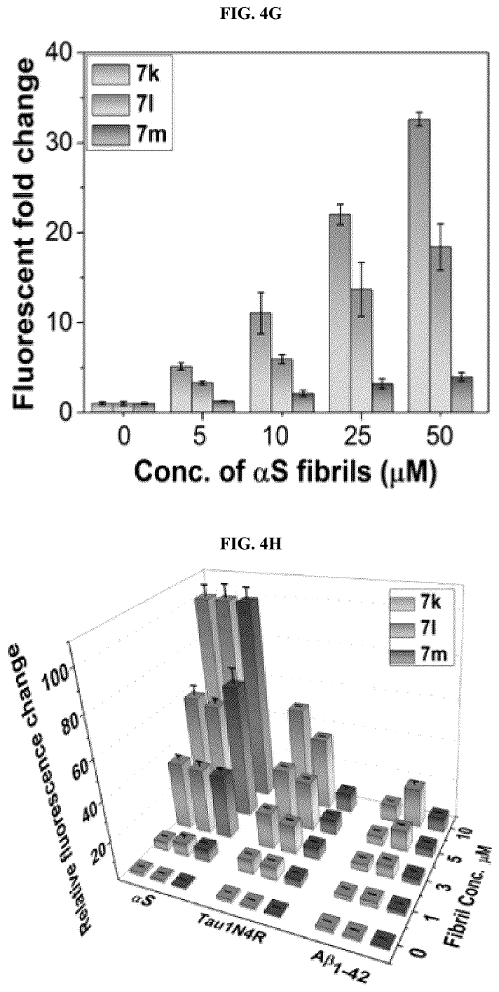

Compounds 7a-7m were screened against in vitro generated αS pre-formed fibrils (PFFs) by fluorescence spectroscopy. Among these probes, only probes 7j-7m showed a high turn on of fluorescence in the presence of PFFs, ranging from 364-476-fold increases in brightness (Tables 10A-10B and FIGS. 4A-4D). These dyes again showed large Stokes shifts in the presence of PFFs: 140, 146, 117 and 136 nm for 7j-7m, respectively. (FIGS. 4A-4D and Tables 10A-10B). For 7j, emission peaks at 450 and 575 nm were observed, presumably corresponding to the protonated and deprotonated states of the molecule, respectively.

Interestingly, increases in both F, and QY contribute to the turn on. The observed λₑₘ values when bound to PFFs are consistent with an ACN-like environment. However, while increases in QY were observed for compounds in ACN compared to ACN/PBS (Tables 2-3), these were accompanied by decreases in F, and were smaller than what was observed for PFF-bound forms of these compounds. Thus, it was concluded that turn on results from contributions from mechanisms based on both solvatochromism and restriction of rotation about the olefin linker. Fluorescence lifetimes when bound to PFFs were similar to the lifetime of 6 (Tables 10A-10B), indicating that the PFF-bound forms represent unquenched bimane fluorescence.

TABLE 10A
Selected spectral properties of 7j-7m in
the absence and presence of αS fibrils

| Dye | PFFs | λ_{abs} (nm)^{a} | λₑₓ (nm)^{b} | λₑₘ (nm)^{c} | ε (M⁻¹cm⁻¹)^{d} |
|---|---|---|---|---|---|
| 7j | − | 372 | — | — | 15385 |
| 7j | + | 359 | 435 | 575 | 45956 |
| 7k | − | 370 | — | — | 17205 |
| 7k | + | 356 | 424 | 570 | 49869 |
| 7l | − | 393 | — | — | 20071 |
| 7l | + | 386 | 463 | 580 | 43864 |
| 7m | − | 416 | — | — | 19024 |
| 7m | + | 412 | 484 | 620 | 52234 |

^{a}Maximum absorption wavelength (λ_{abs}) in the presence of αS PFFs corresponds to a mixture of bound and unbound dye.
^{b}Maximum excitation wavelength (λₑₓ) better represents the bound form of dye.
^{c}Maximum emission wavelength (λₑₘ) corresponds to a bound dye.
^{d}Molar absorptivity (ε) was measured at λ_{abs}.

TABLE 10B
Selected spectral properties of 7j-7m in
the absence and presence of αS fibrils

| | | Stokes | Fluorescence | | Fluorescence |
|---|---|---|---|---|---|
| | | Shift | QY | Relative | Lifetime |
| Dye | PFFs | (nm)^{a} | (%)^{b} | Brightness^{c} | (ns)^{b} |
| 7j | − | — | 0.17 | | — |
| 7j | + | 140 | 20.7 | 364 | 1.90 |
| 7k | − | — | 0.21 | | — |
| 7k | + | 146 | 29.2 | 403 | 2.09 |
| 7l | − | — | 0.15 | | — |
| 7l | + | 117 | 32.7 | 476 | 2.27 |
| 7m | − | | 0.20 | | — |
| 7m | + | 136 | 27.9 | 383 | 1.27 |

^{a}Stokes shift was determined as the difference between λₑₓ and λₑₘ.
Final dye and PFF concentrations were 10 and 50 μM, respectively, for measurements of λ_{abs}, λₑₓ, λₑₘ, and Stokes shift.
^{b}Fluorescence quantum yield (QY) and lifetime measurements were made with 100 μM dye and PFFs.
^{c}Relative brightness was determined as the ratio of ε•QY in the presence and absence of PFFs.
λₑₓ/λₑₘ = 435/575 nm for 7j, 424/570 nm for 7k, 463/580 nm for 7l, and 484/620 nm for 7m.

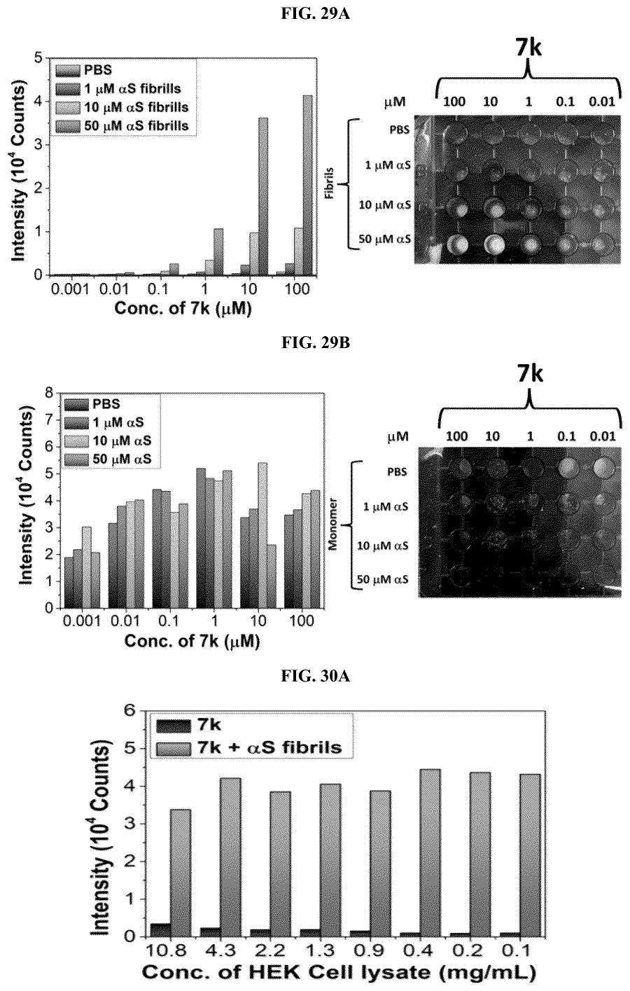

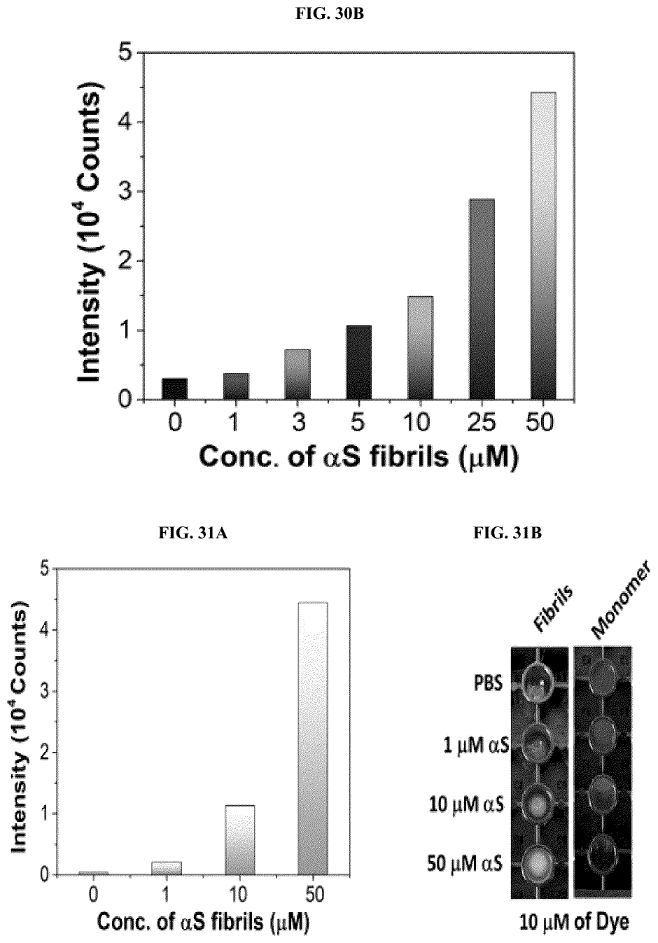

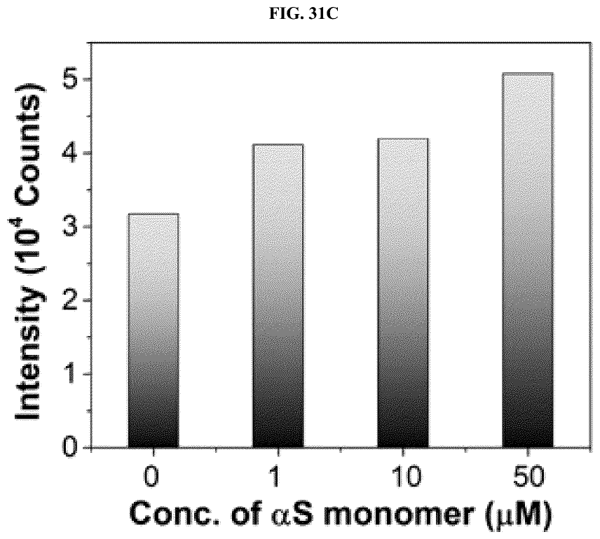

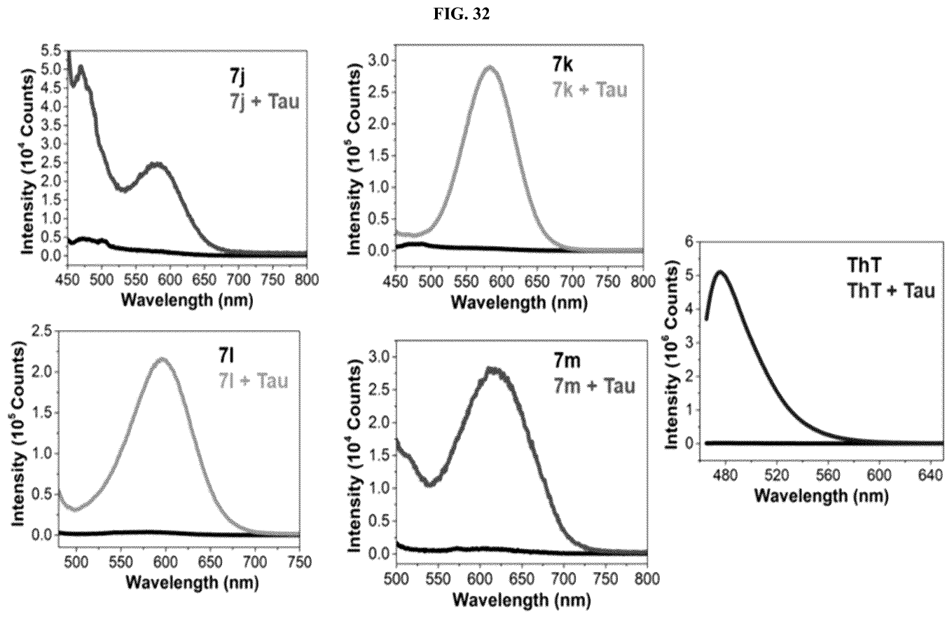

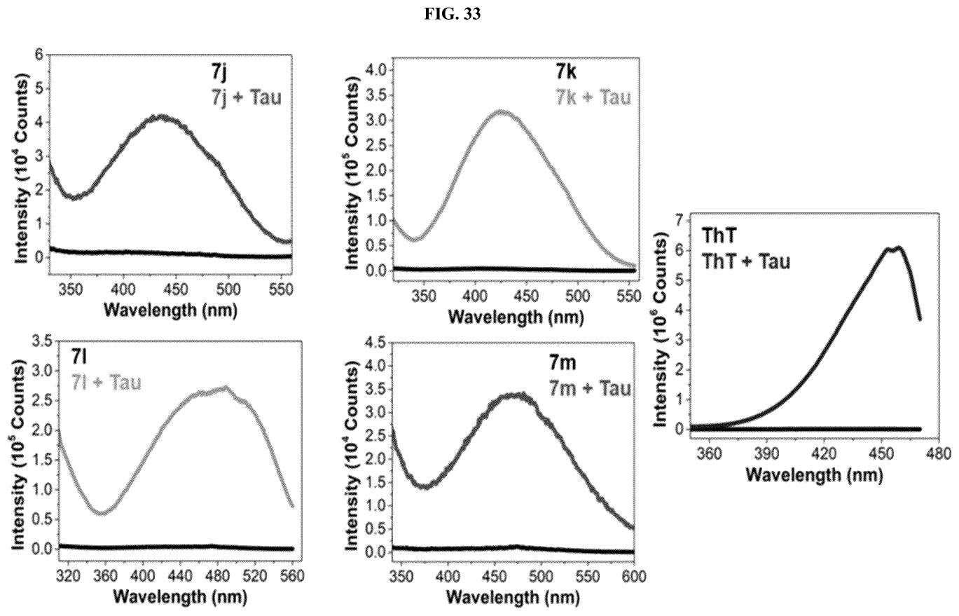

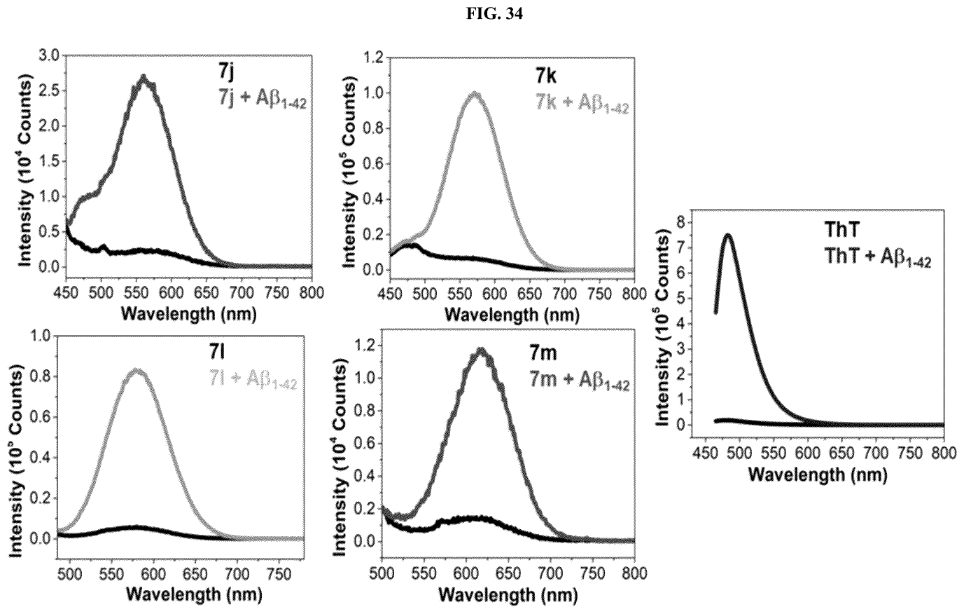

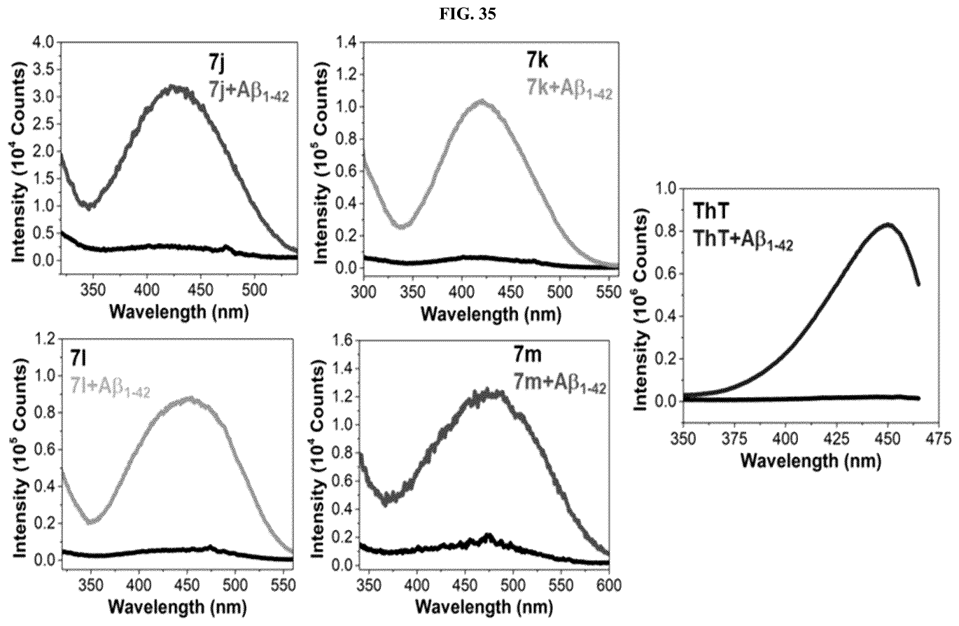

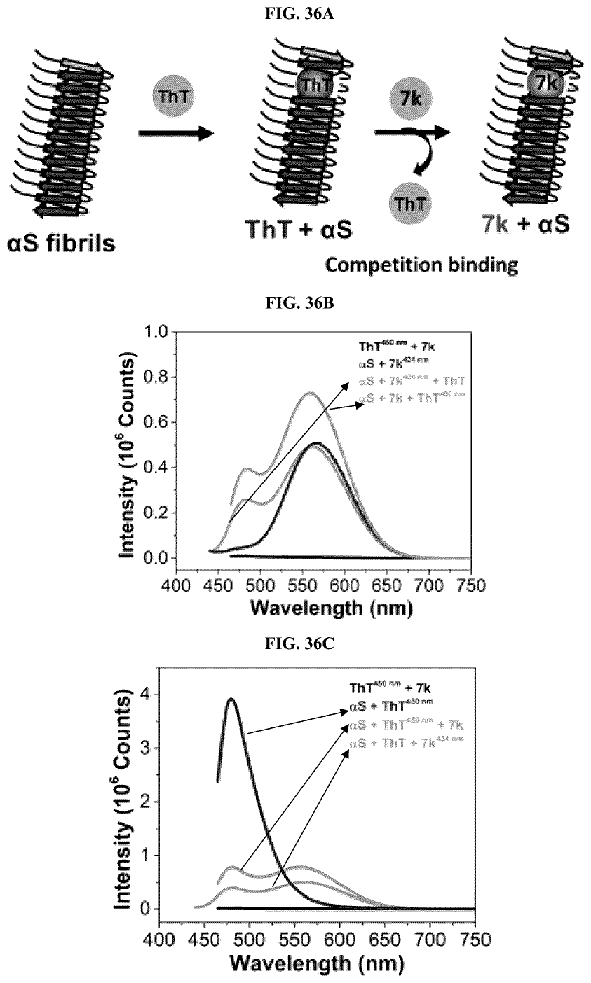

To determine the affinities of 7k-7m for αS PFFs (excluding 7j due to complexities that might arise from the two emission peaks), full titrations of the compounds were conducted (FIG. 4F) and K_{d}'s of 2.45±1.24 μM (7k), 1.45±0.43 μM (7l), and 0.42±0.25 μM (7m) were observed. Similar studies of 7k in the presence of αS monomer showed no significant fluorescence turn on signal with varied probe or protein concentration, demonstrating selectivity for fibrils (FIGS. 29A-29B). To determine whether the compounds described herein bound to the same site as ThT, competition studies were performed which demonstrated that 7k has higher affinity compared than ThT towards αS PFFs and that their binding sites overlap, at least partially (FIGS. 36A-36C).

To assess the probes' potential for imaging in biological samples, the ability of certain exemplary probes to detect varying PFF concentrations in the presence of cytosolic human embryonic kidney (HEK) cell lysate (10 mg/mL total protein) was measured. Similar results to that which was observed with the binding studies in buffer were observed, showing the ability to detect protein in the low μM range and higher fluorescence turn on from 7k and 7l than 7m (FIG. 4G). Considering all of these data, all three compounds were tested for selectivity among fibril types since the competing factors of brightness and affinity did not permit exclusion of any compounds from further study.

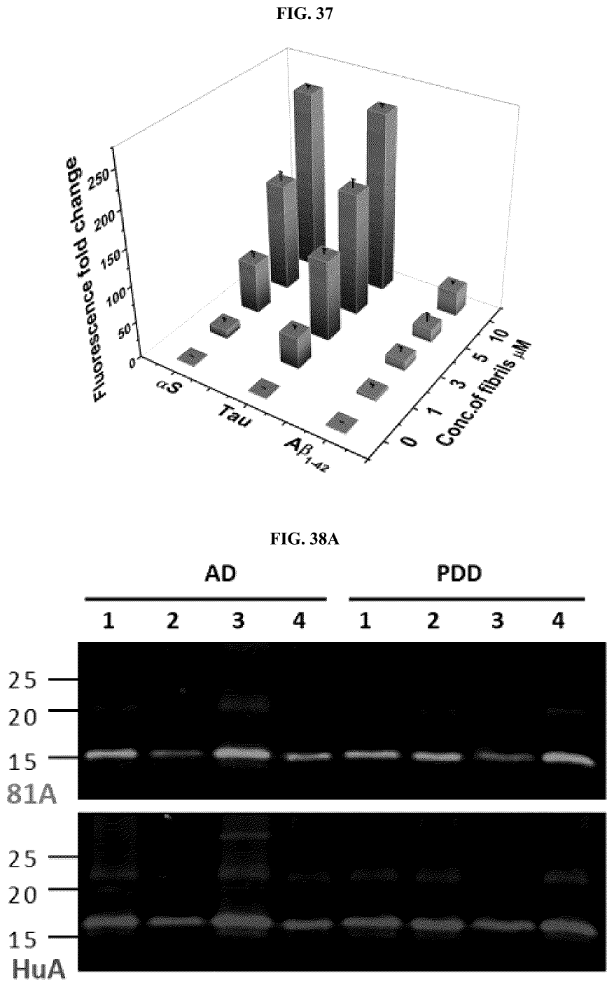

Since the low backgrounds in cell lysates indicated minimal off-target binding to soluble proteins, it was next sought to determine whether certain exemplary probes described herein were selective towards αS PFFs versus fibrils of tau and the 42 amino acid amyloid-β variant (Aβ₁₋₄₂), which are typically observed in AD patient brains. The ability to distinguish αS from tau and Aβ₁₋₄₂ is important to understanding the overlapping pathology of AD, PD, and other related neurodegenerative diseases. Therefore, probes 7k, 7l, and 7m were mixed with varying concentrations of αS, tau, and Aβ₁₋₄₂ fibrils and recorded the fluorescence emission. As shown in FIG. 4H, all three probes show selectivity toward αS PFFs. At 5 μM fibril concentrations, 7k fluorescence turn on when binding to αS is 2-fold higher than tau and 13-fold higher than Aβ₁₋₄₂. For 7l and 7m, fluorescence turn on when binding αS is 2- and 9-fold higher than tau and 5- and 15-fold higher than Aβ₁₋₄₂, respectively. This is in contrast to ThT, which has high turn on with both αS and tau (FIG. 37). Thus, in spite of binding to αS PFFs at a similar site, certain exemplary probes show much more conformational selectivity, particularly 7m.

Given the selectivity of 7k, 7l, and 7m for αS fibrils over fibrils of tau and Aβ₁₋₄₂, as well as their low background fluorescence in cell lysate, it was next sought to determine whether certain exemplary probes could be used to detect different fibril polymorphs, or “strains,” of αS fibrils. In recent years, it has become clear from structural and biochemical studies that different fibril polymorphs are present in different diseases, and that these also differ from those formed in vitro.

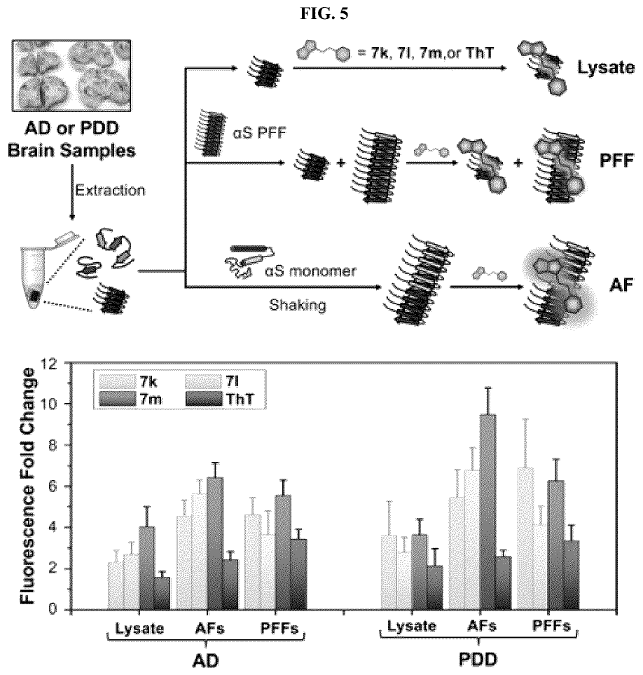

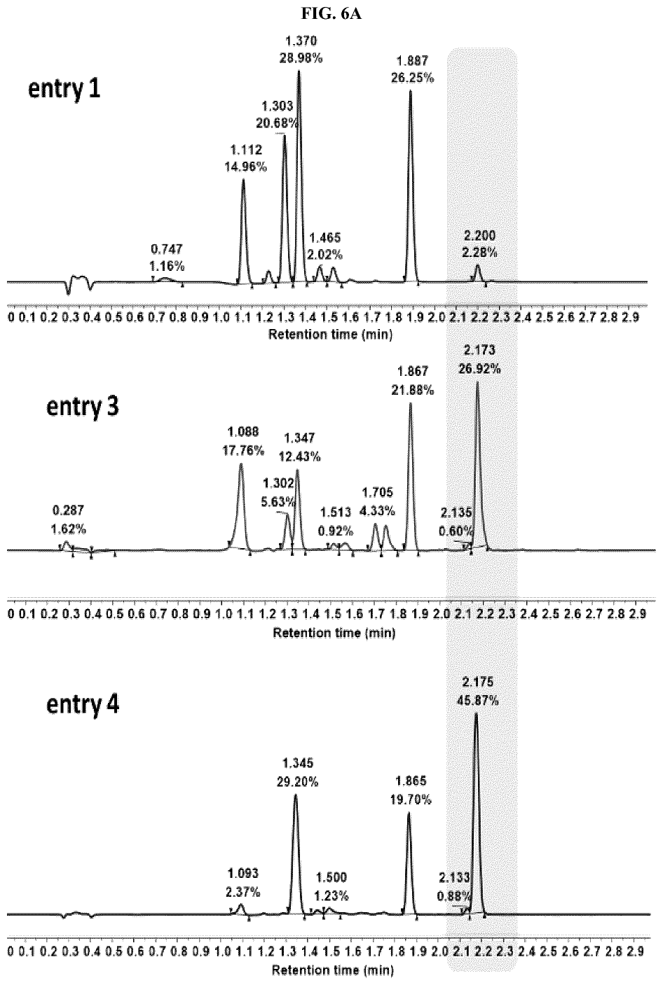

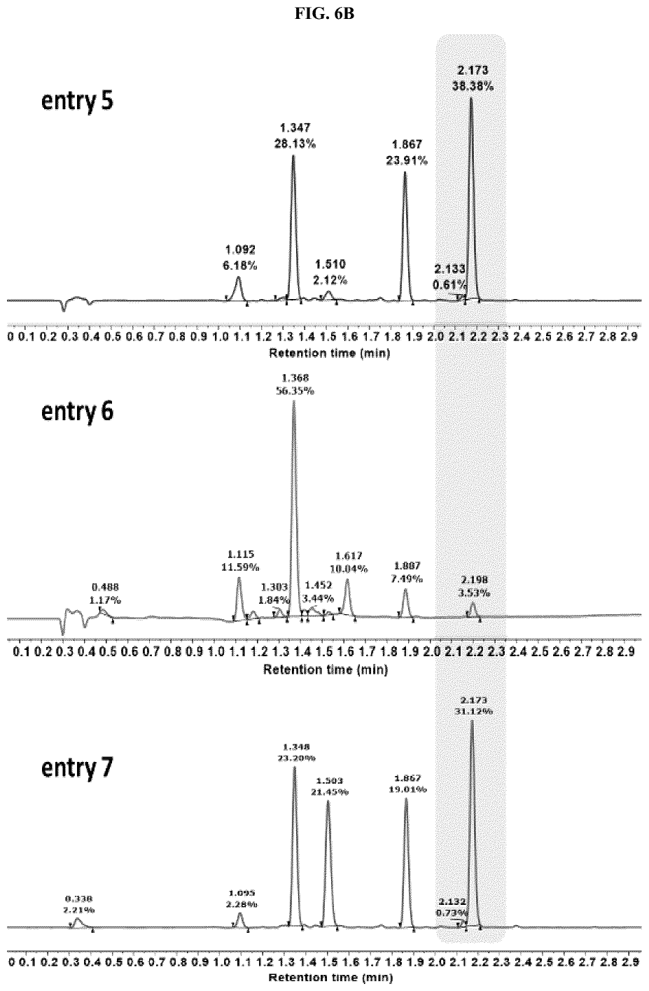

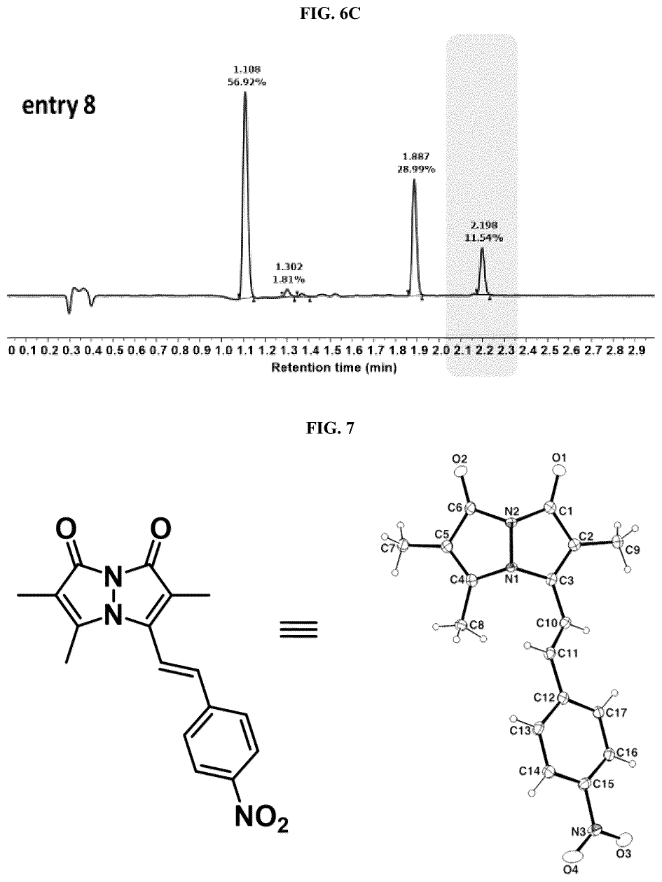

It has been recently shown that fibril strains from Lewy bodies in PD and related synucleinopathies such as PD with dementia (PDD) can be faithfully amplified in vitro using seeds derived from patient material and recombinant αS monomer. However, monitoring these reactions has been challenging because ThT does not effectively detect these amplified fibrils (AFs), as shown in FIG. 5. To determine whether certain exemplary fluorescent probes of the present disclosure could detect AFs, and potentially distinguish between fibrils from different sources, lysates from three AD cases and three PDD cases were prepared. For a portion of each lysate, fibrillar material was amplified with αS monomer. The fluorescence of 7k-7m in the presence of the AFs, in the lysates alone, or in lysates doped with an equivalent amount of in vitro αS PFFs were then compared (FIG. 5). It was found that while there was some fluorescence for the brain lysate samples with endogenous fibrillar material, there was a two-fold or more increase in fluorescence when amplified fibrils were present. In particular, 7m showed a selective increase in fluorescence for PDD AFs compared to AD AFs. The fluorescence of 7m with PDD AFs was also greater than in the controls where αS PFFs were added to the lysates with no amplification. These data show that 7m has selectivity toward the conformation found in PDD AFs.

### Example 7: Bimane Derivatives as “Turn On” Probes for β-Amyloid Peptide (Aβ) in Alzheimer's Disease (AD)

As described elsewhere herein, the structural similarities of the styryl bimane compounds to amyloid binding dyes led to the exploration of the potential for using them in amyloid sensing. Initial efforts in this regard were focused on alpha synuclein (αS) due to its role in PD, and subsequent efforts in this regard were focused on β-amyloid peptide (Aβ) due to its role in AD.

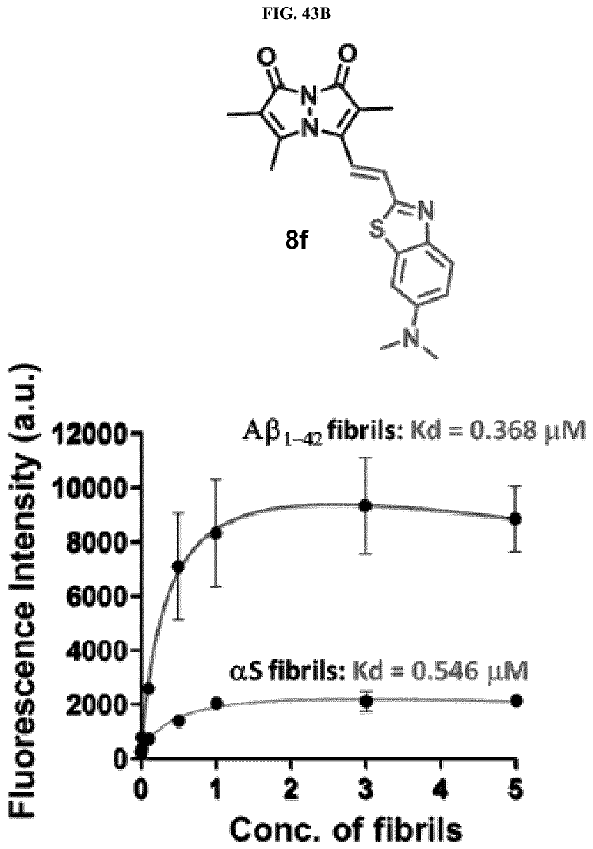

Compounds 8b-8f were screened against in vitro generated alpha synuclein (αS) and β-amyloid peptide (Aβ) pre-formed fibrils (PFFs) and observed by fluorescence spectroscopy. Among these probes, compound 8f demonstrated showed a high turn on of fluorescence in the presence of PFFs (FIG. 42 and FIGS. 43A-43B).

### Example 8: Computational Studies

All calculations were performed employing the APF-D density functional as implemented in the Gaussian16™ suite of programs with the 6-311+G (2d,p) basis set. Geometry optimizations and energies were calculated for selected bimane derivatives (7a, 7b, 7c, 7d, 7e, 7f, 7g, 7h, 7i, 7j, 7l, and 7m compounds. The HOMO-LUMO energy gaps between the ground state (S₀) and the first excited state (S₁) of the bimane derivatives were calculated and are shown below. All structures are ground-state minima according to the analysis of their vibrational frequencies, which showed no negative value.

The correlation between the photophysical properties of bimane derivatives and the electronic properties of the substituents was analyzed to understand the mechanism of fluorescence tuning of the bimane scaffold. It is generally accepted that the absorption wavelength (λ_{abs}) and emission wavelength (λₑₘ) correlate well with the energy gap between HOMO and LUMO as determined by simple electronic structure calculations. Relative energy levels of HOMO and LUMO were calculated using APF-D/6-311+G (2d,p) density functional theory (DFT) calculations. Representative compounds 7f (CH₃), 7g (OCH₃), 7h (OH), 7j (NH₂), and 7l (NMe₂)), with electron donating groups at the para-position, showed a decrease in HOMO-LUMO gap with more electron donating substituents that matched well with red-shifts of the UV-Vis absorbance (Table 11 and FIGS. 15A-15C).

TABLE 11
Calculation of the Energy Gaps of
Bimane Derivatives in Water (H₂O)

| | | Absor- | | | | |
|---|---|---|---|---|---|---|
| | | bance | | | #LUMO- | |
| | Hammett | Wave- | | | #HOMO | LUMO- |
| | Constant | length | HOMO | LUMO | Transi- | HOMO |
| Cmpd | (σₚ) | (nm)^{a} | (eV) | (eV) | tion | Δ(eV) |
| 7a | 0.778 | 335 | −6.547 | −3.232 | 86-85 | 3.315 |
| 7b | 0.66 | 320 | −6.523 | −2.827 | 81-80 | 3.696 |
| 7c | 0.062 | 321 | −6.419 | −2.506 | 79-78 | 3.912 |
| 7e | 0 | 321 | −6.430 | −2.514 | 75-74 | 3.915 |
| 7f | −0.17 | 332 | −6.351 | −2.470 | 79-78 | 3.881 |
| 7h | −0.268 | 348 | −6.146 | −2.420 | 83-82 | 3.726 |
| 7i | −0.37 | 354 | −6.192 | −2.420 | 79-78 | 3.772 |
| 7l | −0.66 | 385 | −5.812 | −2.383 | 79-78 | 3.429 |
| 7m^{b} | −0.83 | 418 | −5.528 | −2.356 | 87-86 | 3.172 |

^{a}Absorbance spectra recorded in ACN:PBS buffer (50:50 v/v),
^{b}Probe with diene linker

Interestingly, compounds with electron withdrawing substituents, 7a (NO₂), 7b (CN), and 7c (F) showed a shallower correlation of UV-Vis absorbance with HOMO-LUMO gap, indicative of a change in excitation mechanism (FIGS. 15A-15C). The change in mechanism can be explained by examination of the HOMO and LUMO of these compounds (FIGS. 16A-16E). For compounds with electron-withdrawing substituents such as 7a, the electrons move from a HOMO centered on the bimane group to a LUMO centered on the styryl group. In contrast, for compounds with electron-donating substituents such as 7l, the electrons move from a HOMO centered on the styryl group to a LUMO centered on the bimane group. This is similar to the observations of the changes in λ_{abs} with the Hammett constant in FIGS. 2A-2G.

### Example 9: Selective Amyloid Binding Dyes

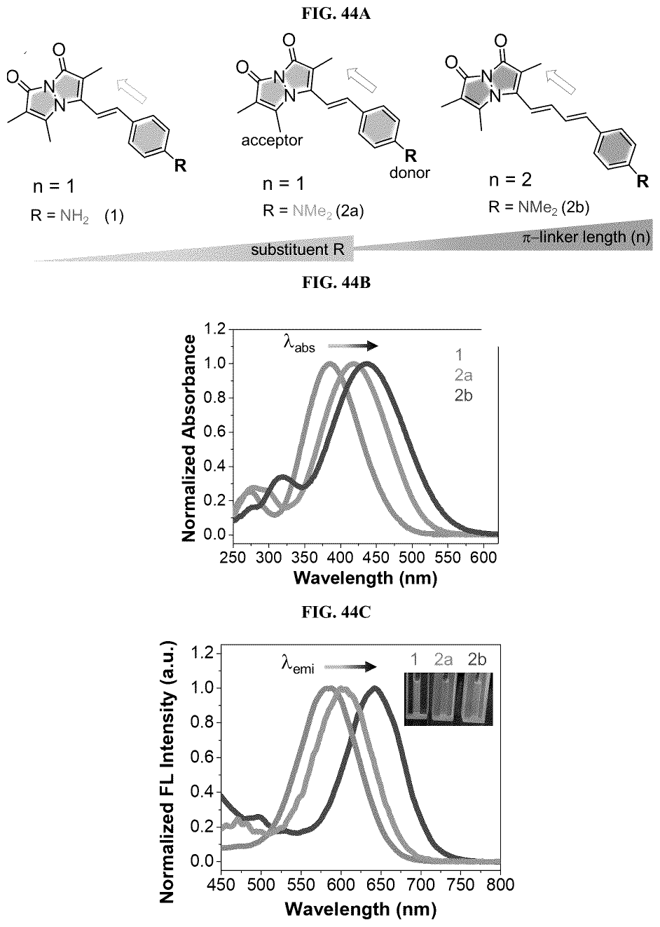

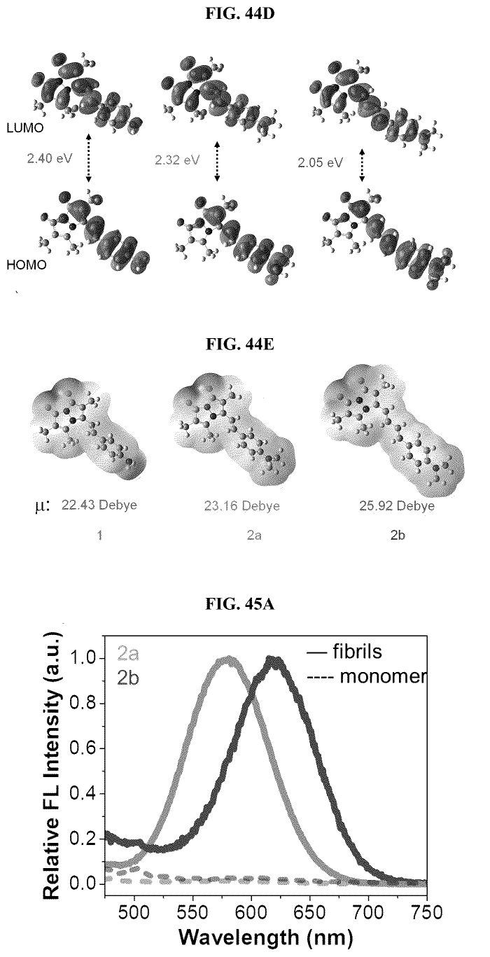

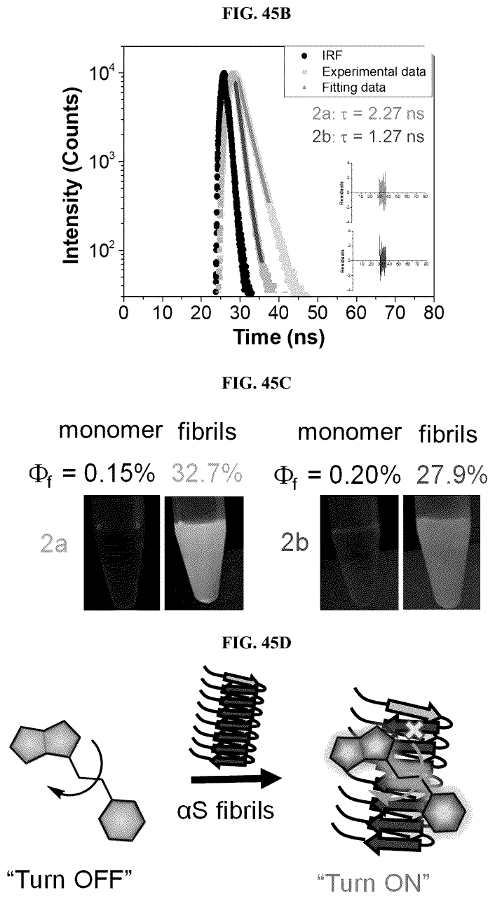

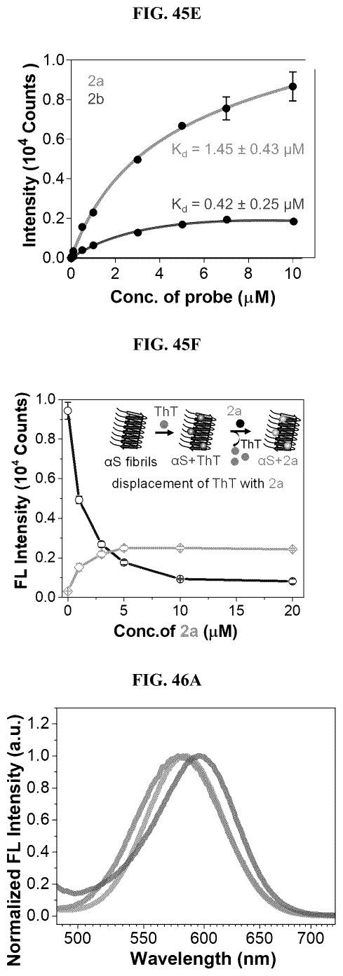

Experimental determination of selective amyloid binding dyes was investigated with respect to compounds of the disclosure. FIG. 44A shows probe structures of compounds 1, 2a, and 2b by varying either the substitution or the linker length (n). FIGS. 44B-44C show comparison of absorbance and fluorescence intensity of the compounds, illustrating the trend in photophysical properties. FIGS. 44D-44E show theoretical models of the compounds helping explain photophysical properties including Debye constant and highest occupied molecular orbital (HOMO). FIG. 45A shows a spectra with and without compounds 2a and 2b and fibrils or monomers, illustrating the shift in wavelength of relative fluorescence intensity. FIG. 45B shows intensity over time of the IRF, experimental data, and fitting data for 2a and 2b. FIG. 45D shows a schematic representation of the “turn on” fluorescence of 2a and 2b probes upon binding to αS PFFs, which resulting from the restricted internal rotation, whereas FIG. 45C shows an image of experimental samples of fibrils with either 2a or 2b. FIG. 45E shows determination of the dissociation constant of probes 2a and 2b (10 nM-10 mM) binding to 100 mM αS-PFFs. FIG. 45F shows displacement of the known amyloid-binding dye ThT by 2a. Excitation and emission wavelength for fluorescence measurements: Excitation (Ex)/Emission (Em): 463 nm/580 nm for 2a, 484 nm/620 nm for 2b, and 450 nm/482 nm for ThT. The results show compounds of the disclosure have appropriate properties to visually or spectroscopically determine the state of fibrils.

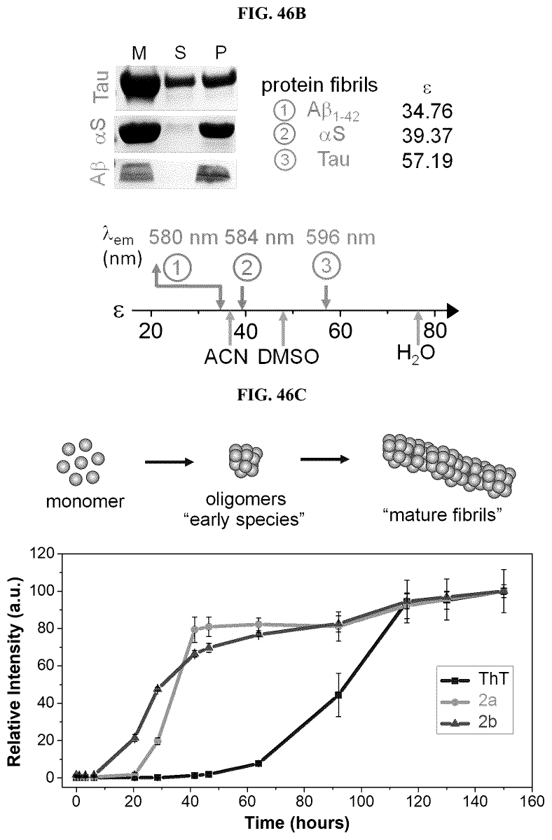

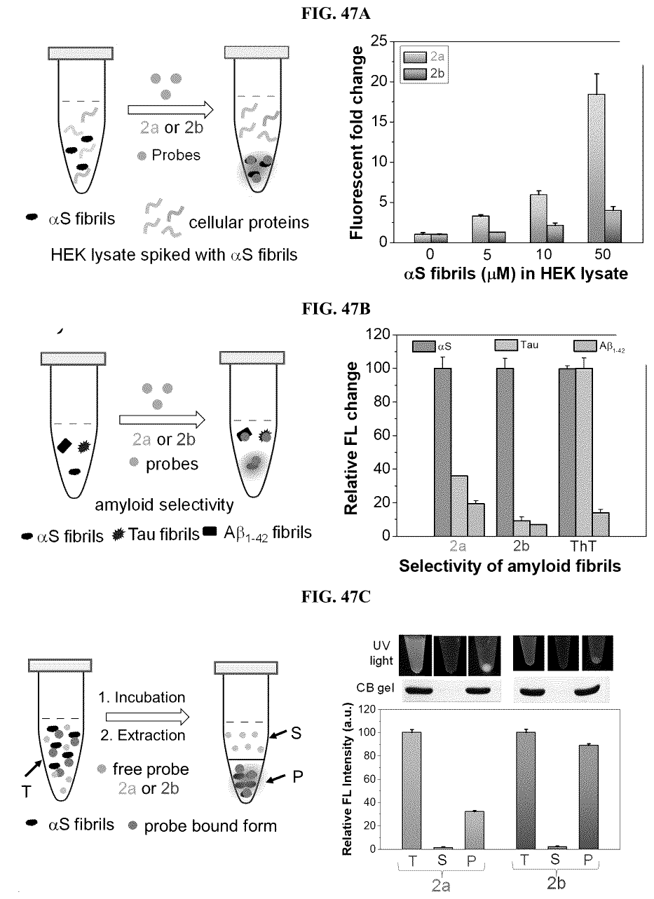

FIGS. 46A-46C show polarity within different aggregated proteins spans over a wide range. FIG. 46A: Fluorescence emission wavelength of 2a quantifies the dielectric constant inside different protein aggregates. 2a (10 μM) was mixed with 50 μM protein fibrils. FIG. 46B has, on the top, a schematic representation of αS fibrils formation involves stepwise pathway. On the bottom is detection of αS aggregation by 2a, 2b and ThT shows RBFs with extended linkages enable the detection of misfolded protein oligomers in vitro. FIG. 46C shows relative intensity (a.u.) of 2a, 2b, and ThT over 160 hour time span, indicating monomer evolution towards mature fibrils.

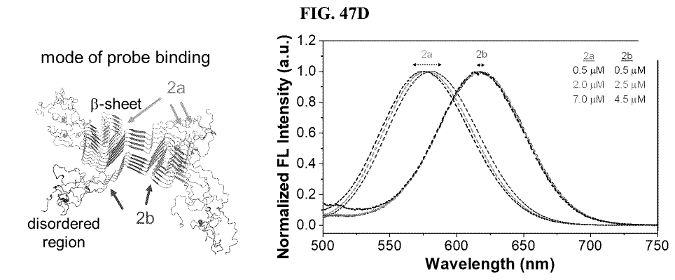

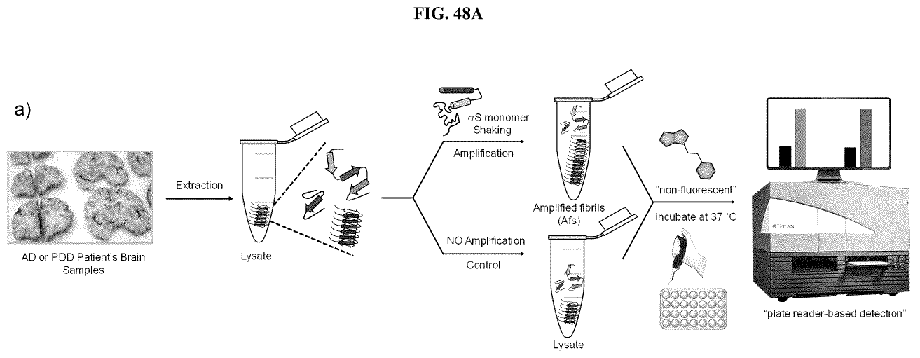

FIG. 47A shows, on the left, a schematic representation of selective detection of αS fibrils in the presence of cellular proteins that are abundant in the cytosolic HEK cell lysate; On the right of FIG. 47A is fluorescence fold change of 2a and 2b (1 μM) with αS PFFs (0-50 μM) at 10 mg/mL HEK lysate concentration. FIG. 47B shows amyloid selectivity of the probes 2a and 2b (1 μM) compared to the standard amyloid binding dye ThT with different amyloid fibrils of αS, tau and Aβ1-42 that are present in patients with neurodegenerative diseases. FIG. 47C shows fractionation of αS-PFFs revealed that 2b retained its fluorescence in the pellet fraction more than 2a, T: total; S: supernatant; P: Pellet; top: Coomassie blue gel of T, S, P fractions and their photographs fractions under handheld UV light (365 nm). FIG. 47D shows, on the left, ssNMR of αS fibrils structure with mode of probes 2a and 2b binding at different regions such as disordered regions and β-sheets, right: Concentration-dependent shift in the emission maximum of 2b with αS fibrils indicates binding at multiple sites, whereas 2a, which shows a minimal shift, suggests binding at limited sites with high affinity. λex/λem=463/580 nm for 2a, and λex/λem=484/620 nm for 2b. λex/λem=450/482 nm for ThT Error bars represent SD of 3 measurements.

### Workflow for Selective Amyloid Interaction of Molecular Probes

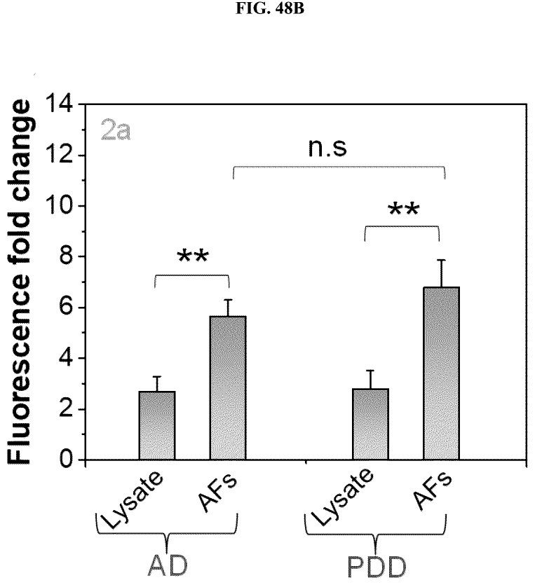

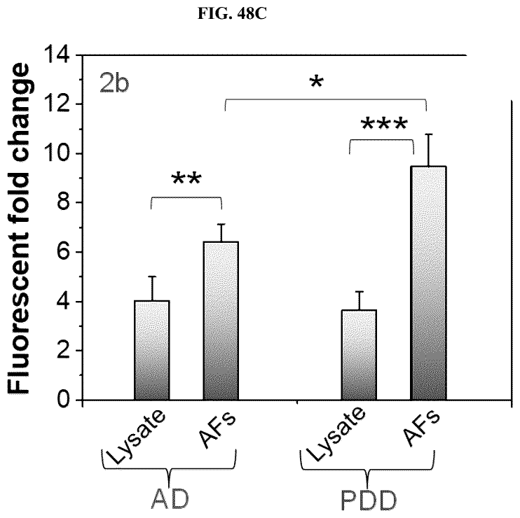

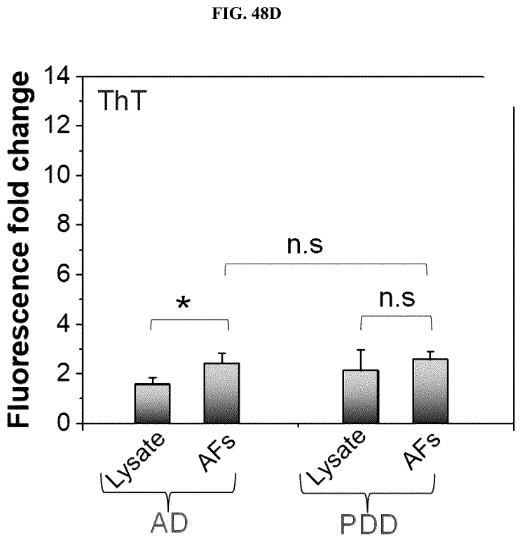

Workflow for patient sample preparation and diagnostic detection of amplified fibrils by a plate reader-based fluorescence assay is shown in FIG. 48A. Three different AD and PDD patient samples were extracted, so-called AD or PDD lysates, used as controls, containing a large number of cellular proteins and a low concentration of amyloid fibrils. Subsequently, amplified fibrils (AFs) were prepared from AD and PDD lysates by adding 1 μM αS monomer. The final brain lysate samples extracted from the brains of AD and PDD patients were characterized by Western blots. FIG. 48B shows fluorescence fold change of the probes 2a with brain lysates and AFs derived from AD and PDD patients. The florescence bar graph represents the averaged data based on three different AD and PDD patients derived AFs and lysates. The final lysate, AFs and probe concentrations were 1 μM each. Fluorescence fold change was subjected to subtraction of free probe fluorescence in PBS buffer. Fluorescence measurements were performed on a Tecan plate reader with the following excitation and emission wavelengths: 463/580 nm for 2a, 484/620 nm for 2b and 450/482 nm for ThT. Error bars represent the SD of 3 measurements. Statistical analysis: *p<0.05, **p<0.01, and ***p<0.005. FIG. 48C shows fluorescence fold change of the probes 2b with brain lysates and AFs derived from AD and PDD patients. The florescence bar graph represents the averaged data based on three different AD and PDD patients derived AFs and lysates. The final lysate, AFs and probe concentrations were 1 μM each. Fluorescence fold change was subjected to subtraction of free probe fluorescence in PBS buffer. Fluorescence measurements were performed on a Tecan plate reader with the following excitation and emission wavelengths: 463/580 nm for 2a, 484/620 nm for 2b and 450/482 nm for ThT. Error bars represent the SD of 3 measurements. Statistical analysis: *p<0.05, **p<0.01, and ***p<0.005. FIG. 48D shows fluorescence fold change of the probes ThT with brain lysates and AFs derived from AD and PDD patients. The fluorescence bar graph represents the averaged data based on three different AD and PDD patients derived AFs and lysates. The final lysate, AFs and probe concentrations were 1 μM each. Fluorescence fold change was subjected to subtraction of free probe fluorescence in PBS buffer. Fluorescence measurements were performed on a Tecan plate reader with the following excitation and emission wavelengths: 463/580 nm for 2a, 484/620 nm for 2b and 450/482 nm for ThT. Error bars represent the SD of 3 measurements. Statistical analysis: *p<0.05, **p<0.01, and ***p<0.005. The results show the value of the workflow to produce a viable determination of molecular state of fibrils or, more generally, proteins of interest.

### Testing of Additional Compounds

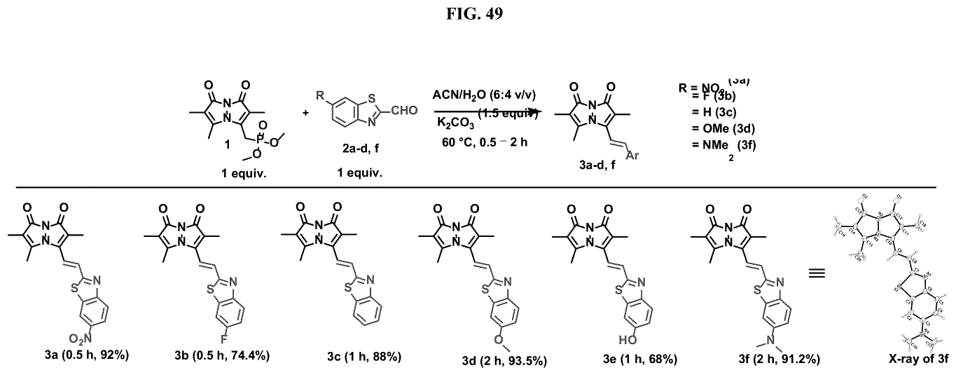

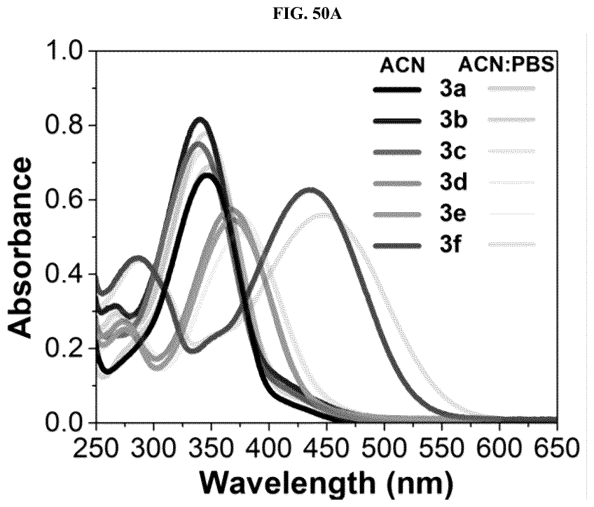

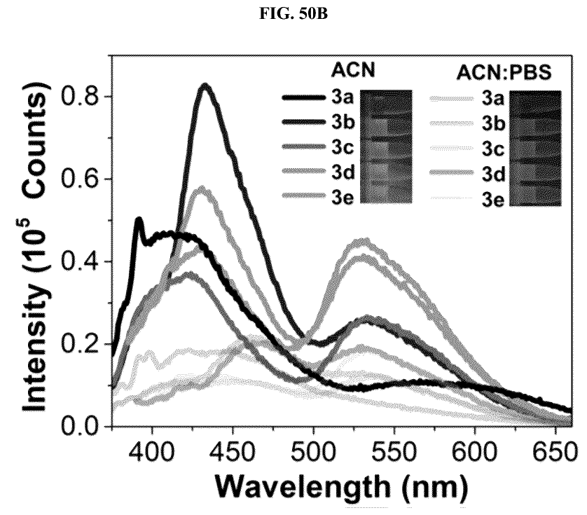

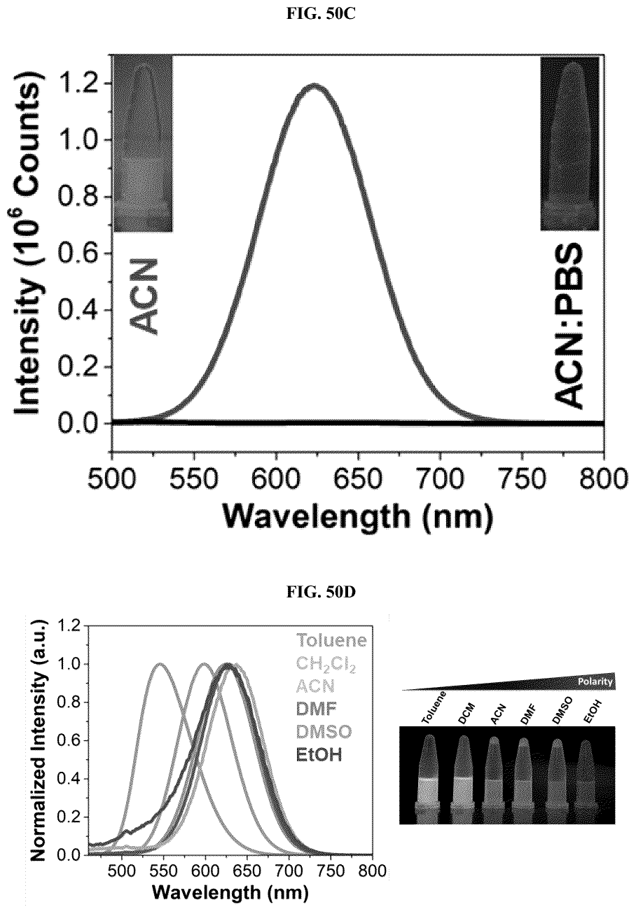

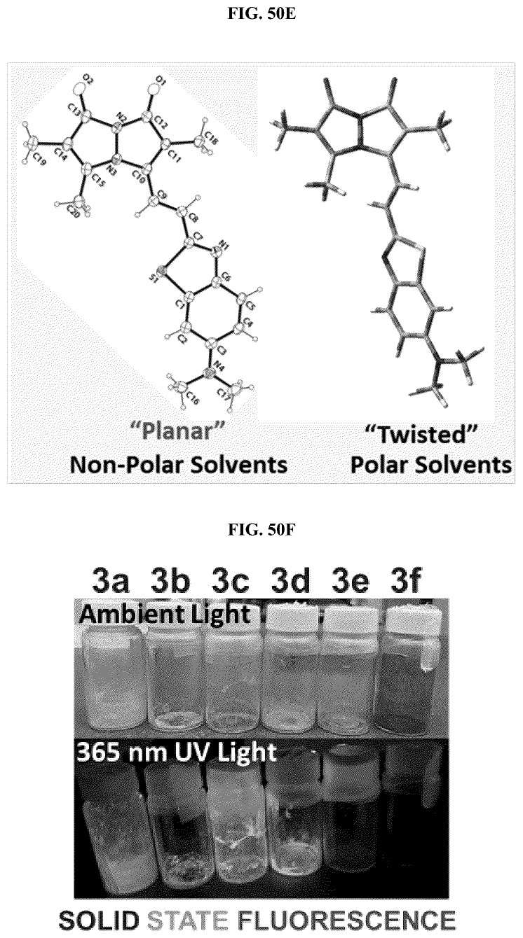

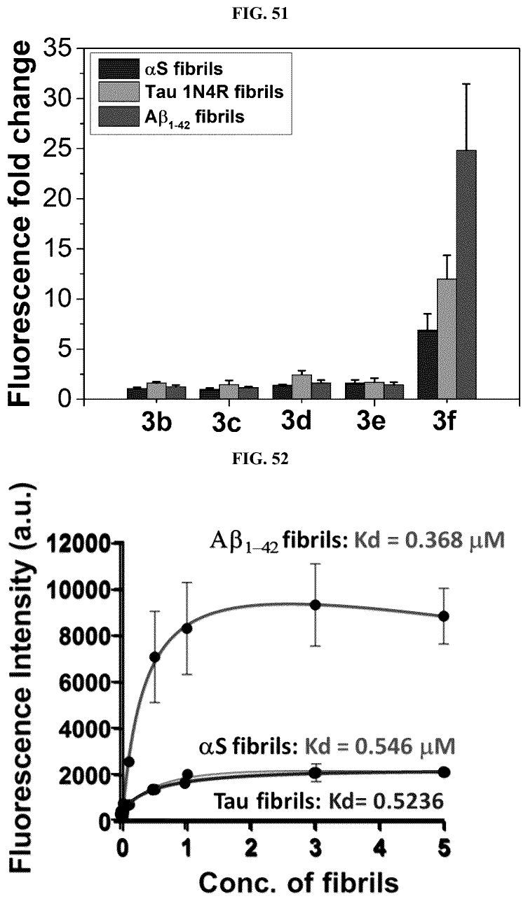

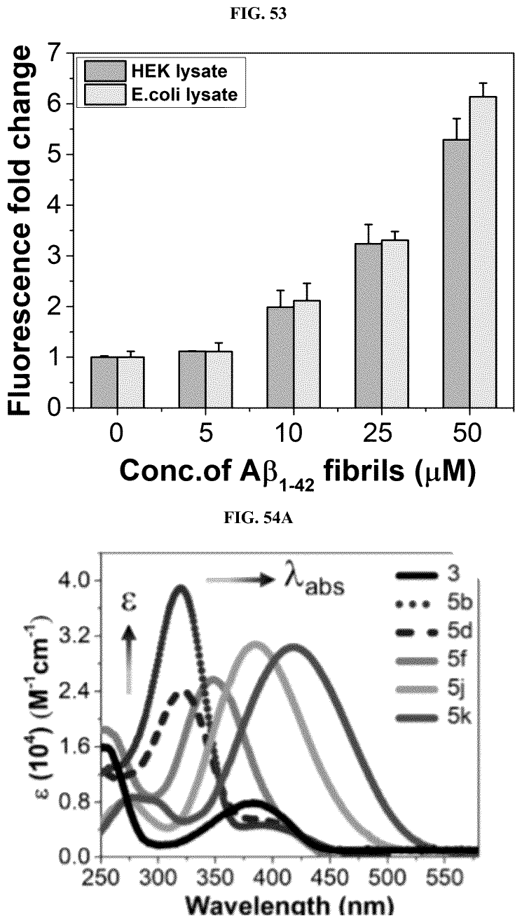

FIG. 49 shows chemical synthesis summary of certain compounds of the disclosure. The compounds were developed to experimentally verify which compound has the best electronic properties as a molecular tool. Absorbance and emission were then analyzed, with results shown in FIG. 50A-50F for compounds alone, in particular solvent, or as a crystal (FIG. 50E). Compound-fibril mixture absorbance and emission results are shown in FIG. 51. FIG. 52 shows the Kd determination of 3f, which was chosen based on promising results in mixtures with fibrils. FIG. 53 shows fluorescence fold change of 3f (1 μM) with Ab1-42 fibrils in the presence of 1 mg/mL cytosolic HEK cell lysate and E. coli lysate. Thus, for this application of investigating the molecular state of Ab1-42 fibrils, 3f is a surprising viable molecular tool.

### Example 10: Development of Photophysical Property Optimization

To pursue the goal of improving photophysical properties and advancing the development of next-generation fluorophores, we present a small but highly tunable bimane scaffold accessible through key intermediate 3 in Scheme 2. This scaffold is designed to be compatible with the rational design and synthesis of libraries of fluorescent probes (5a-5k). The molecular probes offer the capability to design photophysical properties, including (1) visible light excitation, (2) a 3- to 4-fold increase in molar extinction coefficient (F), (3) highly tunable emission spanning from blue to red color, and (4) large (˜200 nm) Stokes shifts. Remarkably, derivatives featuring electron-donating groups (EDGs) demonstrate sensitivity to both polarity and viscosity through intramolecular charge transfer (ICT) and twisted intramolecular charge transfer (TICT) mechanisms, respectively.

### Experimental Details

Materials. Trichloroisocyanuric acid (TCCA), trimethyl phosphite and all other derivatives of aryl aldehydes were purchased from Millipore Sigma (St. Louis, MO, USA). Flash column chromatography was performed using Silicycle silica gel (40-63 m (230-400 mesh), 60 Å irregular pore diameter). Thin-layer chromatography (TLC) was performed on TLC Silica gel 60G F254 plates from Millipore Sigma. Reagents were purchased at the highest commercial quality and used without further purification, unless otherwise stated. The β-amyloid (1-42) peptide, human (Cat. No. RP10017) was purchased from Genscript (Piscataway, NJ).

Instruments. Nuclear magnetic resonance (NMR) spectra were obtained on a Bruker UNI-400 MHz or UNI-600 MHz instrument (Billerica, MA, USA) and are calibrated using peaks from residual protic solvent in deuterated solvent. The following abbreviations were used to denote multiplicities: s=singlet, d=doublet, t=triplet, q=quartet, m=multiplet, br=broad). Low resolution mass spectra (LRMS) were obtained on a Waters Acquity Ultra Performance LC connected to a single quadrupole detector mass spectrometer (Waters Corp.; Milford, MA, USA). High-resolution mass spectra (HRMS) were obtained on a High-resolution electrospray ionization mass spectra (ESI-HRMS) using Waters LCT Premier XE liquid chromatograph/mass spectrometer. Xray diffraction data obtained on a Rigaku XtaLAB Synergy-S diffractometer equipped with an HPC area detector (HyPix-6000HE) and employing confocal multilayer optic-monochromated Mo-Kα radiation (λ=0.71073 Å) or Cu=Kα radiation (λ=1.54184 Å) at a temperature of 100K. Absorbance readings for the DC assay were made on a Tecan M1000 plate reader (Mannedorf, Switzerland). UV-Vis absorption spectra were acquired on a Thermo Scientific Genesys 150 UV-Vis spectrometer (Waltham, MA, USA) using quartz cells with a 1 cm cell path length (Starna Cells, Inc 120 ul UV cells). Fluorescence spectra were acquired on a Tecan M1000 plate reader. Quantum yield (QY) measurements were performed using a Jasco FP-8300 Fluorimeter with ILF-835 integrating sphere attachment (Easton, MD, USA). Fluorescence lifetime measurements were made using a Photon Technology International (PTI) QuantaMaster™ 40 fluorescence spectrometer (Birmingham, NJ, USA).

### Multi-Gram Scale Synthesis of Bimane Core

### Synthesis of 3,4-dimethyl-2-pyrazolin-5-one

Ethyl 2-methylacetoacetate (30 g, 208.3 mmol, 1 equiv) was condensed with 4 equiv. of hydrazine (26.7 g, 833.2 mmol, 35 wt. % in H2O) under sonication for 30 min to give a white precipitate. After filtration of the white precipitate and washing several times with ethyl acetate followed by dichloromethane to get rid of the unreacted starting materials, the precipitate was dried overnight to afford 22.485 g of compound 3,4-dimethyl-2-pyrazolin-5-one in 96.4% isolated yield.

### Synthesis of 3,4-dimethyl-4-chloro-2-pyrazolin-5-one

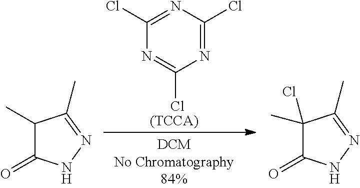

3,4-dimethyl-2-pyrazolin-5-one (20 g, 178.4 mmol) was dissolved in 200 mL of dichloromethane (DCM). The solution was cooled on an ice bath. Trichloroisocyanuric acid (TCCA, 13.7 g, 59.5 mmol) was slowly added to the reaction mixture over a period of 30 min. After completion of the addition, the reaction mixture was stirred overnight at room temperature and the cyanuric acid byproduct was filtered off. The filtrate was concentrated under reduced pressure at 40° C. to afford 22.05 g of 3,4-dimethyl-4-chloro-2-pyrazolin-5-one in 84% isolated yield.

### Synthesis of 2,3,5,6-tetramethyl-1H,7H-pyrazolo[1,2-a]pyrazole-1,7-dione (1)

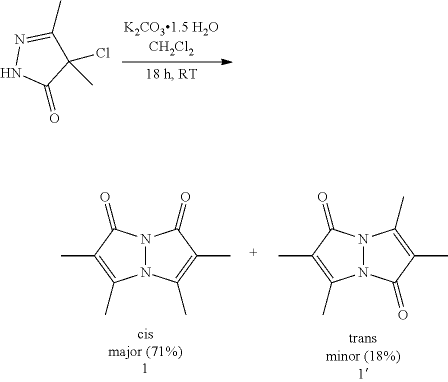

3,4-Dimethyl-4-chloro-2-pyrazolin-5-one (15 g, 102.34 mmol, 1 equiv) was dissolved in 150 mL DCM and cooled on an ice bath. Then add potassium carbonate hydrate (K2CO3·1.5H2O) (59.32 g, 429.83 mmol, 4.2 equiv) and stir for 18 hours at room temperature. Then the K2CO3 was filtered, and the filtrate concentrated to afford a mixture of cis (1) and trans (1′) isomers of bimane. 2,3,5,6-tetramethyl-1H,7H-pyrazolo[1,2-a]pyrazole-1,7-dione, with 1 as the major product. The reaction mixture was purified by column chromatography eluting with 40-70% ethyl acetate in hexane to afford the 71% of compound 1 (Rf=0.1, 70% EtOAc in hexane) and compound 1′ (R_{f}=0.64, 70% EtOAc in hexane) in 18% yield.

### Synthesis of 3-(bromomethyl)-2,5,6-trimethyl-1H,7H-pyrazolo[1,2-a]pyrazole-1,7-dione (2)

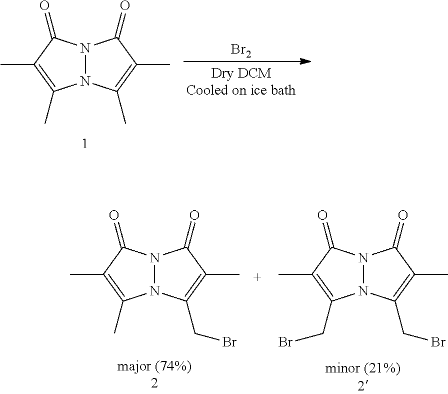

Compound 1 (3 g, 15.61 mmol) was dissolved in dry 120 mL of DCM. The solution was cooled on an ice bath and a bromine solution (0.8 mL, 15.61 mmol) in 30 mL of DCM solvent was added dropwise over a period of 30 min under cold conditions. Upon completion of the addition, the reaction mixture was stirred for another 30 min under cold conditions. The progress of the reaction was monitored by TLC. Upon completion of the reaction, the resulting mixture was neutralized with cold water and then extracted with DCM. The organic layer was collected and concentrated by rotary evaporation. The crude product was purified by flash column chromatography eluting with 30-60% ethyl acetate in hexane to afford the desired monobrominated product 2 (R_{f}=0.17, 70% EtOAc in hexane) in 74% yield and we also isolated the dibrominated product 2′ (R_{f}=0.45, 70% EtOAc in hexane) in 21% yield.

### Synthesis of dimethyl ((2,5,6-trimethyl-1,7-dioxo-1H,7H-pyrazolo[1,2-a]pyrazol-3-yl)methyl)phosphonate (3)

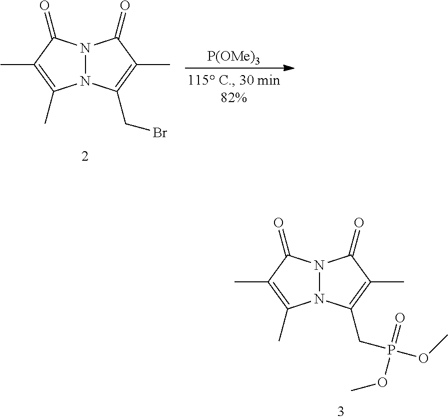

A mixture of monobromobimane (2, 2.0 g, 7.38 mmol) and trimethyl phosphite (2.85 mL) was heated at 115° C. for 30 min until it became homogeneous and then solidified. After trituration with hexane (50 mL), the mixture was slurried in ethyl acetate and filtered to remove the remaining trimethyl phosphite. Then, the solid product was redissolved in DCM and purified by flash column chromatography using 3% methanol in DCM as an eluent to afford 1.82 g of the phosphonate 3 (R_{f}=0.37, 3% methanol in DCM) as a light-yellow solid in 82% isolated yield. 1H NMR (400 MHz, CDCl₃) δ 3.78 (d, J=11.1 Hz, 6H), 3.20 (d, J=22.0 Hz, 2H), 2.42 (s, 3H), 1.84 (d, J=4.1 Hz, 3H), 1.80 (s, 3H). 13C NMR (101 MHz, CDCl₃) δ 160.7, 160.2, 146.5, 140.0, 114.7, 113.2, 53.7, 25.2, 23.8, 11.9, 7.0. HRMS (ESI+) calcd for C₁₂H₁₇N₂O₅P [M+H]+, 301.0953; found: 301.0965.

### H-W-E Procedures

Horner-Wadsworth-Emmons (H-W-E) reactions were performed with one of two general procedures:

### H-W-E General Procedure A

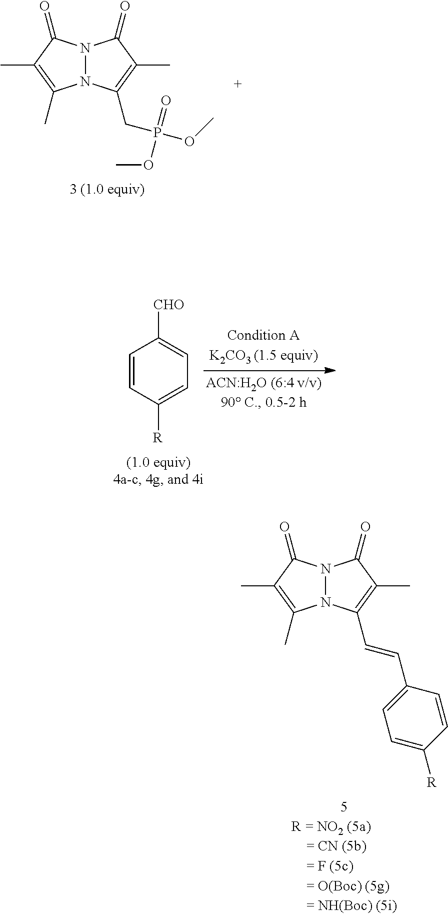

To a solution of dimethyl bimanephosphonate 3 (1 mmol) in acetonitrile (ACN)/H2O (2 mL, 6:4 v/v), add the aryl aldehydes 4a-c, 4g, and 4i (1 mmol) and K₂CO₃ (1.5 mmol) and heat at 90° C. for 30 min-2 h. The reaction progress was checked by TLC, upon completion of the reaction, the solution was cooled to room temperature and the organic layer was extracted with DCM, washed with water (2×50 mL), dried using Na₂SO₄, and concentrated under vacuum to yield the crude mixture. Then, the product was purified by recrystallization from MeOH.

### H-W-E General Procedure B

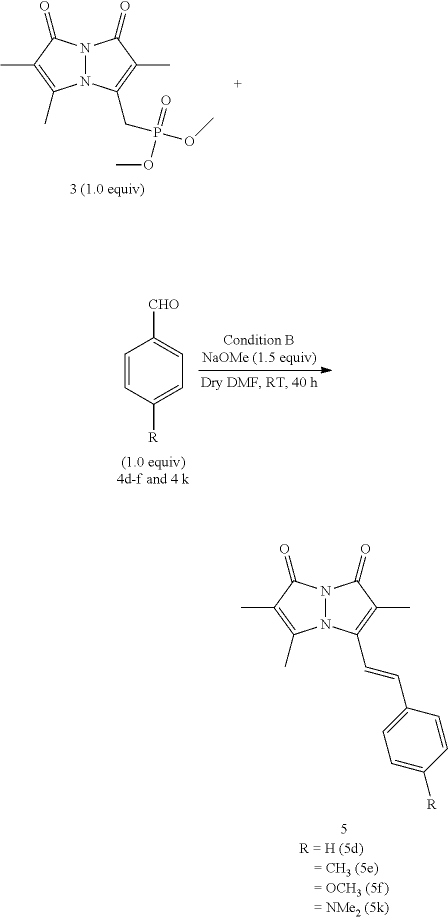

To a solution of phosphonate 3 (1 mmol) in dry DMF, the aryl aldehydes 4d-f and 4k (1 mmol) and NaOMe (1.5 mmol) were added, and the resulting solution was stirred at RT for 40 h under argon atmosphere. The reaction progress was monitored by TLC, and upon completion, the organic layer was extracted with DCM, washed with water (2×50 mL), dried using Na2SO4, and concentrated under vacuum to yield the crude product. Then, the product was purified by flash column chromatography using 3% methanol in DCM as an eluent.

Scheme S1. Optimization of H-W-E reaction of 3 and 4-(dimethylamino)benzaldehydeª

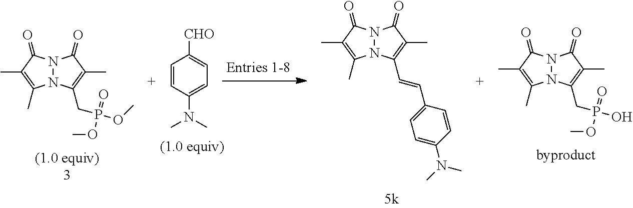

| Entry | Solvent | Base | Temperature | Time (hours) | Yield (%) |
|---|---|---|---|---|---|
| 1 | ACN/H₂O | K₂CO₃ | RT | 40 h | 2.3 |
| 2 | ACN/H₂O | K₂CO₃ | 90º C. | 2 h | 9 |
| 3 | DMF | KOtBu | RT | 40 h | 27 |
| 4 | DMF | NaOMe | RT | 40 h | 46 |
| 5 | DMF | NaOMe | 60° C. | 40 h | 38.4 |
| 6 | DMSO | KOtBu | RT | 40 h | 3.5 |
| 7 | DMSO | NaOMe | RT | 40 h | 31 |
| 8 | DMSO | NaOMe | 60° C. | 40 h | 11.5 |

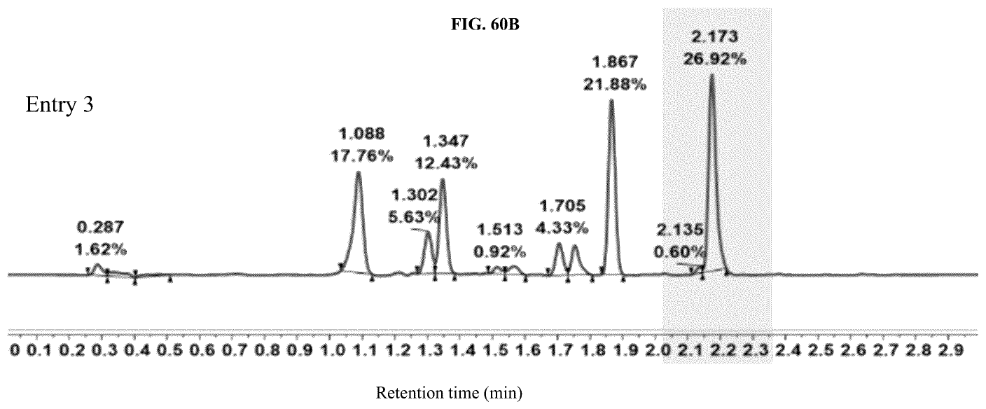

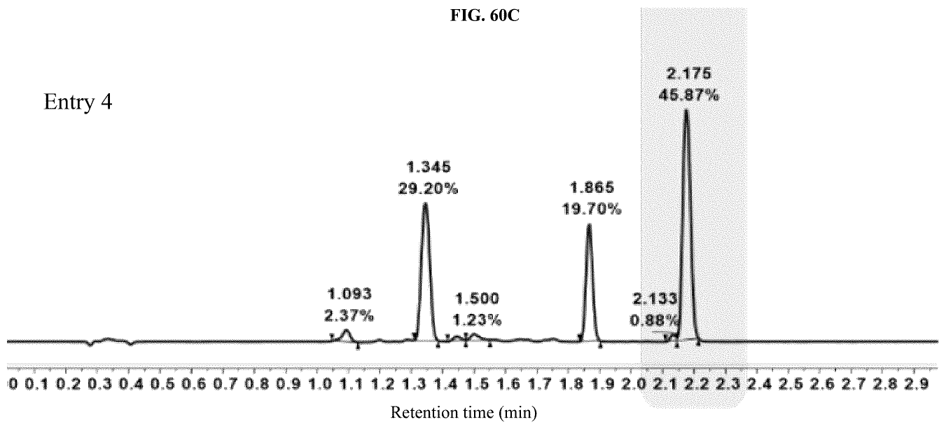

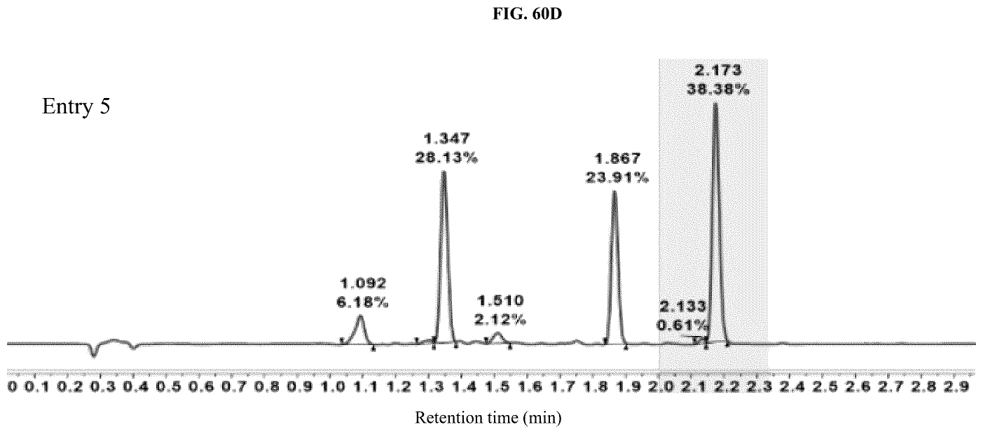

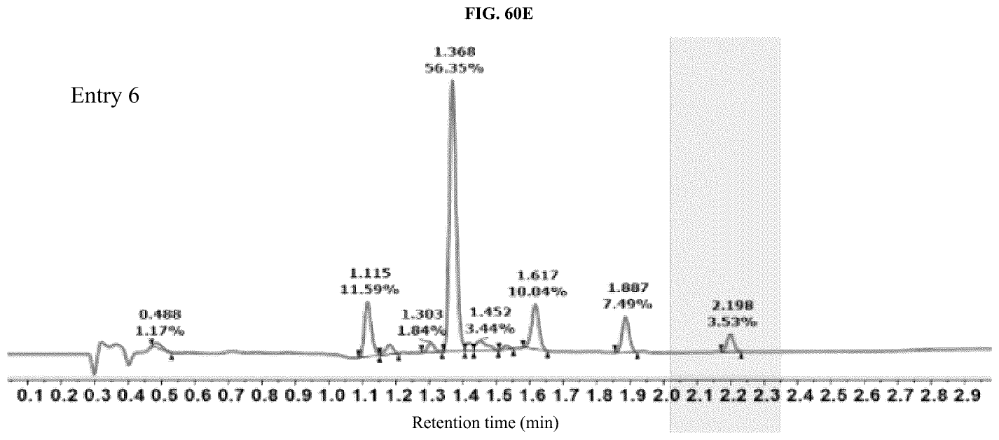

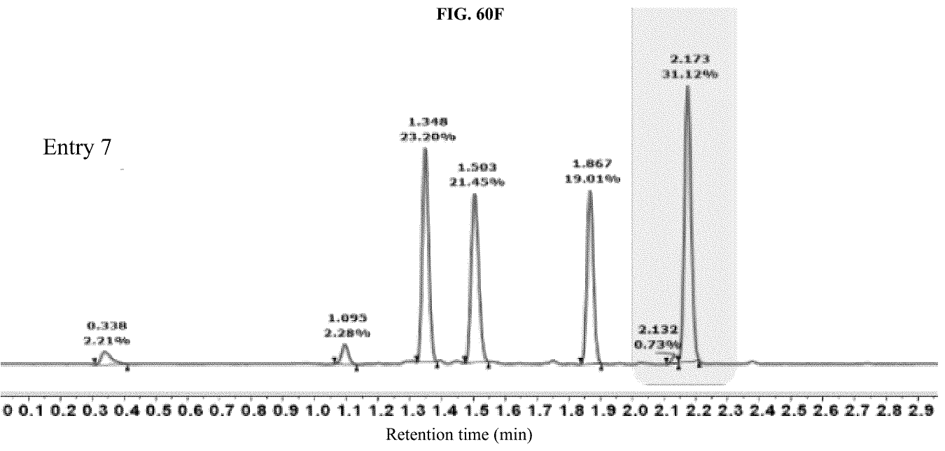

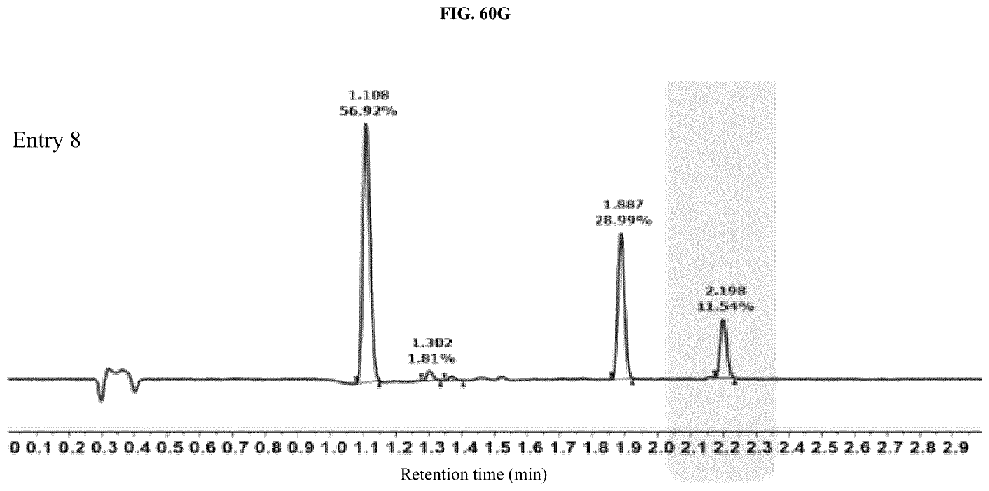

^{a}Reaction conditions: 3 (1 equiv), aryl aldehyde 4k (1 equiv), KOtBu (1.5 equiv) or NaOMe (1.5 equiv), K₂CO₃ (1.5 equiv). FIGs. 60A-60G show liquid chromatography (LC) for the conversion of 5k under different optimized reaction conditions.

Synthesis and Characterization of 5a-5k

### (E)-2,3,6-trimethyl-5-(4-nitrostyryl)-1H,7H-pyrazolo[1,2-a]pyrazole-1,7-dione (5a)

Following General Procedure A with 4-nitrobenzaldehyde (4a, 50.3 mg, 0.33 mmol, 1 equiv, purchased from Aldrich) at 90° C. for 30 min afforded 106 mg (98% isolated yield, yellow solid) of 5a. 1H NMR (400 MHz, CDCl₃) δ 8.29 (d, J=8.9 Hz, 2H), 7.70 (d, J=8.8 Hz, 2H), 7.17 (d, J=16.5 Hz, 1H), 6.96 (d, J=17.3 Hz, 1H), 2.30 (s, 3H), 2.03 (s, 3H), 1.84 (s, 3H). 13C NMR (101 MHz, CDCl₃) δ 161.7, 161.3, 148.5, 146.4, 141.0, 138.5, 128.1, 124.7, 118.3, 115.2, 114.3, 12.7, 8.6, 7.2. HRMS (ESI+) calcd for C₁₇H₁₅N₃O₄ [M+H]+, 326.1141; found: 326.1156.

### (E)-4-(2-(2,5,6-trimethyl-1,7-dioxo-1H,7H-pyrazolo[1,2-a]pyrazol-3-yl)vinyl)benzonitrile (5b)

Following General Procedure A with 4-cyanobenzaldehyde (4b, 43.6 mg, 0.33 mmol, 1 equiv, purchased from Aldrich) at 90° C. for 1 h afforded 85.3 mg (83.6% isolated yield, yellow solid) of 5b. 1H NMR (400 MHz, CDCl₃) δ 7.73 (d, J=8.4 Hz, 2H), 7.63 (d, J=8.5 Hz, 2H), 7.12 (d, J=16.5 Hz, 1H), 6.91 (d, J=16.5 Hz, 1H), 2.28 (s, 3H), 2.02 (s, 3H), 1.84 (s, 3H). 13C NMR (101 MHz, CDCl₃) δ 161.7, 161.3, 148.6, 146.6, 139.1, 139.0, 133.1, 127.9, 118.4, 117.6, 115.0, 114.2, 113.5, 12.7, 8.6, 7.2. HRMS (ESI+) calcd for C₁₈H₁₅N₃O₂ [M+H]+, 306.1243; found: 306.1235.

### (E)-3-(4-fluorostyryl)-2,5,6-trimethyl-1H,7H-pyrazolo[1,2-a]pyrazole-1,7-dione (5c)

Following General Procedure A with 4-fluorobenzaldehyde (4c, 41.3 mg, 0.33 mmol, 1 equiv, purchased from Aldrich) at 90° C. for 2 h afforded 77.2 mg (78% isolated yield, yellow solid) of 5c. 1H NMR (400 MHz, CDCl₃) δ 7.52 (dd, J=8.9, 5.3 Hz, 2H), 7.13 (t, J=8.6 Hz, 2H), 7.07 (d, J=16.4 Hz, 1H), 6.71 (d, J=16.5 Hz, 1H), 2.28 (s, 3H), 2.01 (s, 3H), 1.83 (s, 3H). 13C NMR (101 MHz, CDCl₃) δ 164.0 (d, 1JC-F=252.7 Hz), 161.8, 161.6, 148.6, 147.5, 140.1, 131.2, 129.4, 129.3, 116.6 (d, 2JC-F=22.2 Hz), 113.9, 113.7, 12.7, 8.6, 7.2. HRMS (ESI+) calcd for C₁₇H₁₅FN₂O₂ [M+H]+, 299.1196; found: 299.1193.

### (E)-2,3,6-trimethyl-5-styryl-1H,7H-pyrazolo[1,2-a]pyrazole-1,7-dione (5d)

Following General Procedure B with benzaldehyde (4d, 35.3 mg, 0.33 mmol, 1 equiv, purchased from Aldrich) at room temperature for 40 h afforded 67 mg (72.4% isolated yield, yellow solid) of 5d after purification by column chromatography (3% methanol in dichloromethane, R_{f}=0.46). 1H NMR (400 MHz, CDCl₃) δ 7.53 (dd, J=7.9, 1.9 Hz, 2H), 7.48-7.39 (m, 3H), 7.11 (d, J=16.5 Hz, 1H), 6.79 (d, J=16.4 Hz, 1H), 2.29 (s, 3H), 2.02 (s, 3H), 1.84 (s, 3H). 13C NMR (101 MHz, CDCl₃) δ 161.9, 161.7, 148.6, 147.7, 141.4, 135.0, 130.4, 129.4, 127.5, 114.0, 113.9, 113.8, 12.7, 8.6, 7.2. HRMS (ESI+) calcd for C₁₇H₁₆N₂O₂ [M+H]+, 281.1290; found: 281.1283.

### (E)-2,3,6-trimethyl-5-(4-methylstyryl)-1H,7H-pyrazolo[1,2-a]pyrazole-1,7-dione (5e)

Following General Procedure B with p-tolualdehyde (4e, 40 mg, 0.33 mmol, 1 equiv, purchased from Aldrich) at room temperature for 40 h afforded 67 mg (68% isolated yield, yellow solid) of 5e after purification by column chromatography (3% methanol in dichloromethane, Rf=0.47). 1H NMR (400 MHz, CDCl₃) δ 7.45 (d, J=8.1 Hz, 2H), 7.28 (d, J=2.8 Hz, 2H), 7.10 (d, J=16.5 Hz, 1H), 6.76 (d, J=16.4 Hz, 1H), 2.43 (s, 3H), 2.31 (s, 3H), 2.04 (s, 3H), 1.86 (s, 3H). 13C NMR (101 MHz, CDCl₃) δ 161.9, 161.7, 148.7, 148.0, 141.4, 140.9, 132.2, 130.1, 127.4, 113.9, 113.4, 112.9, 77.5, 77.2, 76.9, 21.7, 12.7, 8.6, 7.2. HRMS (ESI+) calcd for C₁₈H₁₈N₂O₂ [M+H]+, 295.1447; found: 295.1447.

### (E)-3-(4-methoxystyryl)-2,5,6-trimethyl-11H,7H-pyrazolo[1,2-a]pyrazole-1,7-dione (5f)

Following General Procedure B with p-anisaldehyde (4f, 45.3 mg, 0.33 mmol, 1 equiv, purchased from Aldrich) at room temperature for 40 h afforded 61 mg (59% isolated yield, yellow solid) of 5f after purification by column chromatography (3% methanol in dichloromethane, R_{f}=0.45). 1H NMR (400 MHz, CDCl₃) δ 7.47 (d, J=8.8 Hz, 2H), 7.05 (d, J=16.5 Hz, 1H), 6.95 (d, J=8.8 Hz, 2H), 6.63 (d, J=17.1 Hz, 1H), 3.86 (s, 3H), 2.28 (s, 3H), 2.00 (s, 3H), 1.83 (s, 3H). 13C NMR (101 MHz, CDCl₃) δ 161.7, 161.6, 161.3, 148.5, 148.0, 140.9, 128.9, 127.5, 114.6, 113.6, 112.7, 111.3, 55.5, 12.5, 8.4, 7.0. HRMS (ESI+) calcd for C₁₈H₁₈N₂O₃ [M+H]+, 311.1396; found: 311.1408.

### (E)-3-(4-hydroxystyryl)-2,5,6-trimethyl-1H,7H-pyrazolo[1,2-a]pyrazole-1,7-dione (5h)

Following General Procedure A with tert-butyl 4-formylphenyl carbonate (4g, 74 mg, 0.33 mmol, 1 equiv, purchased from Aldrich) at 90° C. for 2 h afforded 100.3 mg (76% isolated yield) of tert-butyloxycarbonyl (Boc) protected 5g after purification by column chromatography (3% methanol in dichloromethane, Rf=0.41). Next, the Boc deprotection of 5g (100 mg, 0.252 mmol, 1 equiv) was carried out under acidic conditions using trifluoroacetic acid (431.4 mg, 3.78 mmol, 15 equiv) in 2 mL DCM solvent for 6 h. The reaction progress was monitored by the TLC and upon completion, it was dried under vacuum to afford 55.3 mg of the 5h (74% isolated yield, yellow solid, R_{f}=0.34 in 3% methanol in dichloromethane). 1H NMR (600 MHz, DMSO) δ 9.97 (s, 1H), 7.59 (d, J=8.7 Hz, 2H), 7.20 (d, J=16.5 Hz, 1H), 6.97 (d, J=16.5 Hz, 1H), 6.83 (d, J=8.7 Hz, 2H), 2.34 (s, 3H), 1.91 (s, 3H), 1.74 (s, 3H). 13C NMR (151 MHz, DMSO) δ 161.1, 160.9, 159.4, 150.1, 148.8, 141.2, 129.5, 126.3, 115.8, 111.8, 110.6, 110.5, 12.1, 8.0, 6.6. HRMS (ESI+) calcd for C₁₇H₁₆N₂O₃ [M+H]+, 297.1239; found: 297.1233.

### tert-butyl (E)-(4-(2-(2,5,6-trimethyl-1,7-dioxo-1H,7H-pyrazolo[1,2-a]pyrazol-3-yl)vinyl)phenyl)carbamate (5i)

Following General Procedure A with 4-(Bocamino)benzaldehyde (4i, 73.6 mg, 0.33 mmol, 1 equiv) at 90° C. for 2 h afforded 113.5 mg (86% isolated yield, yellow solid) of 5i after purification by column chromatography (3% methanol in dichloromethane, R_{f}=0.42). 1H NMR (600 MHz, CDCl₃) δ 7.45 (s, 4H), 7.04 (d, J=16.4 Hz, 1H), 6.73-6.60 (m, 2H), 2.28 (s, 3H), 2.02 (s, 3H), 1.84 (s, 3H), 1.53 (s, 9H). 13C NMR (151 MHz, CDCl₃) δ 161.9, 161.8, 152.5, 148.7, 148.0, 129.6, 128.4, 118.8, 113.9, 113.2, 112.2, 81.4, 28.5, 12.7, 8.6, 7.2. HRMS (ESI+) calcd for C₂₂H₂₅N₃O₄ [M+H]+, 396.1923; found: 396.1930.

### (E)-3-(4-aminostyryl)-2,5,6-trimethyl-1H,7H-pyrazolo[1,2-a]pyrazole-1,7-dione (5j)

The deprotection of a Boc-protected 5i (100 mg, 0.253 mmol, 1 equiv) was carried out under acidic conditions using trifluoroacetic acid (432.5 mg, 3.79 mmol, 15 equiv) in 2 mL DCM solvent for 6 h. After completion of the reaction, the reaction progress was monitored by the TLC and concentrated the solvent and dried under vacuum to afford 62.8 mg of 5j (84% isolated yield, red solid, R_{f}=0.39). 1H NMR (600 MHz, DMSO) δ 7.43 (d, J=8.6 Hz, 2H), 7.13 (d, J=16.3 Hz, 1H), 6.81 (d, J=16.3 Hz, 1H), 6.60 (d, J=8.5 Hz, 2H), 5.76 (s, 2H), 2.35 (s, 3H), 1.91 (s, 3H), 1.74 (s, 3H). 13C NMR (151 MHz, DMSO) δ 161.6, 161.5, 151.7, 150.5, 149.9, 142.6, 130.0, 122.9, 114.1, 112.2, 109.8, 108.0, 12.6, 8.6, 7.0. HRMS (ESI+) calcd for C₁₇H₁₇N₃O₂, [M+H]+, 296.1399; found: 296.1405.

### (E)-3-(4-(dimethylamino)styryl)-2,5,6-trimethyl-1H,7H-pyrazolo[1,2-a]pyrazole-1,7-dione (5k)

Following General Procedure B with 4-(dimethylamino)benzaldehyde (4k, 49.6 mg, 0.33 mmol, 1 equiv, purchased from Aldrich) at room temperature for 40 h afforded 46.3 mg (43% isolated yield, orange solid) of 5k after purification by column chromatography (3% methanol in dichloromethane, R_{f}=0.45). 1H NMR (600 MHz, CDCl₃) δ 7.40 (d, J=8.9 Hz, 2H), 7.03 (d, J=16.3 Hz, 1H), 6.71 (d, J=8.9 Hz, 2H), 6.53 (d, J=16.3 Hz, 1H), 3.04 (s, 6H), 2.29 (s, 3H), 2.01 (s, 3H), 1.84 (s, 3H). 13C NMR (151 MHz, CDCl₃) δ 162.1, 162.0, 151.9, 149.0, 148.7, 141.8, 129.0, 122.8, 113.6, 112.2, 111.8, 108.4, 40.4, 12.8, 8.7, 7.2. HRMS (ESI+) calcd for C₁₉H₂₁N₃O₂, [M+H]+, 324.1712; found: 324.1712.

### X-ray Structure Determination of Compound 5a

Compound 5a, C₁₇H₁₅N₃O₄, crystallizes in the monoclinic space group P21 (systematic absences 0k0: k=odd) with a=3.85501(8)Å, b=13.3190(3)Å, c=14.7962(3)Å, β=94.880(2)°, V=756.96(3)Å³, Z=2, and dcalc=1.427 g/cm³. X-ray intensity data were collected on a Rigaku XtaLAB Synergy-S diffractometer equipped with an HPC area detector (HyPix-6000HE) and employing confocal multilayer optic-monochromated Mo-Kα radiation (λ=0.71073 Å) at a temperature of 100 K. Preliminary indexing was performed from a series of thirty 0.5° rotation frames with exposures of 0.5 sec. A total of 1844 frames (11 runs) were collected employing ω scans with a crystal to detector distance of 34.0 mm, rotation widths of 0.5° and exposures of 5 sec. Rotation frames were integrated using CrysAlisPro, producing a listing of unaveraged F2 and σ(F2) values. A total of 19219 reflections were measured over the ranges 4.122≤2θ≤50.682°, −4≤h≤4, −16≤k≤16, −17≤1≤17 yielding 2782 unique reflections (Rint=0.0272). The intensity data were corrected for Lorentz and polarization effects and for absorption using SCALE3 ABSPACK (minimum and maximum transmission 0.8029, 1.0000). The structure was solved by direct methods—ShelXT. Refinement was by full-matrix least squares based on F2 using SHELXL-2018. All reflections were used during refinement. The weighting scheme used was w=1/[σ²(Fo²)+(0.0390P)²+0.1383P] where P=(Fo²+2Fc²)/3. Non-hydrogen atoms were refined anisotropically and hydrogen atoms were refined using a riding model. Refinement converged to R1=0.0237 and wR2=0.0626 for 2734 observed reflections for which F>4σ(F) and R1=0.0241 and wR2=0.0629 and GOF=1.029 for all 2782 unique, non-zero reflections and 220 variables. The maximum Δ/σ in the final cycle of least squares was 0.000 and the two most prominent peaks in the final difference Fourier were +0.12 and −0.15 e/Å³.

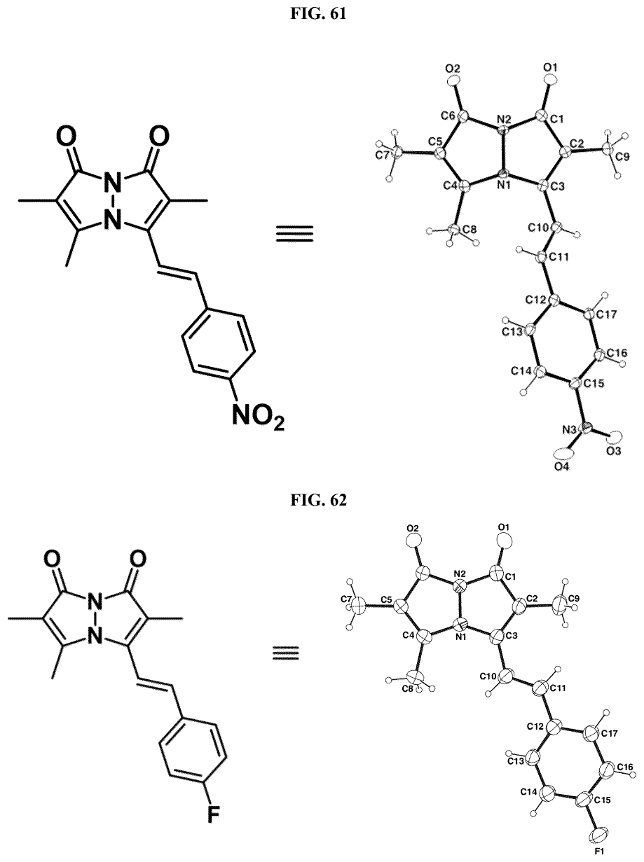

Table S1 lists cell information, data collection parameters, and refinement data. Tables S2. and S3 list bond distances and bond angles. FIG. 61 is an ORTEP representation of the molecule with 50% probability thermal ellipsoids displayed.

TABLE S1
Summary of Structure Determination of 5a.

| Empirical formula | C₁₇H₁₅N₃O₄ | |
|---|---|---|
| Formula weight | 325.32 | |
| Diffractometer | Rigaku XtaLAB Synergy-S | |
| Temperature/K | 100 | |
| Crystal system | monoclinic | |
| Space group | P2₁ | |
| a | 3.85501(8) | Å |
| b | 13.3190(3) | Å |
| c | 14.7962(3) | Å |
| β | 94.880(2)° | |
| Volume | 756.96(3) | Å³ |
| Z | 2 | |
| d_{calc} | 1.427 | g/cm³ |
| μ | 0.104 | mm⁻¹ |

| F(000) | 340.0 |
|---|---|
| Crystal size, mm | 0.2 × 0.07 × 0.07 |
| 2θ range for data collection | 4.122-50.682° |
| Index ranges | −4 ≤ h ≤ 4, −16 ≤ k ≤ 16, −17 ≤ l ≤ 17 |
| Reflections collected | 19219 |
| Independent reflections | 2782[R(int) = 0.0272] |
| Data/restraints/parameters | 2782/1/220 |
| Goodness-of-fit on F² | 1.029 |
| Final R indexes [I >= 2σ | R₁ = 0.0237, wR₂ = 0.0626 |
| (I)] | |
| Final R indexes [all data] | R₁ = 0.0241, wR₂ = 0.0629 |
| Largest diff. peak/hole | 0.12/−0.15 eÅ⁻³ |
| Flack parameter | −0.3(3) |

TABLE S2
Bond Distances in Compound 5a, Å

| | | | | | |
|---|---|---|---|---|---|
| C1—C2 | 1.456(3) | C1—N2 | 1.419(2) | C1—O1 | 1.216(2) |
| C2—C3 | 1.361(3) | C2—C9 | 1.491(3) | C3—C10 | 1.453(2) |
| C3—N1 | 1.416(2) | C4—C5 | 1.354(3) | C4—C8 | 1.484(2) |
| C4—N1 | 1.418(2) | C5—C6 | 1.450(3) | C5—C7 | 1.493(3) |
| C6—N2 | 1.435(2) | C6—O2 | 1.213(2) | C10—C11 | 1.345(3) |
| C11—C12 | 1.463(2) | C12—C13 | 1.405(3) | C12—C17 | 1.404(3) |
| C13—C14 | 1.384(3) | C14—C15 | 1.388(3) | C15—C16 | 1.388(3) |
| C15—N3 | 1.469(2) | C16—C17 | 1.381(3) | N1—N2 | 1.400(2) |
| N3—O3 | 1.230(2) | N3—O4 | 1.227(2) | | |

TABLE S3
Bond Angles in Compound 5a, °

| | | | | | |
|---|---|---|---|---|---|
| N2—C1—C2 | 105.09(15) | O1—C1—C2 | 130.24(18) | O1—C1—N2 | 124.61(17) |
| C1—C2—C9 | 121.50(16) | C3—C2—C1 | 108.59(17) | C3—C2—C9 | 129.84(18) |
| C2—C3—C10 | 127.42(17) | C2—C3—N1 | 109.79(16) | N1—C3—C10 | 122.17(16) |
| C5—C4—C8 | 127.80(17) | C5—C4—N1 | 109.57(16) | N1—C4—C8 | 122.51(16) |
| C4—C5—C6 | 109.23(16) | C4—C5—C7 | 128.84(17) | C6—C5—C7 | 121.91(16) |
| N2—C6—C5 | 104.84(15) | O2—C6—C5 | 131.29(17) | O2—C6—N2 | 123.87(17) |
| C11—C10—C3 | 123.89(16) | C10—C11—C12 | 126.40(17) | C13—C12—C11 | 117.83(16) |
| C17—C12—C11 | 123.28(17) | C17—C12—C13 | 118.89(17) | C14—C13—C12 | 121.02(17) |
| C13—C14—C15 | 118.21(17) | C14—C15—C16 | 122.51(17) | C14—C15—N3 | 118.40(17) |
| C16—C15—N3 | 119.07(16) | C17—C16—C15 | 118.66(17) | C16—C17—C12 | 120.70(17) |
| C3—N1—C4 | 130.54(15) | N2—N1—C3 | 106.78(14) | N2—N1—C4 | 107.10(14) |
| C1—N2—C6 | 128.21(15) | N1—N2—C1 | 109.73(13) | N1—N2—C6 | 109.04(14) |
| O3—N3—C15 | 118.26(17) | O4—N3—C15 | 118.47(16) | O4—N3—03 | 123.26(17) |

### X-Ray Structure Determination of Compound 5c

Compound 5c, C₁₇H_{15.25}FN₂O_{2.125}, crystallizes in the orthorhombic space group Fdd2 (systematic absences hkl: h+k=odd and k+l=odd, h0l: h+l@n, and 0kl: k+l≠4n) with a=29.9395(3)Å, b=27.5788(3)Å, c=7.07968(9)Å, V=5845.66(12)Å³, Z=16, and dcalc=1.366 g/cm³. X-ray intensity data were collected on a Rigaku XtaLAB Synergy-S diffractometer equipped with an HPC area detector (HyPix-6000HE) and employing confocal multilayer optic-monochromated Cu=Kα radiation (λ=1.54184 Å) at a temperature of 100K. Preliminary indexing was performed from a series of sixty 0.5° rotation frames with exposures of 15 sec. for θ=±47.04° and 60 sec. for θ=107.75°. A total of 7042 frames (47 runs) were collected employing ω scans with a crystal to detector distance of 34.0 mm, rotation widths of 0.5° and exposures of 2 sec. for θ=±47.04° and 8 sec. for θ=−86.25 and 107.75°. Rotation frames were integrated using CrysAlisPro, producing a listing of unaveraged F2 and σ(F2) values. A total of 35147 reflections were measured over the ranges 8.718≤2θ≤149.006°, −37≤h≤35, −33≤k≤34, −8≤l≤8 yielding 2976 unique reflections (Rint=0.0563). The intensity data were corrected for Lorentz and polarization effects and for absorption using SCALE3 ABSPACK (minimum and maximum transmission 0.58523, 1.00000). The structure was solved by dual space methods—SHELXT. Refinement was by full-matrix least squares based on F2 using SHELXL. All reflections were used during refinement. The weighting scheme used was w=1/[σ²(Fo²)+(0.0568P)²+4.6832P] where P=(Fo²+2Fc²)/3. Non-hydrogen atoms were refined anisotropically and hydrogen atoms were refined using a riding model. Refinement converged to R1=0.0365 and wR2=0.0978 for 2938 observed reflections for which F>4σ(F) and R1=0.0369 and wR2=0.0982 and GOF=1.087 for all 2976 unique, non-zero reflections and 207 variables. The maximum Δ/σ in the final cycle of least squares was 0.001 and the two most prominent peaks in the final difference Fourier were +0.15 and −0.21 e/A³.

Table S4 lists cell information, data collection parameters, and refinement data. Tables S5 and S6 list bond distances and bond angles. FIG. 62 is an ORTEP representation of the molecule with 50% probability thermal ellipsoids displayed.

TABLE S4
Summary of Structure Determination of 5c.

| Empirical formula | C₁₇H_{15.25}FN₂O_{2.125} | |
|---|---|---|
| Formula weight | 300.56 | |
| Diffractometer | Rigaku XtaLAB Synergy-S (HyPix-6000HE) | |
| Temperature/K | 100 | |
| Crystal system | orthorhombic | |
| Space group | Fdd2 | |
| a | 29.9395(3) | Å |
| b | 27.5788(3) | Å |
| c | 7.07968(9) | Å |
| α | 90° | |
| β | 90° | |
| γ | 90° | |
| Volume | 5845.66(12) | Å³ |
| Z | 16 | |
| d_{calc} | 1.366 | g/cm³ |
| μ | 0.829 | mm⁻¹ |

| F(000) | 2516.0 |
|---|---|
| Crystal size, mm | 0.25 × 0.12 × 0.08 |
| 2θ range for data collection | 8.718-149.006° |
| Index ranges | −37 ≤ h ≤ 35, −33 ≤ k ≤ 34, −8 ≤ l ≤ 8 |
| Reflections collected | 35147 |
| Independent reflections | 2976[R(int) = 0.0563] |
| Data/restraints/parameters | 2976/1/207 |
| Goodness-of-fit on F² | 1.087 |
| Final R indexes [I >= 2σ | R₁ = 0.0365, wR₂ = 0.0978 |
| (I)] | |
| Final R indexes [all data] | R₁ = 0.0369, wR₂ = 0.0982 |
| Largest diff. peak/hole | 0.15/−0.21 eÅ⁻³ |
| Flack parameter | 0.10(6) |

TABLE S5
Bond Distances in Compound 5c, Å.

| | | | | | |
|---|---|---|---|---|---|
| C1—C2 | 1.452(3) | C1—N2 | 1.417(3) | C1—O1 | 1.221(3) |
| C2—C3 | 1.374(3) | C2—C9 | 1.490(3) | C3—C10 | 1.450(3) |
| C3—N1 | 1.387(3) | C4—C5 | 1.368(3) | C4—C8 | 1.483(3) |
| C4—N1 | 1.374(3) | C5—C6 | 1.448(3) | C5—C7 | 1.487(3) |
| C6—N2 | 1.398(3) | C6—O2 | 1.219(3) | C10—C11 | 1.346(3) |
| C11—C12 | 1.464(3) | C12—C13 | 1.403(3) | C12—C17 | 1.392(3) |
| C13—C14 | 1.388(3) | C14—C15 | 1.380(3) | C15—C16 | 1.371(3) |
| C15—F1 | 1.357(3) | C16—C17 | 1.392(3) | N1—N2 | 1.375(3) |

TABLE S6
Bond Angles in Compound 5c, °

| | | | | | |
|---|---|---|---|---|---|
| N2—C1—C2 | 104.22(18) | O1—C1—C2 | 131.6(2) | O1—C1—N2 | 124.2(2) |
| C1—C2—C9 | 121.2(2) | C3—C2—C1 | 109.11(19) | C3—C2—C9 | 129.5(2) |
| C2—C3—C10 | 130.4(2) | C2—C3—N1 | 108.07(19) | N1—C3—C10 | 121.5(2) |
| C5—C4—C8 | 130.0(2) | C5—C4—N1 | 108.1(2) | N1—C4—C8 | 122.0(2) |
| C4—C5—C6 | 109.28(19) | C4—C5—C7 | 127.9(2) | C6—C5—C7 | 122.8(2) |
| N2—C6—C5 | 103.76(18) | O2—C6—C5 | 131.4(2) | O2—C6—N2 | 124.86(19) |
| C11—C10—C3 | 121.2(2) | C10—C11—C12 | 127.4(2) | C13—C12—C11 | 123.9(2) |
| C17—C12—C11 | 117.1(2) | C17—C12—C13 | 118.9(2) | C14—C13—C12 | 120.3(2) |
| C15—C14—C13 | 118.5(2) | C16—C15—C14 | 123.2(2) | F1—C15—C14 | 118.2(2) |
| F1—C15—C16 | 118.6(2) | C15—C16—C17 | 117.7(2) | C12—C17—C16 | 121.4(2) |
| C4—N1—C3 | 142.1(2) | C4—N1—N2 | 108.70(18) | N2—N1—C3 | 109.15(18) |
| C6—N2—C1 | 140.38(18) | N1—N2—C1 | 109.45(18) | N1—N2—C6 | 110.16(17) |

### X-Ray Structure Determination of Compound 5k

Compound 5k, C₂₀H₂₅N₃O₃, crystallizes in the monoclinic space group P21/c (systematic absences 0k0: k=odd and h0l: l=odd) with a=7.0514(6)Å, b=17.1123(10)Å, c=30.720(2)Å, β=91.352(8)°, V=3705.8(5)Å3, Z=8, and dcalc=1.274 g/cm3. X-ray intensity data were collected on a Rigaku XtaLAB Synergy-S diffractometer equipped with an HPC area detector (Dectris Pilatus3 R 200K) and employing confocal multilayer optic-monochromated Mo-Kα radiation (λ=0.71073 Å) at a temperature of 100 K. Preliminary indexing was performed from a series of thirty 0.5° rotation frames with exposures of 10 seconds. A total of 1272 frames (13 runs) were collected employing co scans with a crystal to detector distance of 50.0 mm, rotation widths of 0.5° and exposures of 60 seconds. The crystal grew as a non-merohedral twin. The Ewald Explorer extension in CrysAlisPro was used to index the diffraction images and to determine the twinning mechanism. The crystal was twinned by a rotation of 180° about the 001 real direction. Rotation frames were integrated using CrysAlisPro, producing a listing of unaveraged F2 and σ(F2) values. A total of 49137 reflections were measured over the ranges 4.636≤2θ≤52.95°, −8≤h≤8, −21≤k≤21, −37≤l≤38 yielding 13556 unique reflections (Rint=0.081). The intensity data were corrected for Lorentz and polarization effects and for absorption using SCALE3 ABSPACK (minimum and maximum transmission 0.85,80 1.0000). The structure was solved by dual space methods—SHELXT. Refinement was by full-matrix least squares based on F2 using SHELXL. All reflections were used during refinement. The weighting scheme used was w=1/[σ²(Fo²)+(0.1260P)²+0.9687P] where P=(Fo²+2Fc²)/3. Non-hydrogen atoms were refined anisotropically and hydrogen atoms were refined using a riding model. Refinement converged to R1=0.0719 and wR2=0.1949 for 9577 observed reflections for which F>4σ(F) and R1=0.1047 and wR2=0.2144 and GOF=1.047 for all 13556 unique, non-zero reflections and 484 variables. The maximum Δ/σ in the final cycle of least squares was 0.000 and the two most prominent peaks in the final difference Fourier were +0.40 and −0.33 e/Å3. The twinning parameter refined to a value of 0.5079(16).

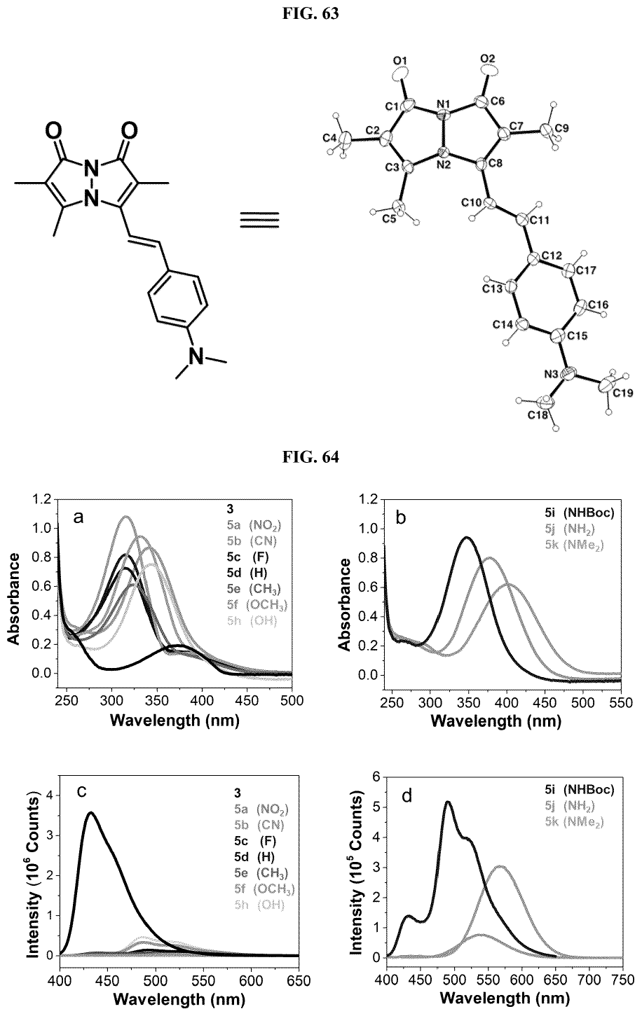

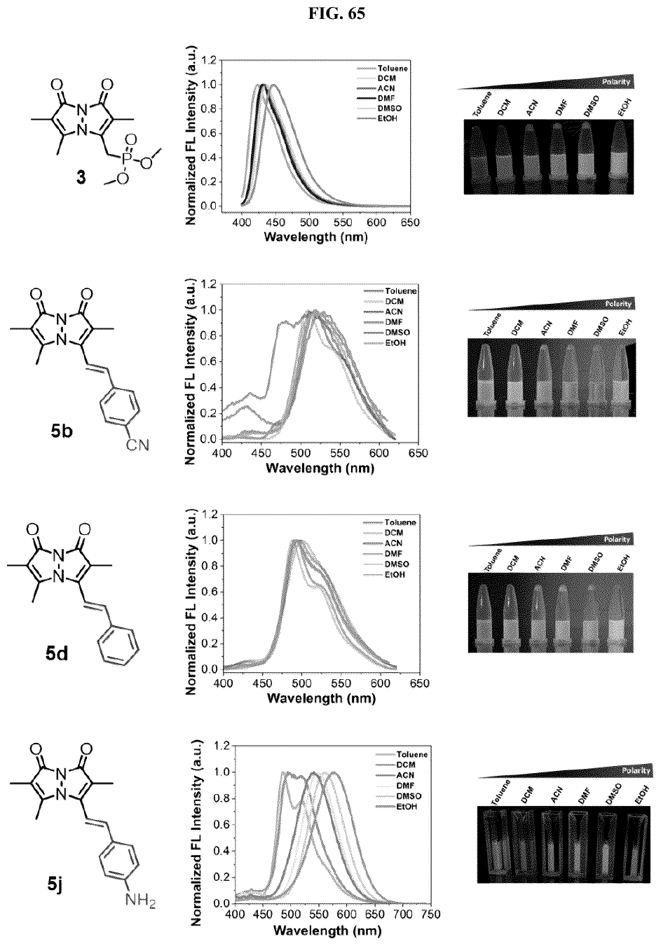

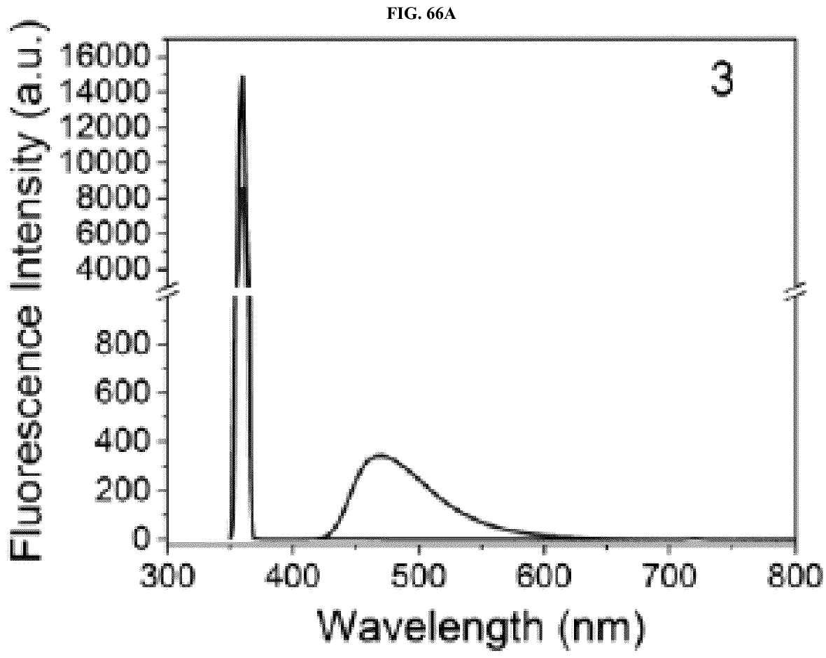

Table S7 lists cell information, data collection parameters, and refinement data. Tables S8 and S9 list bond distances and bond angles. FIG. 63 is an ORTEP representation of the molecule with 50% probability thermal ellipsoids displayed.

TABLE S7
Summary of Structure Determination of Compound 5c.

| Empirical formula | C₂₀H₂₅N₃O₃ | |
|---|---|---|
| Formula weight | 355.43 | |
| Diffractometer | Rigaku XtaLAB Synergy-S | |
| | (Dectris Pilatus3 R 200K) | |
| Temperature/K | 100 | |
| Crystal system | monoclinic | |
| Space group | P2₁/c | |
| a | 7.0514(6) | Å |
| b | 17.1123(10) | Å |
| c | 30.720(2) | Å |
| β | 91.352(8)° | |
| Volume | 3705.8(5) | Å³ |
| Z | 8 | |
| d_{calc} | 1.274 | g/cm³ |
| μ | 0.087 | mm⁻¹ |

| F(000) | 1520.0 |
|---|---|
| Crystal size, mm | 0.26 × 0.19 × 0.04 |
| 2θ range for data collection | 4.636-52.95° |
| Index ranges | −8 ≤ h ≤ 8, −21 ≤ k ≤ 21, −37 ≤ l ≤ 38 |
| Reflections collected | 49137 |
| Independent reflections | 13556[R(int) = 0.081] |
| Data/restraints/parameters | 13556/0/484 |
| Goodness-of-fit on F² | 1.047 |
| Final R indexes [I >= 2σ | R₁ = 0.0719, wR₂ = 0.1949 |
| (I)] | |
| Final R indexes [all data] | R₁ = 0.1047, wR₂ = 0.2144 |
| Largest diff. peak/hole | 0.40/−0.33 eÅ⁻³ |

TABLE S8
Bond Distances in Compound 5k, Å

| | | | | | |
|---|---|---|---|---|---|
| O1—C1 | 1.235(4) | O2—C6 | 1.219(4) | N1—N2 | 1.369(4) |
| N1—C1 | 1.409(4) | N1—C6 | 1.421(4) | N2—C3 | 1.392(4) |
| N2—C8 | 1.394(4) | N3—C15 | 1.373(4) | N3—C18 | 1.438(5) |
| N3—C19 | 1.455(5) | C1—C2 | 1.430(5) | C2—C3 | 1.357(4) |
| C2—C4 | 1.510(5) | C3—C5 | 1.477(5) | C6—C7 | 1.437(5) |
| C7—C8 | 1.364(5) | C7—C9 | 1.508(4) | C8—C10 | 1.445(4) |
| C10—C11 | 1.339(4) | C11—C12 | 1.453(4) | C12—C13 | 1.391(5) |
| C12—C17 | 1.401(4) | C13—C14 | 1.378(5) | C14—C15 | 1.398(5) |
| C15—C16 | 1.414(5) | C16—C17 | 1.380(5) | O1′—C1′ | 1.215(4) |
| O2′—C6′ | 1.225(4) | N1′—N2′ | 1.370(4) | N1′—C1′ | 1.404(4) |
| N1′—C6′ | 1.401(4) | N2′—C3′ | 1.385(4) | N2′—C8′ | 1.378(4) |
| N3′—C15′ | 1.362(5) | N3′—C18′ | 1.445(5) | N3′—C19′ | 1.459(4) |
| C1′—C2′ | 1.459(5) | C2′—C3′ | 1.348(4) | C2′—C4′ | 1.490(5) |
| C3′—C5′ | 1.482(4) | C6′—C7′ | 1.445(5) | C7′—C8′ | 1.373(5) |
| C7′—C9′ | 1.490(5) | C8′—C10′ | 1.444(5) | C10′—C11′ | 1.347(4) |
| C11′—C12′ | 1.452(5) | C12′—C13′ | 1.398(5) | C12′—C17′ | 1.403(4) |
| C13′—C14′ | 1.367(5) | C14′—C15′ | 1.422(4) | C15′—C16′ | 1.409(5) |
| C16′—C17′ | 1.369(5) | O3—C20 | 1.404(5) | O3′—C20′ | 1.372(5) |

TABLE S9
Bond Angles in Compound 5k, °

| | | | | | |
|---|---|---|---|---|---|
| N2—N1—C1 | 109.1(3) | N2—N1—C6 | 109.6(2) | C1—N1—C6 | 139.1(3) |
| N1—N2—C3 | 108.6(2) | N1—N2—C8 | 108.4(3) | C3—N2—C8 | 140.0(3) |
| C15—N3—C18 | 120.4(3) | C15—N3—C19 | 120.1(3) | C18—N3—C19 | 117.6(3) |
| O1—C1—N1 | 122.9(3) | O1—C1—C2 | 132.3(3) | N1—C1—C2 | 104.8(3) |
| C1—C2—C4 | 123.6(3) | C3—C2—C1 | 109.4(3) | C3—C2—C4 | 127.0(3) |
| N2—C3—C5 | 121.6(3) | C2—C3—N2 | 108.0(3) | C2—C3—C5 | 130.4(3) |
| O2—C6—N1 | 123.6(3) | O2—C6—C7 | 132.1(3) | N1—C6—C7 | 104.3(3) |
| C6—C7—C9 | 122.0(3) | C8—C7—C6 | 109.3(3) | C8—C7—C9 | 128.5(3) |
| N2—C8—C10 | 120.6(3) | C7—C8—N2 | 108.3(3) | C7—C8—C10 | 131.0(3) |
| C11—C10—C8 | 122.6(3) | C10—C11—C12 | 126.5(3) | C13—C12—C11 | 123.7(3) |
| C13—C12—C17 | 116.6(3) | C17—C12—C11 | 119.7(3) | C14—C13—C12 | 122.5(3) |
| C13—C14—C15 | 121.0(3) | N3—C15—C14 | 121.0(3) | N3—C15—C16 | 121.9(3) |
| C14—C15—C16 | 117.1(3) | C17—C16—C15 | 121.0(3) | C16—C17—C12 | 121.9(3) |
| N2′—N1′—C1′ | 109.9(3) | N2′—N1′—C6′ | 109.7(2) | C6′—N1′—C1′ | 139.8(3) |
| N1′—N2′—C3′ | 108.5(2) | N1′—N2′—C8′ | 109.1(3) | C8′—N2′—C3′ | 141.9(3) |
| C15′—N3′—C18′ | 120.9(3) | C15′—N3′—C19′ | 121.4(3) | C18′—N3′—C19′ | 117.6(3) |
| O1′—C1′—N1′ | 124.5(3) | O1′—C1′—C2′ | 131.7(3) | N1′—C1′—C2′ | 103.7(3) |
| C1′—C2′—C4′ | 122.7(3) | C3′—C2′—C1′ | 109.1(3) | C3′—C2′—C4′ | 128.1(3) |
| N2′—C3′—C5′ | 122.2(3) | C2′—C3′—N2′ | 108.7(3) | C2′—C3′—C5′ | 129.1(3) |
| O2′—C6′—N1′ | 124.2(3) | O2′—C6′—C7′ | 131.5(3) | N1′—C6′—C7′ | 104.2(3) |
| C6′—C7′—C9′ | 121.2(3) | C8′—C7′—C6′ | 109.1(3) | C8′—C7′—C9′ | 129.1(3) |
| N2′—C8′—C10′ | 120.6(3) | C7′—C8′—N2′ | 107.8(3) | C7′—C8′—C10′ | 131.6(3) |
| C11′—C10′—C8′ | 124.1(3) | C10′—C11′—C12′ | 126.1(3) | C13′—C12′—C11′ | 123.4(3) |
| C13′—C12′—C17′ | 116.7(3) | C17′—C12′—C11′ | 119.9(3) | C14′—C13′—C12′ | 122.0(3) |
| C13′—C14—C15′ | 121.3(3) | N3′—C15′—C14′ | 120.7(3) | N3′—C15′—C16′ | 122.7(3) |
| C16′—C15—C14′ | 116.5(3) | C17′—C16′—C15′ | 121.1(3) | C16′—C17′—C12′ | 122.3(3) |

TABLE S10
Comparison of selected bond lengths
and bond angles of 5a, 5c, and 5k.

| | bond length (Å) | | bond angles ° | |
|---|---|---|---|---|
| probe | C1—O1 | N2—N1 | φ (C1—N2—C6) | packing |
| 5a | 1.216(2) | 1.400(2) | 128.21(15) | face-to-face |
| 5c | 1.221(3) | 1.375(3) | 140.38(18) | head-to-tail |
| 5k | 1.235(4) | 1.369(4) | 139.1(3) | head-to-tail |

### General Photophysical Characterization Methods

Absorbance measurements. Key precursor 3 and bimane derivatives 5a-5k were dissolved in DMSO to make 10 mM starting stock solutions. Samples were individually diluted to the final concentration of 25 μM (500 μL) using 50:50 acetonitrile in phosphate buffered saline (ACN/PBS). The absorbance spectrum of each derivative (150 μL) was measured on a Thermo Scientific Genesys 150 UV-Vis spectrometer, using 50:50 ACN/PBS as a blank.

Fluorescence measurements. 10 mM stock solutions of 3 and 5a-5k in DMSO were individually diluted to the final concentration of 25 μM (500 μL) using 50:50 ACN/PBS. The fluorescence spectrum of each derivative (150 μL) was measured on a Photon Technology International (PTJ) QuantaMaster™ 40 fluorescence spectrometer by excitation at the maximum absorption wavelength for each compound.

Molar absorptivity determination. 10 mM stock solutions of 3 and 5a-5k in DMSO were individually diluted to the final concentration of 25 μM (500 μL) using 50:50 ACN/PBS. The absorbance spectrum of each derivative (150 μL) was measured on a Thermo Scientific Genesys 150 UV-Vis spectrometer, using 50:50 ACN/PBS as a blank. Then, the molar absorptivity of each derivative was calculated by using the Beer-Lambert law for solutions, A=elc, where A=absorbance at maximum wavelength, l=optical path length in cm, c=concentration of the solution (25 μM), e=molar absorptivity.

Solvatochromic measurements. 10 mM stock solution of 3, 5b, 5d, 5j, and 5k in DMSO were individually diluted into six different organic solvents (toluene, dichloromethane, ACN, dimethyl formamide, dimethyl sulfoxide, and ethanol) to the final concentration of 25 μM (1000 μL). The fluorescence spectrum of each derivative (150 μL) was measured on a PTI QuantaMaster™ 40 fluorescence spectrometer by excitation at the maximum absorption wavelength for each compound. The remaining 850 μL solution was used to obtain fluorescence images under a handheld 365 nm UV lamp.

pH measurements. 10 mM stock solutions of 5h and 5j in DMSO were individually diluted into ACN/PBS buffer (50:50 v/v) having different pH ranging from pH 2-11 with 25 M (500 μL) of final concentration. For pH screening of 5h and 5j, PBS buffer solutions were prepared, then adjusted the pH 2, 4, and 7 by adding 6 M hydrochloric acid, pH 9 and 11 by adding 5 M sodium hydroxide. Then, we have recorded the absorption and fluorescence spectra by using Thermo Scientific Genesys 150 UV-Vis spectrometer, followed by PTI QuantaMaster™ 40 fluorescence spectrometer by excitation with maximum absorption wavelength.

Viscosity measurements. As a representative example, we have measured the viscosity of 5k (25 μM, 10 mM stock in DMSO) in glycerol/ethylene glycol (G/EG) mixtures, varying the glycerol percentage 0-90%. Then, the emission spectra were recorded using a PTI QuantaMaster™ 40 fluorescence spectrometer by excitation with 385 nm wavelength.

Photophysical Properties of 3 and 5a-5k in ACN

TABLE S12
Photophysical properties of the bimane
derivatives 3 and 5a-5k in ACN.

| | | | | Molar | | Fluores- |
|---|---|---|---|---|---|---|
| | | Absor- | Emis- | Absorp- | | cence |
| | Substit- | bance | sion | tivity | Stokes | Quantum |
| Com- | uent | λabs | λem | (M⁻¹ | Shift | Yield |
| pound | (Ph-R) | (nm)^{a} | (nm)^{b} | cm⁻¹)^{c} | (nm)^{d} | (%)^{e} |
| 3 | — | 375 | 432 | 6121 | 57 | 77.7 |
| 5a | NO₂ | 332 | 432 | 37871 | 100 | 0.2 |
| 5b | CN | 316 | 522 | 43238 | 206 | 1.4 |
| 5c | F | 315 | 492, 520 | 28940 | 177 | 2.8 |
| 5d | H | 316 | 492, 522 | 32724 | 176 | 3.1 |
| 5e | CH₃ | 325 | 490, 520 | 24426 | 165 | 3.4 |
| 5f | OCH₃ | 342 | 488, 518 | 34554 | 146 | 3.5 |
| 5h | OH | 342 | 486, 518 | 29910 | 144 | 3.1 |
| 5i | NH(Boc) | 348 | 490, 520 | 37555 | 142 | 3.4 |
| 5j | NH₂ | 378 | 540 | 32091 | 162 | 1.5 |
| 5k | N(CH₃)₂ | 402 | 568 | 24742 | 166 | 7.5 |

^{a}Maximum absorption wavelength,
^{b}Maximum emission wavelength,
^{c}Molar absorption coefficients at maximum absorption wavelength,
^{d}Difference between maximum absorption wavelength and maximum emission wavelength,
^{e}Fluorescence quantum yield (error limit within ±5).
Final probe concentration is 25 μM in ACN. FIG. 64A shows absorption spectra of 3 and 5a-5f and 5h. FIG. 64B shows absorption spectra of 5i-5k, FIG. 64C shows fluorescence spectra of 3 and 5a-5f and 5h. FIG. 64D Fluorescence spectra of 5i-5k. Final probe concentration is 25 μM. Fluorescence spectra were measured at their maximum absorption wavelength..

Fluorescent Quantum Yield Measurements of 3 and 5a-5k

Fluorescent quantum yield (QY) measurements. For each QY measurement, the incident excitation light spectrum was collected with 1 mL of solvent. After measuring the incident light intensity, dye (3 and 5a-5k) was added to the solvent from a concentrated stock solution to a final concentration of 500 aM and the new spectrum (fluorescence intensity and new incident light intensity) was collected. Using the JASCO Quantum Yield Software, the dye QY was calculated by dividing the dye fluorescence intensity by the difference in incident light intensity in the presence and absence of dye. The minimum excitation wavelength for a full excitation incident spectrum using this setup is 360 nm.

Fluorescence Lifetime Measurements of 3 and 5c-5f

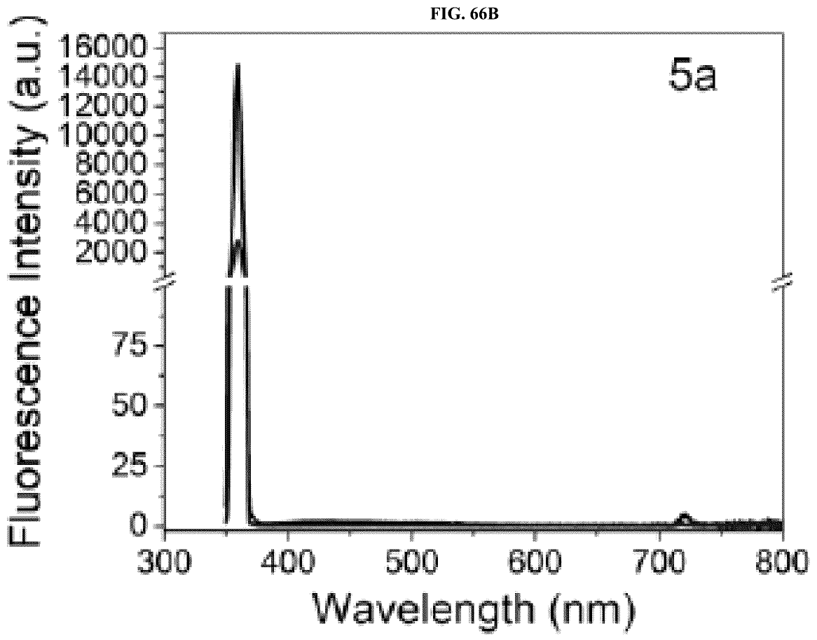

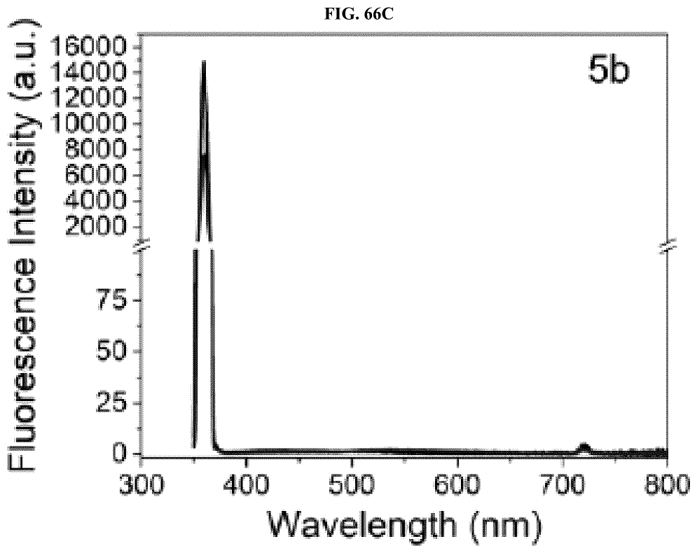

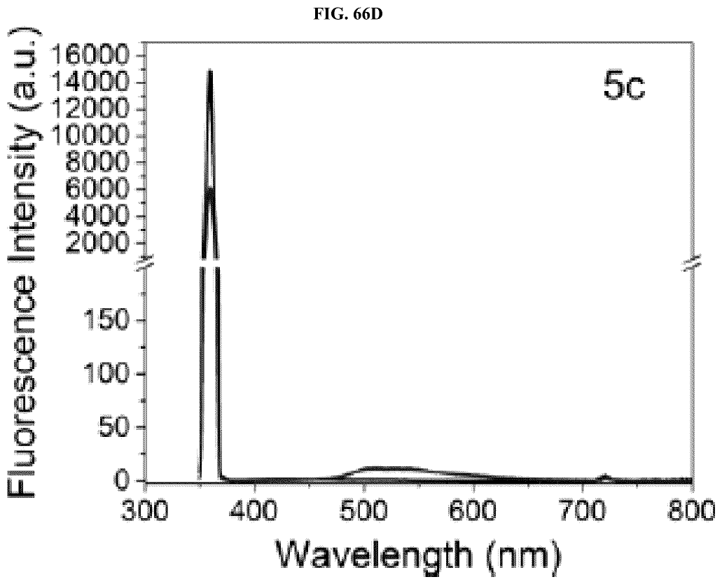

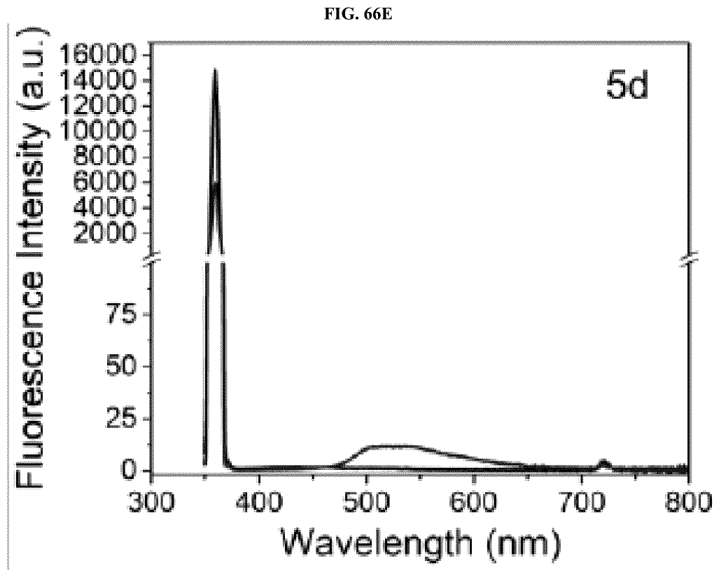

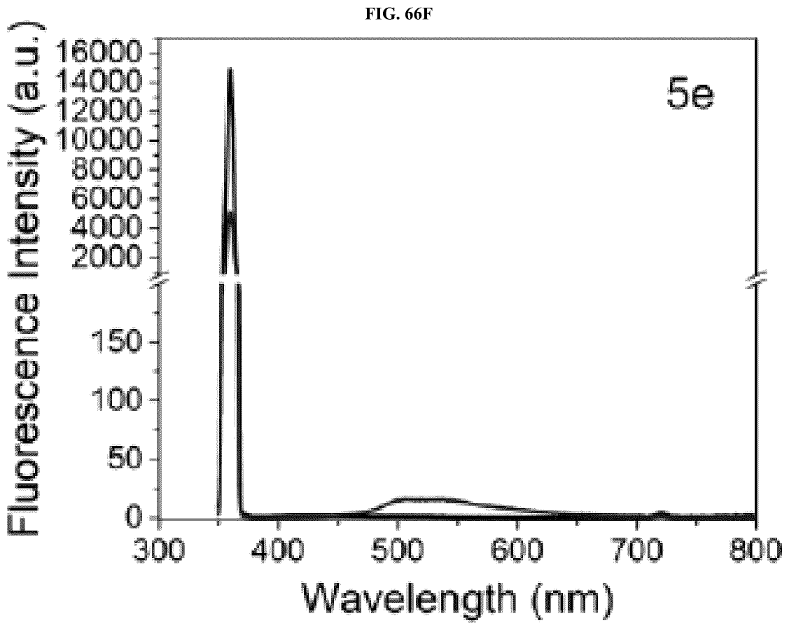

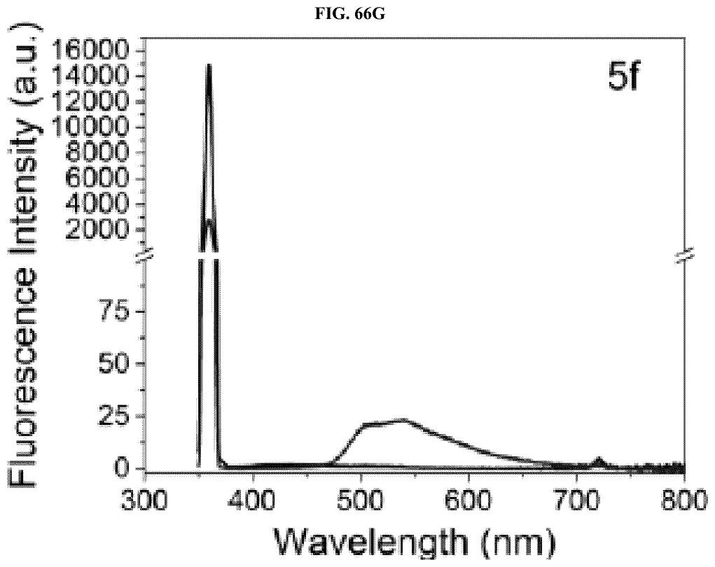

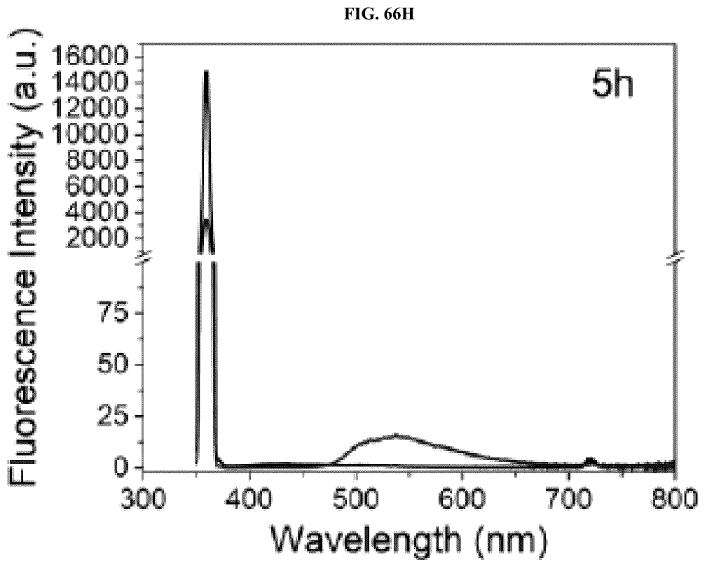

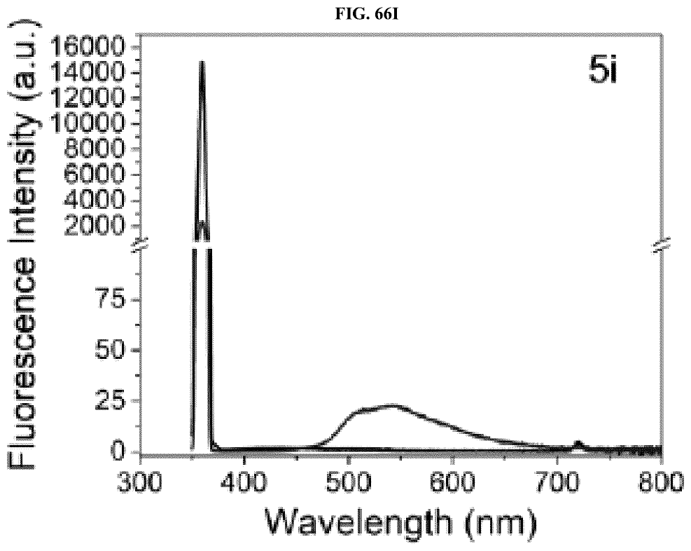

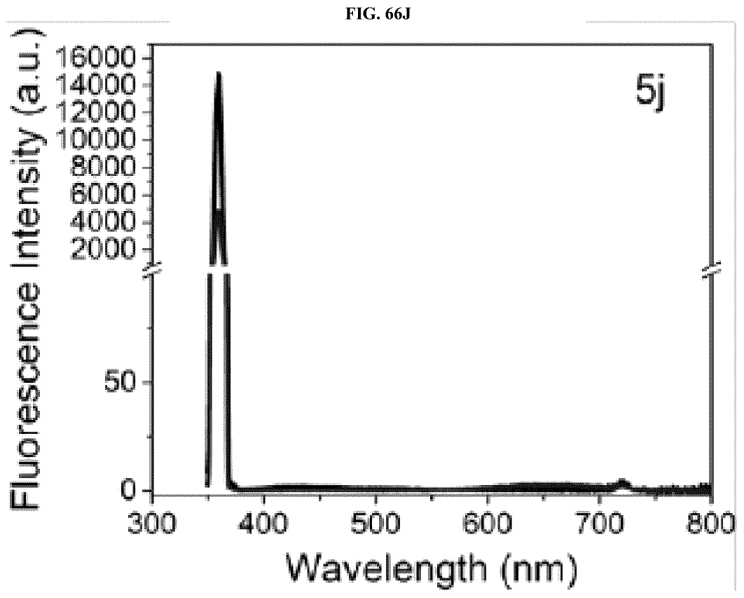

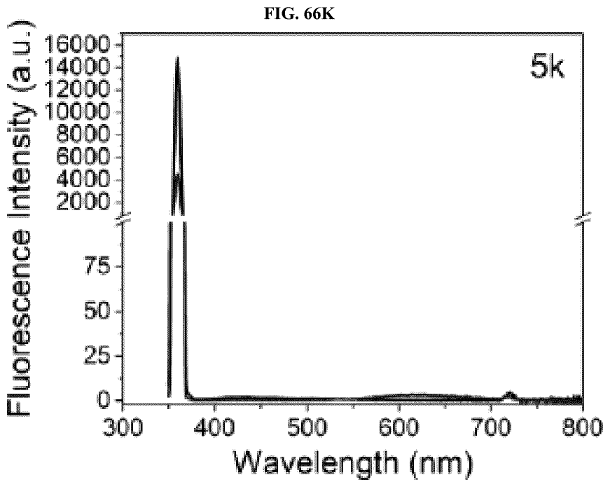

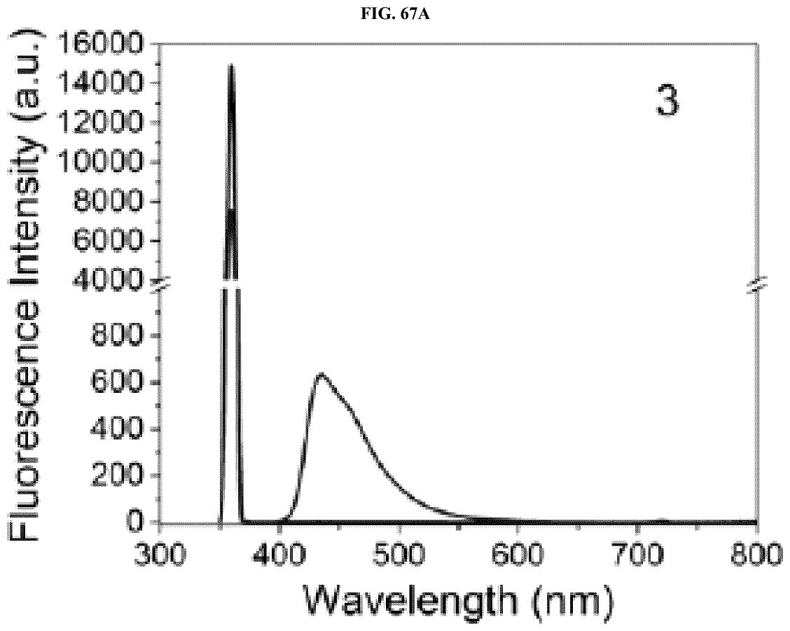

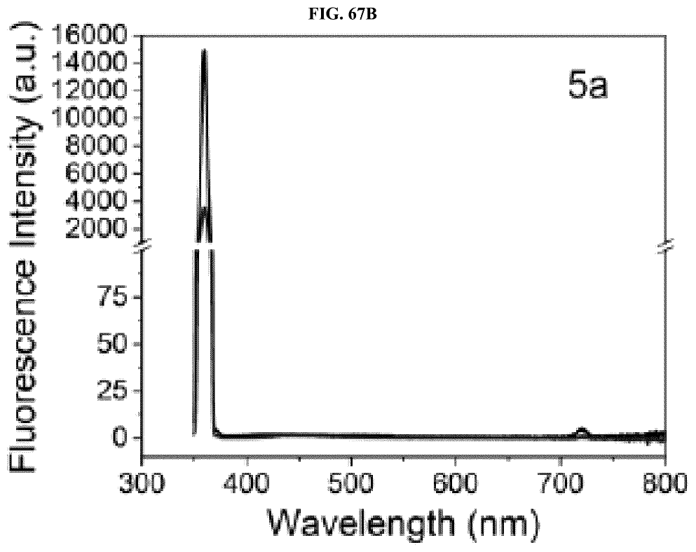

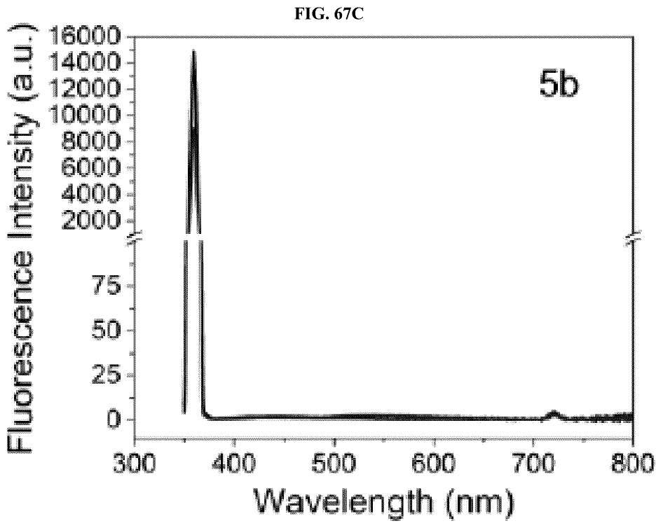

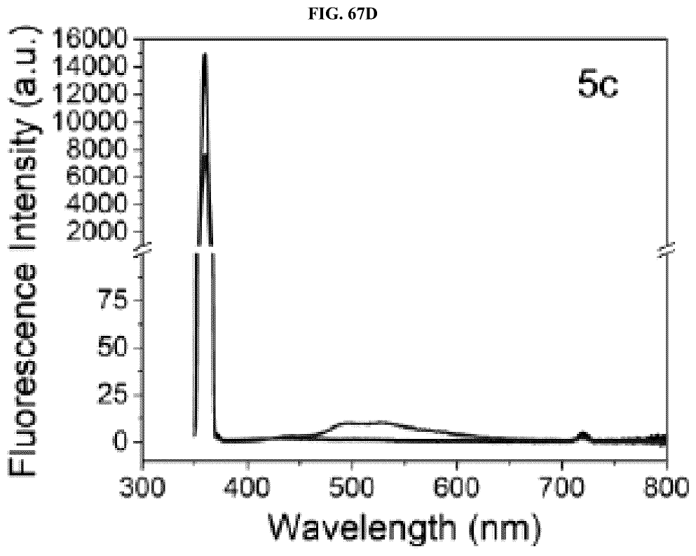

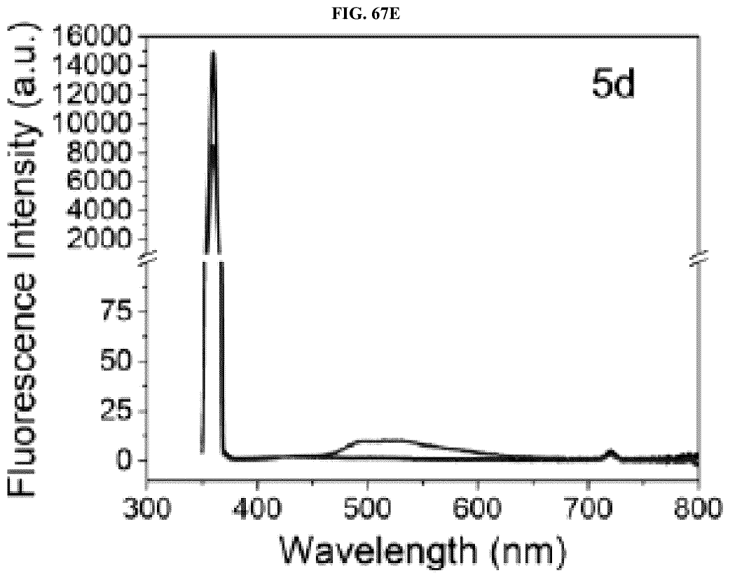

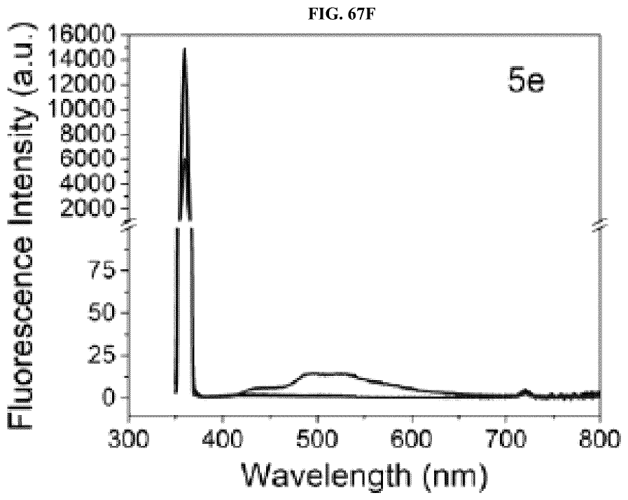

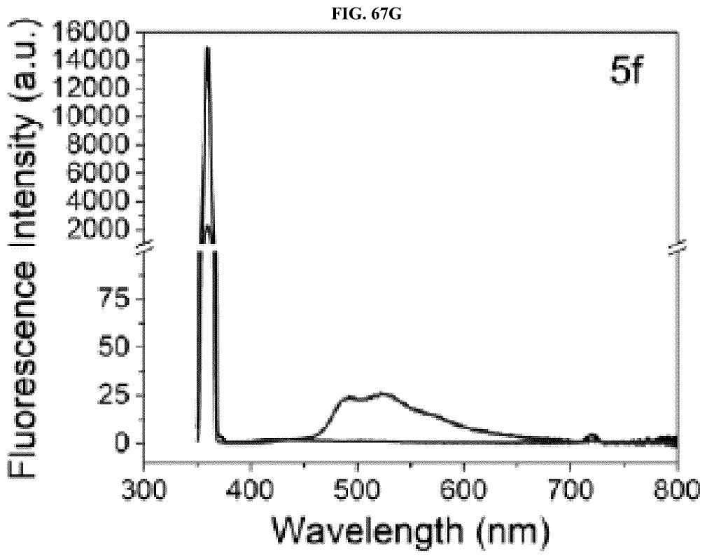

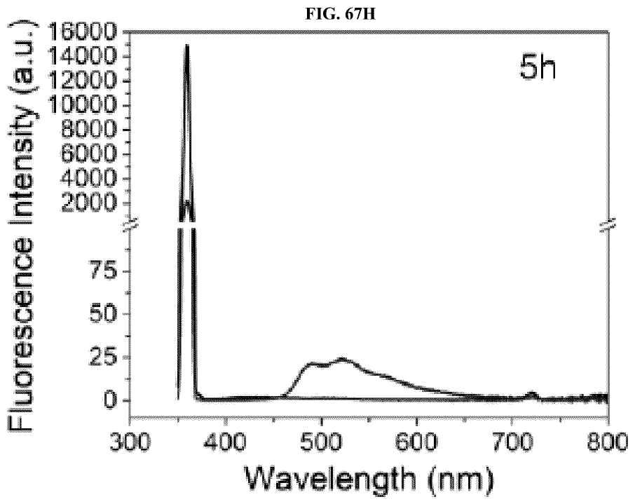

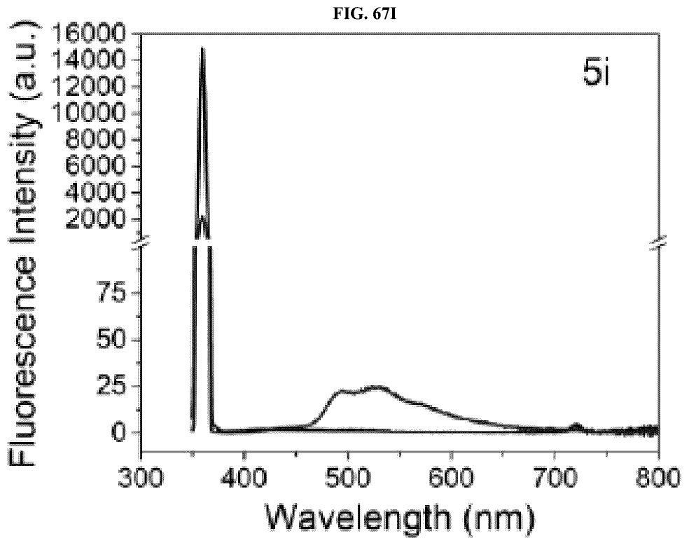

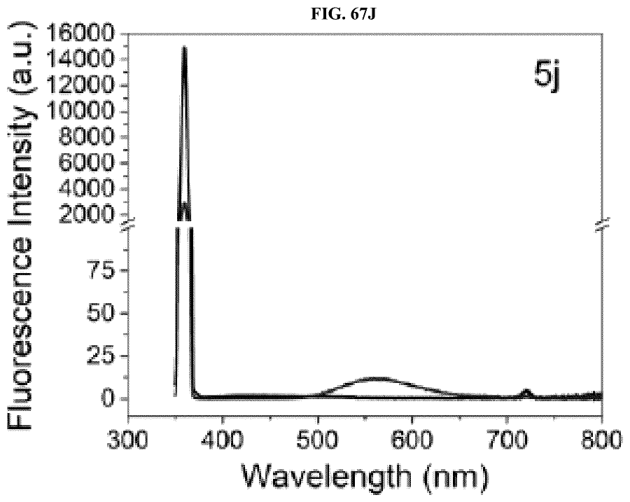

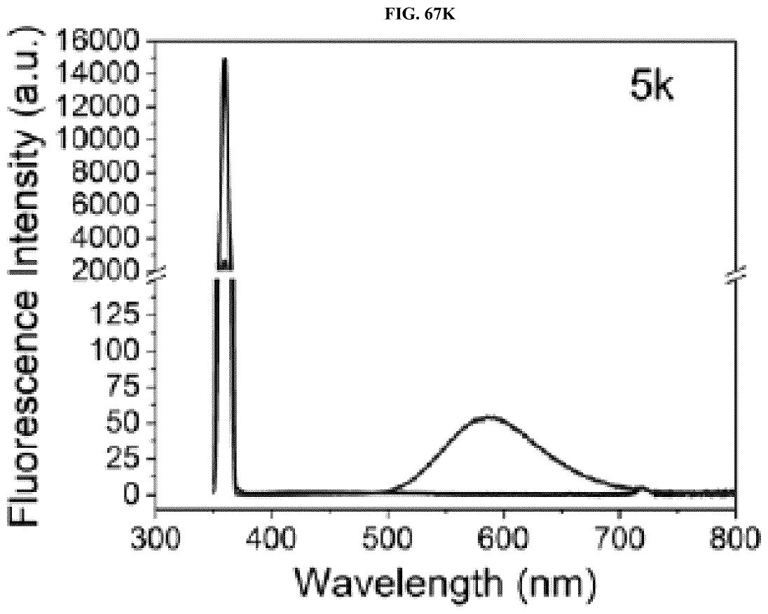

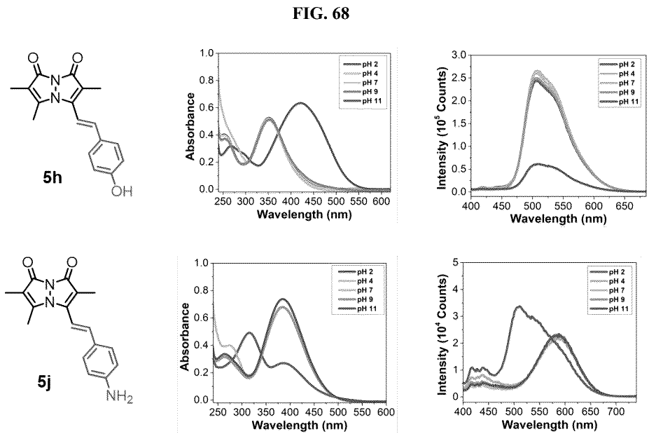

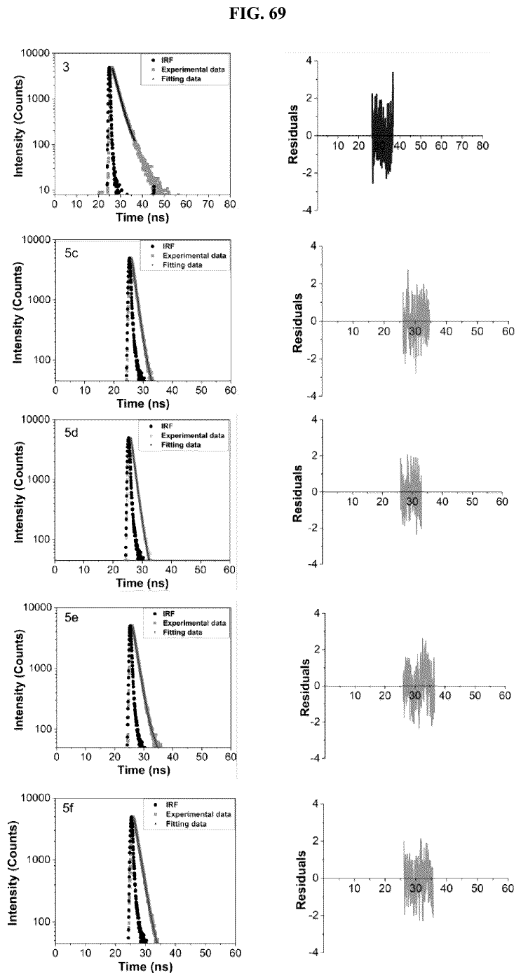

Fluorescence lifetime measurements. Time correlated single photon counting (TCSPC) measurements of fluorescence lifetime decays for 100 μM solutions of dyes (3 and 5c-f) in ACN/PBS buffer (50:50 v/v) were collected with the PTI Quantamaster™ 40 using a pulsed LED with a maximum emission at 340 nm. Fluorescence emission was collected at the indicated wavelength for each dye with 20 nm slit widths. The instrument response function (TRF) was collected under identical conditions. Data analysis was performed with FluoFit software (PicoQuant GmbH; Berlin, Germany) using an exponential decay model. FIG. 64A shows absorption spectra of 3 and 5a-5f and 5h. FIG. 64B shows absorption spectra of 5i-5k, FIG. 64C shows fluorescence spectra of 3 and 5a-5f and 5h. FIG. 64D Fluorescence spectra of 5i-5k. Final probe concentration is 25 μM. Fluorescence spectra were measured at their maximum absorption wavelength. FIGS. 66A-66K show representative QY acquisitions of 3 and 5a-f, 5h-k in ACN/PBS buffer (50:50). All had consistent excitation (360 nm) and spectral collection (350-800 nm) parameters. Final concentration of the probe is 500 μM. Black line indicates incident light and blue line indicates sample spectrum. FIGS. 67A-67K shows representative QY acquisitions of 3 and 5a-f, 5h-k in ACN solvent. All had consistent excitation (360 nm, slit width 10 nm) and spectral collection (350-800 nm) parameters. Final concentration of the probe is 500 M. Black line indicates incident light and blue line indicates sample spectrum. FIG. 68: Effect of pH on absorption and emission of 5h and 5j. Final concentration of the probe is 25 μM. For pH screening of 5h and 5j, PBS buffer solutions were prepared, then adjusted the pH 2, 4, and 7 by adding 6 M hydrochloric acid, pH 9 and 11 by adding 5 M sodium hydroxide. Emission spectra were measured by the excitation wavelength 354 nm for 5h and 385 nm for 5j. FIG. 69 shows fluorescence lifetime measurements in ACN/PBS buffer (50:50 v/v). Lifetime measurements with exponential fits shown at left and residuals after fitting shown at right.

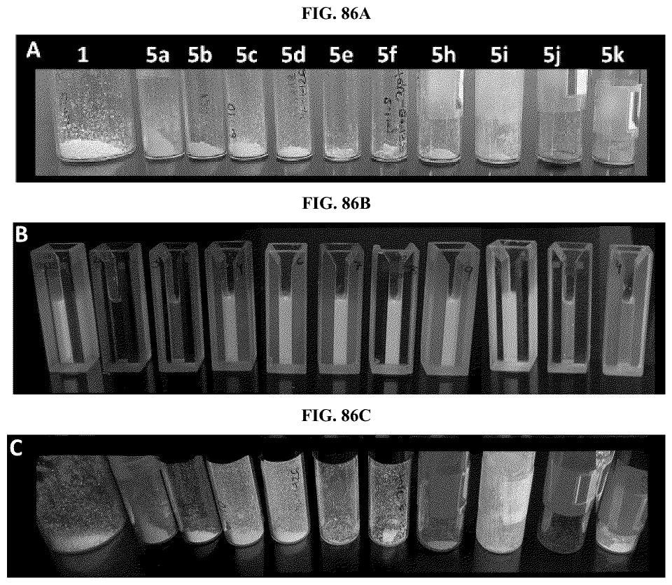

TCSPC data were collected at 540 nm emission for 5c-5f and 464 nm emission for 3. Excitation wavelength 340 nm. FIGS. 86A-86C shows representative photophysical properties of 1 and 5a-f, 5h-k. FIG. 86A shows a photograph under ambient light. FIG. 86B Solution state fluorescence of 25 μM probe in ACN/PBS buffer (50:50 v/v) under handheld UV lamp (365 nm). FIG. 86C shows amorphous solid-state fluorescence under handheld UV lamp (365 nm).

TABLE S13
Fluorescence lifetime measurements of 3 and selected probes
5c-f in ACN/PBS buffer (50:50 v/v) and fit χ2 values.
TCSPC data were collected at 540 nm emission for 5c-f and
464 nm emission for 3. Excitation wavelength 340 nm.

| Probe | Ar—R | Lifetime (ns) | χ² |
|---|---|---|---|
| 3 | — | 2.32 ± 0.01 | 1.099 |
| 5c | F | 1.07 ± 0.003 | 1.017 |
| 5d | H | 1.02 ± 0.004 | 0.942 |
| 5e | CH₃ | 1.28 ± 0.004 | 1.089 |
| 5f | OCH₃ | 1.31 ± 0.004 | 0.932 |

### Computational Studies

### Methods

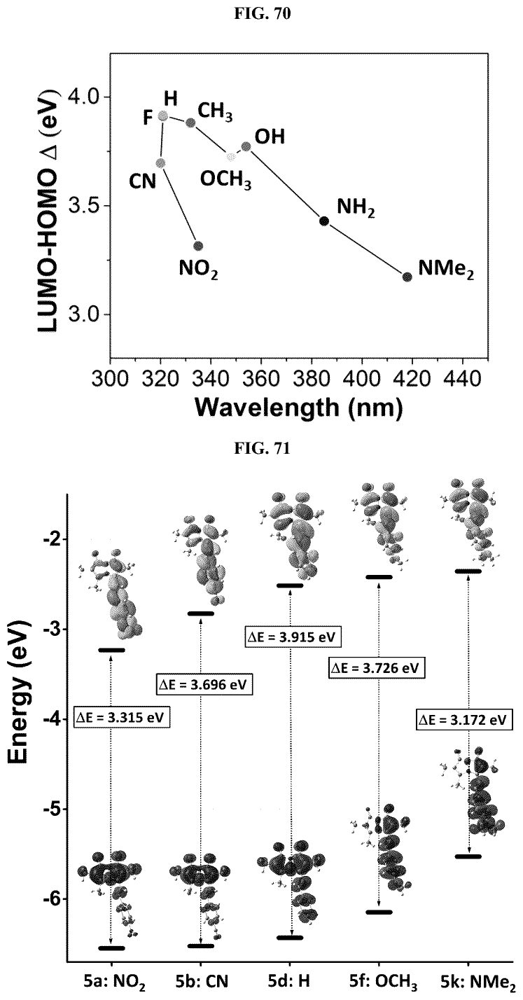

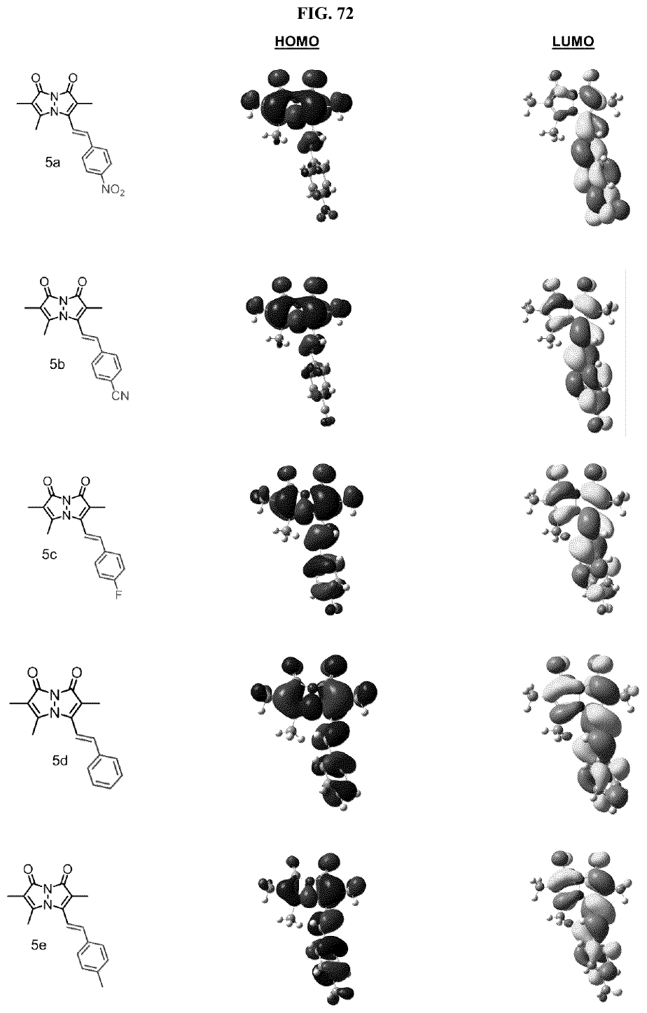

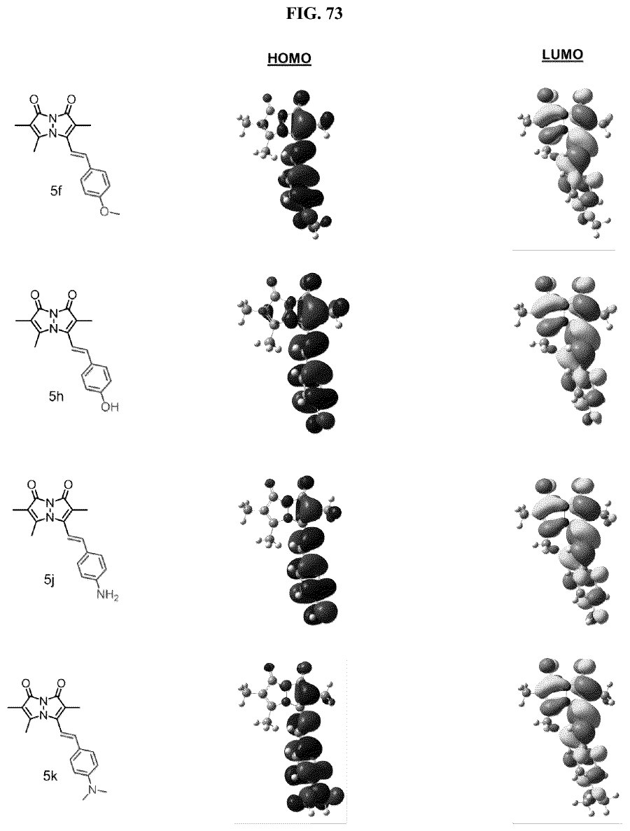

All calculations were performed employing the APF-D density functional as implemented in the Gaussian16™ suite of programs with the 6-311+G (2d,p) basis set. Geometry optimizations and energies were calculated for selected bimane derivatives 5a-f, 5h, 5j, and 5k compounds. The HOMO-LUMO energy gaps between the ground state (S₀) and the first excited state (S₁) of the bimane derivatives were calculated and are shown below. All structures are ground-state minima according to the analysis of their vibrational frequencies, which showed no negative value. FIG. 70 shows correlation between HOMO-LUMO energy gap (eV) compared to experimentally measured UV-Vis absorption maximum (nm) of 5a-5f, 5h, 5j, and 5k. Maximum UV-Vis absorption wavelengths were obtained in ACN/PBS buffer (50:50 v/v). FIG. 71 shows relative HOMO-LUMO energy gap (eV) of selected bimane derivatives. FIG. 72 shows HOMO and LUMO Mos of 5a-5e. FIG. 73 shows HOMO and LUMO MOs of 5f, 5h, 5j, and 5k.

TABLE S14
Calculation of the Energy Gaps of Bimane Derivatives in Water
(H₂O). Absorbance spectra recorded in ACN:PBS buffer (50:50 v/v).

| | | Absor- | | | | |
|---|---|---|---|---|---|---|
| | | bance | | | #LUMO- | |
| | Hammett | Wave- | | | #HOMO | LUMO- |
| Com- | Constant | length | HOMO | LUMO | Transi- | HOMO |
| pound | (σₚ) | (nm)^{a} | (eV) | (eV) | tion | Δ(eV) |
| 5a | 0.778 | 335 | −6.547 | −3.232 | 86-85 | 3.315 |
| (NO₂) | | | | | | |
| 5b | 0.66 | 320 | −6.523 | −2.827 | 81-80 | 3.696 |
| (CN) | | | | | | |
| 5c | 0.062 | 321 | −6.419 | −2.506 | 79-78 | 3.912 |
| (F) | | | | | | |
| 5d | 0 | 321 | −6.430 | −2.514 | 75-74 | 3.915 |
| (H) | | | | | | |
| 5e | −0.17 | 332 | −6.351 | −2.470 | 79-78 | 3.881 |
| (CH₃) | | | | | | |
| 5f | −0.268 | 348 | −6.146 | −2.420 | 83-82 | 3.726 |
| (OCH₃) | | | | | | |
| 5h | −0.37 | 354 | −6.192 | −2.420 | 79-78 | 3.772 |
| (OH) | | | | | | |
| 5j | −0.66 | 385 | −5.812 | −2.383 | 79-78 | 3.429 |
| (NH₂) | | | | | | |
| 5k | −0.83 | 418 | −5.528 | −2.356 | 87-86 | 3.172 |
| (NMe₂) | | | | | | |

HOMO-LUMO gap calculations. We have analyzed the correlation between the photophysical properties of bimane derivatives and the electronic properties of the substituents to understand the mechanism of fluorescence tuning of the bimane scaffold. It is generally accepted that the absorption wavelength (λabs) and emission wavelength (λem) correlate well with the energy gap between HOMO and LUMO as determined by simple electronic structure calculations. Relative energy levels of HOMO and LUMO were calculated using APF-D/6-311+G (2d,p) density functional theory (DFT) calculations. Representative compounds 5e (CH₃), 5f (OCH₃), 5h (OH), 5j (NH₂), and 5k (NMe₂), with electron donating groups at the para-position, showed a decrease in HOMO-LUMO gap with more electron donating substituents that matched well with red-shifts of the UV-Vis absorbance (Table S3). Interestingly, compounds with electron withdrawing substituents, 5a (NO₂), 5b (CN), and 5c (F) showed a shallower correlation of UV-Vis absorbance with HOMO-LUMO gap, indicative of a change in excitation mechanism. The change in mechanism can be explained by examination of the HOMO and LUMO of these compounds. For compounds with electron-withdrawing substituents such as 5a, the electrons move from a HOMO centered on the bimane group to a LUMO centered on the styryl group. In contrast, for compounds with electron-donating substituents such as 5k, the electrons move from a HOMO centered on the styryl group to a LUMO centered on the bimane group. This is similar to our observations of the changes in λabs with the Hammett constant.

### Production of α-Synuclein and 1N4R Tau

α-Synuclein production. α-Synuclein (αS) was expressed and purified. Human αS with a C-terminal intein-His₆ fusion was transformed into Escherichia coli (E. coli) BL21 cells and plated on ampicillin plates (100 μg/mL). A single colony was then inoculated into a 5 ml primary culture containing ampicillin (100 μg/mL) in Luria-Bertain (LB) media and grown for 5-6 h with shaking (250 rpm) at 37° C. The primary culture was then transferred to 1 L LB containing ampicillin (100 μg/mL) and grown until reaching an optical density (OD600) of 0.8-1.0. At this stage, protein production was induced by the addition of isopropyl β-D-1-thiogalactopyranoside (IPTG) to 1 mM final concentration, and the culture was grown with shaking (250 rpm) overnight at 18° C. The following day, cells were harvested by centrifugation at 4° C. for 20 min at 4000 rpm (Sorvall GS3 rotor). Pellets were then re-suspended in 20 mL/L culture of 40 mM Tris, pH 8.3 supplemented with EDTA-free protease inhibitor tablets (Pierce Biotechnology; Waltham, MA, USA) and transferred to a metal cup for sonication. Cells were lysed by sonication on ice with a Q700 probe sonicator (QSonica LLC; Newtown, CT, USA) with the following settings: Amplitude 50, Process Time 2-3 min, Pulse-ON Time 1 s, Pulse-OFF Time 1 s. Crude lysate was then transferred to 50 mL centrifugation tubes and clarified via centrifugation at 14,000 rpm for 45 min (Sorvall SS34 rotor). Following centrifugation, supernatant was removed and transferred to a 50 mL Falcon tube. Then, 5 ml of nickel agarose resin (GoldBio; St. Louis, MO, USA) was added, and the lysate-nickel mixture was incubated with nutation at 4° C. for 1 h. The lysate-nickel mixture was then poured into a 20 mL fritted column, and the flowthrough was saved. The remaining resin was then washed with ˜20 mL Wash Buffer 1 (50 mM HEPES buffer, pH 7.5), ˜20 mL Wash Buffer 2 (50 mM HEPES, 5 mM imidazole, pH 7.5), and eluted with 12 mL Elution Buffer (50 mM HEPES, 300 mM imidazole, pH 7.5). Then 2-mercaptoethanol (Bio-Rad Laboratories; Hercules, CA, USA) was added to crude lysate (200 mM final concentration), and the mixture was allowed to incubate with nutation at room temperature overnight. The resulting cleaved protein was dialyzed against 20 mM Tris pH 8.0 for 8-10 h. The resulting dialysate was then treated with 5 mL nickel agarose resin and incubated with nutation at 4° C. for 1-2 h. The mixture was then applied to a 20 mL fritted column and flowthrough containing αS was collected in a 15 mL Falcon tube. The resulting enriched protein mixture was then dialyzed against 20 mM Tris, pH 8.0 overnight and purified via FPLC using a 5 mL HiTrap Q-HP column (Cytiva; Marlborough, MA, USA) using the following method: Buffer A: 20 mM Tris, pH 8.0; Buffer B: 20 mM Tris, 1 M NaCl, pH 8.0; Gradient: 0% Buffer B—5 column volumes, 0-10% Buffer B—5 column volumes, 20-30% Buffer B—20 column volumes, 30-100% Buffer B—10 column volumes; flow rate 3 mL/min. The resulting fractions were then assessed for purity via MALDI MS, and pure fractions were combined. Protein was then concentrated, and buffer exchanged into PBS (NaCl 137 mM, KCl 2.7 mM, Na₂HPO₄ 10 mM, KH₂PO₄ 1.8 mM) to a final concentration of 100-200 μM via Amicon 3 kDa MWCO filters (Millipore Sigma; St. Louis, MO, USA). Purified protein was aliquoted into 1.5 mL tubes and stored at −80° C. until further use. MALDI MS [M+H]+ calcd: 14460, found: 14457.

### Tau 1N4R Protein Production

Tau (1N4R) (Tau) was expressed and purified. Using a fresh transformation of Tau 1N4R plasmid transformed into E. coli BL21 cells and plated on ampicillin plates (100 μg/mL), single colony was inoculated into a 5 mL primary culture containing ampicillin (100 μg/ml) in LB media and grown for 5-6 h with shaking (250 rpm) at 37° C. The primary culture was then transferred to 1 L LB containing ampicillin (100 μg/ml) and grown until reaching an OD600 of 0.4-0.6. At this stage, protein production was induced by the addition of IPTG to 1 mM final concentration, and the culture was grown with shaking (250 rpm) overnight at 16° C. The following day, cells were harvested by centrifugation at 4° C. for 20 min at 4000 rpm (Sorvall GS3 rotor). Pellets were then re-suspended in 15 mL Ni-NTA Buffer A (50 mM Tris pH 8, 500 mM NaCl, and 10 mM Imidazole) with 1 mg/mL (chicken egg white) lysozyme, 1 tablet of EDTA-free protease inhibitor and 1 mM PMSF. The suspension was sonicated on ice for 1 minute 40 seconds, 1 second on/2 seconds off, power set to 50 W. Cellular debris was then transferred to 50 mL centrifugation tubes and clarified via centrifugation at 20,000×g for 30 min (Sorvall SS34 rotor). Following centrifugation, supernatant was removed and filtered with a 0.22 m syringe filter into a 50 mL Falcon tube. Then, 5-7 mL of nickel agarose resin was prepared and equilibrated with ˜30 mL of Ni Buffer A. Then supernatant-nickel mixture was incubated with nutation at 4° C. for 1 h. Supernatant-nickel mixture was then poured into a 20 mL fritted column, and the flowthrough was saved. The remaining resin was then washed with ˜30 mL Ni-NTA Buffer A and eluted with ˜15 mL Ni-NTA Buffer B (50 mM Tris pH 8, 500 mM NaCl, and 400 mM imidazole). The nickel column was cleaned by running more Ni-NTA Buffer B through the column and then reequilibrated with Ni-NTA Buffer A. The elution was then concentrated to ˜1 mL in an Amicon concentrator (10 kDa MWCO) and the following were added 25 μM TEV, and 1 mM DTT from fresh 1 M DTT stock. The mixture was allowed to incubate with nutation at 4° C. overnight and then the buffer was exchanged back to Ni-NTA Buffer A (2 cycles of 15 mL) using an Amicon concentrator. The resulting dialysate was then treated with 5-7 mL nickel agarose resin and incubated with nutation at 4° C. for 1 h. The mixture was then applied to a 20 mL fritted column and flowthrough containing 1N4R tau was collected in a 15 mL Falcon tube. The resulting enriched protein mixture was then concentrated, and buffer exchanged two times into Ni-NTA Buffer C (25 mM Tris pH 8, 100 mM NaCl, 1 mM EDTA, and 1 mM TCEP) using an Amicon concentrator and concentrated down to 1 mL before filtering the solution using a 0.22 m filter. Then, the protein was purified via FPLC using a size exclusion column (S200, HiLoadc16/60cSuperdex 200 μg) using the following method: isocratic elution: 100% Ni-NTA Buffer C,—1.2 column volumes with flowrate 0.5 mL/min. The purity of the fractions was checked by SDS-PAGE gel and the clean fractions were combined, concentrated, and the buffer exchanged into PBS to a final concentration of 100-200 μM via Amicon 10 kDa MWCO filters and stored at −80° C. until further use. MALDI MS [M+H]+ calcd: 43111, found: 43109.

### Fibril Preparation

### αS Fibril Preparation

100 μM final concentration of αS monomer in PBS buffer (500 μL, 1.5 mL tube) at pH 7 was sealed with Teflon tape followed by parafilm and then incubated at 37° C. with shaking at 1300 rpm for 7 days in an IKA MS3 control orbital shaker (Wilmington, NC, USA) to get αS fibrils. 1N4R Tau fibril preparation. 50 μM final concentration of 1N4R tau monomer in PBS buffer (200 μL) at pH 7.4 along with 100 μM of DTT and 12.5 μM of heparin was sealed with Teflon tape followed by parafilm incubated at 37° C. with shaking at 1300 rpm for 72 h in an IKA MS3 control orbital shaker (Wilmington, NC, USA) to get tau fibrils. Ab1-42 fibril preparation. Using 1 mg of the commercially available Aβ1-42 (0-Amyloid (1-42), human, Genscript Cat. No. RP10017) 1 mg, a 1 mM stock solution was made by adding 222 μL hexafluoroisopropanol (HIP) directly to the vial containing lyophilized powder through the rubber septum. After the peptide completely dissolved, the septum was pierced with a syringe needle to release the vacuum. The Aβ1-42-HFIP solution was incubated for 30 min at room temperature. Using a positive displacement pipette, 100 μL aliquots of the solution (0.45 mg) were transferred into Eppendorf tubes and the HFIP was allowed to evaporate in the open tubes in a fume hood. The tubes were then dried on a vacuum centrifuge for 1 h without heating to remove any remaining trace amount of HFIP. To make a 1 mM stock solution of Aβ1-42 fibrils in 10 mM Phosphate buffer at pH 7.4, 0.45 mg of Aβ1-42 was dissolved in 10 μL DMSO with addition of 10 μL of 10 mM of NaOH and sealed in an Eppendorf tube with Teflon tape followed by parafilm, then incubated at 37° C. with shaking at 500 rpm for 5 days in an IKA MS3 control orbital shaker (Wilmington, NC, USA).

### Gel-Based Quantification of Protein in Fibrils

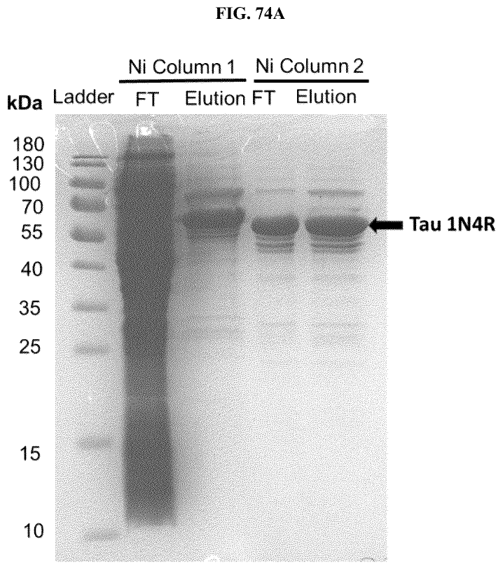

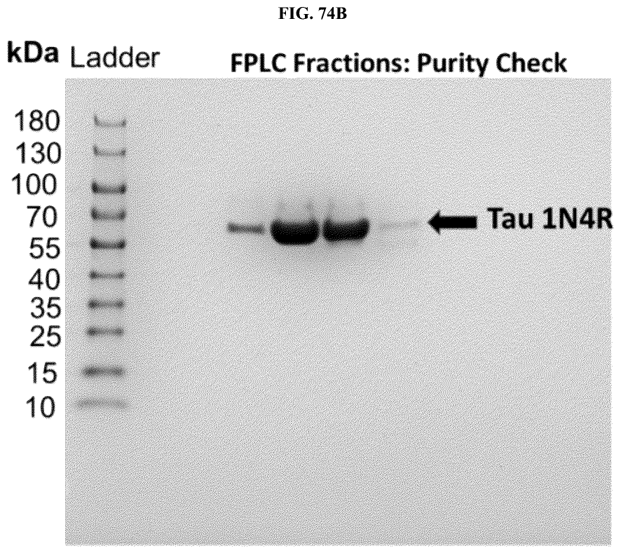

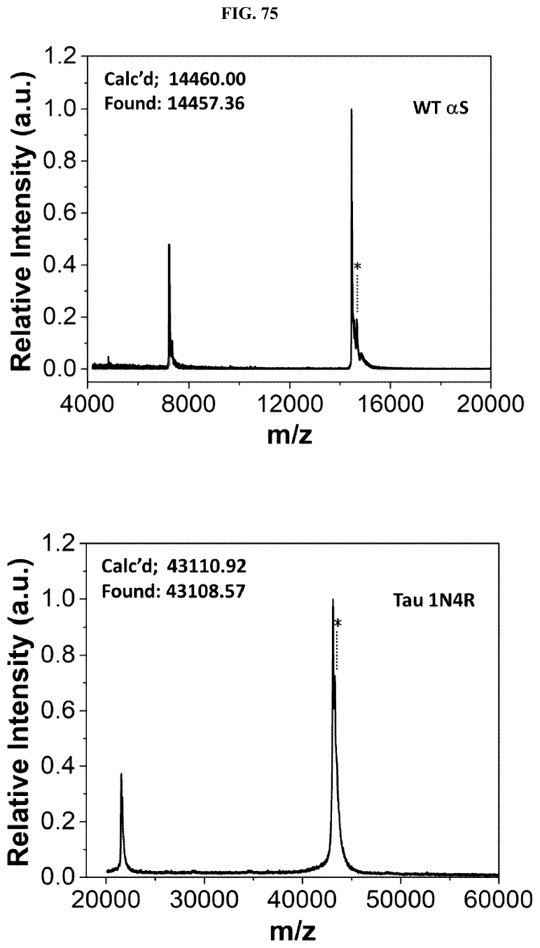

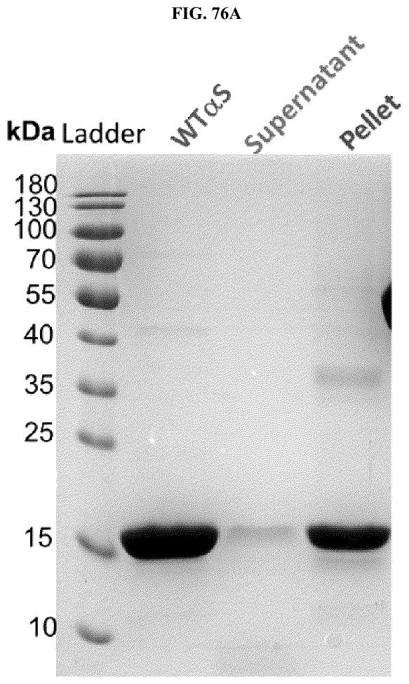

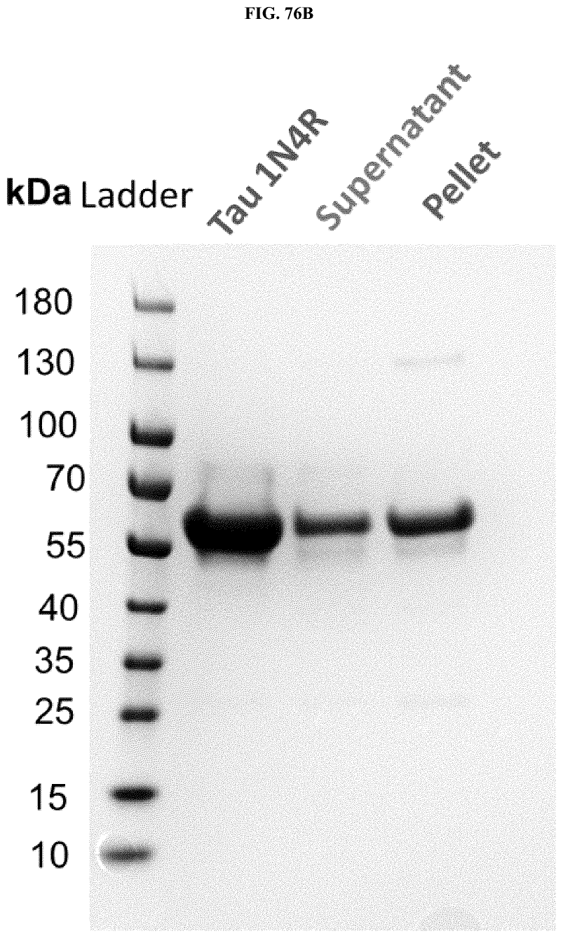

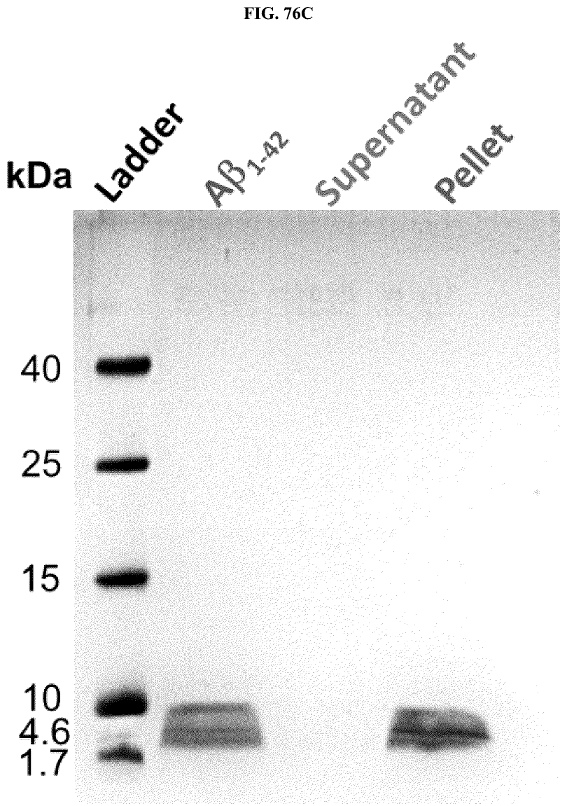

Following completion of aggregation, fibrils were pelleted by centrifugation in a bench top centrifuge at 13,200 rpm for 90 min at 4° C. The supernatant was then carefully removed, and the fibril pellet resuspended in aggregation buffer with vortexing. Then 10 μL from each sample was transferred into an individual 0.6 mL Eppendorf tube and 2 μL 150 mM SDS in water was added (final concentration: 25 mM SDS). The tubes were then capped and heated to −100° C. for 15-20 min. Then the tubes were placed on ice to cool for 5-10 min, and 3 μL 4× gel loading was added and the supernatant and pellet were run on an SDS-PAGE along with a monomer standard at a known concentration. After running the gel, the bands were quantified by ImageJ software to estimate the percentage in fibrils (pellet). This was used with the initial monomer concentration in the aggregation reactions to determine the concentration of fibrils for dye binding experiments. FIGS. 74A-74B: Protein purification and cleavage of 1N4R tau. FIG. 74A shows nickel purification visualized by a Coomassie stained 10% SDS-Page gel. FIG. 74B shows FPLC purification fractions by SDS-PAGE gel. FIG. 75 shows MALDI MS characterization of WT αS (Top) and tau 1N4R (Bottom). On each plot matrix adduct peaks are marked with *. FIGS. 76A-76C shows gel-based quantification of proteins.

### Fibril Binding Characterization

Probe Binding (Kd) Measurements with αS Fibrils.

αS fibrils (100 μM) in PBS buffer were incubated with varying 5k concentrations (0, 0.01, 0.1, 0.5, 1, 3, 5, 7, and 10 μM) from concentrated 10 mM DMSO stock solutions in Greiner 96 well flat black 12 area plates at 37° C. with shaking at 500 rpm for 15 min in an IKA MS3 control orbital shaker (Wilmington, NC, USA). After 15 min incubation, fluorescence intensity measurements were obtained with a Tecan Spark plate reader (Mannedorf, Switzerland) by excitation with λex/λem=463/580 nm for 5k, using the following parameters: excitation and emission bandwidth 5 nm, delay time 0 μs, integration time 40 μs. Averages and standard deviations were calculated from at least 3 independent measurements at each probe concentration. The resulting binding curve was fit to the following equation (modified from the One site—Total, accounting for ligand depletion model) in Graphpad Prism 9 (San Diego, CA, USA), from which the Kd was determined.

$$Y = \frac{a}{2} ( b - {( {b}^{2} - 4 c )}^{\frac{1}{2}} )$$

$$b = {R}_{t} + X + {K}_{d} and C = {R}_{t} X$$

X=total probe added in M, Y: measured fluorescence emission, Kd: Dissociation constant in μM, a: fluorescence counts/μM probe bound, and Rt=maximum probe occupancy (set to 3.3 μM based on Bmax observed in prior radioligand binding studies).7 We note that variation of Rt from 1 to 10 does not significantly change the fitted Kd value (less than 2-fold change).

Absorbance and Fluorescence Measurements with αS Fibrils

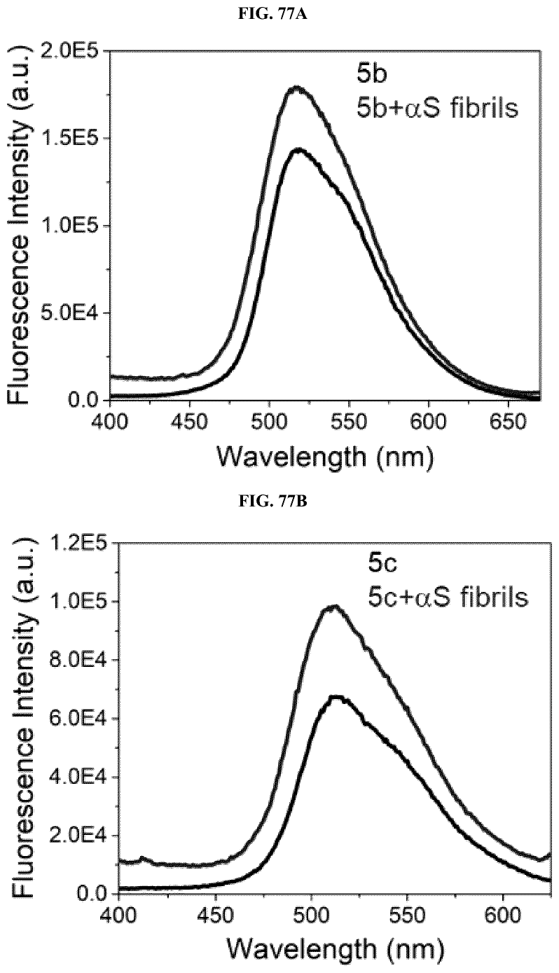

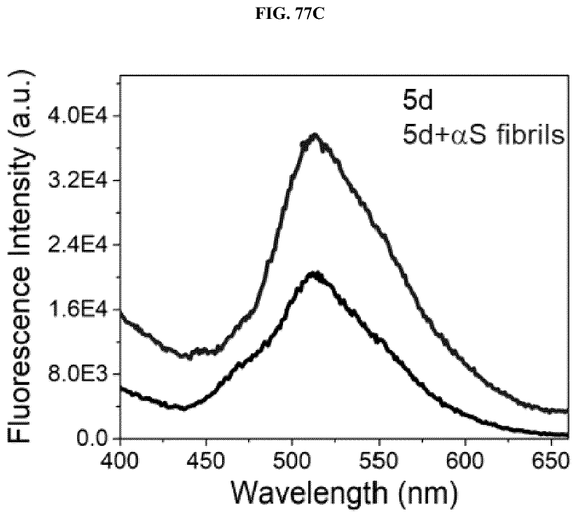

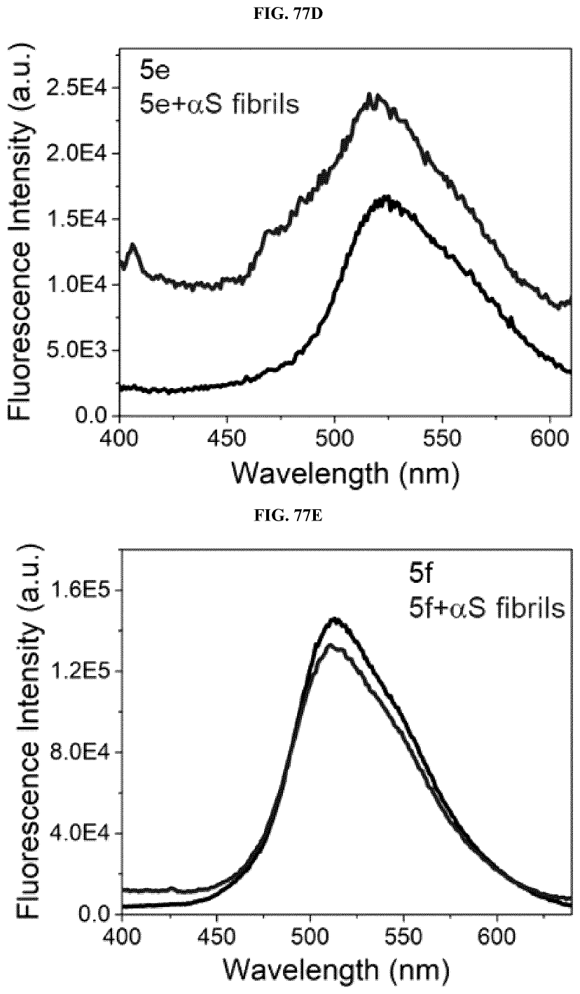

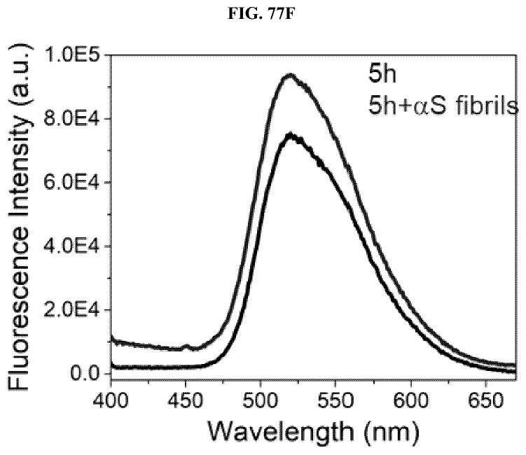

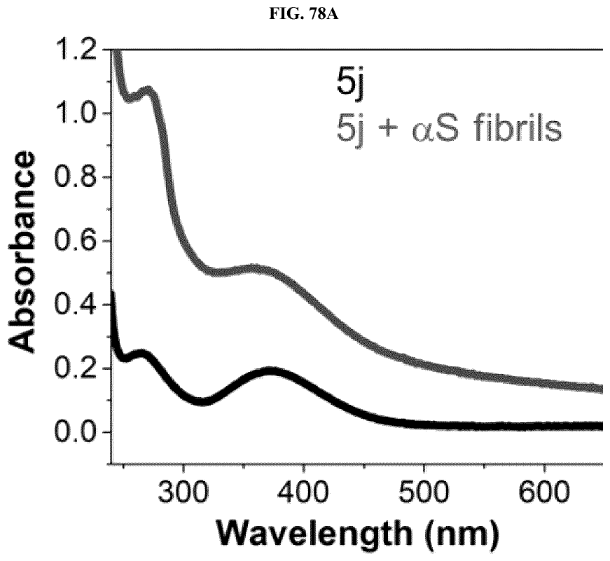

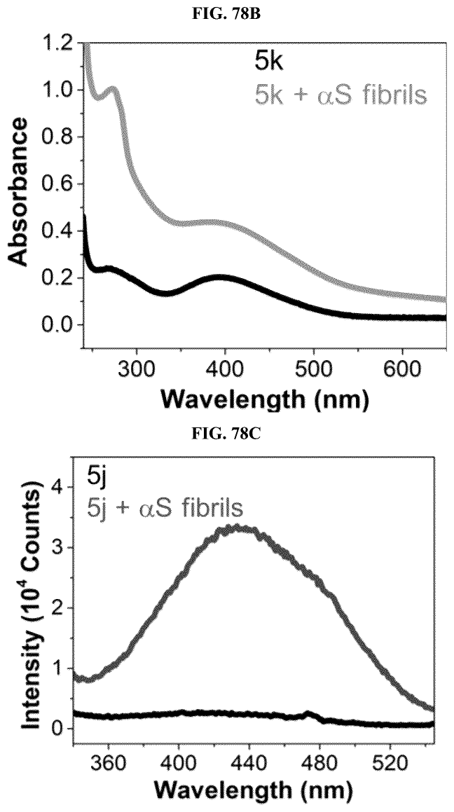

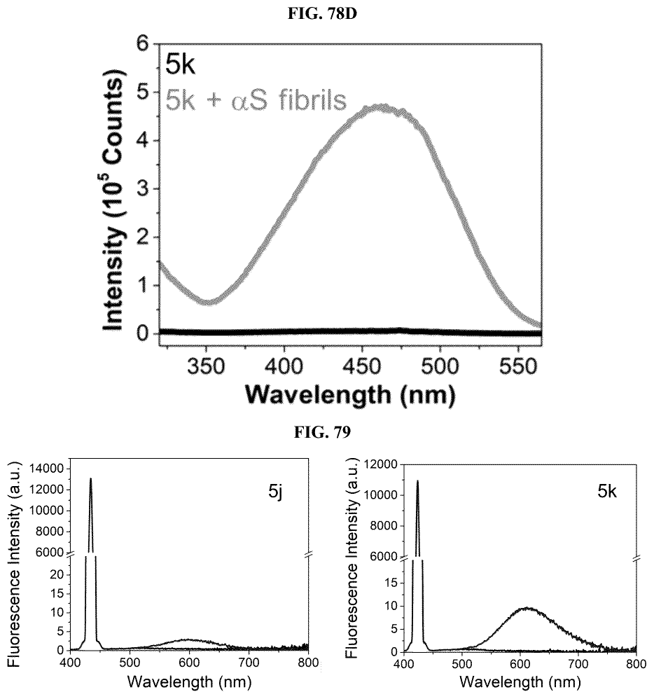

Fluorescence spectra with αS fibrils. Fluorescence measurements were performed using a PTI QuantaMaster 40 fluorescence spectrometer. Fluorescence spectra of 10 μM probes (concentrated stock solution prepared as 10 mM in DMSO) (5b-5f and 5h) in PBS buffer were measured by excitation at their absorbance maximum wavelength in the absence and presence of 50 μM αS fibrils (stock solution 100 μM αS fibrils in PBS buffer). FIG. 76A is protein in αS, FIG. 76B shows 1N4R tau, and FIG. 76C shows Ab1-42 fibrils. FIGS. 77A-77F show fluorescence spectra of probes (5b-5f and 5h) with and without αS fibrils. Final concentrations of probes and αS fibrils were 10 μM and 50 μM, respectively. Emission spectra collected at each dye's maximum absorption wavelength. Ex/Em slit widths: 3 nm/3 nm. FIGS. 78A-78D shows photonic properties of compounds tested. Absorbance is shown in FIG. 78A and FIG. 78B, whereas FIG. 78C and FIG. 78D show excitation spectra of probes 5j and 5k with and without αS fibrils. Final probe and fibrils concentrations were 10 μM and 50 μM, respectively. Final probe and fibril concentrations were 10 μM and 50 μM, respectively. Emission wavelengths were 575 nm for 5j, 580 nm for 5k. Ex/Em slit widths: 3 nm/3 nm. FIG. 79 shows representative QY acquisitions of 5j and 5k with αS fibrils. Black line indicates incident light (αS fibrils without probe) and blue line indicates sample (αS fibrils with probe). Excitation at λexc=435 nm for 5j and λexc=463 nm for 5k and spectral collection (400-800 nm). Final concentrations of the probe and αS fibrils are the 100 μM. Ex/Em slit widths: 5 nm/5 nm.

Absorbance Spectra with αS Fibrils

Absorption measurements were performed using Thermo Scientific Genesys 150 UV Vis spectrometer. Absorption spectra of 10 μM probes (concentrated stock solution prepared as 10 mM in DMSO) (5j and 5k) in PBS buffer were measured in the absence and presence of 50 μM αS fibrils (stock solution 100 μM αS fibrils in PBS buffer).

Excitation spectra with αS fibrils. Excitation spectral measurements were performed using a PTI QuantaMaster 40 fluorescence spectrometer. Excitation spectra of 10 μM probes (concentrated stock solution prepared as 10 mM in DMSO) (5j and 5k) in PBS buffer were measured in the absence and presence of 50 μM αS fibrils (stock solution 100 μM αS fibrils in PBS buffer).

Fluorescent QY Measurements of 5j and 5k with αS Fibrils.

For each QY measurement, the incident excitation light spectrum was collected with 100 L of probes (100 μM) in PBS buffer. After measuring the incident light intensity, probes (5j and 5k) were added to the αS fibrils (100 μM) from a concentrated stock solution (10 mM in DMSO) to a final concentration of 100 μM and the new spectrum (fluorescence intensity and new incident light intensity) was collected. Using the JASCO Quantum Yield Software, the dye QY was calculated by dividing the dye fluorescence intensity by the difference in incident light intensity in the presence and absence of fibrils.

Fluorescence Lifetime Measurements of Probes with αS Fibrils.

TCSPC measurements of fluorescence lifetime decays for 100 μM dyes in the presence of 100 μM αS fibrils were collected with the PTI Quantamaster™ 40 using a pulsed LED with a maximum emission at 486 nm. Fluorescence emission was collected at the indicated wavelength for each dye with 20 nm slit widths. The IRF was collected under identical conditions. Data analysis was performed with FluoFit software using an exponential decay model.

Fluorescence Measurements with Tau Fibrils
Fluorescence Spectra with Tau Fibrils

Fluorescence measurements were performed using a PTI QuantaMaster 40 fluorescence spectrometer. Fluorescence spectra of 5 μM probes (concentrated stock solution prepared as 10 mM in DMSO) (5j, 5k, and ThT) in PBS buffer were measured in the absence and presence of 25 μM tau fibrils (stock solution 32 μM tau fibrils in PBS buffer).

Excitation Spectra with Tau Fibrils

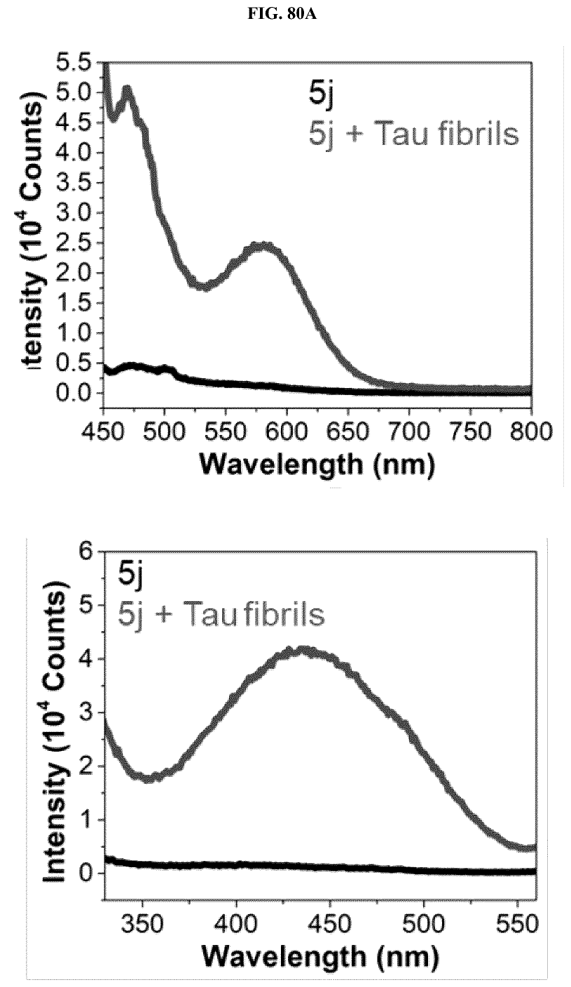

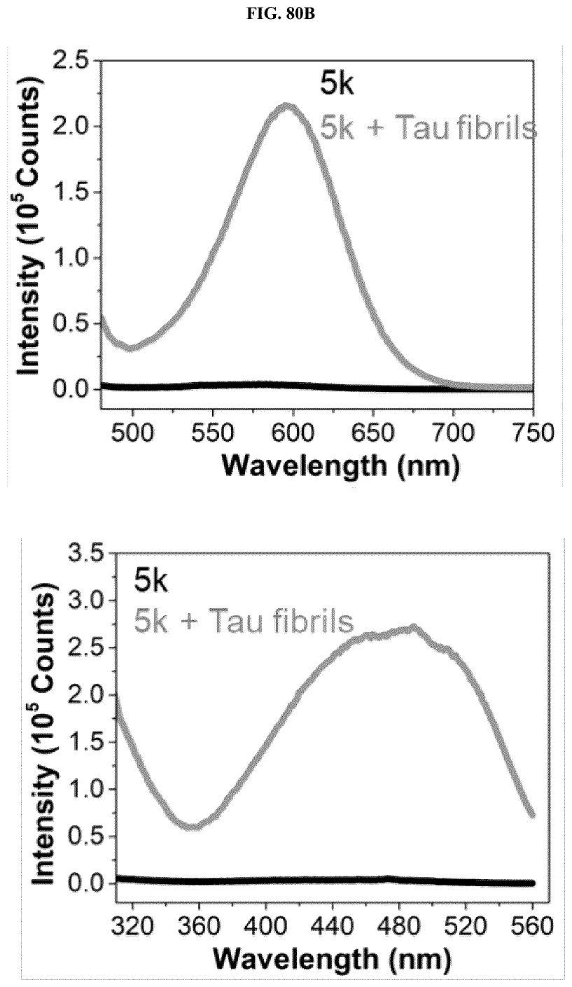

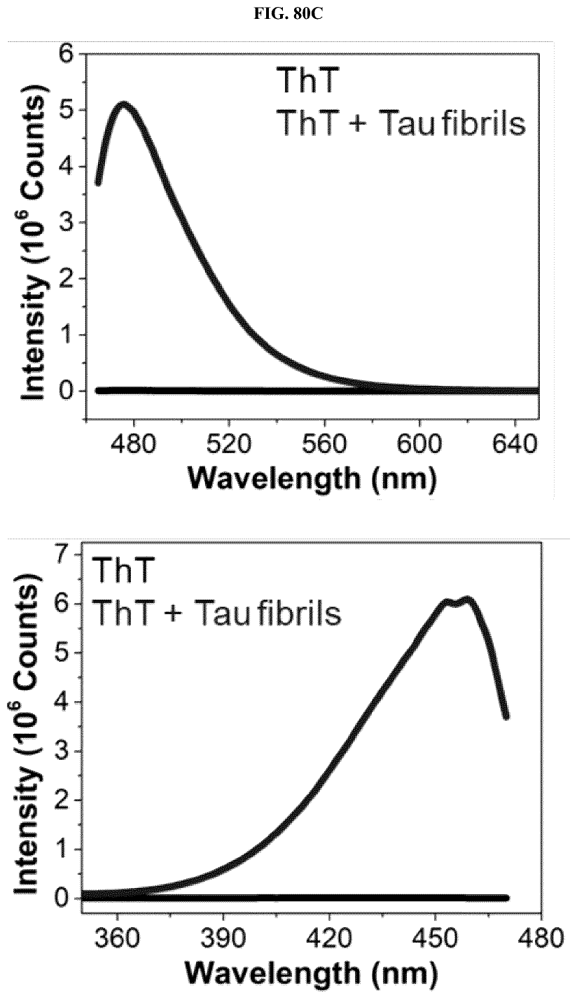

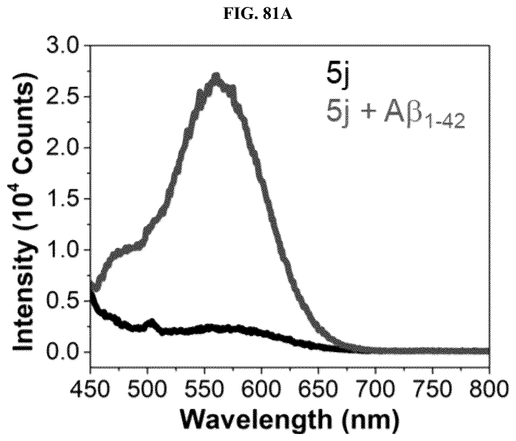

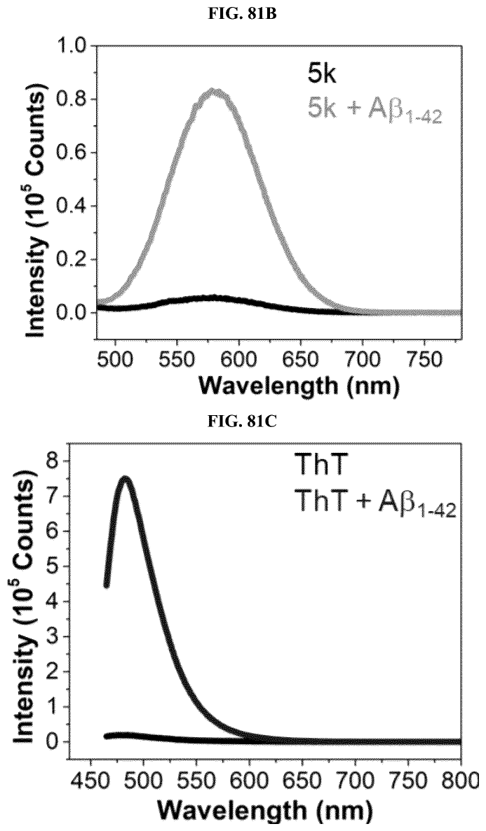

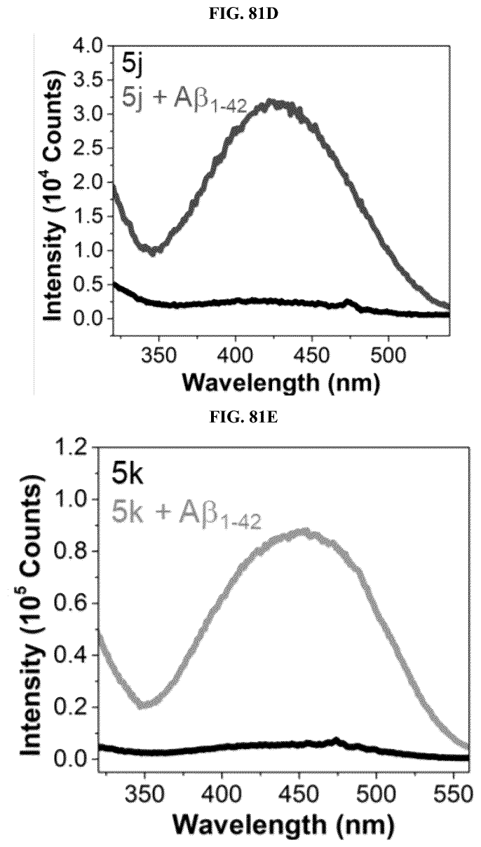

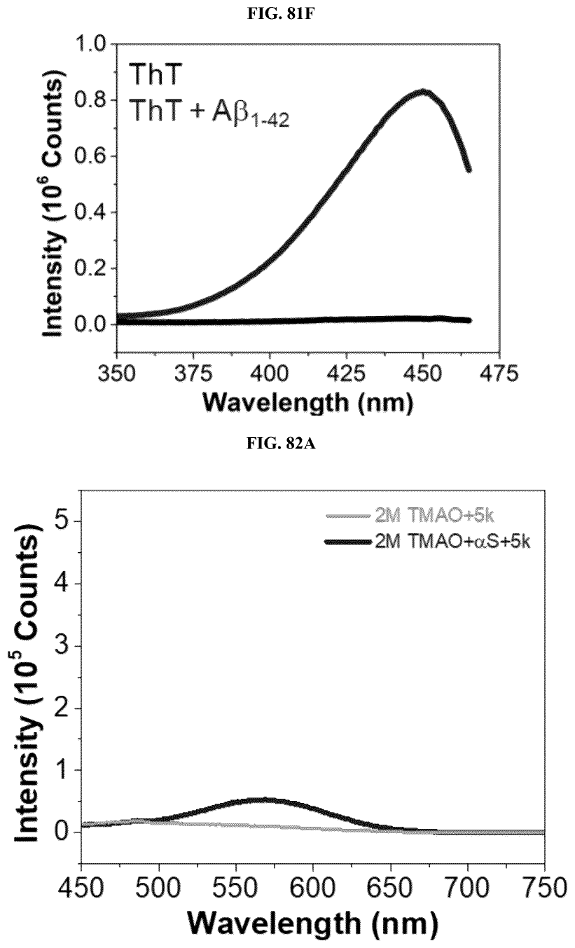

Excitation spectral measurements were performed using a PTI QuantaMaster™ 40 fluorescence spectrometer. Excitation spectra of 5 μM probes (concentrated stock solution prepared as 10 mM in DMSO) (5j, 5k, and ThT) in PBS buffer were measured in the absence and presence of 25 μM tau fibrils (stock solution 32 μM tau fibrils in PBS buffer). FIGS. 80A-80C show fluorescence spectra (top row) and excitation spectra (bottom row) of 5j, 5k and ThT with and without tau fibrils. Final probe and tau fibril concentrations were 5 μM and 25 μM, respectively. Excitation at λexc=435 nm for 5j, λexc=463 nm for 5k, λexc=450 nm for ThT. Emission wavelengths were 575 nm for 5j, 580 nm for 5k, and 482 nm for ThT. Ex/Em slit widths: 3 nm/3 nm. FIGS. 81A-81F show fluorescence spectra (top row) and excitation spectra (bottom row) of 5j (FIG. 81A, FIG. 81D), 5k (FIG. 81B, FIG. 81E) and ThT (FIG. 81C, FIG. 81F) with and without Ab1-42 fibrils. Final probe and fibrils concentrations were 10 μM and 50 M. Excitation at λexc=435 nm for 5j, λexc=463 nm for 5k, and λexc=450 nm fot ThT. Emission wavelengths were 575 nm for 5j, 580 nm for 5k, and 482 nm for ThT. Ex/Em slit widths: 3 nm/3 nm.

Fluorescence Measurements with Ab1-42 Fibrils
Fluorescence Spectra with Ab1-42 Fibrils

Fluorescence measurements were performed using a PTI QuantaMaster™ 40 fluorescence spectrometer. Fluorescence spectra of 10 μM probes (concentrated stock solution prepared as 10 mM in DMSO) (5j, 5k and ThT) in PBS buffer were measured in the absence and presence of 50 μM Ab1-42 fibrils (stock solution 1 mM Ab1-42 in 10 mM phosphate buffer, pH 7.4).

Excitation Spectra with Ab1-42 Fibrils

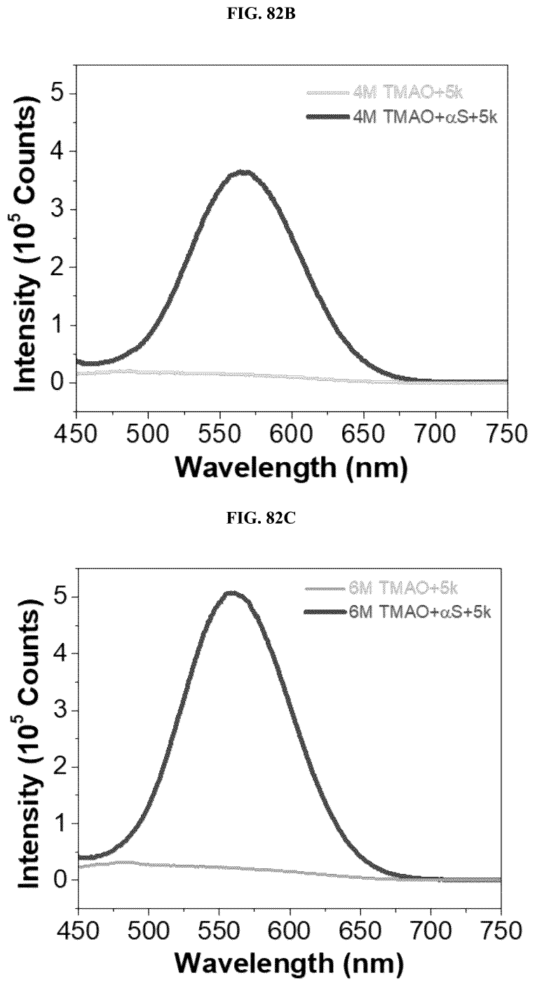

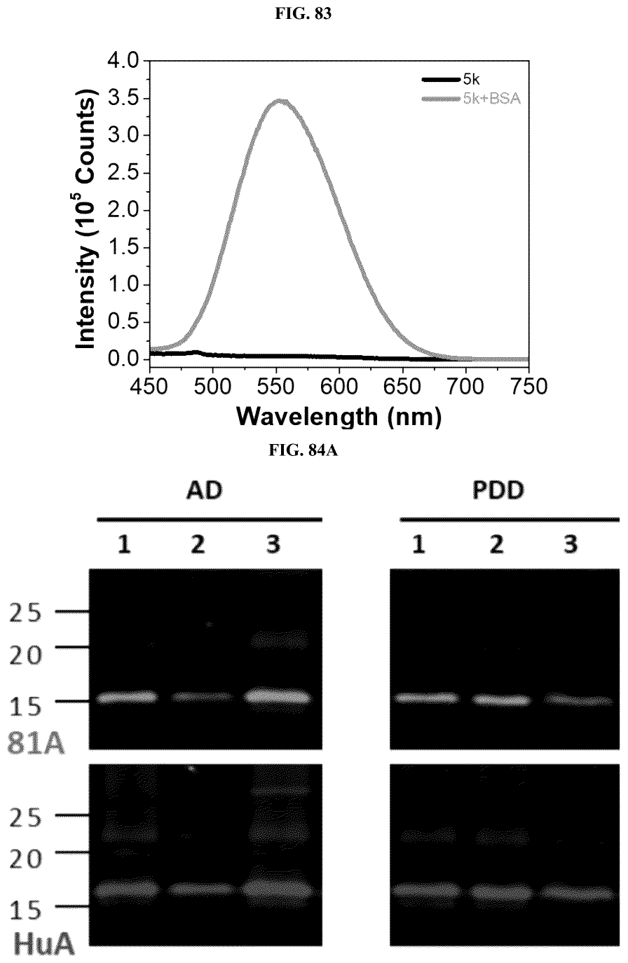

Excitation spectral measurements were performed using a PTI QuantaMaster 40 fluorescence spectrometer. Excitation spectra of 10 μM probes (concentrated stock solution prepared as 10 mM in DMSO) (5j, 5k and ThT) in PBS buffer were measured in the absence and presence of 50 μM Ab1-42 fibrils (stock solution 1 mM Ab1-42 in phosphate buffer). FIGS. 82A-82C: Fluorescence spectra of probe 5k (10 μM) with and without αS monomer (50 μM) in different TMAO concentrations. FIG. 82A shows 2 M TMAO, FIG. 82B shows 4 M TMAO, FIG. 82C shows 6 M TMAO, and tabulated λmax (λem) of 5k with increasing TMAO concentrations. Excitation wavelength for 5k is 418 nm. λmax (nm) of 5k was 567, 566, and 560 respectively. Ex/Em slit widths: 3 nm/3 nm. FIG. 83 shows fluorescence spectra of probe 5k (10 μM) with BSA (50 μM). λmax (λem) of 5k found was 555 nm. Excitation wavelength for 5k is 418 nm. Ex/Em slit widths: 3 nm/3 nm.

### HEK Cell Lysate Preparation.

HEK 293T cells were grown on 150 mm dishes in sterile-filtered Dulbecco's modified Eagle's medium (Gibco, Thermo Fisher) supplemented with 10% fetal bovine serum (FBS), 1% penicillin/streptomycin (Mediatech, 10 mg/ml each). Cells were then washed with PBS and harvested in 5 mL PBS by scraping. Cells were pelleted by centrifugation at 1000 rpm for 5 min, and the supernatant was removed. The resulting cell pellet was re-suspended in 300-500 μL PBS buffer with 0.1% Triton X-100 (Bio-Rad) and 1×HALT protease inhibitor cocktail without EDTA (Thermo Fisher), then lysed by sonication for two 30 s cycles (cycle 1: 2 s on 2 s off, amp 50; cycle 2: 2 s on 2 s off, amp 55) using a QSonica Q700 sonicator fitted with a microtip. Following sonication, lysate was centrifuged at 13,200 rpm for 60 min to separate membrane and cytosolic protein fractions. The resulting lysate cytosolic or membrane fraction was then used immediately after concentration determination by DC protein assay (Bio-Rad).

Selectivity of Probe 5k Binding with αS Fibrils in HEK Cell Lysate.

Probe 5k in PBS buffer (1 μM, the stock solution prepared as 10 mM in DMSO) were incubated with different αS fibril concentrations (0, 5, 10, 25 and 50 μM) in the presence of 10 mg/mL HEK cell lysate in Greiner 96 well flat black ½ area plates at 37° C. with shaking at 500 rpm for 15 min in an IKA MS3 control orbital shaker. After 15 min incubation, fluorescence intensity measurements were obtained with a Tecan Spark plate reader (Mannedorf, Switzerland) by excitation with λex/λem=463/580 nm for 5k, using the following parameters: excitation and emission bandwidth 5 nm, delay time 0 μs, integration time 40 μs.

Experimental Procedure for the Fluorescent Measurements of 5k with TMAO:

A 1 mM stock solution of 5k in DMSO and a 6.5 M stock solution of trimethylamine N-oxide (TMAO) in Tris buffer at pH 7.4 were prepared. From these stocks, the fluorescence measurements of probe 5k (10 μM) were carried out with and without αS monomer (50 μM) at different concentrations of TMAO (2 M, 4 M and 6 M) diluted with the Tris buffer. The solutions were mixed and then the spectra were measured using a Photon Technology International (PTI) QuantaMaste 40 fluorescence spectrometer with excitation at 418 nm.

### Patient Tissue Characterization and Amplified Fibril Preparation

Preparation of Sarkosyl-Insoluble αS from Disease and Control Brains

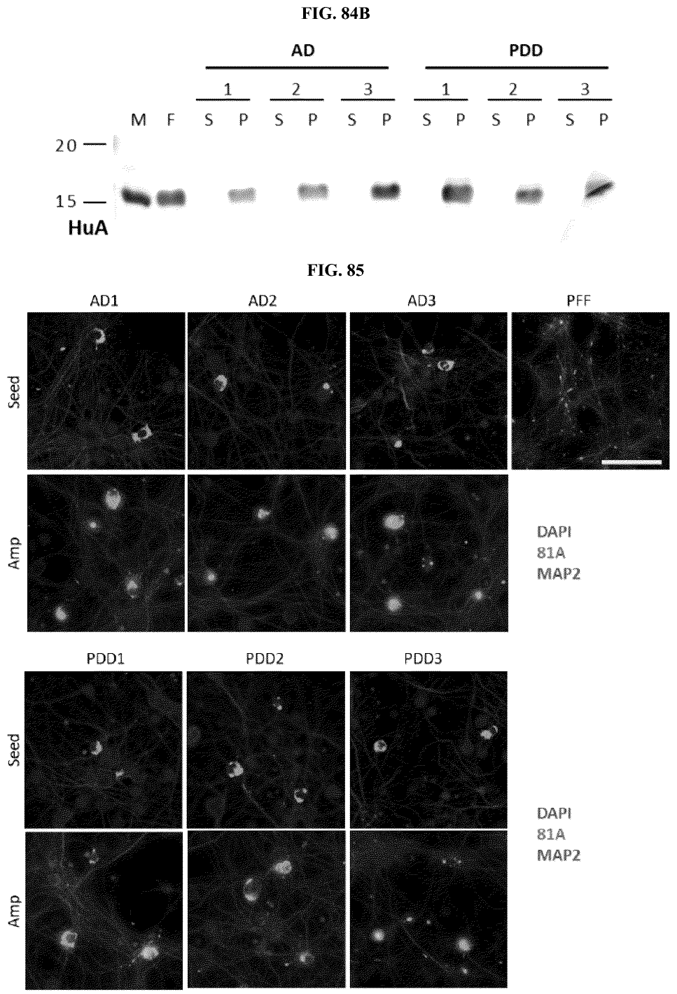

Frozen postmortem human frontal cortex brain tissues from three patients diagnosed with Alzheimer's disease (AD) and three patients diagnosed with Parkinson's disease with dementia (PDD) were selected for sequential extraction of αS aggregates based on a high burden of αS pathology determined by immunohistochemical staining. In brief, 5-10 g of frontal cortical gray matter were homogenized in five volumes (W/V) of high-salt (HS) buffer (50 mM Tris.HCl pH 7.4, 750 mM NaCl, 10 mM NaF, 5 mM EDTA) with protease and protein phosphatase inhibitors, incubated on ice for 20 min and centrifuged at 100,000×g for 30 min. The pellets were then re-extracted with HS buffer, followed by sequential extractions with five volumes of 1% Triton X-100-containing HS buffer and 1% Triton X-100-containing HS buffer with 30% sucrose. The pellets were then re-suspended and homogenized in 1% sarkosyl-containing HS buffer, rotated at room temperature for 2 h or at 4° C. overnight and centrifuged at 100,000×g for 30 min. The resulting sarkosyl-insoluble pellets were washed once with Dulbecco's PBS (DPBS) and re-suspended in DPBS by sonication (QSonica Microson XL-2000; 50 pulses; setting 2; 0.5 s per pulse). These final sarkosyl-insoluble fractions are referred to as “brain extracts.: The amount of αS in the brain extracts was determined by sandwich ELISA using Syn9027, a mono-clonal antibody (Mab) to αS, and the protein concentrations were examined by bicinchoninic acid (BCA) assay. Total protein in AD cases ranged from 5 to 13 mg/mL and total protein in PDD cases ranged from 9 to 12 mg/mL. αS concentration in AD cases ranged from 36 to 43 μg/mL and total protein in PDD cases ranged from 24 to 33 μg/mL. FIGS. 84A-84B. Preparation of fibrils amplified from patient-derived α-synuclein aggregates. FIG. 84A shows Western blots of the final brain lysate samples extracted from AD and PDD patient brains. Membranes were probed with anti-pSer-129 αS (81A) or total αS (HuA). FIG. 84B shows Western blot of sedimentation results for amplified fibrils (AFs) after seven days of incubation. Supernatant (S) and pellet (P) fractions were run for each reaction with monomer (M) and PFF (F) as loading controls. The membrane was probed with HuA.

In Vitro Amplification of αS Fibrils from Brain Extracts.

Brain-derived αS aggregates in extract samples from three AD and three PDD cases (FIG. 38A) were used as seeds for templated aggregation of recombinant αS monomer. Brain lysates were sonicated on high for 20 cycles of 30 s on, followed by 30 s off (Diagenode Biorupter UCD-300 bath sonicator) and diluted to 350 nM (based on sandwich ELISA) in DPBS (pH 7.4, without Mg2+ and Ca2+) containing sufficient monomer for a final αS concentration of 35 μM (1% patient-derived seed, 99% monomer) and incubated at 37° C. with constant shaking at 1000 rpm for 7 days. The resulting aggregates were termed amplified fibrils (AFs). After seven days of incubation, amplification progress was determined by removing an aliquot and diluting tenfold in DPBS to a final αS concentration of 3.5 μM. Samples were centrifuged at 45k rpm for 30 min at 10° C. in a TLA-55 rotor (Beckman Coulter). The supernatants were removed, and the pellets were resuspended in DPBS. Equal volumes of supernatant and pellet fractions were analyzed on SDS-PAGE gels and Western blot, with amplified aggregates migrating to the pellet fraction (FIG. 38B). Primary mouse hippocampal neurons were then treated with the AFs to confirm they retained the characteristic pathological phenotype of the original brain lysate and were distinct from recombinant PFFs. Primary mouse neurons were prepared from the hippocampus of embryonic day E16-E18 CD1 mouse embryos. Primary mouse hippocampal neurons were plated at 17.5k cells per well in 96-well plates and were treated with 50 ng per well of brain-derived, AFs, or recombinant PFFs at seven days in vitro and maintained for two weeks post-treatment. Cells were grown in Neurobasal medium supplemented with B27, 2 mM GlutaMax, and 100 U/ml penicillin/streptomycin (ThermoFisher). Following fixation with 4% paraformaldehyde, 4% sucrose, and 1% Triton-X100, cultures were probed with antibodies for MAP2 (17028) and pSer-129 αS (81A) Patient-derived fibrils and AFs induce discrete, dense inclusions in cell bodies with little neuritic pathology while PFF induce predominantly small neuritic puncta and threads.

### Control Sample Preparation.

Control samples for comparison to AFs in fluorescence binding experiments below were prepared as follows. “Lysate” controls used a portion of the same brain extracts from AD cases 1-3 and PDD cases 1-3, but were not subjected to amplification. These should contain the same concentration of patient-derived αS and other insoluble proteins as the AF samples, but no seeded fibrils. Pre-formed fibril (PFF) controls took these lysates and added αS fibrils, generated as described above for binding studies of the pure fibrils, to match the αS monomer concentration used in generating AFs (35 μM). Thus, they would have the same fibril content as an amplification reaction which went to completion, but with a fibril morphology that is not templated by the patient-derived αS aggregates. FIG. 85 shows representative images of pathology induced in primary mouse hippocampal neurons for seeds and AFs (Amp) for each case, or PFFs. Probed with 81A (green), anti-MAP2 (red) and DAPI (blue). Scale bar 50 m.

Fluorescence Measurements with Patient Tissue Lysate Samples Fluorescence measurements of 5k and ThT with AD and PDD patient cases Probe binding was tested with 7 μM stock concentrations of patient tissue samples from 3 different AD patient cases (AD1, AD2, and AD3) with 3 different conditions: lysate only, amplified αS fibrils (AFs), preformed αS fibrils (PFFs), all prepared as described above. Final samples were prepared in a Greiner 384-well small volume microplate by diluting to 1 μM final concentration with PBS buffer from each condition's stock in a 5 μL final volume. To this was added bimane probes 5k and ThT at 1 μM probe concentration (10 mM stock of 5k and ThT in DMSO and 5 mM stock of ThT in PBS buffer), then incubated at 37° C. with shaking at 500 rpm for 15 min in an IKA MS3 control orbital shaker. After 15 min of incubation, fluorescence intensity measurements were obtained with a Tecan Spark plate reader by excitation λex/λem=463/580 nm for 5k and λex/λem=450/482 nm for ThT, using the following parameters: excitation and emission bandwidth 5 nm, delay time 0 μs, integration time 40 μs. Identical experiments were performed for 7 μM stock concentrations of patient tissue samples from 3 different PDD patient cases (PDD1, PDD2, and PDD3) with 3 different conditions: lysate only, amplified αS fibrils (AFs), preformed αS fibrils (PFFs).

To modulate the photophysical properties of the bimane core, the 3-position was derivatized, since the 3-bromomethyl bimane 2 convert to a thioether by reaction with thiols leads to blueshifted absorption and fluorescence turn-on. To access bimane derivatives, bromide 2 was synthesized and converted it to phosphonate ester 3 (Scheme 2) for use in Horner-Wadsworth-Emmons (H-W-E) reactions with diverse aldehydes, one of the most useful methods for C═C bond formation with predominantly E configuration (Scheme 3). Initially, the gram-scale synthesis of the bimane core 1 was achieved in three sequential steps with modifications of a previously established milligram scale procedure. 19 It involves 1) condensation under sonication (96.4% yield), 2) chlorination by TCCA (84% yield), and 3) cyclization under heterogeneous basic conditions (71% yield), resulting in improved yields. Then, the key precursor methyl bimane phosphonate 3 was synthesized in two steps as shown in Scheme 2, bromination followed by an Arbuzov reaction. Synbimane 1 was treated with a bromine solution to afford bromobimane 2 in 74% yield. Subsequent reaction with neat trimethyl phosphite led to the formation of compound 3 in 82% yield.

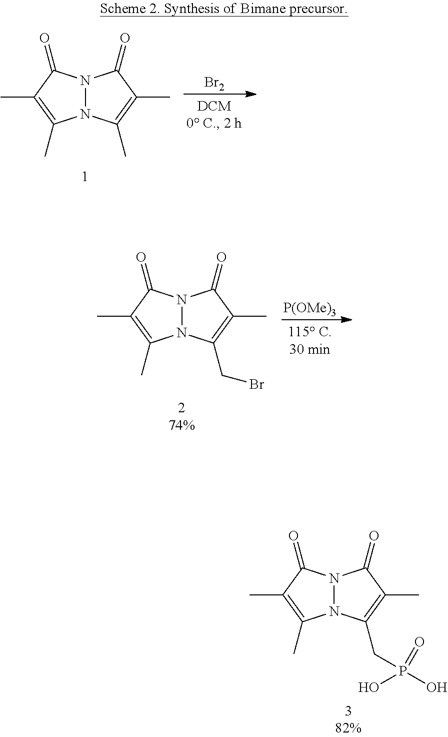

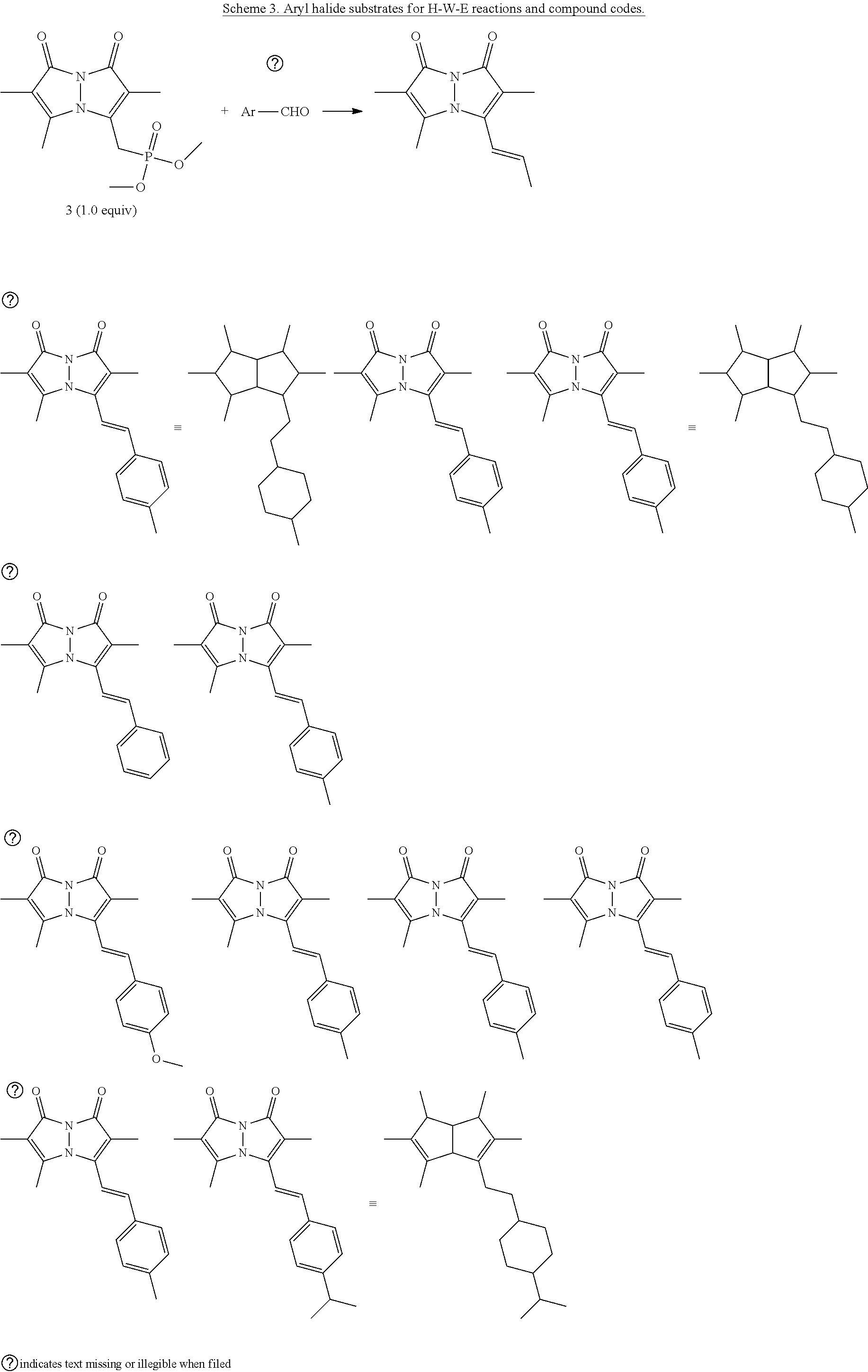

### Synthesis of Arylated Bimane Probes

A set of commercially available aryl aldehydes with a range of electron-withdrawing to electron-donating para-substituents were subjected to the H-W-E reaction with 3 under Condition A, K₂CO₃ in an acetonitrile (ACN)/H₂O mixture (6.4 v/v) at reflux (Scheme 3). All the electron-poor aryl aldehydes (4a-4c) afforded good to excellent isolated yields (>78%) of 5a-c with short reaction times, but electron-rich aryl aldehyde 4k afforded a low isolated yield (<10%). Reactions of protected aldehydes 4g and 4i proceeded with Condition A to give excellent yields of 5g and 5i. Next, deprotection of 5g and 5i by trifluoroacetic acid (TFA) yielded free alcohol and amine derivatives 5h and 5j. To improve conversions for electron-rich aldehydes, the reaction conditions were optimized to prepare the dimethylamino analogue 5k as a model reaction by varying the base, solvent, and temperature as shown in Table 12. Optimized Condition B using NaOMe as a base in dry DMF at room temperature (entry 4 in Table 12) was then applied to other electron-rich aryl aldehydes (4d-4f, 4k), improving the yields to 43-72% of 5d-5f and 5k. All probes were characterized by 1H, 13C NMR and high-resolution mass spectrometry; 5a, 5c, and 5j were also characterized by X-ray analysis (FIG. 50E).

### Substituent Effects on Photophysical Properties

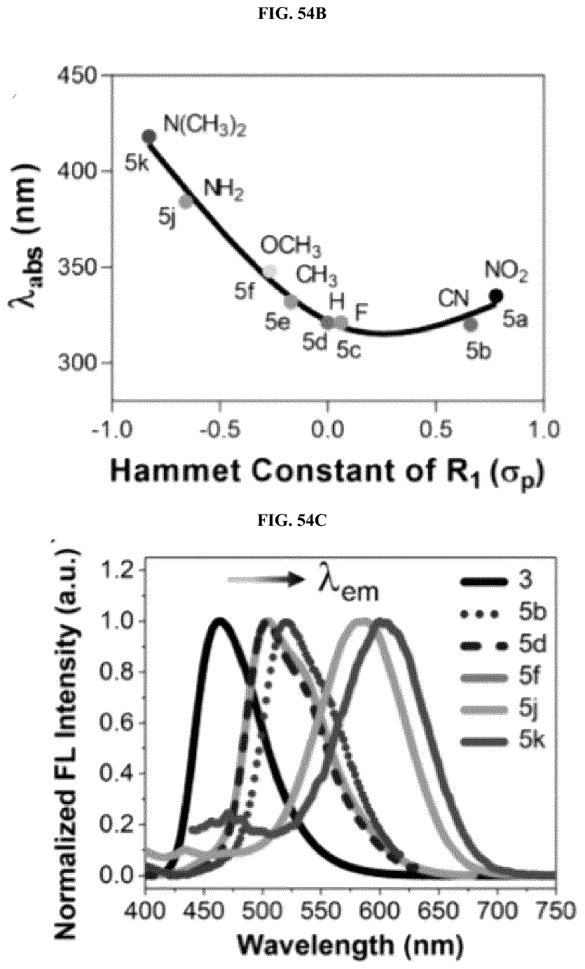

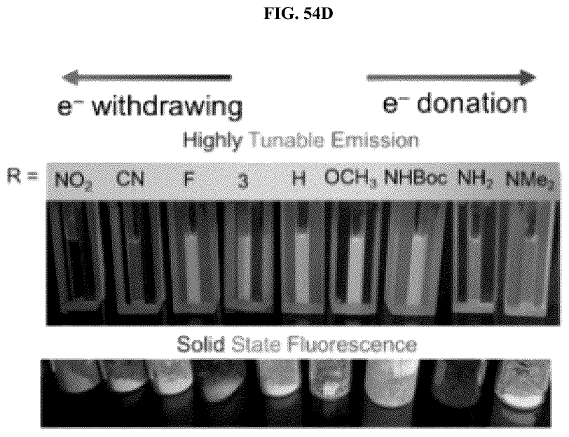

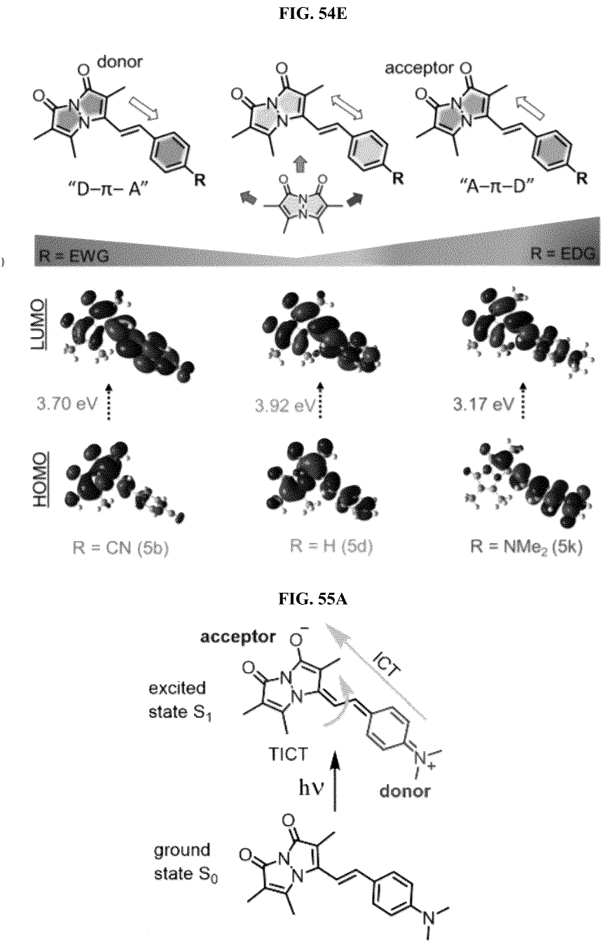

After successful synthesis of a styryl bimane probe library with varied substitution, it was examined whether the photophysical properties of each probe in 50:50 ACN/phosphate buffered saline (PBS). As shown in absorption spectra FIG. 54A, a more electron-donating aryl substitution generated a more red-shifted absorption maximum (λabs) compared to λabs of parent compound 3, whereas a more electron-withdrawing aryl substitution generated a blue-shifted λabs, suggesting that there is significant electronic communication through the styryl bimane scaffold.

In addition to the λabs shifts, a 3-5-fold increase was observed in molar absorptivity (F) relative to 3, which is attributed to the increase in π-conjugation through the styryl group. These results encouraged us to establish systematic guidelines to aid the deliberate selection of substituents for future bimane derivatives. In this context, 5a-f, 5j, and 5k were analyzed in terms of the Hammett substituent constant (sp) for the para-functional group on the phenyl ring (FIG. 54B). Electron-donation showed a positive correlation between the sp and λabs, but electron-withdrawing groups changed the slope of the Hammett plot, indicating a change in mechanism.

This analysis was further supported by analysis of HOMO-LUMO density functional theory (DFT) calculations of 5b (R=CN), 5d (R=H), and 5k (R=NMe₂) (FIG. 54E). In the case of 5k, the movement of electron density from the N,N-dimethylamino group of 5k to the bimane core upon excitation from S₀ to S₁. (λabs=418 nm, E=3.17 eV). For 5d there is no significant change in electron density movement upon excitation from S₀ to S₁ (λabs=321 nm, E=3.92 eV). In contrast, for 5b the movement of electron density from the bimane group to the electron withdrawing group on the phenyl ring is seen upon excitation from S₀ to S₁ (λabs=320 nm, E=3.70 eV). Again, for 5a (R=NO₂), redshifted absorption was absorbed at λabs=335 nm with E=3.32 eV. Based on this combination of experimental results and computational analysis, in FIG. 54E, the potential to design “push-pull” systems of both the donor-spacer-acceptor (D-π-A) and acceptor-spacer-donor (A-π-D) variety was illustrated. This design is facilitated by the bimane core, which acts as either a donor or acceptor, its functionality depending on the specific aryl substituent R.

TABLE 12

| | ACN:PBS (50:50 v/v) | | | | | ACN only | | | | |
|---|---|---|---|---|---|---|---|---|---|---|
| | Abs. | Em. | ε | Stokes | | Abs. | Em. | ε | Stokes | |
| | λabs | λem | (M⁻¹ | shift | Q.Y. | λabs | λem | (M⁻¹ | shift | Q.Y. |
| Compound | (nm)^{a} | (nm)^{b} | cm⁻¹) | (nm)^{d} | (%)^{e} | (nm)^{a} | (nm)^{b} | cm⁻¹) | (nm)^{d} | (%)^{e} |
| 1 | 384 | 464 | 7902 | 80 | 64.6 | 375 | 432 | 6121 | 57 | 77.7 |
| 3a | 349 | 448 | 27587 | 99 | 0.06 | 346 | 412, | 26667 | 229 | 0.1 |
| | | | | | | | 575 | | | |
| 3b | 345 | 462, | 31088 | 185 | 0.21 | 340 | 432, | 32612 | 192 | 0.56 |
| | | 530 | | | | | 532 | | | |
| 3c | 344 | 452, | 29304 | 192 | 0.16 | 339 | 423, | 29974 | 195 | 0.4 |
| | | 536 | | | | | 534 | | | |
| 3d | 373 | 466, | 22575 | 157 | 0.27 | 367 | 430, | 22961 | 163 | 0.54 |
| | | 530 | | | | | 530 | | | |
| 3e | 375 | 445, | 20460 | 159 | 0.26 | 369 | 430, | 21094 | 162 | 0.52 |
| | | 534 | | | | | 531 | | | |
| 3f | 447 | 512, | 22370 | 195 | 0.77 | 435 | 624 | 25212 | 189 | 44.2 |
| | | 642 | | | | | | | | |

^{a}Maximum absorption wavelength,
^{b}Maximum emission wavelength,
^{c}Molar absorption coefficients at maximum absorption wavelength,
^{d}Difference between maximum absorption wavelength and maximum emission wavelength,
^{e}Fluorescence quantum yield (error limit within ±5). Final concentration of the probe is 25 μM.

TABLE 13
Screening of H-W-E reaction conditions.

| Entry | Solvent | Base | Temperature | Time | | Yield (%) |
|---|---|---|---|---|---|---|
| 1 | ACN/H₂O | K₂CO₃ | RT | 40 | h | 2 |
| 2 | ACN/H₂O | K₂CO₃ | 90° C. | 2 | h | 9 |
| 3 | DMF | KOtBu | RT | 40 | h | 27 |
| 4 | DMF | NaOMe | RT | 40 | h | 46 |
| 5 | DMF | NaOMe | 60° C. | 40 | h | 38 |
| 6 | DMSO | KOtBu | RT | 40 | h | 4 |
| 7 | DMSO | NaOMe | RT | 40 | h | 31 |
| 8 | DMSO | NaOMe | 60° C. | 40 | h | 12 |

^{a}Reaction conditions: 3 (1 equiv), aryl aldehyde 4k (1 equiv), KOtBu (1.5 equiv), NaOMe (1.5 equiv), K₂CO₃ (1.5 equiv), Yields based on chromatogram peak areas.

Next, the emission spectrum of each compound was measured in ACN/PBS (50:50) with excitation at λabs (Table 12). As shown in FIG. 54C, analysis of the emission spectra of selected bimane derivatives 5b, 5d, 5f, 5j, 5k and 3 with different substituents showed tunable emission with a significant bathochromic shift of the emission maximum (λem) compared to the parent phosphonate 3 (λem=464 nm). This tunable emission is seen both in solution and the solid state (FIG. 54D). Notably, derivatives 5a-c, featuring electron-withdrawing substituents on the aryl ring, exhibited green emission within the range of 502-520 nm. In contrast, derivatives 5j (R=NH₂, λem=583 nm) and 5k (R=NMe₂, λem=604 nm), possessing electron-donating substituents, displayed larger bathochromic shifts in λem. Further comparison of 5h (R=NHBoc, λem=505 nm) and 5i (R=NH₂, λem=583 nm), which differ only by removal of the tert-butoxycarbonyl (Boc) group, highlighted the influence of the amine lone pair on the emission wavelength shift (Table 13). Taken together, these results further support the idea that the tunable emission mainly stems from the design of “push-pull” D-R-A or A-R-D systems. Moreover, the Stokes shifts was calculated for 5a-5k and observed values ranging from 133 to 217 nm (Table 13). These interesting fluorescent properties of styryl bimanes, such as high tunability and large Stokes shift, will make them valuable probes for a broad spectrum of biological applications. Measurement of the fluorescence quantum yield (QY) of each compound in ACN/PBS revealed dramatic changes for the styryl bimanes 5a-5k (0.1-3.4%) compared to parent phosphonate 3 (64.6%). The changes in QY are also reflected in shortened fluorescent lifetimes (t) for the derivatives (1.02-1.31 ns) compared to 3 (2.32 ns) (Table 14). In pure ACN, F values were not dramatically affected, but the QY of some compounds increased significantly, particularly tertiary amine probe 5k, with a QY of 7.5% in ACN. Notably, primary amine 5j did not experience as large an increase in QY (2%) (Table 12). The large effect of solvent on QY prompted us to examine the environmental sensitivity of the probes.

### Environmental Sensitivity of Probes: Polarity and Viscosity

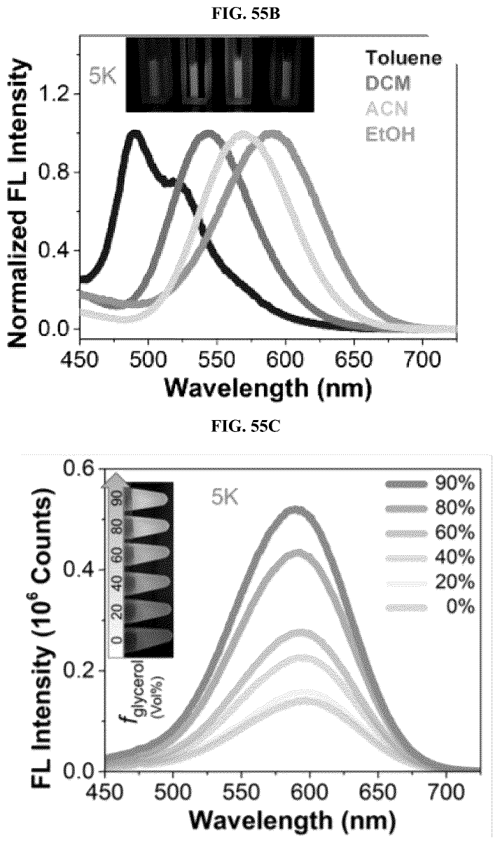

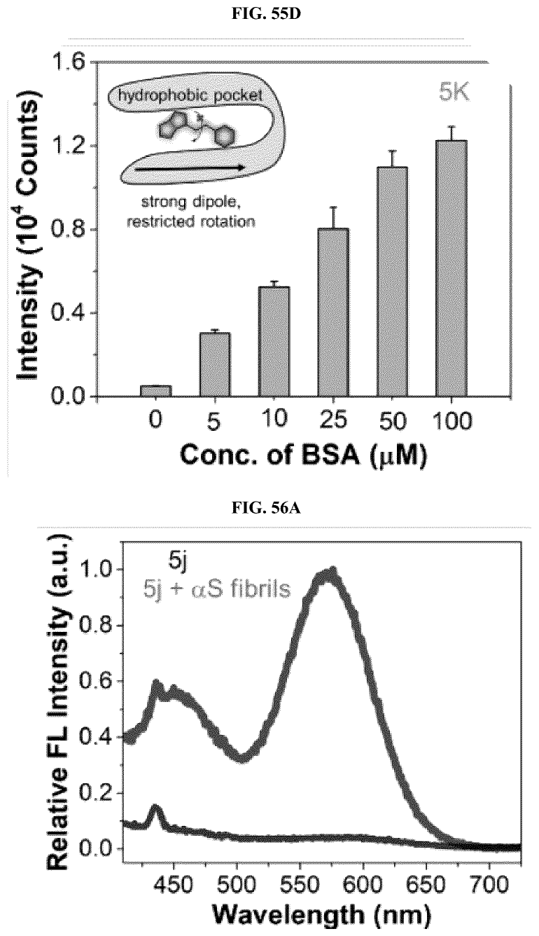

Solvatochromism was investigated more generally, obtaining emission spectra in four organic solvents with different polarity: nonpolar aprotic (toluene, DCM), polar aprotic (ACN), and polar protic (EtOH). As illustrated for 5k (R=NMe₂) in FIG. 55B, the probe showed a high level of solvatochromism, with a red-shift in more polar solvents and emission spanning 490-590 nm. Other derivatives having electron-donating groups (5j, R=NH₂) showed sensitivity to solvent polarity, while parent compound 3 showed no significant environmental sensitivity. Probe 5b with an electron-withdrawing group (R=CN) and 5d (R=H) displayed no significant solvatochromism. Overall, these results suggest that probes with electron donating groups have the ability to exhibit solvatochromism.

pH effects were also studied and found that although some compounds exhibited pH effects, these were not significant in physiological pH ranges. For example, 5h (R=OH) has red-shifted absorption at pH 11 with a reduced QY, but no shift in emission. Probe 5j (R=NH₂) has blue-shifted absorption and emission at pH 2. Taken together with the solvent polarity effects, these data show that environmental effects are tunable based on the aryl substituent and motivate the use of amine containing derivatives as probes of the local environment on a protein surface or in a cellular compartment. The lack of pH sensitivity in the physiological range or changes in protic solvents implies that these effects are modulated more by the general polarity of the environment than by specific hydrogen bonding or protonation. Moreover, in nonpolar solvents 5k showed a blue-shifted emission with enhanced intensity, while in polar solvents it displayed a red-shifted emission accompanied by a reduction in intensity. It was hypothesized that styryl bimane probes in polar solvents undergo TICT through non-radiative pathways, resulting from free rotation around the stryryl linker that connects the π-systems in the excited state. To investigate this, the emission spectra of 5k was recorded in various binary mixtures of ethylene glycol and glycerol (FIG. 55C). Since ethylene glycol and glycerol have similar polarities, the fluorescence intensity measured in mixtures of the two solvents should be influenced solely by the solvent viscosity. The increased viscosity of glycerol serves to restrict free rotation leading to non-radiative TICT states. A 3.7-fold increase in fluorescence intensity with increased % glycerol indicates that, indeed, rotational restriction planarizes the π system, increasing radiative emission. It was supposed that the combination of solvatochromic and rotor effects should lead to a dramatic fluorescence turn-on when binding occurs within a hydrophobic cavity on a protein. Indeed, testing 5k with bovine serum albumin (BSA) showed that turn-on was possible as shown in FIG. 55D, prompting us to test binding to targets of more physiological significance.

### Bimane Derivatives as “Turn-On” Binding Probes for αS Fibrils

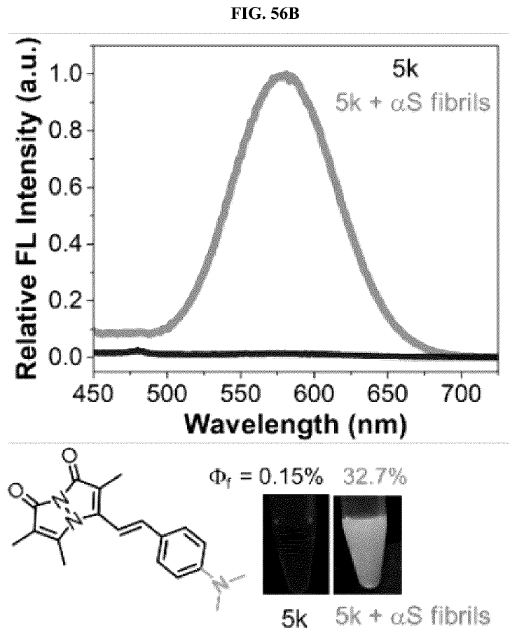

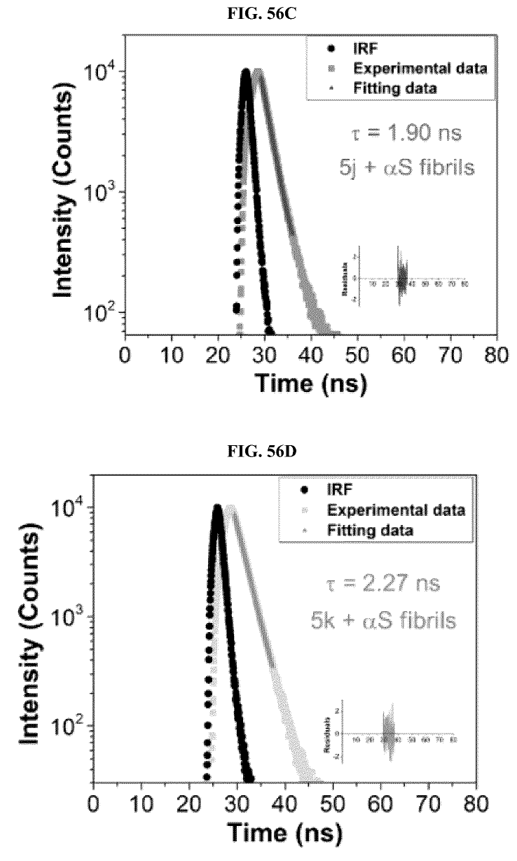

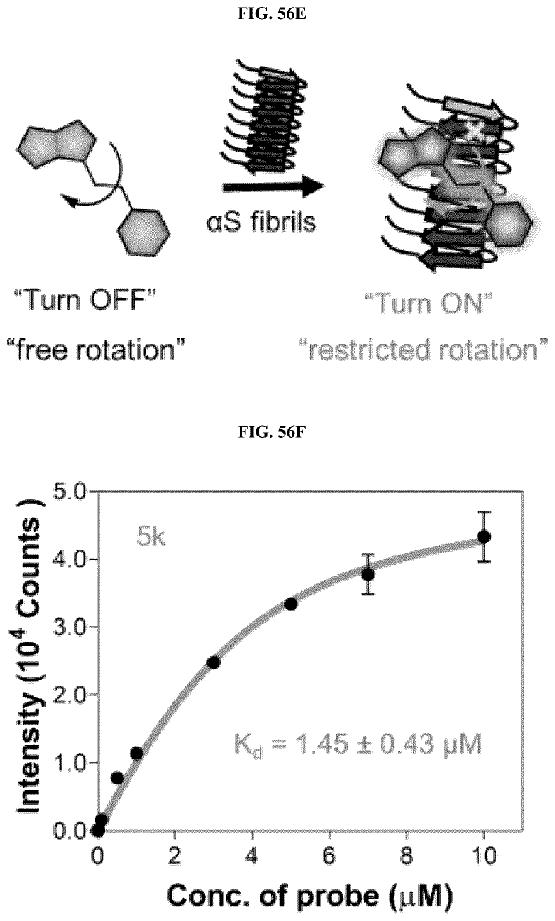

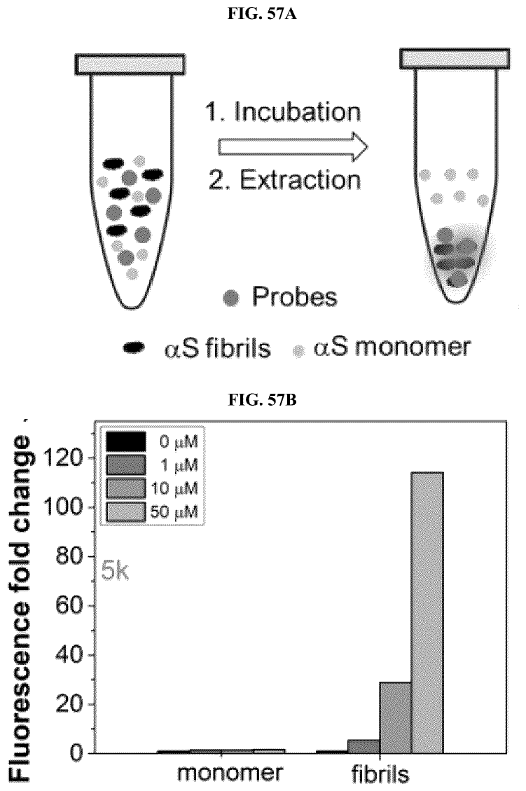

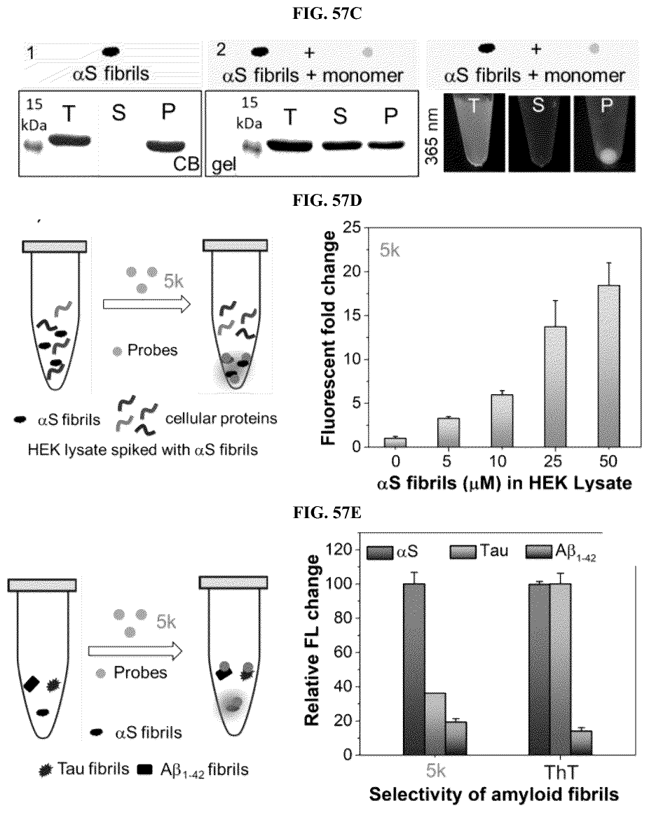

The intriguing photophysical features of our bimane probes, such as solvatochromism and viscosity sensitivity exhibited by 5j and 5k, along with their structural resemblance to amyloid binding dyes currently under investigation in our laboratory and others, prompted us to explore their potential application in amyloid sensing. Initial focus was centered on the αS protein that aggregates to form amyloid fibrils which play an important role in PD. 5j and 5k were screened against in vitro generated αS preformed fibrils (PFFs) by fluorescence spectroscopy. Notably, probes 5j and 5k demonstrated a significant turn-on of orange fluorescence in the presence of PFFs, exhibiting emission maxima at 575 nm and 580 nm, respectively (FIGS. 56A-56C). Probes 5b-5f and 5h didn't show “turn on” fluorescence, as expected, due to lack of environmental effects. Analysis of the tabulated photophysical properties of 5j and 5k in the presence and absence of αS fibrils revealed an increase in both F (3-fold for 5j and 2.2-fold for 5k) and QY (122-fold for 5j and 218-fold for 5k). These enhancements contributed to a turn-on effect with 364 and 476-fold increases in brightness for 5j and 5k, respectively (Table 14). Furthermore, these dyes exhibited large Stokes shifts in the presence of PFFs: 140 nm and 117 nm for 5j and 5k, respectively, making them suitable for imaging applications with heightened sensitivity by minimizing background fluorescence at the excitation wavelength.

TABLE 14
Spectral properties of 5j and 5k in the absence and presence of fibrils.

| | | | | | E | Stokes | | | Fluorescence |
|---|---|---|---|---|---|---|---|---|---|
| | | λabs | λex | λem | (M⁻¹ | Shift | Fluorescence | Relative | Lifetime |
| Dye | PFFs | (nm)^{a} | (nm)^{b} | (nm)^{c} | cm⁻¹)^{d} | (nm)^{e} | QY (%) | brightness^{g} | (ns)^{f} |
| 5j | − | 372 | − | − | 15385 | − | 0.17 | − | − |
| 5j | + | 359 | 435 | 575 | 45956 | 140 | 20.7 | 364 | 1.90 |
| 5k | − | 393 | − | − | 20071 | − | 0.15 | − | − |
| 5k | + | 386 | 463 | 580 | 43864 | 117 | 117 | 476 | 2.27 |

^{a}Maximum absorption wavelength (λabs) in the presence of αS PFFs corresponds to a mixture of bound and unbound dye,
^{b}Maximum excitation wavelength (λex) better represents the bound form of dye,
^{c}Maximum emission wavelength (λem) corresponds to a bound dye,
^{d}Molar absorptivity (ε) was measured at λabs.
^{e}Stokes shift was determined as the difference between lex and λem. Final dye and PFF concentrations were 10 and 50 μM, respectively, for measurements of λabs, λex, λem, and Stokes shift.
^{f}Fluorescence quantum yield (QY) and lifetime measurements were made with 100 μM dye and PFFs.
^{g}Relative brightness was determined as the ratio of ε•QY in the presence and absence of PFFs. λex/λem = 435/575 nm for 5j and 463/580 nm for 5k.

Additionally, the observed λem values when bound to PFFs are consistent with an ACN-like environment. However, in comparing ACN and ACN/PBS results, these enhancements in ACN were accompanied by decreases in F and were smaller than those observed for PFF-bound forms of the compounds (Table 14). Thus, it was interpreted that turn on results from contributions involving both solvatochromism and restriction of free rotation. Additionally, the probe's binding to αS fibrils was characterized through fluorescence lifetime measurements. As depicted in FIGS. 56C-56D, the probes exhibited fluorescence lifetimes (T) of 1.90 ns for 5j and 2.27 ns for 5k when bound to PFFs, similar to the lifetime of the key precursor 3 (2.32 ns). This suggests that the PFF-bound forms represent unquenched bimane fluorescence.

For 5j, emission peaks at 450 and 575 nm were observed, likely corresponding twisted and planar states of the molecule, respectively, where the twisted state has emission arising from the bimane core (˜464 nm). Such a double peak would complicate interpretation of fibril binding data for 5j. Together with the larger change in brightness upon fibril binding, these photophysical properties imply that probe 5k is a superior probe for αS fibrils compared to 5j. The binding affinity of 5k was evaluated through fluorometric titration in the presence of 100 M αS PFFs. The total intensity was plotted against the probe concentration and fit to a Kd of 1.45±0.43 μM (FIG. 56F), a reasonably high affinity meriting its further investigation as an αS imaging probe.

### Probe Selectivity for αS Fibrils

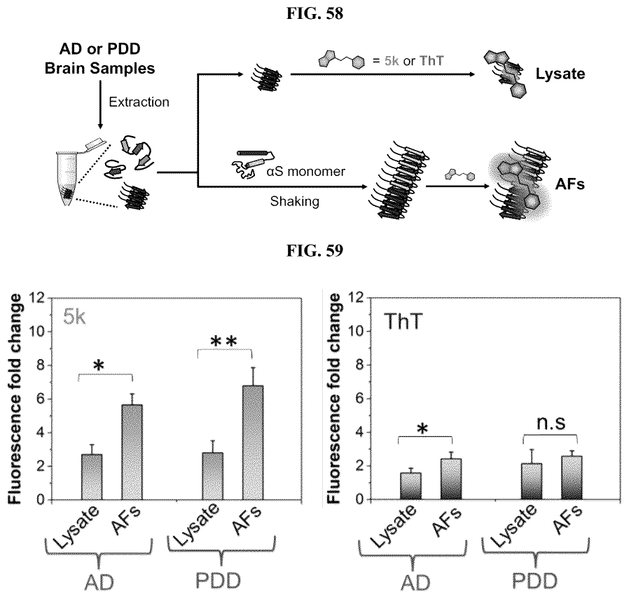

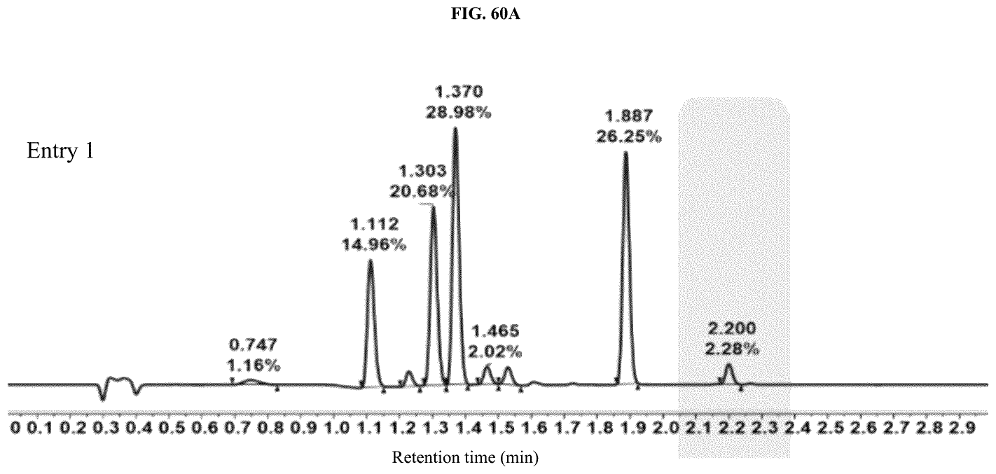

To be useful for detecting αS fibrils in the study of PD and AD, a probe must detect αS amyloid forms while minimizing nonspecific binding in complex biological environments. The dose dependent increase in the fluorescence intensity of 5k occurs only with αS fibrils, not monomers (FIG. 58B). Interestingly, upon binding of 5k with αS fibrils, immediate precipitation occurred, enabling the separation and isolation of fibrils not only from buffer solutions but also from unincorporated monomer through simple centrifugation. This is confirmed by SDS-PAGE gel analysis, suggesting that probe 5k selectively binds to the fibrils and could be used to isolate fibrils (FIG. 58C). A technique known as amyloid precipitation to isolate protein aggregates can be used to verify isolate protein aggregates. This method involves an amyloid-binding ligand attached to a biotin moiety, facilitating surface immobilization through streptavidin-coated magnetic beads. It was anticipated anticipate that 5k could be used in a similar fashion.

To assess the probe's potential for imaging in biological samples, the ability of 5k to detect varying PFF concentrations was measured in the presence of cytosolic human embryonic kidney (HEK) cell lysate (10 mg/mL total protein). Similar results were observed to the binding studies in buffer, showing the ability to detect protein in the low M range (FIG. 58D). Since the low backgrounds in cell lysates indicated minimal off-target binding to soluble proteins, to determine whether our probe was selective towards αS PFFs versus fibrils of tau and the 42 amino acid amyloid-β variant (Aβ1-42), which are typically observed in AD patient brains. The ability to distinguish αS from tau and Aβ1-42 is important to understanding the overlapping pathology of AD, PD, and other related neurodegenerative diseases. 5k was mixed with αS, tau, and Aβ1-42 fibrils and recorded the fluorescence intensity. As one can see from FIG. 58E, at 10 M fibril concentrations, 5k fluorescence turn-on when binding to αS is 2.8-fold higher than tau and 5.2-fold higher than Aβ1-42. The emission maximum is blue shifted for 5k bound to αS (584 nm) vs. tau (596 nm), implying that the dye is in a different environment. So, although 5k binds to both αS and tau, it has a greater fluorescence activation based on the specific mechanism of binding to αS. This is in contrast to ThT, which has high turn-on with both αS and tau (FIG. 58E). Thus, our probe shows much more conformational selectivity than ThT.

### Detecting αS Fibrils in Clinical Samples

Given the selectivity of 5k for αS fibrils over monomer and fibrils of tau and Aβ1-42, as well as its low background fluorescence in cell lysate, to determine whether the probe could be used to detect of αS fibrils in clinical samples. There has been great recent excitement about a fibril amplification assay for use as a PD biomarker. However, this enzyme-linked assay is somewhat operationally complex and there are questions about its response to different αS fibril conformations, or “strains.” In recent years, it has become clear from structural and biochemical studies that different αS fibril polymorphs are present in different diseases, and that these also differ from those formed in vitro. It has been shown that fibril strains from Lewy bodies in PD and related synucleinopathies such as PDD can be faithfully amplified in vitro using seeds derived from patient material and recombinant αS monomer. However, monitoring these reactions has been challenging because ThT does not effectively detect these amplified fibrils (AFs), as one can see in FIG. 58. AD often features significant αS co-pathology with tau, leading to background fluorescence from ThT that should be less of an issue for 5k given its greater selectivity for αS. Therefore, it was thought that an assay using 5k could have significant value if it were selective, as implied by our in vitro PFF studies.

To determine whether our fluorescent probe could detect AFs, and potentially distinguish between fibrils strains from different sources, prepared lysates from three AD cases and three PDD cases were used. For a portion of each lysate, fibrillar material was amplified with αS monomer. The fluorescence of 5k was compared in the presence of the AFs, or in the lysates alone (FIG. 58). It was found that while there was some fluorescence from endogenous fibrillar material in the brain lysate samples, there was a clear increase in fluorescence upon fibril amplification. Interestingly, probe 5k showed higher sensitivity to PDD derived AFs (significance of p<0.005) than AFs of AD (significance of p<0.05). In contrast, ThT is unable to differentiate the lysates and AFs of PDD samples and barely registers significance for detection of AFs in AD samples. While further optimization is necessary, these data show that 5k has the potential to be used in a clinical biomarker assay that is specific for αS in spite of abundant co-pathology of tau and amyloid-O in AD samples.

### Comments

Probes with EDGs (5j and 5k) showcase applications as “turn on” fluorescent probes for the selective binding to the α-synuclein (αS) protein that aggregates to form amyloid fibrils in PD and related neurodegenerative disorders. Furthermore, 5k demonstrates selective binding to αS fibrils over 1) αS monomers, 2) cellular proteins in lysates, and 3) amyloid fibrils of other proteins such as tau and amyloid-0. Finally, the diagnostic potential of 5k was exemplified by selectively detecting polymorphs or “strains” of αS fibrils from PD with dementia (PDD) patient samples.

In the studies herein it has been demonstrated advances in design and synthesis of the underexplored bimane fluorescent scaffold enabling a rational approach to creating novel styryl bimane dyes. Bimane scaffold 1 was synthesized on a multi-gram scale. Key precursor 3 can be utilized to access styryl bimane analogues through the H-W-E reaction with diverse aryl aldehydes. Detailed investigations of a set of styryl bimane probes with varied electronic properties showed tunable absorption and emission in the visible region with large (>200 nm) Stokes shifts. With this library, insights into the structure-photophysical relationship of parasubstituted derivatives by Hammett and DFT analysis were gained to design future bimane probes with predictable photophysical properties. Interestingly, 5j and 5k with electron-donating groups show rotor effects with sensitivity to both polarity and viscosity through ICT and TICT mechanisms, respectively. The characteristics of 5j and 5k enable their application as turn-on fluorescent probes for detecting fibrillar aggregates of the αS protein that are a hallmark of PD. In particular, probe 5k demonstrated selective binding to αS PFFs in three key aspects: 1) over monomers and enabling a method to isolate αS fibrils, 2) in cell lysates with minimal off-target binding, and 3) over other protein fibrils such as tau and amyloid-0. Excitingly, it shows the ability to detect αS AFs from PDD with higher sensitivity than AD, so it can be used in a clinical diagnostic with a simple operational workflow.

In certain non-limiting embodiments, the restriction of rotation that leads to fluorescence turn-on when binding amyloids can also be exploited in the solid state. Indeed, preliminary characterization of amorphous forms of styryl bimanes indicates that they can be used in organic materials (FIG. 54D).

### Enumerated Embodiments

The following exemplary embodiments are provided, the numbering of which is not to be construed as designating levels of importance:

Embodiment 1 provides a compound of Formula (I), or a salt, solvate, stereoisomer, or isotopologue thereof:

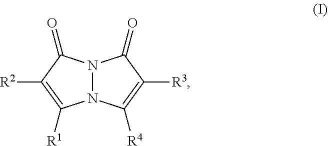

wherein:
R¹, R², and R³ are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{A}, N(R^{A})(R^{B}), SR^{A}, C(═O)R^{A}, C(═O)OR^{A}, C(═O)N(R^{A})(R^{B}), OC(═O)R^{A}, OC(═O)OR^{A}, OC(═O)N(R^{A})(R^{B}), NC(═O)R^{A}, NC(═O)OR^{A}, NC(═O)N(R^{A})(R^{B}), S(═O)R^{A}, S(═O)OR^{A}, S(═O)N(R^{A})(R^{B}), S(═O)₂R^{A}, S(═O)₂OR^{A}, and S(═O)₂N(R^{A})(R^{B});
R⁴ is

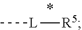

L is selected from the group consisting of optionally substituted C₂-C₆ alkenylenyl and optionally substituted C₂-C₆ alkynylenyl;
R⁵ is selected from the group consisting of optionally substituted C₆-C₁₀ aryl and optionally substituted C₂-C₁₀ heteroaryl;
* indicates a bond between L and R⁵; and
R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

Embodiment 2 provides the compound of Embodiment 1, wherein at least one of the following applies:
(a) at least one of R¹, R², and R³ is C₁-C₆ alkyl;
(b) at least two of R¹, R², and R³ are independently C₁-C₆ alkyl; and
(c) each of R¹, R², and R³ are independently C₁-C₆ alkyl.

Embodiment 3 provides the compound of Embodiment 1 or 2, wherein at least one of the following applies:
(a) at least one of R¹, R², and R³ is CH₃;
(b) at least two of R¹, R², and R³ are CH₃; and
(c) each of R¹, R², and R³ are CH₃.

Embodiment 4 provides the compound of any one of Embodiments 1-3, wherein L is selected from the group consisting of:

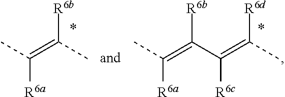

wherein each occurrence of R^{6a}, R^{6b}, R^{6c}, and R^{6d}, if present, is independently selected from the group consisting of H and optionally substituted C₁-C₆ alkyl.

Embodiment 5 provides the compound of any one of Embodiments 1-4, wherein L is selected from the group consisting of

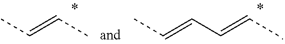

Embodiment 6 provides the compound of any one of Embodiments 1-5, wherein R⁵ is selected from the group consisting of:

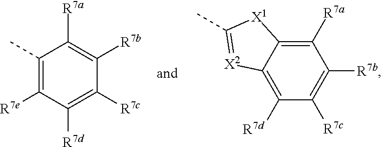

wherein:
X¹, if present, is selected from the group consisting of S, O, and NR^{E};
X², if present, is selected from the group consisting of N and CR^{7e};
R^{7a}, R^{7b}, R^{7c}, R^{7d}, and R^{7e}, if present, are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{C}, N(R^{C})(R^{D}), SR^{C}, C(═O)R^{C}, C(═O)OR^{C}, C(═O)N(R^{C})(R^{D}), OC(═O)R^{C}, OC(═O)OR^{C}, OC(═O)N(R^{C})(R^{D}), NC(═O)R^{C}, NC(═O)OR^{C}, NC(═O)N(R^{C})(R^{D}), S(═O)R^{C}, S(═O)OR^{C}, S(═O)N(R^{C})(R^{D}), S(═O)₂R^{C}, S(═O)₂OR^{C}, and S(═O)₂N(R^{C})(R^{D}); and
R^{C}, R^{D}, and R^{E} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

Embodiment 7 provides the compound of Embodiment 6, wherein each occurrence of R^{7a}, R^{7b}, R^{7c}, R^{7d}, and R^{7e} is independently selected from the group consisting of H, F, NO₂, CN, N₃, CH₃, OH, OCH₃, NH₂, N(CH₃)₂, morpholinyl, and NHC(═O)OC(CH₃)₃.

Embodiment 8 provides the compound of Embodiment 6 or 7, wherein X¹ is S.

Embodiment 9 provides the compound of any one of Embodiments 6-8, wherein X² is N.

Embodiment 10 provides the compound of any one of Embodiments 1-9, wherein R⁵ is selected from the group consisting of

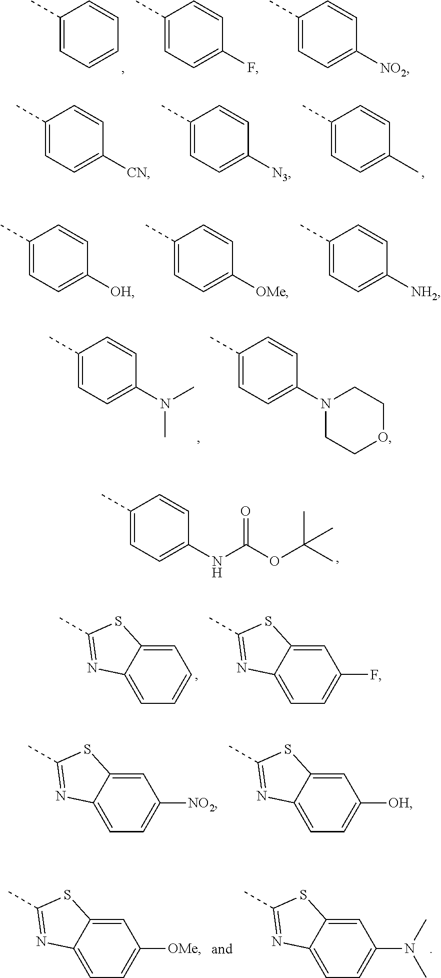

Embodiment 11 provides the compound of any one of Embodiments 1-10, wherein R⁴ is selected from the group consisting of:

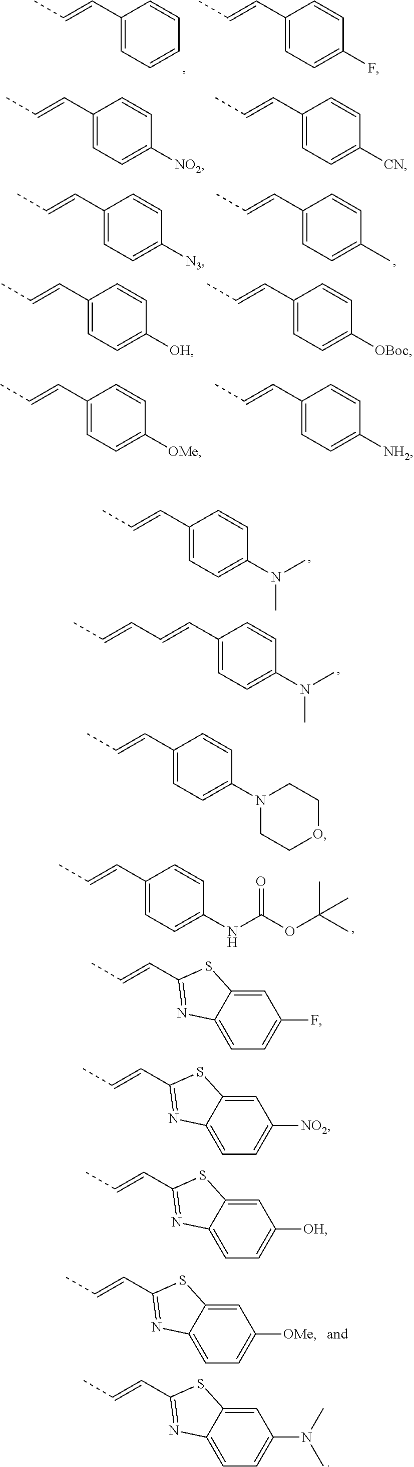

Embodiment 12 provides the compound of any one of Embodiments 1-11, wherein each occurrence of optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₂-C₆ alkenylenyl, optionally substituted C₂-C₆ alkynylenyl, optionally substituted aryl, and optionally substituted heteroaryl is independently optionally substituted with at least one selected from the group consisting of C₁-C₆ alkyl, C₃-C₈ cycloalkyl, C₂-C₁₂ heterocycloalkyl, C₁-C₆ hydroxyalkyl, halogen, CN, N₃, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

Embodiment 13 provides the compound of any one of Embodiments 1-12, which is selected from the group consisting of:
(E)-2,3,6-trimethyl-5-(4-nitrostyryl)-1H,7H-pyrazolo[1,2-a]pyrazole-1,7-dione;
(E)-4-(2-(2,5,6-trimethyl-1,7-dioxo-1H,7H-pyrazolo[1,2-a]pyrazol-3-yl)vinyl)benzonitrile;
(E)-3-(4-fluorostyryl)-2,5,6-trimethyl-1H,7H-pyrazolo[1,2-a]pyrazole-1,7-dione;
(E)-3-(4-azidostyryl)-2,5,6-trimethyl-1H,7H-pyrazolo[1,2-a]pyrazole-1,7-dione;
(E)-2,3,6-trimethyl-5-styryl-1H,7H-pyrazolo[1,2-a]pyrazole-1,7-dione;
(E)-2,3,6-trimethyl-5-(4-methylstyryl)-1H,7H-pyrazolo[1,2-a]pyrazole-1,7-dione;
(E)-3-(4-methoxystyryl)-2,5,6-trimethyl-1H,7H-pyrazolo[1,2-a]pyrazole-1,7-dione;
(E)-3-(4-hydroxystyryl)-2,5,6-trimethyl-1H,7H-pyrazolo[1,2-a]pyrazole-1,7-dione; tert-butyl (E)-(4-(2-(2,5,6-trimethyl-1,7-dioxo-1H,7H-pyrazolo[1,2-a]pyrazol-3-yl)vinyl)phenyl)carbamate;
(E)-3-(4-aminostyryl)-2,5,6-trimethyl-1H,7H-pyrazolo[1,2-a]pyrazole-1,7-dione;
(E)-2,3,6-trimethyl-5-(4-morpholinostyryl)-1H,7H-pyrazolo[1,2-a]pyrazole-1,7-dione;
(E)-3-(4-(dimethylamino)styryl)-2,5,6-trimethyl-1H,7H-pyrazolo[1,2-a]pyrazole-1,7-dione;
3-((1E,3E)-4-(4-(dimethylamino)phenyl)buta-1,3-dien-1-yl)-2,5,6-trimethyl-1H,7H-pyrazolo[1,2-a]pyrazole-1,7-dione;
(E)-2,3,6-trimethyl-5-(2-(6-nitrobenzo[d]thiazol-2-yl)vinyl)-1H,7H-pyrazolo[1,2-a]pyrazole-1,7-dione;
(E)-3-(2-(6-fluorobenzo[d]thiazol-2-yl)vinyl)-2,5,6-trimethyl-1H,7H-pyrazolo[1,2-a]pyrazole-1,7-dione;
(E)-3-(2-(benzo[d]thiazol-2-yl)vinyl)-2,5,6-trimethyl-1H,7H-pyrazolo[1,2-a]pyrazole-1,7-dione;
(E)-3-(2-(6-methoxybenzo[d]thiazol-2-yl)vinyl)-2,5,6-trimethyl-1H,7H-pyrazolo[1,2-a]pyrazole-1,7-dione;
(E)-3-(2-(6-hydroxybenzo[d]thiazol-2-yl)vinyl)-2,5,6-trimethyl-1H,7H-pyrazolo[1,2-a]pyrazole-1,7-dione; and
(E)-3-(2-(6-(dimethylamino)benzo[d]thiazol-2-yl)vinyl)-2,5,6-trimethyl-1H,7H-pyrazolo[1,2-a]pyrazole-1,7-dione.

Embodiment 14 provides a method for identifying a protein aggregation disease in a subject, the method comprising:
(a) contacting a sample obtained from a subject with at least one compound of any one of Embodiments 1-13 to prepare a mixture; and
(b) detecting whether a fluorescence signal is present in the mixture;
   wherein, if a fluorescent signal is present in the mixture, the subject is identified as having a protein aggregation disease.

Embodiment 15 provides the method of Embodiment 14, wherein the protein aggregation disease is a neurodegenerative disease.

Embodiment 16 provides the method of Embodiment 14 or 15, wherein the detecting comprises:
(a) measuring fluorescence of the mixture to obtain a sample fluorescence value;
(b) measuring fluorescence of a first control sample, which lacks a biomarker of interest, to obtain a first control fluorescence value, optionally wherein the first control sample comprises one or more protein aggregates which are distinct from the biomarker of interest; and
(c) comparing the sample fluorescence value and the first control fluorescence value, wherein a sample fluorescence value greater than the first control fluorescence value is indicative of the presence of a protein aggregate disease in the subject.

Embodiment 17 provides the method of Embodiment 14 or 15, wherein the detecting comprises:
(a) measuring fluorescence of the mixture to obtain a sample fluorescence value;
(b) measuring fluorescence of a first control sample, which lacks a biomarker of interest, to obtain a first control fluorescence value, optionally wherein the first control sample comprises one or more protein aggregates which are distinct from the biomarker of interest;
(c) measuring fluorescence of a second control sample which comprises the biomarker of interest to obtain a second control fluorescence value; and
(d) comparing the sample fluorescence value and the first control fluorescence value, wherein a sample fluorescence value greater than the first control fluorescence value and the second control fluorescence value is indicative of the protein aggregate disease in the subject if the second control fluorescence value is positive.

Embodiment 18 provides the method of Embodiment 16 or 17, wherein the biomarker of interest is α-synuclein (αS).

Embodiment 19 provides the method of Embodiment 18, wherein the α-synuclein (αS) is fibrillar.

Embodiment 20 provides the method of Embodiment 18 or 19, wherein the one or more protein aggregates which are distinct from the biomarker of interest comprise amyloid-beta (Aβ) and/or tau protein, optionally wherein the Aβ is Aβ₁₋₄₂, and optionally wherein the amyloid-beta (Aβ) and/or tau protein is/are fibrillar aggregate(s).

Embodiment 21 provides the method of Embodiment any one of Embodiments 18-20, wherein the first control sample further comprises non-aggregated and/or non-fibrillar α-synuclein (αS) (e.g., monomeric αS).

Embodiment 22 provides the method of any one of Embodiments 18-21, wherein the protein aggregation disease is Parkinson's disease (PD).

Embodiment 23 provides the method of Embodiment 16 or 17, wherein the biomarker of interest is amyloid-beta (Aβ), optionally wherein the Aβ is Aβ₁₋₄₂.

Embodiment 24 provides the method of Embodiment 23, wherein the amyloid-beta (Aβ) is fibrillar.

Embodiment 25 provides the method of Embodiment 23 or 24, wherein the one or more protein aggregates which are distinct from the biomarker of interest comprise α-synuclein (αS) and/or tau protein, optionally wherein the αS and/or tau protein are fibrillar aggregates.

Embodiment 26 provides the method of any one of Embodiments 23-25, wherein the first control sample further comprises non-aggregated and/or non-fibrillar amyloid-beta (Aβ) (e.g., monomeric Aβ).

Embodiment 27 provides the method of any one of Embodiments 23-26, wherein the protein aggregation disease is Alzheimer's disease (AD).

Embodiment 28 provides a method of measuring a concentration of a fibrillar protein aggregate in a sample, the method comprising:
(a) contacting a sample comprising the fibrillar protein aggregate with at least one compound of any one of Embodiments 1-13 to prepare a mixture; and
(b) measuring fluorescence in the mixture to obtain a mixture fluorescence value;
(c) contacting each of two or more control samples with at least one compound of any one of Embodiments 1-13 to prepare two or more control sample mixtures, wherein each control sample comprises a known concentration of the fibrillar protein aggregate of interest;
(d) measuring fluorescence of each of the two or more control samples to obtain two or more control sample fluorescence values;
(e) calculating the concentration of fibrillar protein aggregate in the sample by comparing the mixture fluorescence value to the two or more control sample fluorescence values.

Embodiment 29 provides the method of Embodiment 28, wherein the fibrillar protein aggregate is α-synuclein (αS).

Embodiment 30 provides the method of Embodiment 28, wherein the fibrillar protein aggregate is amyloid-beta (Aβ).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A compound of Formula (I), or a salt, solvate, stereoisomer, or isotopologue thereof: 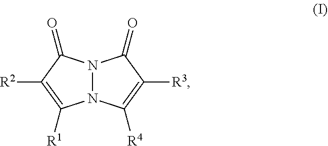
wherein:
R¹, R², and R³ are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{A}, N(R^{A})(R^{B}), SR^{A}, C(═O)R^{A}, C(═O)OR^{A}, C(═O)N(R^{A})(R^{B}), OC(═O)R^{A}, OC(═O)OR^{A}, OC(═O)N(R^{A})(R^{B}), NC(═O)R^{A}, NC(═O)OR^{A}, NC(═O)N(R^{A})(R^{B}), S(═O)R^{A}, S(═O)OR^{A}, S(═O)N(R^{A})(R^{B}), S(═O)₂R^{A}, S(═O)₂OR^{A} and S(═O)₂N(R^{A})(R^{B});
R⁴ is 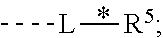
L is selected from the group consisting of optionally substituted C₂-C₆ alkenylenyl and optionally substituted C₂-C₆ alkynylenyl;
R⁵ is selected from the group consisting of optionally substituted C₆-C₁₀ aryl and optionally substituted C₂-C₁₀ heteroaryl;
* indicates a bond between L and R⁵; and
R^{A} and R^{B} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

2. The compound of claim 1, wherein at least one of the following applies:
(a) at least one of R¹, R², and R³ is C₁-C₆ alkyl;
(b) at least two of R¹, R², and R³ are independently C₁-C₆ alkyl; and
(c) each of R¹, R², and R³ are independently C₁-C₆ alkyl.

3. The compound of claim 1, wherein at least one of the following applies:
(a) at least one of R¹, R², and R³ is CH₃;
(b) at least two of R¹, R², and R³ are CH₃; and
(c) each of R¹, R², and R³ are CH₃.

4. The compound of claim 1, wherein at least one of the following applies:
(a) L is selected from the group consisting of: 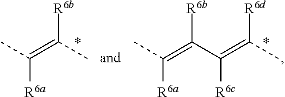
wherein each occurrence of R^{6a}, R^{6b}, R^{6c}, and R^{6d}, if present, is independently selected from the group consisting of H and optionally substituted C₁-C₆ alkyl; and
(b) R⁵ is selected from the group consisting of: 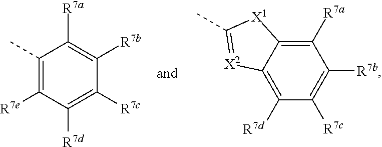
wherein:
X¹, if present, is selected from the group consisting of S, O, and NR^{E};
X², if present, is selected from the group consisting of N and CR^{7e};
R^{7a}, R^{7b}, R^{7c}, R^{7d}, and R^{7e}, if present, are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, N₃, NO₂, OR^{C}, N(R^{C})(R^{D}), SR^{C}, C(═O)R^{C}, C(═O)OR^{C}, C(═O)N(R^{C})(R^{D}), OC(═O)R^{C}, OC(═O)OR^{C}, OC(═O)N(R^{C})(R^{D}), NC(═O)R^{C}, NC(═O)OR^{C}, NC(═O)N(R^{C})(R^{D}), S(═O)R^{C}, S(═O)OR^{C}, S(═O)N(R^{C})(R^{D}), S(═O)₂R^{C}, S(═O)₂OR^{C}, and S(═O)₂N(R^{C})(R^{D}); and
R^{C}, R^{D}, and R^{E} are each independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, and optionally substituted C₂-C₁₀ heteroaryl.

5. The compound of claim 1, wherein L is selected from the group consisting of

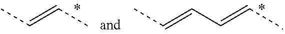

6. The compound of claim 4, wherein at least one of the following applies:
(a) each occurrence of R^{7a}, R^{7b}, R^{7c}, R^{7d}, and R^{7e} is independently selected from the group consisting of H, F, NO₂, CN, N₃, CH₃, OH, OCH₃, NH₂, N(CH₃)₂, morpholinyl, and NHC(═O)OC(CH₃)₃;
(b) X¹ is S;
(c) X² is N.

7. The compound of claim 1, wherein R⁵ is selected from the group consisting of:

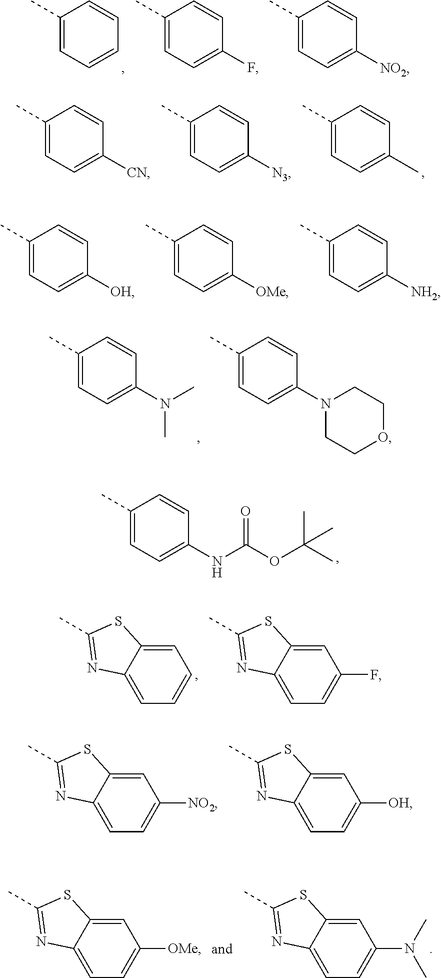

8. The compound of claim 1, wherein R⁴ is selected from the group consisting of:

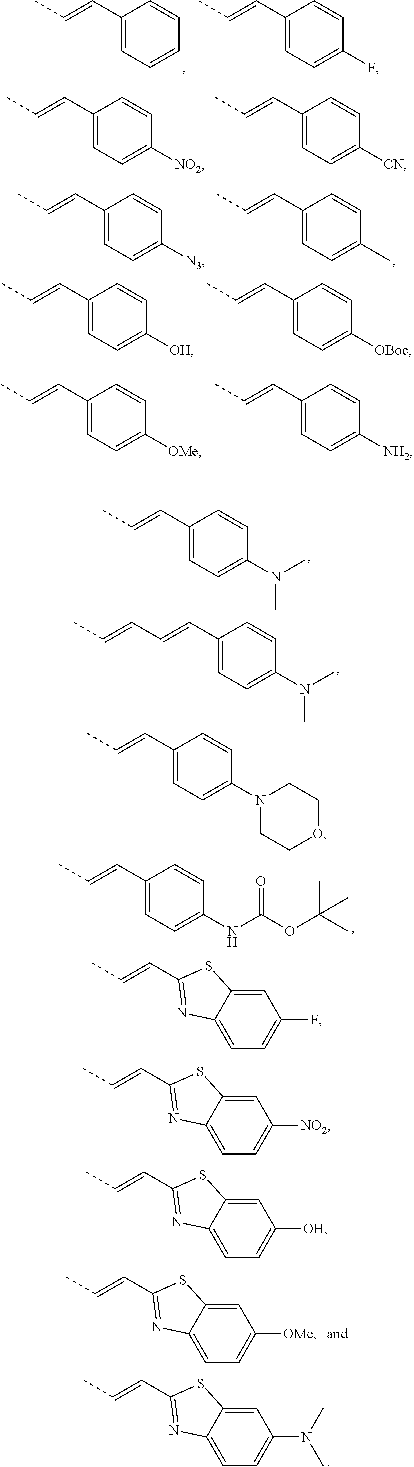

9. The compound of claim 1, wherein each occurrence of optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₂-C₆ alkenylenyl, optionally substituted C₂-C₆ alkynylenyl, optionally substituted aryl, and optionally substituted heteroaryl is independently optionally substituted with at least one selected from the group consisting of C₁-C₆ alkyl, C₃-C₈ cycloalkyl, C₂-C₁₂ heterocycloalkyl, C₁-C₆ hydroxyalkyl, halogen, CN, N₃, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

10. The compound of claim 1, which is selected from the group consisting of:
(E)-2,3,6-trimethyl-5-(4-nitrostyryl)-1H,7H-pyrazolo[1,2-a]pyrazole-1,7-dione;
(E)-4-(2-(2,5,6-trimethyl-1,7-dioxo-1H,7H-pyrazolo[1,2-a]pyrazol-3-yl)vinyl)benzonitrile;
(E)-3-(4-fluorostyryl)-2,5,6-trimethyl-1H,7H-pyrazolo[1,2-a]pyrazole-1,7-dione;
(E)-3-(4-azidostyryl)-2,5,6-trimethyl-1H,7H-pyrazolo[1,2-a]pyrazole-1,7-dione;
(E)-2,3,6-trimethyl-5-styryl-1H,7H-pyrazolo[1,2-a]pyrazole-1,7-dione;
(E)-2,3,6-trimethyl-5-(4-methylstyryl)-1H,7H-pyrazolo[1,2-a]pyrazole-1,7-dione;
(E)-3-(4-methoxystyryl)-2,5,6-trimethyl-1H,7H-pyrazolo[1,2-a]pyrazole-1,7-dione;
(E)-3-(4-hydroxystyryl)-2,5,6-trimethyl-1H,7H-pyrazolo[1,2-a]pyrazole-1,7-dione;
tert-butyl (E)-(4-(2-(2,5,6-trimethyl-1,7-dioxo-1H,7H-pyrazolo[1,2-a]pyrazol-3-yl)vinyl)phenyl)carbamate;
(E)-3-(4-aminostyryl)-2,5,6-trimethyl-1H,7H-pyrazolo[1,2-a]pyrazole-1,7-dione;
(E)-2,3,6-trimethyl-5-(4-morpholinostyryl)-1H,7H-pyrazolo[1,2-a]pyrazole-1,7-dione;
(E)-3-(4-(dimethylamino)styryl)-2,5,6-trimethyl-1H,7H-pyrazolo[1,2-a]pyrazole-1,7-dione;
3-((1E,3E)-4-(4-(dimethylamino)phenyl)buta-1,3-dien-1-yl)-2,5,6-trimethyl-1H,7H-pyrazolo[1,2-a]pyrazole-1,7-dione;
(E)-2,3,6-trimethyl-5-(2-(6-nitrobenzo[d]thiazol-2-yl)vinyl)-1H,7H-pyrazolo[1,2-a]pyrazole-1,7-dione;
(E)-3-(2-(6-fluorobenzo[d]thiazol-2-yl)vinyl)-2,5,6-trimethyl-1H,7H-pyrazolo[1,2-a]pyrazole-1,7-dione;
(E)-3-(2-(benzol[d]thiazol-2-yl)vinyl)-2,5,6-trimethyl-1H,7H-pyrazolo[1,2-a]pyrazole-1,7-dione;
(E)-3-(2-(6-methoxybenzo[d]thiazol-2-yl)vinyl)-2,5,6-trimethyl-1H,7H-pyrazolo[1,2-a]pyrazole-1,7-dione;
(E)-3-(2-(6-hydroxybenzo[d]thiazol-2-yl)vinyl)-2,5,6-trimethyl-1H,7H-pyrazolo[1,2-a]pyrazole-1,7-dione; and
(E)-3-(2-(6-(dimethylamino)benzo[d]thiazol-2-yl)vinyl)-2,5,6-trimethyl-1H,7H-pyrazolo[1,2-a]pyrazole-1,7-dione.

11. A method for identifying a protein aggregation disease in a subject, the method comprising:
(a) contacting a sample obtained from a subject with at least one compound of claim 1 to prepare a mixture; and
(b) detecting whether a fluorescence signal is present in the mixture;
wherein, if a fluorescent signal is present in the mixture, the subject is identified as having a protein aggregation disease,
optionally wherein the protein aggregation disease is a neurodegenerative disease, optionally wherein the neurodegenerative disease is Parkinson's Disease.

12. The method of claim 11, wherein the detecting comprises:
(a) measuring fluorescence of the mixture to obtain a sample fluorescence value;
(b) measuring fluorescence of a first control sample, which lacks a biomarker of interest, to obtain a first control fluorescence value, optionally wherein the first control sample comprises one or more protein aggregates which are distinct from the biomarker of interest; and
(c) comparing the sample fluorescence value and the first control fluorescence value, wherein a sample fluorescence value greater than the first control fluorescence value is indicative of the presence of a protein aggregate disease in the subject.

13. The method of claim 11, wherein the detecting comprises:
(a) measuring fluorescence of the mixture to obtain a sample fluorescence value;
(b) measuring fluorescence of a first control sample, which lacks a biomarker of interest, to obtain a first control fluorescence value, optionally wherein the first control sample comprises one or more protein aggregates which are distinct from the biomarker of interest;
(c) measuring fluorescence of a second control sample which comprises the biomarker of interest to obtain a second control fluorescence value; and
(d) comparing the sample fluorescence value and the first control fluorescence value, wherein a sample fluorescence value greater than the first control fluorescence value and the second control fluorescence value is indicative of the protein aggregate disease in the subject if the second control fluorescence value is positive, optionally wherein the first control sample further comprises non-aggregated or non-fibrillar α-synuclein (αS) (e.g., monomeric αS).

14. The method of claim 12, wherein the biomarker of interest is α-synuclein (αS), optionally wherein the α-synuclein (αS) is fibrillar.

15. The method of claim 14, wherein the one or more protein aggregates which are distinct from the biomarker of interest comprise amyloid-beta (Aβ) or tau protein, optionally wherein the Aβ is Aβ₁₋₄₂, and optionally wherein the amyloid-beta (Aβ) or tau protein is fibrillar aggregate.

16. The method of claim 14, wherein the biomarker of interest is amyloid-beta (Aβ), optionally wherein the Aβ is Aβ₁₋₄₂, and optionally wherein the Aβ is fibrillar.

17. The method of claim 14, wherein the one or more protein aggregates which are distinct from the biomarker of interest comprise α-synuclein (αS) or tau protein, optionally wherein the αS or tau protein is fibrillar aggregate.

18. The method of claim 14, wherein at least one of the following applies:
(a) the first control sample further comprises non-aggregated or non-fibrillar amyloid-beta (Aβ) (e.g., monomeric Aβ); and
(b) the protein aggregation disease is Alzheimer's disease (AD).

19. A method of measuring a concentration of a fibrillar protein aggregate in a sample, the method comprising:
(a) contacting a sample comprising the fibrillar protein aggregate with at least one compound of claim 1 to prepare a mixture; and
(b) measuring fluorescence in the mixture to obtain a mixture fluorescence value;
(c) contacting each of two or more control samples with at least one compound of claim 1 to prepare two or more control sample mixtures, wherein each control sample comprises a known concentration of the fibrillar protein aggregate of interest;
(d) measuring fluorescence of each of the two or more control samples to obtain two or more control sample fluorescence values;
(e) calculating the concentration of fibrillar protein aggregate in the sample by comparing the mixture fluorescence value to the two or more control sample fluorescence values.

20. The method of claim 19, wherein at least one of the following applies:
(a) the fibrillar protein aggregate is α-synuclein (αS)
(b) the fibrillar protein aggregate is amyloid-beta (Aβ).
